SEQUENCE LISTING (176 sequences)

SEQ ID NO: 1 - DNA, 705 bp, Artificial Sequence
  Description of Artificial Sequence Synthetic polynucleotide
atgaggctccctgctcagctcctggggctcctgctgctctggttcccaggttccagatgcgacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - DNA, 60 bp, Artificial Sequence
  Description of Artificial Sequence Synthetic oligonucleotide
atgaggctccctgctcagctcctggggctcctgctgctctggttcccaggttccagatgc

SEQ ID NO: 3 - DNA, 320 bp, Artificial Sequence
  Description of Artificial Sequence Synthetic polynucleotide
gacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaa

SEQ ID NO: 4 - protein, 234 aa, Artificial Sequence
  Description of Artificial Sequence Synthetic polypeptide
MRLPAQLLGLLLLWFPGSRCDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 20 aa, Artificial Sequence
  Description of Artificial Sequence Synthetic peptide
MRLPAQLLGLLLLWFPGSRC

SEQ ID NO: 6 - protein, 107 aa, Artificial Sequence
  Description of Artificial Sequence Synthetic polypeptide
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 7 - DNA, 1416 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 9 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcgtggggggaccgtcagtcttcctcctgcccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcctgaggagcagtacaacagcacgctgcgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctctggtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 10 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 11 - DNA, 1428 bp, Artificial Sequence
  Description of Artificial Sequence Synthetic polynucleotide
atggactggacctggaggatcctcttcttggtggcagcagccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacgccacgtaccgtgtggtcagcgtcctcaccgtcctgcaccaggactggctgaatggcaaggagtacaagtgcaaggtctccaacaaagccctcccagcccccatcgccgctaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 12 - DNA, 57 bp, Artificial Sequence
  Description of Artificial Sequence Synthetic oligonucleotide
atggactggacctggaggatcctcttcttggtggcagcagccacaggagcccactcc

SEQ ID NO: 13 - DNA, 379 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctcag

SEQ ID NO: 14 - protein, 471 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 17 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 475 aa, Artificial Sequence
  Description of Artificial Sequence Synthetic polypeptide
MDWTWRILFLVAAATGAHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 19 aa, Artificial Sequence
  Description of Artificial Sequence Synthetic peptide
MDWTWRILFLVAAATGAHS

SEQ ID NO: 20 - protein, 126 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 21 - protein, 5 aa, Artificial Sequence
  Description of Artificial Sequence Synthetic peptide
  MOD_RES (2)..(2): Any amino acid
LXPTG

SEQ ID NO: 22 - protein, 456 aa, Artificial Sequence
  Description of Artificial Sequence Synthetic polypeptide
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 7 aa, Artificial Sequence
  Description of Artificial Sequence Synthetic peptide
GYTFTYY

SEQ ID NO: 24 - protein, 8 aa, Artificial Sequence
  Description of Artificial Sequence Synthetic peptide
INPDSGGT

SEQ ID NO: 25 - protein, 19 aa, Artificial Sequence
  Description of Artificial Sequence Synthetic peptide
ARDQPLGYCTNGVCSYFDY

SEQ ID NO: 26 - protein, 214 aa, Artificial Sequence
  Description of Artificial Sequence Synthetic polypeptide
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 6 aa, Artificial Sequence
  Description of Artificial Sequence Synthetic peptide
QGIYSW

SEQ ID NO: 28 - protein, 3 aa, Artificial Sequence
  Description of Artificial Sequence Synthetic peptide
TAS

SEQ ID NO: 29 - protein, 9 aa, Artificial Sequence
  Description of Artificial Sequence Synthetic peptide
QQANIFPLT

SEQ ID NO: 30 - protein, 449 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 8 aa, Artificial Sequence
  Description of Artificial Sequence Synthetic peptide
GFTFTDYT

SEQ ID NO: 32 - protein, 8 aa, Artificial Sequence
  Description of Artificial Sequence Synthetic peptide
VNPNSGGS

SEQ ID NO: 33 - protein, 12 aa, Artificial Sequence
  Description of Artificial Sequence Synthetic peptide
ARNLGPSFYFDY

SEQ ID NO: 34 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 6 aa, Artificial Sequence
  Description of Artificial Sequence Synthetic peptide
QDVSIG

SEQ ID NO: 36 - protein, 3 aa, Artificial Sequence
  Description of Artificial Sequence Synthetic peptide
SAS

SEQ ID NO: 37 - protein, 9 aa, Artificial Sequence
  Description of Artificial Sequence Synthetic peptide
QQYYIYPYT

SEQ ID NO: 38 - protein, 444 aa, Artificial Sequence
  Description of Artificial Sequence Synthetic polypeptide
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 8 aa, Artificial Sequence
  Description of Artificial Sequence Synthetic peptide
GYSFTGYY

SEQ ID NO: 40 - protein, 8 aa, Artificial Sequence
  Description of Artificial Sequence Synthetic peptide
VIPNAGGT

SEQ ID NO: 41 - protein, 7 aa, Artificial Sequence
  Description of Artificial Sequence Synthetic peptide
AREGIYW

SEQ ID NO: 42 - protein, 219 aa, Artificial Sequence
  Description of Artificial Sequence Synthetic polypeptide
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 11 aa, Artificial Sequence
  Description of Artificial Sequence Synthetic peptide
QSLVHSNGNTF

SEQ ID NO: 44 - protein, 3 aa, Artificial Sequence
  Description of Artificial Sequence Synthetic peptide
TVS

SEQ ID NO: 45 - protein, 9 aa, Artificial Sequence
  Description of Artificial Sequence Synthetic peptide
SQTTHVPWT

SEQ ID NO: 46 - protein, 448 aa, Artificial Sequence
  Description of Artificial Sequence Synthetic polypeptide
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 10 aa, Artificial Sequence
  Description of Artificial Sequence Synthetic peptide
GGSISSPGYY

SEQ ID NO: 48 - protein, 7 aa, Artificial Sequence
  Description of Artificial Sequence Synthetic peptide
IYKSGST

SEQ ID NO: 49 - protein, 13 aa, Artificial Sequence
  Description of Artificial Sequence Synthetic peptide
TRPVVRYFGWFDP

SEQ ID NO: 50 - protein, 213 aa, Artificial Sequence
  Description of Artificial Sequence Synthetic polypeptide
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 6 aa, Artificial Sequence
  Description of Artificial Sequence Synthetic peptide
QGISSA

SEQ ID NO: 52 - protein, 3 aa, Artificial Sequence
  Description of Artificial Sequence Synthetic peptide
DAS

SEQ ID NO: 53 - protein, 8 aa, Artificial Sequence
  Description of Artificial Sequence Synthetic peptide
QQFNSYPT

SEQ ID NO: 54 - protein, 450 aa, Artificial Sequence
  Description of Artificial Sequence Synthetic polypeptide
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPASKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 8 aa, Artificial Sequence
  Description of Artificial Sequence Synthetic peptide
GFTFSSYG

SEQ ID NO: 56 - protein, 8 aa, Artificial Sequence
  Description of Artificial Sequence Synthetic peptide
ISYEESNR

SEQ ID NO: 57 - protein, 13 aa, Artificial Sequence
  Description of Artificial Sequence Synthetic peptide
ARDGGIAAPGPDY

SEQ ID NO: 58 - protein, 219 aa, Artificial Sequence
  Description of Artificial Sequence Synthetic polypeptide
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 11 aa, Artificial Sequence
  Description of Artificial Sequence Synthetic peptide
QSLLYSNGYNY

SEQ ID NO: 60 - protein, 3 aa, Artificial Sequence
  Description of Artificial Sequence Synthetic peptide
LGS

SEQ ID NO: 61 - protein, 9 aa, Artificial Sequence
  Description of Artificial Sequence Synthetic peptide
MQARQTPFT

SEQ ID NO: 62 - protein, 447 aa, Artificial Sequence
  Description of Artificial Sequence Synthetic polypeptide
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPELLGGPSVFLFPPKPKDTLMISRTPEVTCNAVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 8 aa, Artificial Sequence
  Description of Artificial Sequence Synthetic peptide
GFTFSTYG

SEQ ID NO: 64 - protein, 8 aa, Artificial Sequence
  Description of Artificial Sequence Synthetic peptide
ISGGSSYI

SEQ ID NO: 65 - protein, 12 aa, Artificial Sequence
  Description of Artificial Sequence Synthetic peptide
ARILRGGSGMDL

SEQ ID NO: 66 - protein, 217 aa, Artificial Sequence
  Description of Artificial Sequence Synthetic polypeptide
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 67 - protein, 9 aa, Artificial Sequence
  Description of Artificial Sequence Synthetic peptide
SSNIGAGYN

SEQ ID NO: 68 - protein, 3 aa, Artificial Sequence
  Description of Artificial Sequence Synthetic peptide
GNI

SEQ ID NO: 69 - protein, 11 aa, Artificial Sequence
  Description of Artificial Sequence Synthetic peptide
AAWDKSISGLV

SEQ ID NO: 70 - protein, 450 aa, Artificial Sequence
  Description of Artificial Sequence Synthetic polypeptide
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 8 aa, Artificial Sequence
  Description of Artificial Sequence Synthetic peptide
GFSFSSTY

SEQ ID NO: 72 - protein, 8 aa, Artificial Sequence
  Description of Artificial Sequence Synthetic peptide
IYTGDGTN

SEQ ID NO: 73 - protein, 14 aa, Artificial Sequence
  Description of Artificial Sequence Synthetic peptide
ARPDITYGFAINFW

SEQ ID NO: 74 - protein, 215 aa, Artificial Sequence
  Description of Artificial Sequence Synthetic polypeptide
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 6 aa, Artificial Sequence
  Description of Artificial Sequence Synthetic peptide
QSISSR

SEQ ID NO: 76 - protein, 3 aa, Artificial Sequence
  Description of Artificial Sequence Synthetic peptide
RAS

SEQ ID NO: 77 - protein, 10 aa, Artificial Sequence
  Description of Artificial Sequence Synthetic peptide
QCTGYGISWP

SEQ ID NO: 78 - protein, 452 aa, Artificial Sequence
  Description of Artificial Sequence Synthetic polypeptide
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 8 aa, Artificial Sequence
  Description of Artificial Sequence Synthetic peptide
GYTFTEYI

SEQ ID NO: 80 - protein, 8 aa, Artificial Sequence
  Description of Artificial Sequence Synthetic peptide
IIPNNGGT

SEQ ID NO: 81 - protein, 15 aa, Artificial Sequence
  Description of Artificial Sequence Synthetic peptide
TRREVYGRNYYALDY

SEQ ID NO: 82 - protein, 214 aa, Artificial Sequence
  Description of Artificial Sequence Synthetic polypeptide
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 6 aa, Artificial Sequence
  Description of Artificial Sequence Synthetic peptide
QGINNY

SEQ ID NO: 84 - protein, 3 aa, Artificial Sequence
  Description of Artificial Sequence Synthetic peptide
YTS

SEQ ID NO: 85 - protein, 9 aa, Artificial Sequence
  Description of Artificial Sequence Synthetic peptide
QQYSNLPYT

SEQ ID NO: 86 - DNA, 1415 bp, Artificial Sequence
  Description of Artificial Sequence Synthetic polynucleotide
atggagtttgggctgagctgggttttcctcgttgctcttttaagaggtgtccatgtcaggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcagttatatcaaaggatggaggtaataaataccatgcagactccgtgaagggccgattcaccatctccagagacaattccaagaatgcgctgtatctgcaaatgaatagcctgagagttgaagacacggctgtgtattactgtgtgagaagagggcatcagctggttctgggatactactactacaacggtctggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 87 - protein, 471 aa, Artificial Sequence
  Description of Artificial Sequence Synthetic polypeptide
MEFGLSWVFLVALLRGVQCQVQLVESGGGVVQPGRSLRLSCAASGFTFSSYGMHWVRQAPGKGLEWVAVISKDGGNKYHADSVKGRFTISRDNSKNALYLQMNSLRVEDTAVYYCVRRGHQLVLGYYYYNGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 88 - DNA, 719 bp, Artificial Sequence
  Description of Artificial Sequence Synthetic polynucleotide
atgaggctccctgctcagctcctggggctgctaatgctctgggtctctggatccagtggggatattgtgctgactcagtctccactctccctgcccgtcacccctggagagccggcctccatctcctgcaggtctagtcagagcctcttgtatagtaatggatacaactttttggattggtacctgcagaagccagggcagtctccacagctcctgatctatttgggttctaatcgggcctccggggtccctgacaggttcagtggcagtggatcaggcacagattttacactgaaaatcagcagattggaggctgaggatgttggggtttattactgcatgcaagctctacaaactcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaaggcttcaacaggggagagtgttag

SEQ ID NO: 89 - protein, 239 aa, Artificial Sequence
  Description of Artificial Sequence Synthetic polypeptide
MRLPAQLLGLLMLWVSGSSGDIVLTQSPLSLPVTPGEPASISCRSSQSLLYSNGYNFLDWYLQKPGQSPQLLIYLGSNRASGVPDRFSGSGSGTDFTLKISRLEAEDVGVYYCMQALQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - DNA, 378 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcagttatatcaaaggatggaggtaataaataccatgcagactccgtgaagggccgattcaccatctccagagacaattccaagaatgcgctgtatctgcaaatgaatagcctgagagttgaagacacggctgtgtattactgtgtgagaagagggcatcagctggttctgggatactactactacaacggtctggacgtctggggccaagggaccacggtcaccgtctcctca

SEQ ID NO: 91 - protein, 126 aa, Artificial Sequence
  Description of Artificial Sequence Synthetic polypeptide
QVQLVESGGGVVQPGRSLRLSCAASGFTFSSYGMHWVRQAPGKGLEWVAVISKDGGNKYHADSVKGRFTISRDNSKNALYLQMNSLRVEDTAVYYCVRRGHQLVLGYYYYNGLDVWGQGTTVTVSS

SEQ ID NO: 92 - DNA, 336 bp, Artificial Sequence
  Description of Artificial Sequence Synthetic polynucleotide
gatattgtgctgactcagtctccactctccctgcccgtcacccctggagagccggcctccatctcctgcaggtctagtcagagcctcttgtatagtaatggatacaactttttggattggtacctgcagaagccagggcagtctccacagctcctgatctatttgggttctaatcgggcctccggggtccctgacaggttcagtggcagtggatcaggcacagattttacactgaaaatcagcagattggaggctgaggatgttggggtttattactgcatgcaagctctacaaactcctcggacgttcggccaagggaccaaggtggaaatcaaa

SEQ ID NO: 93 - protein, 112 aa, Artificial Sequence
  Description of Artificial Sequence Synthetic polypeptide
DIVLTQSPLSLPVTPGEPAAISCRSSQSLLYSNGYNFLDWYLQKPGQSPQLLIYLGSNRASGVPDPYSGSGSGTDFTLKISRLEAEDVGVYYCMQALQTPRTFGQGTKVEIK

SEQ ID NO: 94 - DNA, 377 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcagttatatcaaaggatggaggtaataaataccatgcagactccgtgaagggccgattcaccatctccagagacaattccaagaatacgctgttctgcaaatgaatagcctgagagttgaagacacggctgtgtattactgtgtgagaagagggcatcagctggttctgggatactactactacaacggtctggacgtctggggccaagggaccacggtcaccgtctcctca

SEQ ID NO: 95 - protein, 126 aa, Artificial Sequence
  Description of Artificial Sequence Synthetic polypeptide
QVQLVESGGGVVQPGRSLRLSCAASGFTFSSYGMHWVRQAPGKGLEWVAVISKDGGNKYHADSVKGRFTISRDNSKNTLYLQMNSLRVEDTAVYYCVRRGHQLVLGYYYYNGLDVWGQGTTVTVSS

SEQ ID NO: 96 - DNA, 378 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcagttatatcaaaggatggaggtaataaataccatgcagactccgtgaagggccgattcaccatctccagagacaattccaagaatacgctgtatctgcaaatgaatagcctgagagctgaagacacggctgtgtattactgtgcgagaagagggcatcagctggttctgggatactactactacaacggtctggacgtctggggccaagggaccacggtcaccgtctcctca

SEQ ID NO: 97 - protein, 126 aa, Artificial Sequence
  Description of Artificial Sequence Synthetic polypeptide
QVQLVESGGGVVQPGRSLRLSCAASGFTFSSYGMHWVRQAPGKGLEWVAVISKDGGNKYHADSVKGRFTISRDNSKNTLYLQMNSLRAEDTAVYYCARRGHQLVLGYYYYNGLDVWGQGTTVTVSS

SEQ ID NO: 98 - DNA, 336 bp, Artificial Sequence
  Description of Artificial Sequence Synthetic polynucleotide
gatattgtgatgactcagtctccactctccctgcccgtcacccctggagagccggcctccatctcctgcaggtctagtcagagcctcttgtatagtaatggatacaactttttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaa

SEQ ID NO: 99 - protein, 112 aa, Artificial Sequence
  Description of Artificial Sequence Synthetic polypeptide
DIVMTQSPLSLPVTPGEPASISCRSSQSLLYSNGYNFLDWYLQKPGQSPQLLIYLGSNRASGVPDRFSGSGSGTDFTLKISRVEAEDVGVYYCMQALQTPRTFGQGTKVEIK

SEQ ID NO: 100 - DNA, 1425 bp, Artificial Sequence
  Description of Artificial Sequence Synthetic polynucleotide
atggagtttgggctgagctgggttttcctcgttgctcttttaagaggtgtccagtgtcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggagataataaataccatgcagactccgtgtggggccgattcaccatctccagagacaattccaggagcacgctttatctgcaaatgaacagcctgagagctgaggacacggctgtatattactgtgcgagaagaggcatggggtctagtgggagccgtggggattactactactactacggtttggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 101 - protein, 474 aa, Artificial Sequence
  Description of Artificial Sequence Synthetic polypeptide
MEFGLSWVFLVALLRGVQCQVQLVESGGGVVQPGRSLRLSCAASGFTFSSYGMHWVRQAPGKGLEWVAVISNDGDNKYHADSVWGRFTISRDNSRSTLYLQMNSLRAEDTAVYYCARRGMGSSGSRGDYYYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 102 - DNA, 720 bp, Artificial Sequence
  Description of Artificial Sequence Synthetic polynucleotide
atgaggctccctgctcagctcctggggctgctaatgctctgggtctctggatccagtggggatattgtgatgactcagtctccactctccctgcccgtcacccctggagagccggcctccatctcctgcaggtctagtcagagcctcttgtatagtaatggatacaactttttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 103 - protein, 238 aa, Artificial Sequence
  Description of Artificial Sequence Synthetic polypeptide
MRLPAQLLGLLMLWVSGSSGDIVMTQSPLSLPVTPGEPASISCRSSQSLLYSNGYNFLDWYLQKPGQSPQLLIYLGSNRASGVPDRFSGSGSGTDFTKISRVEAEDVGVYYCMQALQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - DNA, 387 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggagataataaataccatgcagactccgtgtggggccgattcaccatctccagagacaattccaggagcacgctttatctgcaaatgaacagcctgagagctgaggacacggctgtatattactgtgcgagaagaggcatggggtctagtgggagccgtggggattactactactactacggtttggacgtctggggccaagggaccacggtcaccgtctcctca

SEQ ID NO: 105 - protein, 129 aa, Artificial Sequence
  Description of Artificial Sequence Synthetic polypeptide
QVQLVESGGGVVQPGRSLRLSCAASGFTFSSYGMHWVRQAPGKGLEWVAVISNDGDNKYHADSVWGRFTISRDNSRSTLYLQMNSLRAEDTAVYYCARRGMGSSGSRGDYYYYYGLDVWGQGTTVTVSS

SEQ ID NO: 106 - DNA, 336 bp, Artificial Sequence; identical to SEQ ID NO: 98
  Description of Artificial Sequence Synthetic polynucleotide

SEQ ID NO: 107 - protein, 112 aa, Artificial Sequence; identical to SEQ ID NO: 99
  Description of Artificial Sequence Synthetic polypeptide

SEQ ID NO: 108 - DNA, 1395 bp, Artificial Sequence
  Description of Artificial Sequence Synthetic polynucleotide
atgaaacacctgtggttcttcctcctgctggtggcagctcccagatgggtcctgtcccaggtgcagctgcaggagtcgggcccaggactggtgaagccttcggagaccctgtccctcacctgcactgtctctggtggctccatcagtagttactactggatctggatccggcagcccgccgggaagggactggaatggattgggcgtgtctataccagtgggagcaccaactacaacccctccctcaagagtcgagtcaccatgtcagtagacacgtccaagaaccagttctccctgaagctgagctctgtgaccgccgcggacacggccgtgtattactgtgcgagagatggtctttacagggggtacggtatggacgtctggggccaagggaccacggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 109 - protein, 464 aa, Artificial Sequence
  Description of Artificial Sequence Synthetic polypeptide
MKHLWFFLLLVAAPRWVLSQVQLQESGPGLVKPSETLSLTCTVSGGSISSYYWIWIRQPAGKGLEWIGRVYTSGSTNYNPSLKSRVTMSVDTSKNQFSLKLSSVTAADTAVYYCARDGLYRGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 110 - DNA, 705 bp, Artificial Sequence
  Description of Artificial Sequence Synthetic polynucleotide
atgaggctccctgctcagctcctggggctcctgctgctctggttcccaggttccagatgcgacatccagatgacccagtctccatcttccgtgtctgcatctgtaggagacagagtcaccatcacttgtcgggcgagtcagcctattagcagctggttagcctggtatcagcagaaaccagggaaagcccctaaactcctgatttattctgcctccggtttgcaaagtggggtcccatcaaggttcagcggcagtggatctgggacagatttcactctcaccatcagcagcctgcagcctgaagattttgcaacttactattgtcaacagactgacagtttcccgctcactttcggcggcgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 111 - protein, 234 aa, Artificial Sequence
  Description of Artificial Sequence Synthetic polypeptide
MRLPAQLLGLLLLWFPGSRCDIQMTQSPSSVSASVGDRVTITCRASQPISSWLAWYQQKPGKAPKLLIYSASGLQSGVPSRFSGSGSGTDFTLTISSLQPEDFATYYCQQTDSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - DNA, 357 bp, Artificial Sequence
  Description of Artificial Sequence Synthetic polynucleotide
caggtgcagctgcaggagtcgggcccaggactggtgaagccttcggagaccctgtccctcacctgcactgtctctggtggctccatcagtagttactactggatctggatccggcagcccgccgggaagggactggaatggattgggcgtgtctataccagtgggagcaccaactacaacccctccctcaagagtcgagtcaccatgtcagtagacacgtccaagaaccagttctccctgaagctgagctctgtgaccgccgcggacacggccgtgtattactgtgcgagagatggtctttacagggggtacggtatggacgtctggggccaagggaccacggtcaccgtctcctca

SEQ ID NO: 113 - protein, 119 aa, Artificial Sequence
  Description of Artificial Sequence Synthetic polypeptide
QVQLQESGPGLVKPSETLSLTCTVSGGSISSYYWIWIRQPAGKGLEWIGRVYTSGSTNYNPSLKSRVTMSVDTSKNQFSLKLSSVTAADTAVYYCARDGLYRGYGMDVWGQGTTVTVSS

SEQ ID NO: 114 - DNA, 321 bp, Artificial Sequence
  Description of Artificial Sequence Synthetic polynucleotide
gacatccagatgacccagtctccatcttccgtgtctgcatctgtaggagacagagtcaccatcacttgtcgggcgagtcagcctattagcagctggttagcctggtatcagcagaaaccagggaaagcccctaaactcctgatttattctgcctccggtttgcaaagtggggtcccatcaaggttcagcggcagtggatctgggacagatttcactctcaccatcagcagcctgcagcctgaagattttgcaacttactattgtcaacagactgacagtttcccgctcactttcggcggcgggaccaaggtggagatcaaa

SEQ ID NO: 115 - protein, 107 aa, Artificial Sequence
  Description of Artificial Sequence Synthetic polypeptide
DIQMTQSPSSVSASVGDRVTITCRASQPISSWLAWYQQKPGKAPKLLIYSASGLQSGVPSRFSGSGSGTDFTLTISSLQPEDFATYYCQQTDSFPLTFGGGTKVEIK

SEQ ID NO: 116 - DNA, 1401 bp, Artificial Sequence
  Description of Artificial Sequence Synthetic polynucleotide
atgaaacatctgtggttcttccttctcctggtggcagctcccagatgggtcctgtcccaggtgcagctgcaggagtcgggcccaggactggtgaagccttcggagaccctgtccctcacctgcactgtctctggtggctccatcagaagttactactggacctggatccggcagcccccagggaagggactggagtggattggatatatctattacagtgggagcaccaactacaatccctccctcaagagtcgagtcaccatatcagtagacatgtccaagaaccagttctccctgaagctgagttctgtgaccgctgcggacacggccgtttattactgtgcgagaaagggtgactacggtggtaattttaactactttcaccagtggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagtcacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 117 - protein, 466 aa, Artificial Sequence
  Description of Artificial Sequence Synthetic polypeptide
MKHLWFFLLLVAAPRWVLSQVQLQESGPGLVKPSETLSLTCTVSGGSIRSYYWTWIRQPPGKGLEWIGYIYYSGSTNYNPSLKSRVTISVDMSKNQFSLKLSSVTAADTAVYYCARKGDYGGNFNYFHQWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 118 - DNA, 720 bp, Artificial Sequence
  Description of Artificial Sequence Synthetic polynucleotide
atgaggctccctgctcagctcctggggctgctaatgctctgggtctctggatccagtggggatattgtgatgactcagtctccactctccctgcccgtcacccctggagagccggcctccatctcctgcaggtctagtcagagcctcctacatactaatggatacaactatttcgattggtacctgcagaagccagggcagtctccacaactcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtacagttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 119 - protein, 239 aa, Artificial Sequence
  Description of Artificial Sequence Synthetic polypeptide
MRLPAQLLGLLMLWVSGSSGDIVMTQSPLSLPVTPGEPASISCRSSQSLLHTNGYNYFDWYLQKPGQSPQLLIYLGSNRASGVPDRFSGSGSGTDFTLKISRVEAEDVGVYYCMQALQTPYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - DNA, 363 bp, Artificial Sequence
  Description of Artificial Sequence Synthetic polynucleotide
caggtgcagctgcaggagtcgggcccaggactggtgaagccttcggagaccctgtccctcacctgcactgtctctggtggctccatcagaagttactactggacctggatccggcagcccccagggaagggactggagtggattggatatatctattacagtgggagcaccaactacaatccctccctcaagagtcgagtcaccatatcagtagacatgtccaagaaccagttctccctgaagctgagttctgtgaccgctgcggacacggccgtttattactgtgcgagaaagggtgactacggtggtaattttaactactttcaccagtggggccagggaaccctggtcaccgtctcctca

SEQ ID NO: 121 - protein, 121 aa, Artificial Sequence
  Description of Artificial Sequence Synthetic polypeptide
QVQLQESGPGLVKPSETLSLTCTVSGGSIRSYYWTWIRQPPGKGLEWIGYIYYSGSTNYNPSLKSRVTISVDMSKNQFSLKLSSVTAADTAVYYCARKGDYGGNFNYFHQWGQGTLVTVSS

SEQ ID NO: 122 - DNA, 336 bp, Artificial Sequence
  Description of Artificial Sequence Synthetic polynucleotide
gatattgtgatgactcagtctccactctccctgcccgtcacccctggagagccggcctccatctcctgcaggtctagtcagagcctcctacatactaatggatacaactatttcgattggtacctgcagaagccagggcagtctccacaactcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtacagttttggccaggggaccaagctggagatcaaa

SEQ ID NO: 123 - protein, 112 aa, Artificial Sequence
  Description of Artificial Sequence Synthetic polypeptide
DIVMTQSPLSLPVTPGEPASISCRSSQSLLHTNGYNYFDWYLQKPGQSPQLLIYLGSNRASGVPDRFSGSGSGTDFTLKISRVEAEDVGVYYCMQALQTPYSFGQGTKLEIK

SEQ ID NO: 124 - DNA, 1416 bp, Artificial Sequence; identical to SEQ ID NO: 7
  Description of Artificial Sequence Synthetic polynucleotide

SEQ ID NO: 125 - protein, 471 aa, Artificial Sequence; identical to SEQ ID NO: 14
  Description of Artificial Sequence Synthetic polypeptide

SEQ ID NO: 126 - DNA, 705 bp, Artificial Sequence; identical to SEQ ID NO: 1
  Description of Artificial Sequence Synthetic polynucleotide

SEQ ID NO: 127 - protein, 234 aa, Artificial Sequence; identical to SEQ ID NO: 4
  Description of Artificial Sequence Synthetic polypeptide

SEQ ID NO: 128 - DNA, 377 bp, Artificial Sequence
  Description of Artificial Sequence Synthetic polynucleotide
aggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctgacagtggtggcacaaactatgcacagaagtttcagggcagggtcaccatgaccagggacacgtccatcagcacagcctacatggagctgaacaggctgagatctgacgacacggccgtgtattactgtgcgagagatcagcccctaggatattgtactaatggtgtatgctcctactttgactactggggccagggaaccctggtcaccgtctcctca

SEQ ID NO: 129 - protein, 126 aa, Artificial Sequence; identical to SEQ ID NO: 20
  Description of Artificial Sequence Synthetic polypeptide

SEQ ID NO: 130 - DNA, 321 bp, Artificial Sequence
  Description of Artificial Sequence Synthetic polynucleotide
gacatccagatgacccagtctccatcttccgtgtctgcatctgtaggagacagagtcaccatcacttgtcgggcgagtcagggtatttacagctggttagcctggtatcagcagaaaccagggaaagcccctaacctcctgatctatactgcatccactttacaaagtggggtcccatcaaggttcagcggcagtggatctgggacagatttcactctcaccatcagcagcctgcaacctgaagattttgcaacttactattgtcaacaggctaacattttcccgctcactttcggcggagggaccaaggtggagatcaaa

SEQ ID NO: 131 - protein, 107 aa, Artificial Sequence; identical to SEQ ID NO: 6
  Description of Artificial Sequence Synthetic polypeptide

SEQ ID NO: 132 - DNA, 361 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagctatgtcatgcactgggtccgccaggctccaggcaaggggctggagtgggtggcagttatgtcatatgatggaagtagtaaatactatgcaaactccgtgaagggccgattcaccatctccagagacaattccaagaacacgctgtatctgcaaataaacagcctgagagctgaggacacggctgtgtattactgtgcgagagatgggggtaaagcagtgcctggtcctgactactggggccagggaatcctggtcaccgtctcctcag

SEQ ID NO: 133 - protein, 120 aa, Artificial Sequence
  Description of Artificial Sequence Synthetic polypeptide
QVQLVESGGGVVQPGRSLRLSCAASGFTFSSYVMHWVRQAPGKGLEWVAVMSYDGSSKYYANSVKGRFTISRDNSKNTLYLQINSLRAEDTAVYYCARDGGKAVPGPDYWGQGILVTVSS

SEQ ID NO: 134 - DNA, 337 bp, Artificial Sequence
  Description of Artificial Sequence Synthetic polynucleotide
gatattgtgatgactcagtctccactctccctgcccgtcacccctggagagccggcctccatctcctgcaggtctagtcagagtgttctgtatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagttttacaaactccattcactttcggccctgggaccaaagtggatatcaaac

SEQ ID NO: 135 - protein, 112 aa, Artificial Sequence
  Description of Artificial Sequence Synthetic polypeptide
DIVMTQSPLSLPVTPGEPASISCRSSQSVLYSNGYNYLDWYLQKPGQSPQLLIYLGSNRASGVPDRFSGSGSGTDFTLKISRVEAEDVGVYYCMQVLQTPFTFGPGTKVDIK

SEQ ID NO: 136 - DNA, 373 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtcgctatggcatgcactgggtccgccaggctccaggcaaggggctggagtgggtggcagttatatcatctgatggaggtaataaatactatgcagactccgtgaagggccgattcaccatctccagagacaattccaagaacacgctgtatctgcaaatgaacagcctgagagctgaggacacggctgtgtattactgtacgagaagagggactggaaagacttactaccactactgtggtatggacgtctggggccaagggaccacggtcaccgtctcctcag

SEQ ID NO: 137 - protein, 124 aa, Artificial Sequence
  Description of Artificial Sequence Synthetic polypeptide
QVQLVESGGGVVQPGRSLRLSCAASGFTFSRYGMHWVRQAPGKGLEWVAVISSDGGNKYYADSVKGRFTISRDNSKNTLYLQMNSLRAEDTAVYYCTRRGTGKTYYHYCGMDVWGQGTTVTVSS

SEQ ID NO: 138 - DNA, 337 bp, Artificial Sequence
  Description of Artificial Sequence Synthetic polynucleotide
gatattgtgatgactcagtctccactctccctgcccgtcacccctggagagccggcctccatctcctgcaggtctagtcagagcctcctgtatagtaatggatataactatttggattggtacctgcagaagccagggcagtctccacacctcctgatctatttgggttctaatcgggcctccggggtccctgacaggttcagtggcagtggttcaggcactgattttacactgaaaatcagcagagtggaggctgaggatgttggggtttattactgcatgcaagctctacaaactcctcggacgttcggccaagggaccaaggtggaaatcaaac

SEQ ID NO: 139 - protein, 112 aa, Artificial Sequence
  Description of Artificial Sequence Synthetic polypeptide
DIVMTQSPLSLPVTPGEPASISCRSSQSLLYSNGYNYLDWYLQKPGQSPHLLIYLGSNRASGVPDRFSGSGSGTDFTLKISRVEAEDVGVYYCMQALQTPRTFGQGTKVEIK

SEQ ID NO: 140 - DNA, 373 bp, Artificial Sequence
  Description of Artificial Sequence Synthetic polynucleotide
caggtgcagctggtggagtctgggggaggcgtccagcctgggaggtccctgagactctcctgtgtagcctctggattcaccttcagtaactatggcatgcactgggtccgccaggctccaggcaaggggctggagtgggtggcaattatatcatatgatggaagtaataaatactatgcagactccgtgaagggccgattcaccatctccagagacaattccaagaacacgctgtatgtgcaaatgaacagcctgagagctgaggacacggctgtgtattactgtgcgagacgcggtcactacgggagggattactactcctactacggtttggacgtctggggccaagggaccacggtcaccgtctcctcag

SEQ ID NO: 141 - protein, 125 aa, Artificial Sequence
  Description of Artificial Sequence Synthetic polypeptide
QVQLVESGGGVVQPGRSLRLSCVASGFTFSNYGMHWVRQAPGKGLEWVAIISYDGSNKYYADSVKGRFTISRDNSKNTLYVQMNSLRAEDTAVYYCARRGHYGRDYYSYYGLDVWGQGTTVTVSS

SEQ ID NO: 142 - DNA, 337 bp, Artificial Sequence
  Description of Artificial Sequence Synthetic polynucleotide
gatattgtgatgactcagtctccactctccctgcccgtcacccctggagagccggcctccatctcctgcaggtctagtcagagcctcctgcctggtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacgttcggccaagggaccaaggtggaaatcaaac

SEQ ID NO: 143 - protein, 112 aa, Artificial Sequence
  Description of Artificial Sequence Synthetic polypeptide
DIVMTQSPLSLPVTPGEPASISCRSSQSLLPGNGYNYLDWYLQKPGQSPQLLIYLGSNRASGVPDRFSGSGSGTDFTLKISRVEAEDVGVYYCMQALQTPRTFGQGTKVEIK

SEQ ID NO: 144 - DNA, 364 bp, Artificial Sequence
  Description of Artificial Sequence Synthetic polynucleotide
caggtgcagctgcaggagtcgggcccaggactggtgaagccttcggacaccctgtccctcacctgcactgtctctggtggctccatcagaggttactactggagctggatccggcagccccctgggaagggactggagtggattgggtatatctattacagtgggagcaccaactacaacccctccctcaagagtcgagtcaccatatcagtagacacgtccaagaaccagttctccctgaagctgaactctgtgaccgctgcggacacggccgtgtattattgtgcgagaaaggggggcctctacggtgactacggctggttcgccccctggggccagggaaccctggtcaccgtctcctcag

SEQ ID NO: 145 - protein, 122 aa, Artificial Sequence
  Description of Artificial Sequence Synthetic polypeptide
QVQLQESGPGLVKPSDTLSLTCTVSGGSIRGYYWSWIIRQPPGKGLEWIGYIYYSGSTNYNPSLKSRVTISVDTSKNQFSLKLNSVTAADTAVYYCARKGGLYGDYGWFAPWGQGTLVTVSS

SEQ ID NO: 146 - DNA, 322 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcagcgacttagcctggcaccagcagaaacctggccaggctcccagactcctcatctatggtgcatccagcagggccactggcatcccagacaggttcagtggcagtgggtctgggacagacttcactctcaccatcagcagactggagcctgaagattttgcagtgtattactgtcagcactgtcgtagcttattcactttcggccctgggaccaaagtggatatcaaac

SEQ ID NO: 147 - protein, 107 aa, Artificial Sequence
  Description of Artificial Sequence Synthetic polypeptide
EIVLTQSPGTLSLSPGERATLSCRASQSVSSSDLAWHQQKPGQAPRLLIYGASSRATGIPDRFSGSGSGTDFTLTISRLEPEDFAVYYCQHCRSLFTFGPGTKVDIK

SEQ ID NO: 148 - DNA, 364 bp, Artificial Sequence
  Description of Artificial Sequence Synthetic polynucleotide
caggtgcagctgcaggagtcgggcccaggactggtgaagccttcggagaccctgtccctcacctgcactgtctctggtggctccatcagaggttactactggagctggatccggcagccccctgggaagggactggagtggattgggtatatctattacagtgggagcaccaactacaacccctccctcaagagtcgagtcaccatatcagtagacacgtccaagaaccagttctccctgaagctgaactctgtgaccgctgcggacacggccgtgtattattgtgcgagaaaggggggcctctacggtgactacggctggttcgccccctggggccagggaaccctggtcaccgtctcctcag

SEQ ID NO: 149 - protein, 121 aa, Artificial Sequence
  Description of Artificial Sequence Synthetic polypeptide
QVQLQESGPGLVKPSLTLSLTCTVSGGSIRGYYWSWIRQPPGKGLEWIGYIYYSGSTNYNPSLKSRVTISVDTSKNQFSLKLNSVTAADTAVYYCARKGGLYGDYGWFAPWGQGTLVTVSS

SEQ ID NO: 150 - DNA, 376 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagctatgccatgcactgggtccgccaggctccaggcaaggggctggagtgggtggcagttatatcatatgatggaagtaataaatactatgcagactccgtgaagggccgattcaccatctacagagacaattccaagaacacgctgtatctgcaaatgaacagcctgagagctgaggacacggctgtgtattactgtgcgagacgcggtcactacgggaataattactactcctattacggtttggacgtctggggccaagggaccacggtcaccgtctcctcag

SEQ ID NO: 151 - protein, 125 aa, Artificial Sequence
  Description of Artificial Sequence Synthetic polypeptide
QVQLVESGGGVVQPGRSLRLSCAASGFTFSSYAMHWVRQAPGKGLEWVAVISYDGSNKYYADSVKGRFTIYRDNSKNTLYLQMNSLRAEDTAVYYCARRGHYGNNYYSYYGLDVWGQGTTVTVSS

SEQ ID NO: 152 - DNA, 336 bp, Artificial Sequence
  Description of Artificial Sequence Synthetic polynucleotide
gatattgtgatgactcagtctccactctccctgcccgtcacccctggagagccggcctccatctcctgcaggtctagtcagagcctcctgcctggtaatggatacaactatttggattggtacctgcagaagccagggcagtctccacagctcctgatctatttgggttctaatcgggcctccggggtccctgcaggttcagtggcagtggctcaggcacagattttacactgaaaatcagcagagtggaggctgaggatgttgggatttattactgcatgcaagctctacaaactcctcggacgttcggccaagggaccaaggtggaaatcaaac

SEQ ID NO: 153 - protein, 112 aa, Artificial Sequence
  Description of Artificial Sequence Synthetic polypeptide
DIVMTQSPLSLPVTPGEPASISCRSSQSLLPGNGYNYLDWYLQKPGQSPQLLIYLGSNRASGVPDRFSGSGSGTDFTLKISRVEAEDVGIYYCMQALQTPRTFGQGTKVEIK

SEQ ID NO: 154 - DNA, 364 bp, Artificial Sequence
  Description of Artificial Sequence Synthetic polynucleotide
caggtgcagctgcaggagtcgggcccaggactggtgaagccttcggagaccctgtccctcacctgcactgtctctggtggctccatcagaggttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagttctgtgaccgctgcggacacggccgtgtattactgtgcgagaagggggggcctctacggtgactacggctggttcgccccctggggccagggaaccctggtcaccgtctcctcag

SEQ ID NO: 155 - protein, 121 aa, Artificial Sequence
  Description of Artificial Sequence Synthetic polypeptide
QVQLQESGPGLVKPSETLSLTCTVSGGSIRGYYWSWIRQPPGKGLEWIGYIYYSGSTNYNPSLKSRVTISVDTSKNQFSLKLSSVTAADTAVYYCARRGGLYGDYGWFAPWGQGTLVTVSS

SEQ ID NO: 156 - DNA, 322 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcacctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatagtagcttattcactttcggccctgggaccaaagtggatatcaaac

SEQ ID NO: 157 - protein, 108 aa, Artificial Sequence
  Description of Artificial Sequence Synthetic polypeptide
ETVLTQSPGTLSLSPGERATLSCRASQSVSSTYLAWYQQKPGQAPRLLIYGASSRATGIIPDRFSGSGSGTDFTLTISRLEPEDFAVYYCQQYSSLFTFGPGTKVDIK

SEQ ID NO: 158 - DNA, 1398 bp, Artificial Sequence
  Description of Artificial Sequence Synthetic polynucleotide
atggagtttgggctgagctgggttttcctcgttgctcttttaagaggtgtccagtgtcaggtgcagctggtggagtctgggggaggcgtggtccagcctgggaggtccctgagactctcctgtgcagcctctggattcaccttcagtagctatgtcatgcactgggtccgccaggctccaggcaaggggctggagtgggtggcagttatgtcatatgatggaagtagtaaatactatgcaaactccgtgaagggccgattcaccatctccagagacaattccaagaacacgctgtatctgcaaataaacagcctgagagctgaggacacggctgtgtattactgtgcgagagatgggggtaaagcagtgcctggtcctgactactggggccagggaatcctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 159 - protein, 466 aa, Artificial Sequence
  Description of Artificial Sequence Synthetic polypeptide
MEFGLSWVFLVALLRGVQCQVQLVESGGGVVQPGRSLRLSCAASGFTFSSYVMHWVRQAPGKGLEWVAVMSYDGSSKYYANSVKGRIFTISRDNSKNTLYLQINSLRAEDTAVYYCARDGGKAVPGPDYWGQGILVTVSSASTKGPSVFPLAPCSRSTSESTAALGCLVKDYFPEPVTVSWNSGALTSGVHTFPAVLQSSGLYSLSSVVTVPSSNTGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 160 - DNA, 720 bp, Artificial Sequence
  Description of Artificial Sequence Synthetic polynucleotide
atgaggctccctgctcagctcctggggctgctaatgctctgggtctctggatccagtggggatattgtgatgactcagtctccactctccctgcccgtcacccctggagagccggcctccatctcctgcaggtctagtcagagtgttctgtatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagttttacaaactccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 161 - protein, 238 aa, Artificial Sequence
  Description of Artificial Sequence Synthetic polypeptide
MRLPAQLLGLLMLWVSGSSGDIVMTQSPLSLPVTPGEPASISCRSSQSVLYSNGYNYLDWYLQKPGQSPQLLIYLGSNRASGVPDRFSGSGSGTDFTLKISRVEAEDVGVYYCMQVLQTPFTFGPGTKVDWRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 114 aa, Artificial Sequence [sequence omitted]

SEQ ID NO: 164 - protein, 112 aa, Artificial Sequence [sequence omitted]

SEQ ID NO: 165 - protein, 121 aa, Artificial Sequence
  Description of Artificial Sequence Synthetic polypeptide
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSS

SEQ ID NO: 166 - protein, 106 aa, Artificial Sequence
  Description of Artificial Sequence Synthetic polypeptide
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIK

SEQ ID NO: 167 - protein, 120 aa, Artificial Sequence
  Description of Artificial Sequence Synthetic polypeptide
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 168 - protein, 112 aa, Artificial Sequence
  Description of Artificial Sequence Synthetic polypeptide
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 169 - protein, 119 aa, Artificial Sequence
  Description of Artificial Sequence Synthetic polypeptide
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSS

SEQ ID NO: 170 - protein, 111 aa, Artificial Sequence
  Description of Artificial Sequence Synthetic polypeptide
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVL

SEQ ID NO: 171 - protein, 120 aa, Artificial Sequence
  Description of Artificial Sequence Synthetic polypeptide
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 172 - protein, 108 aa, Artificial Sequence
  Description of Artificial Sequence Synthetic polypeptide
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIK

SEQ ID NO: 173 - protein, 117 aa, Artificial Sequence
  Description of Artificial Sequence Synthetic polypeptide
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTL

SEQ ID NO: 174 - protein, 107 aa, Artificial Sequence
  Description of Artificial Sequence Synthetic polypeptide
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIK

SEQ ID NO: 175 - protein, 4 aa, Artificial Sequence
  Description of Artificial Sequence Synthetic peptide
GFLG

SEQ ID NO: 176 - protein, 4 aa, Artificial Sequence
  Description of Artificial Sequence Synthetic peptide
ALAL

(19) United States
(12) Patent Application Publication
(10) Pub. No.: US 2021/0139604 A1
(43) Pub. Date: May 13, 2021

(54) COMPOSITIONS OF ANTIBODY CONSTRUCT-AGONIST CONJUGATES AND METHODS OF USE THEREOF

(71) Applicant: OPI VI - IP HoldCo LLC, New York, NY (US)

(72) Inventors: Peter Armstrong THOMPSON, Bellevue, WA (US); Badreddin EDRIS, San Jose, CA (US)

(21) Appl. No.: 17/066,776

(22) Filed: Oct. 9, 2020

Related U.S. Application Data
(63) Continuation of application No. 15/624,441, filed on Jun. 15, 2017, now abandoned.
Application No. 15/624,441 is a continuation of application No. PCT/US2016/065353, filed on Dec. 7, 2016.
Application No. PCT/US2016/065353 is a continuation of application No. 15/173,075, filed on Jun. 3, 2016, now abandoned.
(60) Provisional application No. 62/371,141, filed on Aug. 4, 2016.
(60) Provisional application No. 62/264,260, filed on Dec. 7, 2015.

(30) Foreign Application Priority Data
Dec. 7, 2016 (GB) 1620828.2

(51) Int. Cl. C07K 16/30; C07K 16/28; A61K 47/68; A61K 47/64

(52) U.S. Cl. CPC C07K 16/3015; C07K 16/2878; A61K 47/6849; A61K 47/6803; A61K 2039/505; C07K 2317/92; C07K 2317/526; C07K 2317/72; C07K 2317/524; A61K 47/646

(57) ABSTRACT

Various antibody construct compositions are disclosed. The compositions of antibody construct-immune stimulatory compound conjugates are also provided. Additionally provided are the methods of preparation and used of the antibody construct-immune stimulatory compound conjugates. This includes methods for treating disorders, such as cancer. A genus of STING agonist compounds and method of synthesis is also disclosed.

### CROSS REFERENCE

This application is a continuation of U.S. patent application Ser. No. 15/624,441, filed on Jun. 15, 2017, which is a continuation of International Patent Application No. PCT/US2016/065353, filed on Dec. 7, 2016, which claims the benefit of U.S. Provisional Patent Application No. 62/371,141 filed on Aug. 4, 2016, U.S. patent application Ser. No. 15/173,075, filed on Jun. 3, 2016, and U.S. Provisional Patent Application No. 62/264,260, filed on Dec. 7, 2015, each of which is incorporated herein by reference in its entirety.

This application also claims the benefit of United Kingdom Patent Application No. 1620828.2, filed Dec. 7, 2016, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jan. 12, 2017, is named 50358_704_601_SL.txt and is 218,668 bytes in size.

### BACKGROUND

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

The composition described herein relates to different embodiments of a conjugate. In various embodiments, a conjugate comprises a) an immune-stimulatory compound; b) an antibody construct comprising an antigen binding domain and an Fc domain, wherein said antigen binding domain binds to a first antigen and wherein a K_{d} for binding of said Fc domain to an Fc receptor in the presence of said immune-stimulatory compound is no greater than about 100 times a K_{d} for binding of said Fc domain to said Fc receptor in the absence of the immune stimulatory compound; and c) a linker, wherein said linker attaches said antibody construct to said immune-stimulatory compound. In some aspects, said antigen binding domain binds said first antigen in a presence of said immune-stimulatory compound. In some aspects, a K_{d} for binding of said antigen binding domain to said first antigen in a presence of said immune-stimulatory compound is less than about 100 nM and no greater than about 100 times a K_{d} for binding of said antigen binding domain to said first antigen in the absence of said immune-stimulatory compound. In some aspects, said K_{d} for binding of said antigen binding domain to said first antigen in the presence of said immune-stimulatory compound is less than about 100 nM and is no greater than about 10 times the K_{d} of the binding of the antigen binding domain to said first antigen in the absence of the immune-stimulatory compound; and said K_{d} for binding of said Fc domain to said Fc receptor in the presence of said immune-stimulatory compound is no greater than about 10 times said K_{d} for the binding of said Fc domain to said Fc receptor in the absence of said immune stimulatory compound. In some aspects, a molar ratio of immune-stimulatory compound to antibody construct is less than 9, less than 8, less than 7, less than 6, less than 5, less than 4, less than 3, or less than 2.

In some aspects, said conjugate further comprises a targeting binding domain, wherein said targeting domain is attached to said antibody construct. In some aspects, said targeting binding domain binds a second antigen. In some aspects, said targeting binding domain is attached to said antibody construct at a C-terminal end of said Fc domain.

In some aspects, said antigen binding domain is from an antibody or non-antibody scaffold.

The conjugate of any of claims 1-9, wherein said antigen binding domain is at least 80% homologous to an antigen binding domain from an antibody or non-antibody scaffold. In some aspects, said non-antibody scaffold is a DARPin, affimer, avimer, knottin, monobody, or affinity clamp. In some aspects, said antigen binding domain is at least 80% homologous to an antigen binding domain from a DARPin, affimer, avimer, knottin, monobody, or affinity clamp.

In some aspects, said antigen binding domain recognizes a single antigen. In some aspects, said antigen binding domain recognizes two or more antigens. In some aspects, said first antigen is a tumor antigen. In some aspects, said first antigen that is at least 80% homologous to CD5, CD19, CD20, CD25, CD37, CD30, CD33, CD45, CAMPATH-1, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MART, Ras mutant, gp100, p53 mutant, PR1, bcr-ab1, tyronsinase, survivin, PSA, hTERT, Sarcoma translocation breakpoints, EphA2, PAP, ML-IAP, AFP, ERG, NA17, PAX3, ALK, androgen receptor, cyclin B1, polysialic acid, MYCN, RhoC, TRP-2, fucosyl GM1, mesothelin, PSCA, MAGE A1, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL, MUC16, MAGE A4, MAGE C2, GAGE, EGFR, CMET, HER3, MUC1, MUC15, MSLN, CA6, NAPI2B, TROP2, CLDN18.2, RON, LY6E, FRA, DLL3, PTK7, LIV1, ROR1, MAGE-A3, or Fos-related antigen 1. In some aspects, wherein said first antigen is expressed on an immune cell. In some aspects, said first antigen is expressed on an antigen-presenting cell. In some aspects, said first antigen is expressed on a dendritic cell, a macrophage, or a B-cell. In some aspects, wherein said first antigen is CD40. In some aspects, said antigen binding domain is a CD40 agonist.

In some aspects, said antibody construct is an antibody. In some aspects, said antibody construct is a human antibody or a humanized antibody. In some aspects, said antibody construct comprises a light chain sequence that is at least 80%, 90%, or 100% homologous to SEQ ID NO: 4, at least 80%, 90%, or 100% homologous to SEQ ID NO: 26, or at least 80%, 90%, or 100% homologous to SEQ ID NO: 34. In some aspects, said antibody construct comprises a light chain variable domain sequence that is at least 80%, 90%, or 100% homologous to SEQ ID NO: 6. In some aspects, said antibody construct comprises: a) a heavy chain sequence that is at least 80%, 90%, or 100% homologous to SEQ ID NO: 15; b) a heavy chain sequence that is at least 80%, 90%, or 100% homologous to SEQ ID NO: 16; c) a heavy chain sequence that is at least 80%, 90%, or 100% homologous to SEQ ID NO: 17; d) a heavy chain sequence that is at least 80%, 90%, or 100% homologous to SEQ ID NO: 18; e) a heavy chain sequence that is at least 80%, 90%, or 100% homologous to SEQ ID NO: 22; or f) heavy chain sequence that is at least 80%, 90%, or 100% homologous to SEQ ID NO: 30. In some aspects, said antibody construct comprises a heavy chain variable domain that is at least 80%, 90%, or 100% homologous to SEQ ID NO: 20. In some aspects, said antibody binding domain comprises at least 80%, 90%, or 100% homology to: a) HC CDR1 comprising an amino acid sequence of SEQ ID NO: 23, HC CDR2 comprising an amino acid sequence of SEQ ID NO: 24, a HC CDR3 comprising an amino acid sequence of SEQ ID NO: 25, LC CDR1 comprising an amino acid sequence of SEQ ID NO: 27, LC CDR1 comprising an amino acid sequence of SEQ ID NO: 28, and LC CDR3 comprising an amino acid sequence of SEQ ID NO: 29; or b) HC CDR1 comprising an amino acid sequence of SEQ ID NO: 31, HC CDR2 comprising an amino acid sequence of SEQ ID NO: 32, a HC CDR3 comprising an amino acid sequence of SEQ ID NO: 33, LC CDR1 comprising an amino acid sequence of SEQ ID NO: 35, LC CDR1 comprising an amino acid sequence of SEQ ID NO: 36, and LC CDR3 comprising an amino acid sequence of SEQ ID NO: 37.

In some aspects, said Fc domain is from an antibody. In some aspects, said Fc domain is at least 80% homologous to an Fc domain from an antibody. In some aspects, said Fc domain binding to said Fc receptor in the presence of said immune-stimulatory compound results in Fc-receptor-mediated signaling. In some aspects, said Fc domain binding to said Fc receptor in the presence of said immune-stimulatory compound results increased antigen presentation on an immune cell. In some aspects, said Fc domain is a human Fc domain. In some aspects, said Fc domain is selected from a group consisting of a human IgG1 Fc domain, a human IgG2 Fc domain, a human IgG3 Fc domain, and a human IgG4 Fc domain. In some aspects, wherein said Fc domain is an Fc domain variant comprising at least one amino acid residue change as compared to a wild type sequence of said Fc domain. In some aspects, said Fc domain binds said Fc receptor with altered affinity as compared to a wild type Fc domain. In some aspects, wherein said Fc receptor is selected from a group consisting of CD16a, CD16b, CD32a, CD32b, and CD64. In some aspects, said Fc receptor is a CD16a F158 variant or a CD16a V158 variant. In some aspects, said Fc domain binds said Fc receptor with higher affinity than a wild type Fe domain. In some aspects, said Fc receptor is selected from a group consisting of: CD16a, CD16b, CD32a, CD32b, or CD64. In some aspects, said Fc receptor is a CD16a F158 variant or a CD16a V158 variant. In some aspects, said Fc domain has at least one amino acid residue change as compared to wildtype, wherein said at least one amino acid residue change is: a) F243L, R292P, Y300L, L235V, and P396L, wherein numbering of amino acid residues in said Fc domain is according to the EU index as in Kabat; b) S239D and 332E, wherein numbering of amino acid residues in said Fc domain is according to the EU index as in Kabat; or c) S298A, E333A, and K334A, wherein numbering of amino acid residues in said Fc domain is according to the EU index as in Kabat.

In some aspects, said immune-stimulatory compound is a damage-associated molecular pattern molecule or a pathogen associated molecular pattern molecule In some aspects, said immune-stimulatory compound is a toll-like receptor agonist, STING agonist, or RIG-I agonist. In some aspects, said immune-stimulatory compound is a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, or a TLR10 agonist. In some aspects, said immune-stimulatory compound is selected from a group consisting of: S-27609, CL307, UC-IV150, imiquimod, gardiquimod, resiquimod, motolimod, VTS-1463GS-9620, GSK2245035, TMX-101, TMX-201, TMX-202, isatoribine, AZD8848, MEDI9197, 3M-051, 3M-852, 3M-052, 3M-854A, S-34240, KU34B, and CL663.

In some aspects, said immune-stimulatory compound comprises one or more rings selected from carbocyclic and heterocyclic rings. In some aspects, said linker is covalently attached to said antibody construct. In some aspects, said linker is covalently attached to said immune-stimulatory compound. In some aspects, said linker is not attached to an amino acid residue of said Fc domain selected from a group consisting of: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, or 428, wherein numbering of amino acid residues in said Fc domain is according to the EU index as in Kabat. In some aspects, said linker is attached to an amino acid residue of said antibody construct by a THIOMAB linker, or a Sortase A-catalyzed linker. In some aspects, said linker is attached to said antibody construct via a sulfhydryl group, a primary amine, a hinge cysteine, a C_{L} lysine, an engineered cysteine in a light chain, an engineered light chain glutamine, or an unnatural amino acid engineered into a light chain or heavy chain. In some aspects, said linker does not interfere with said Fe domain binding to said Fc receptor when said linker is attached to said antibody construct at an amino acid residue. In some aspects, said linker does not interfere with Fc-receptor-mediated signaling resulting from said Fc domain binding to said Fc receptor when said linker is attached to said Fc domain at an amino acid residue. In some aspects, said linker is attached to said immune-stimulatory compound via an exocyclic nitrogen or carbon atom of said immune-stimulatory compound. In some aspects, said immune-stimulatory compound is covalently attached to said linker by a bond to an exocyclic carbon or nitrogen atom on said immune-stimulatory compound.

In some aspects, wherein said linker is a peptide. In some aspects, said linker is a cleavable linker. In some aspects, said linker selected from a group consisting of: a) a valine-citrulline linker; b) a valine-citrulline linker containing a pentafluorophenyl group; c) a valine-citrulline linker containing a succinimide group; d) a valine-citrulline linker containing a para aminobenzoic acid group; e) a valine-citrulline linker containing a para aminobenzoic acid group and a pentafluorophenyl group; and f) a valine-citrulline linker containing a para aminobenzoic acid group and a succinimide group.

In some aspects, said linker is a non-cleavable linker. In some aspects, said linker selected from a group consisting of: a) a maleimidocaproyl linker; b) a combination of a maleimidocaproyl group and one or more polyethylene glycol molecules; c) a maleimide-PEG4 linker; d) a maleimidocaproyl linker containing a succinimide group; e) a maleimidocaproyl linker containing a pentafluorophenyl group; f) a combination of a maleimidocaproyl linker containing a succinimide group and one or more polyethylene glycol molecules; and g) a combination of a maleimidocaproyl linker containing a pentafluorophenyl group and one or more polyethylene glycol molecules.

In some aspects, said conjugate induces the secretion of cytokine by an antigen presenting cell. In some aspects, said cytokine is IFN-γ, IL-8, IL-12, IL-2, or a combination thereof. In some aspects, said conjugate induces antigen presentation on an antigen presenting cell.

In some aspects, said conjugate is formulated to treat tumors.

In some aspects, wherein said conjugate is in a pharmaceutical formulation.

In some embodiments, a pharmaceutical composition comprises said conjugate of any of the proceeding embodiments and a pharmaceutically acceptable carrier.

In some embodiments, a method of producing the conjugate of any of the preceding embodiments, comprises: a) selecting an antibody construct; b) selecting an immune-stimulatory compound; and c) attaching said antibody construct to said immune-stimulatory compound, wherein said immune-stimulatory compound is attached to said antibody construct via a linker and said antibody construct comprises an antigen binding domain and an Fc domain, wherein said antigen binding domain specifically binds an antigen in the presence of said immune-stimulatory compound and said Fc domain specifically binds an Fc receptor in the presence of said immune-stimulatory compound.

In some embodiments, a method of producing the conjugate of any of the preceding embodiments, comprises: a) selecting an antibody construct; b) selecting an immune-stimulatory compound; c) selecting a targeting binding domain; d) attaching said targeting binding domain to said antibody construct; and e) attaching said antibody construct to said immune-stimulatory compound, wherein said immune-stimulatory compound is attached to said antibody construct via a linker, wherein said antigen binding domain specifically binds a first antigen in the presence of said immune-stimulatory compound and said targeting binding specifically binds a second antigen in the presence of said immune-stimulatory compound.

In some embodiments, a method for treating a subject in need thereof, comprises administering a therapeutic dose of said conjugate of any one of the preceding embodiments or said pharmaceutical composition of any of the preceding embodiments. In some aspects, said subject has cancer. In some aspects, said composition is administered intravenously, cutaneously, subcutaneously, or injected at a site of affliction.

In some embodiments, a kit comprises said conjugate of any of the preceding embodiments.

A composition comprising a light chain sequence that is at least 80%, 90%, or 100% homologous to SEQ ID NO: 4 and heavy chain sequence that is at least 80%, 90%, or 100%

A composition comprising:
a) a light chain sequence that is at least 80%, 90%, or 100% homologous to SEQ ID NO: 4 or at least 80%, 90%, or 100% homologous to SEQ ID NO: 26; and
b) a heavy chain sequence that is at least 80%, 90%, or 100% homologous to SEQ ID NO: 16, at least 80%, 90%, or 100% homologous to SEQ ID NO: 17, or at least 80%, 90%, or 100% homologous to SEQ ID NO: 18.

The composition of claim 75, wherein said composition binds to an Fc receptor with greater affinity than an antibody comprising a heavy chain sequence of SEQ ID NO: 15 or SEQ ID NO: 22.

In some aspects, the present disclosure provides a compound represented by the structure of Formula (I):

[chemical structure]

or a pharmaceutically acceptable salt thereof, wherein:

X¹ is selected from —OR² and —SR²;

X² is selected from —OR³ and —SR³;

B¹ and B² are independently selected from optionally substituted nitrogenous bases;

Y is selected from —OR⁴, —NR⁴R⁴, and halogen;

R¹, R², R³ and R⁴ are independently selected at each occurrence from hydrogen, —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹, R², R³ and R⁴ is independently optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, and C₂₋₆ alkynyl; and R¹⁰⁰ at each occurrence is independently selected from hydrogen; and C₁₋₁₀ alkyl, C₂₋₁₀ alkenyl, C₂₋₁₀ alkynyl, C₃₋₁₀ carbocycle, and 3- to 10-membered heterocycle each of which is independently optionally substituted at each occurrence with one or more substituents selected from halogen, —CN, —NO₂, ═O, ═S, and haloalkyl.

In some embodiments, the compound of Formula (I) is represented by Formula (IA):

[chemical structure]

or pharmaceutically acceptable salts thereof.

In an alternative embodiment, the compound of Formula (I) is represented by Formula (IB):

[chemical structure]

or a pharmaceutically acceptable salt thereof.

In various embodiments, B¹ and B² are independently selected from optionally substituted purines, such as optionally substituted adenine, optionally substituted guanine, optionally substituted xanthine, optionally substituted hypoxanthine, optionally substituted theobromine, optionally substituted caffeine, optionally substituted uric acid, and optionally substituted isoguanine. In a preferred embodiment, B¹ and B² are independently selected from optionally substituted adenine and optionally substituted guanine.

In some embodiments, B¹ and B² are independently optionally substituted with one or more substituents, wherein the optional substituents on B¹ and B² are independently selected at each occurrence from halogen, ═O, ═S, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, —P(O)(OR¹⁰⁰)₂, —OP(O)(OR¹⁰⁰)₂ and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle is independently optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, and C₂₋₆ alkynyl. In a preferred embodiment, B¹ and B² are independently optionally substituted with one or more substituents, wherein the optional substituents on B¹ and B² are independently selected at each occurrence from halogen, ═O, ═S, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, —P(O)(OR¹⁰⁰)₂, —OP(O)(OR¹⁰⁰)₂, —CN and C₁₋₁₀ alkyl. In some embodiments, B¹ is an optionally substituted guanine. In some embodiments, B² is an optionally substituted guanine.

In various embodiments, X¹ is selected from —OH and —SH. For example, X¹ may be —OH. In various embodiments, X² is selected from —OH and —SH. For example, X² may be —OH.

In various embodiments, Y is selected from —OH, —O—C₁₋₁₀ alkyl, —NH(C₁₋₁₀ alkyl), and —NH₂. For example, Y may be —OH.

In various embodiments, R¹⁰⁰ is independently selected at each occurrence from hydrogen and C₁₋₁₀ alkyl optionally substituted at each occurrence with one or more substituents selected from halogen, —CN, —NO₂, ═O, and ═S.

In various embodiments, the compound of Formula (I) is represented by Formula (IC):

[chemical structure]

or a pharmaceutically acceptable salt thereof. In some embodiments, the compound of Formula (IC) is represented by Formula (ID):

[chemical structure]

or a pharmaceutically acceptable salt thereof.

In various embodiments, the compound is a pharmaceutically acceptable salt. The compound or salt may agonize a stimulator of interferon genes (STING).

In some aspects, the present disclosure provides an antibody drug conjugate, comprising a compound or salt previously described, an antibody, and a linker group, wherein the compound or salt is linked to the antibody through the linker group. The linker group may be selected from a cleavable or non-cleavable linker.

In some aspects, the present disclosure provides a compound represented by the structure of Formula (II):

[chemical structure]

or a pharmaceutically acceptable salt thereof, wherein:
X¹ is selected from —OR² and —SR²;
X² is selected from —OR³ and —SR³;
B¹ and B² are independently selected from optionally substituted nitrogenous bases,
   wherein each optional substituent is independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, R⁶, and —X³;
Y is selected from —OR⁴, —SR⁴, —NR⁴R⁴, and halogen;
Z is selected from —OR^{5′}—SR⁵, and —NR⁵R⁵;
R¹, R², R³, R⁴, and R⁵ are independently selected from a —X³; hydrogen, —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹, R², R³, R⁴, and R⁵ is optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, C₂₋₆ alkynyl; R⁶ is independently selected from —C(═O)R¹⁰⁰, —C(═O)OR¹ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R⁶ is optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, C₂₋₆ alkynyl;
R¹⁰⁰ at each occurrence is independently selected from hydrogen; and C₁₋₁₀ alkyl, C₂₋₁₀ alkenyl, C₂₋₁₀ alkynyl, C₃₋₁₀ carbocycle, and 3- to 10-membered heterocycle each of which is independently optionally substituted at each occurrence with one or more substituents selected from halogen, —CN, —NO₂, ═O, ═S, and haloalkyl; and
X³ is a linker moiety, wherein at least one of R¹, R², R³, R⁴, R⁵, X¹, X², a B¹ substituent and a B² substituent is —X³.

In various embodiments, the compound of Formula (II) is represented by a structure of Formula (IIA):

[chemical structure]

or pharmaceutically acceptable salts thereof.

In various embodiments, the compound of Formula (II) is represented by a structure of Formula (IIB):

[chemical structure]

or a pharmaceutically acceptable salt thereof.

In various embodiments, B¹ and B² are independently selected from optionally substituted purines. B¹ and B² may be each, independently selected from one another, adenine, guanine, and derivatives thereof. B¹ and B² may be independently selected from optionally substituted adenine, optionally substituted guanine, optionally substituted xanthine, optionally substituted hypoxanthine, optionally substituted theobromine, optionally substituted caffeine, optionally substituted uric acid, and optionally substituted isoguanine. In a preferred embodiment, B¹ and B² are independently selected from optionally substituted adenine and optionally substituted guanine.

In various embodiments, B¹ is substituted by X³ and optionally one or more additional substituents independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, and R⁶. For example, B¹ may be represented by:

[chemical structure]

and wherein B¹ is optionally further substituted by one or more substituents.

In various embodiments, B² is substituted by X³ and optionally one or more additional substituents independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, and R⁶. For example, B² may be represented by:

[chemical structure]

and wherein B² is optionally further substituted by one or more substituents.

In various embodiments, X¹ is selected from —O—X³ and —S—X³. In some embodiments, X¹ is selected from —OH and —SH.

In various embodiments, X² is selected from —O—X³ and —S—X³. In some embodiments, X² is selected from —OH and —SH.

In various embodiments, Y is selected from —NR⁴X³, —S—X³, and —O—X³. In some embodiments, Y is selected from —OH, —SH, —O—C₁₋₁₀ alkyl, —NH(C₁₋₁₀ alkyl), and —NH₂.

In various embodiments, Z is selected from —NR⁴X³, —S—X³, and —O—X³. In some embodiments, Z is selected from —OH, —SH, —O—C₁₋₁₀ alkyl, —NH(C₁₋₁₀ alkyl), and —NH₂.

In various embodiments, —X³ is a represented by the formula:

[chemical structure]

In some embodiments, —X³ is represented by the formula:

[chemical structure]

wherein RX comprises a reactive moiety, such a maleimide.

In some embodiments, —X³ is represented by the formula:

[chemical structure]

wherein RX* is a reactive moiety that has reacted with a moiety on an antibody to form an antibody drug conjugate.

In some embodiments, —X³ is represented by the formula:

[chemical structure]

wherein RX is a reactive moiety, such as a maleimide.

In some embodiments, —X³ is represented by the formula:

[chemical structure]

wherein RX* is a reactive moiety that has reacted with a moiety on an antibody to form an antibody drug conjugate.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound is represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically actable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1A illustrates a DNA sequence (SEQ ID NO: 1) of a light chain of a human CD40 monoclonal antibody SBT-040. Furthermore, SEQ ID NO: 1 illustrates a DNA sequence containing a signal sequence (SEQ ID NO: 2) as shown in FIG. 1B and a variable domain sequence (SEQ ID NO: 3) as shown in FIG. 1C.

FIG. 1B illustrates a DNA sequence of a signal sequence (SEQ ID NO: 2) of a light chain of a human CD40 monoclonal antibody SBT-040.

FIG. 1C illustrates a DNA sequence of a variable domain (SEQ ID NO: 3) in a light chain of a human CD40 monoclonal antibody SBT-040.

FIG. 2A illustrates an amino acid sequence (SEQ ID NO: 4) of a light chain of a human CD40 monoclonal antibody SBT-040. Furthermore, SEQ ID NO: 4 illustrates an amino acid sequence containing a signal sequence (SEQ ID NO: 5) as shown in FIG. 2B and a variable domain sequence (SEQ ID NO: 6) as shown in FIG. 2C.

FIG. 2B illustrates an amino acid sequence of a signal sequence (SEQ ID NO: 5) of a light chain of a human CD40 monoclonal antibody SBT-040.

FIG. 2C illustrates an amino acid sequence of a variable domain (SEQ ID NO: 6) in a light chain of a human CD40 monoclonal antibody SBT-040.

FIG. 3A illustrates a DNA sequence (SEQ ID NO: 7) of a wildtype IgG2 isotype heavy chain of a human CD40 monoclonal antibody SBT-040, wherein this heavy chain of the SBT-040 antibody can also be referred to as SBT-040-G2. Furthermore, SEQ ID NO: 7 illustrates a DNA sequence containing a signal sequence (SEQ ID NO: 12) as shown in FIG. 3F and a variable domain sequence (SEQ ID NO: 13) as shown in FIG. 3G.

FIG. 3B illustrates a DNA sequence (SEQ ID NO: 8) of a wild type IgG1 isotype heavy chain of a human CD40 monoclonal antibody SBT-040, wherein this heavy chain of the SBT-040 antibody can also be referred to as SBT-040-G1WT. Furthermore, SEQ ID NO: 8 illustrates a DNA sequence containing a signal sequence (SEQ ID NO: 12) as shown in FIG. 3F and a variable domain sequence (SEQ ID NO: 13) as shown in FIG. 3G.

FIG. 3C illustrates a DNA sequence (SEQ ID NO: 9) of an IgG1 isotype heavy chain of a human CD40 monoclonal antibody SBT-040 containing DNA nucleotide modifications corresponding to L235V, F243L, R292P, Y300L, and P396L amino acid residue modifications of a wild type IgG1 Fc domain, wherein this heavy chain of the SBT-040 antibody can also be referred to as SBT-040-G1VLPLL. The modified DNA nucleotides corresponding to the L235V, F243L, R292P, Y300L, and P396L amino acid residue modifications are in bold. Furthermore, SEQ ID NO: 9 illustrates a DNA sequence containing a signal sequence (SEQ ID NO: 12) as shown in FIG. 3F and a variable domain sequence (SEQ ID NO: 13) as shown in FIG. 3G.

FIG. 3D illustrates a DNA sequence (SEQ ID NO: 10) of an IgG1 isotype heavy chain of a human CD40 monoclonal antibody SBT-040 containing DNA nucleotide modifications corresponding to S239D and I332E amino acid residue modifications of a wild type IgG1 Fc domain, wherein this heavy chain of the SBT-040 antibody can also be referred to as SBT-040-GIDE. The modified DNA nucleotides corresponding to the S239D and I332E amino acid residue modifications are in bold. Furthermore, SEQ ID NO: 10 illustrates a DNA sequence containing a signal sequence (SEQ ID NO: 12) as shown in FIG. 3F and a variable domain sequence (SEQ ID NO: 13) as shown in FIG. 3G.

FIG. 3E illustrates a DNA sequence (SEQ ID NO: 11) of an IgG1 isotype heavy chain of human CD40 monoclonal antibody SBT-040 containing DNA nucleotide modifications corresponding to S298A, E333A, and K334A amino acid residue modifications of a wild type IgG1 Fc domain, wherein this heavy chain of the SBT-040 antibody can also be referred to as SBT-040-G1AAA. The modified DNA nucleotides corresponding to the S298A, E333A, and K334A amino acid residue modifications are in bold. Furthermore, SEQ ID NO: 11 illustrates a DNA sequence containing a signal sequence (SEQ ID NO: 12) as shown in FIG. 3F and a variable domain sequence (SEQ ID NO: 13) as shown in FIG. 3G.

FIG. 3F illustrates a DNA sequence of a signal sequence (SEQ ID NO: 12) of a heavy chain of a human CD40 monoclonal antibody SBT-040.

FIG. 3G illustrates a DNA sequence of a variable domain (SEQ ID NO: 13) in a heavy chain of a human CD40 monoclonal antibody SBT-040.

FIG. 4A illustrates an amino acid sequence (SEQ ID NO: 14) of a wildtype IgG2 isotype heavy chain of a human CD40 monoclonal antibody SBT-040, wherein this heavy chain of the SBT-040 antibody can also be referred to as SBT-040-G2. Furthermore, SEQ ID NO: 14 illustrates an amino acid sequence containing a signal sequence (SEQ ID NO: 19) as shown in FIG. 4F and a variable domain sequence (SEQ ID NO: 20) as shown in FIG. 4G.

FIG. 4B illustrates an amino acid sequence (SEQ ID NO: 15) of a wild type IgG1 isotype heavy chain of a human CD40 monoclonal antibody SBT-040, wherein this heavy chain of the SBT-040 antibody can also be referred to as SBT-040-G1WT. Furthermore, SEQ ID NO: 15 illustrates an amino acid sequence containing a signal sequence (SEQ ID NO: 19) as shown in FIG. 4F and a variable domain sequence (SEQ ID NO: 20) as shown in FIG. 4G.

FIG. 4C illustrates an amino acid sequence (SEQ ID NO: 16) of an IgG1 isotype heavy chain of a human CD40 monoclonal antibody SBT-040 containing L235V, F243L, R292P, Y300L, and P396L amino acid residue modifications of a wild type IgG1 Fc domain, wherein this heavy chain of the SBT-040 antibody can also be referred to as SBT-040-G1VLPLL. The amino acid residues corresponding to the L235V, F243L, R292P, Y300L, and P396L amino acid residue modifications are in bold. Furthermore, SEQ ID NO: 16 illustrates an amino acid sequence containing a signal sequence (SEQ ID NO: 19) as shown in FIG. 4F and a variable domain sequence (SEQ ID NO: 15) as shown in FIG. 4G.

FIG. 4D illustrates an amino acid sequence (SEQ ID NO: 17) of an IgG1 isotype heavy chain of a human CD40 monoclonal antibody SBT-040 containing S239D and I332 amino acid residue modifications of a wild type IgG1 Fc domain, wherein this heavy chain of the SBT-040 antibody can also be referred to as SBT-040-G1DE. The amino acid residues corresponding to the S239D and I332E amino acid residue modifications are in bold. Furthermore, SEQ ID NO: 17 illustrates an amino acid sequence containing a signal sequence (SEQ ID NO: 19) as shown in FIG. 4F and a variable domain sequence (SEQ ID NO: 20) as shown in FIG. 4G.

FIG. 4E illustrates an amino acid sequence (SEQ ID NO: 18) of an IgG1 isotype heavy chain of a human CD40 monoclonal antibody SBT-040 containing S298A, E333A, and K334A amino acid residue modifications of a wild type IgG1 Fc domain, wherein this heavy chain of the SBT-040 antibody can also be referred to as SBT-040-G1AAA. The amino acid residues corresponding to the S298A, E333A, and K334A amino acid modifications are in bold. Furthermore, SEQ ID NO: 11 illustrates an amino acid sequence containing a signal sequence (SEQ ID NO: 19) as shown in FIG. 4F and a variable domain sequence (SEQ ID NO: 20) as shown in FIG. 4G.

FIG. 4F illustrates an amino acid sequence of a signal sequence (SEQ ID NO: 19) of a heavy chain of a human CD40 monoclonal antibody SBT-040.

FIG. 4G illustrates an amino acid sequence of a variable domain (SEQ ID NO: 20) in a heavy chain of a human CD40 monoclonal antibody SBT-040.

FIGS. 5A, 5B, & 5C illustrate a CLUSTAL O(1.2.1) multiple DNA sequence alignment of the DNA sequences of SBT-040-G1VLPLL (SEQ ID NO: 9), SBT-040-G1AAA (SEQ ID NO: 11), SBT-040-G1WT (SEQ ID NO: 8), and SBT-040-G1DE (SEQ ID NO: 10). The SBT-040-G1VLPLL sequence is a DNA sequence of an IgG1 isotype heavy chain of a human CD40 monoclonal antibody SBT-040 containing DNA nucleotide modifications corresponding to L235V, F243L, R292P, Y300L, and P396L amino acid residue modifications of a wild type IgG1 Fc domain. The modified DNA nucleotides corresponding to the L235V, F243L, R292P, Y300L, and P396L amino acid residue modifications are in bold. The SBT-040-G1AAA sequence is a DNA sequence of an IgG1 isotype heavy chain of a human CD40 monoclonal antibody SBT-040 containing DNA nucleotide modifications corresponding to S298A, E333A, and K334A amino acid residue modifications of a wild type IgG1 Fc domain. The modified DNA nucleotides corresponding to the S298A, E333A, and K334A amino acid residue modifications are boxed. The SBT-040-G1WT sequence is a DNA sequence of an IgG1 isotype heavy chain of a human CD40 monoclonal antibody SBT-040. The SBT-040-G1AAA sequence is a DNA sequence of an IgG1 isotype heavy chain of a human CD40 monoclonal antibody SBT-040 containing DNA nucleotide modifications corresponding to S239D and I332E amino acid residue modifications of a wild type IgG1 Fc domain. The modified DNA nucleotides corresponding to the S239D and I332E amino acid residue modifications are in bold italics. FIG. 5A shows the start of the sequence alignment. FIG. 5B shows the middle of the sequence alignment as a continuation of FIG. 5A. FIG. 5C shows the end of the sequence alignment as continuation of FIG. 5C.

FIG. 6 illustrates a CLUSTAL O(1.2.1) multiple amino acid sequence alignment of the amino acid sequences of SBT-040-G1VLPLL (SEQ ID NO: 16), SBT-040-G1AAA (SEQ ID NO: 18), SBT-040-G1WT (SEQ ID NO: 15), and SBT-040-G1DE (SEQ ID NO: 17). The SBT-040-G1VLPLL sequence is an amino acid sequence of an IgG1 isotype heavy chain of a human CD40 monoclonal antibody SBT-040 containing L235V, F243L, R292P, Y300L, and P396L amino acid residue modifications of a wild type IgG1 Fc domain. The L235V, F243L, R292P, Y300L, and P396L amino acid residue modifications are in bold. The SBT-040-G1AAA sequence is an amino acid sequence of an IgG1 isotype heavy chain of a human CD40 monoclonal antibody SBT-040 containing S298A, E333A, and K334A amino acid residue modifications of a wild type IgG1 Fc domain. The S298A, E333A, and K334A amino acid residue modifications are italics. The SBT-040-G1WT sequence is an amino acid sequence of an IgG1 isotype heavy chain of a human CD40 monoclonal antibody SBT-040. The SBT-040-G1AAA sequence is an amino acid sequence of an IgG1 isotype heavy chain of a human CD40 monoclonal antibody SBT-040 containing S239D and I332E amino acid residue modifications bold italics. Additionally, the hinge region of each amino acid sequence is differentiated from other regions of the amino acid sequence by brackets. The left bracket indicates the upper portion of the hinge region (UH). The four residues between the brackets are the middle portion of the hinge region. The right bracket indicates the lower portion of the hinge region (LH).

FIG. 7 illustrates a schematic of an antibody. An antibody contains two heavy chains as shown in gray and two light chains as shown in light gray. A portion of the heavy chains contain Fc domains (705 and 720). An antibody contains two antigen binding sites (710 and 715).

FIG. 8 illustrates a schematic of an exemplary conjugate. An antibody construct is an antibody, which contains two heavy chains as shown in gray and two light chains as shown in light gray. The antibody comprises two antigen binding sites (810 and 815), and a portion of the heavy chains contain Fc domains (805 and 820). The immune-stimulatory compounds (830 and 840) are conjugated to the antibody by linkers (860 and 870).

FIG. 9 illustrates a schematic of an exemplary conjugate. An antibody construct is an antibody, which contains two heavy chains as shown in gray and two light chains as shown in light gray. The antibody comprises two antigen binding sites (910 and 915), and a portion of the heavy chains contain Fc domains (905 and 920). The immune-stimulatory compounds (930 and 940) are conjugated to the antibody by linkers (960 and 970). Targeting binding domains are conjugated to the antibody (980 and 985).

FIG. 10 illustrates a schematic of an exemplary conjugate. An antibody construct contains the Fc region of an antibody with the heavy chains shown in gray, and two scaffolds as shown in light gray. The antibody construct comprises two antigen binding sites (1010 and 1015) in the scaffolds, and a portion of the heavy chains contain Fc domains (1005 and 1020). The immune-stimulatory compounds (1030 and 1040) are conjugated to the antibody construct by linkers (1060 and 1070).

FIG. 11 illustrates a schematic of an exemplary conjugate. An antibody construct contains the Fc region of an antibody with the heavy chains shown in gray, and two scaffolds as shown in light gray. The antibody construct comprises two antigen binding sites (1110 and 1115) in the scaffolds, and a portion of the heavy chains contain Fc domains (1105 and 1120). The immune-stimulatory compounds (1130 and 1140) are conjugated to the antibody construct by linkers (1160 and 1170). Targeting binding domains are conjugated to the antibody construct (1180 and 1185).

FIG. 12 illustrates a schematic of an exemplary conjugate. An antibody construct contains the F(ab′)₂ region of an antibody with heavy chains shown in gray and light chains shown in light gray, and two scaffolds as shown in dark gray. The antibody construct comprises two antigen binding sites (1210 and 1215), and a portion of two scaffolds contain Fc domains (1220 and 1245). The immune-stimulatory compounds (1230 and 1240) are conjugated to the antibody construct by linkers (1260 and 1270).

FIG. 13 illustrates a schematic of an exemplary conjugate. An antibody construct contains the F(ab′)₂ region of an antibody with heavy chains shown in gray and light chains shown in light gray, and two scaffolds as shown in dark gray. The antibody construct comprises two antigen binding sites (1310 and 1315), and a portion of two scaffolds contain Fc domains (1320 and 1345). The immune-stimulatory compounds (1330 and 1340) are conjugated to the antibody construct by linkers (1360 and 1370). Targeting binding domains are conjugated to the antibody construct (1380 and 1385).

FIG. 14 illustrates a schematic of an exemplary conjugate. An antibody construct contains two scaffolds as shown in light gray and two scaffolds as shown in dark gray. The antibody construct comprises two antigen binding sites (1410 and 1415), and a portion of the two dark gray scaffolds contain Fc domains (1420 and 1445). The immune-stimulatory compounds (1430 and 1440) are conjugated to the antibody construct by linkers (1460 and 1470).

FIG. 15 illustrates a schematic of an exemplary conjugate. An antibody construct contains two scaffolds as shown in light gray and two scaffolds as shown in dark gray. The antibody construct comprises two antigen binding sites (1510 and 1515), and a portion of the two dark gray scaffolds contain Fc domains (1520 and 1545). The immune-stimulatory compounds (1530 and 1540) are conjugated to the antibody construct by linkers (1560 and 1570). Targeting binding domains are conjugated to the antibody construct (1580 and 1585).

FIG. 16 is the two-dimensional structure of the heavy chain of Dacetuzumab. Figure discloses SEQ ID NO: 163.

FIG. 17 is the two-dimensional structure of the light chain of Dacetuzumab. Figure discloses SEQ ID NO: 164.

FIG. 18 is the two-dimensional structure of the heavy chain of Bleselumab. Figure discloses SEQ ID NO: 165.

FIG. 19 is the two-dimensional structure of the light chain of Bleselumab. Figure discloses SEQ ID NO: 166.

FIG. 20 is the two-dimensional structure of the heavy chain of Lucatumumab. Figure discloses SEQ ID NO: 167.

FIG. 21 is the two-dimensional structure of the light chain of Lucatumumab. Figure discloses SEQ ID NO: 168.

FIG. 22 is the two-dimensional structure of the heavy chain of ADC-1013. Figure discloses SEQ ID NO: 169.

FIG. 23 is the two-dimensional structure of the light chain of ADC-1013. Figure discloses SEQ ID NO: 170.

FIG. 24 is the two-dimensional structure of the heavy chain of humanized rabbit antibody APX005. Figure discloses SEQ ID NO: 171.

FIG. 25 is the two-dimensional structure of the light chain of humanized rabbit antibody APX005. Figure discloses SEQ ID NO: 172.

FIG. 26 is the two-dimensional structure of the heavy chain of Chi Lob 7/4. Figure discloses SEQ ID NO: 173.

FIG. 27 is the two-dimensional structure of the light chain of Chi Lob 7/4. Figure discloses SEQ ID NO: 174.

FIG. 28 shows HPLC analysis of SBT-040-G1WT conjugated to a Cys-targeted drug linker tool compound.

FIG. 29 shows HPLC analysis of SBT-040-G1WT conjugated to ATAC2.

FIG. 30 shows HPLC analysis of SBT-040-G2WT conjugated to ATAC2.

FIG. 31A shows the concentration of IL-12p70 produced by dendritic cells (DCs) from donor 358 after incubation with SBT-040-WT-ATAC23 or SBT-040-WT-ATAC17 as compared with SBT-050-WT.

FIG. 31B shows the concentration of IL-12p70 produced by DCs from donor 363 after incubation with SBT-040-WT-ATAC23 or SBT-040-WT-ATAC17 as compared with SBT-050-WT.

FIG. 31C shows the concentration of TNFα produced by DCs from donor 358 after incubation with SBT-040-WT-ATAC23 or SBT-040-WT-ATAC17 as compared with SBT-050-WT.

FIG. 31D shows the concentration of TNFα produced by DCs from donor 363 after incubation with SBT-040-WT-ATAC23 or SBT-040-WT-ATAC17 as compared with SBT-050-WT.

FIG. 32A shows the concentration of IL-12p70 produced by DCs after incubation with SBT-040-WT-ATAC4, SBT-040-WT-ATAC3, SBT-040-G2-ATAC4, SBT-040-G2-ATAC3, SBT-040-AAA-ATAC22, SBT-040-VLPLL-ATAC22, SBT-040-WT-ATAC1, SBT-040-G2-ATAC1, SBT-040-WT-ATAC12, SBT-040-G2-ATAC12, SBT-040-WT-ATAC30, SBT-040-G1AAA-ATAC11, SBT-040-VLPLL-ATAC11, SBT-040-VLPLL-ATAC12, SBT-040-AAA-ATAC12, SBT-040-VLPLL-ATAC23, and SBT-040-AAA-ATAC23 as compared with SBT-050-G2 or CD40 ligand.

FIG. 32B shows the concentration of IL-6 produced by DCs from donor 2 after incubation with SBT-040-WT-ATAC4, SBT-040-WT-ATAC3, SBT-040-G2-ATAC4, SBT-040-G2-ATAC3, SBT-040-AAA-ATAC22, SBT-040-VLPLL-ATAC22, SBT-040-WT-ATAC1, SBT-040-G2-ATAC1, SBT-040-WT-ATAC12, SBT-040-G2-ATAC12, SBT-040-WT-ATAC30, SBT-040-AAA-ATAC11, SBT-040-VLPLL-ATAC11, SBT-040-VLPLL-ATAC12, SBT-040-AAA-ATAC12, SBT-040-VLPLL-ATAC23, and SBT-040-AAA-ATAC30 compared with SBT-050-G2 or CD40 ligand. Results are shown for the immune stimulatory cytokines IL-12p70 and IL-6.

FIG. 33A shows a dose dependent increase in CD86 expression on dendritic cells after treatment with SBT-040-WT-ATAC23, SBT-040-WT-ATAC17, SBT-040-VLPLL-ATAC22, SBT-040-AAA-ATAC23 as compared to treatment a control SBT-050-WT or staining with an isotype control.

FIG. 33B shows a dose dependent increase in CD83 expression on dendritic cells after treatment with SBT-040-WT-ATAC23, SBT-040-WT-ATAC17, SBT-040-VLPLL-ATAC23, SBT-040-AAA-ATAC23 as compared to treatment a control SBT-050-WT or staining with an isotype control.

FIG. 33C shows a dose dependent increase in MHC class II expression on dendritic cells after treatment with SBT-040-WT-ATAC23, SBT-040-WT-ATAC17, SBT-040-VLPLL-ATAC23, SBT-040-AAA-ATAC23 as compared to treatment a control SBT-050-WT or staining with an isotype control.

### DETAILED DESCRIPTION

Additional aspects and advantages of the present disclosure will become apparent to those skilled in this art from the following detailed description, wherein illustrative aspects of the present disclosure are shown and described. As will be realized, the present disclosure is capable of other and different aspects, and its several details are capable of modifications in various respects, all without departing from the disclosure. Accordingly, the drawings and description are to be regarded as illustrative in nature, and not as restrictive.

Cancer is one of the leading causes of death in the United States. Conventional methods of cancer treatment like chemotherapy, surgery or radiation therapy, can be limited in their efficacy since they are often nonspecific to the cancer. In many cases tumors, however, can specifically express genes whose products are required for inducing or maintaining the malignant state. These proteins may serve as antigen markers for the development and establishment of efficient anti-cancer treatments.

As used herein, “homologous” or “homology” can refer to the similarity between a DNA, RNA, nucleotide, amino acid, or protein sequence to another DNA, RNA, nucleotide, amino acid, or protein sequence. Homology can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “antibody” can refer to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, humanized, heteroconjugate, bispecific, diabody, triabody, or tetrabody. The antigen binding fragment can include, for example, Fab′, F(ab′)₂, Fab, Fv, rIgG, and scFv.

As used herein, “recognize” can refer to the association or binding between an antigen binding domain and an antigen.

As used herein, a “tumor antigen” can be an antigenic substance associated with a tumor or cancer cell, and can trigger an immune response in a host.

As used herein, an “antibody construct” can refer to a construct that contains an antigen binding domain and an Fc domain.

As used herein, an “antigen binding domain” can refer to an antigen binding domain from an antibody or from a non-antibody that can bind to the antigen.

As used herein, a “Fc domain” can be an Fc domain from an antibody or from a non-antibody that can bind to an Fc receptor.

As used herein, a “target binding domain” can refer to a construct that contains an antigen binding domain from an antibody or from a non-antibody that can bind to the antigen.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond respectively.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary carbocycles include cyclopentyl, cyclohexyl, cyclohexenyl, adamantyl, phenyl, indanyl, and naphthyl.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings wherein at least one of the rings includes a heteroatom. In an exemplary embodiment, an aromatic ring, e.g., pyridyl, may be fused to a saturated or unsaturated ring, e.g., cyclohexane, cyclopentane, morpholine, piperidine or cyclohexene. The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also include polycyclic ring systems having two or more cyclic rings in which two or more carbons are common to two adjoining rings wherein at least one of the rings is heteroaromatic, e.g., the other cyclic rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include

[chemical structure]

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

“Antibody drug conjugate” (“ADC”) can refer to an antibody construct immune-stimulatory compound conjugate. An ADC can comprise any embodiment as described herein for an antibody construct immune-stimulatory compound conjugate. Therefore, ADC and antibody construct immune-stimulatory compound conjugate can be used interchangeably herein.

An antigen can elicit an immune response. An antigen can be a protein, polysaccharide, lipid, or glycolipid, which can be recognized by an immune cell, such as a T cell or a B cell. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

In cancer, there are four general groups of tumor antigens: (i) viral tumor antigens which can be identical for any viral tumor of this type, (ii) carcinogenic tumor antigens which can be specific for patients and for the tumors, (iii) isoantigens of the transplantation type or tumor-specific transplantation antigens which can be different in all individual types of tumor but can be the same in different tumors caused by the same virus; and (iv) embryonic antigens.

As a result of the discovery of tumor antigens, tumor antigens have become important in the development of new cancer treatments that can specifically target the cancer. This has led to the development of antibodies directed against these tumor antigens.

In addition to the development of antibodies against tumor antigens for cancer treatment, antibodies that target immune cells to boost the immune response have also been developed. For example, an anti-CD40 antibody that is a CD40 agonist can be used to activate dendritic cells to enhance the immune response.

Cluster of Differentiation 40 (CD40) is a member of the Tumor Necrosis Factor Receptor (TNF-R) family. CD40 can be a 50 kDa cell surface glycoprotein that can be constitutively expressed in normal cells, such as monocytes, macrophages, B lymphocytes, dendritic cells, endothelial cells, smooth muscle cells, fibroblasts and epithelium, and in tumor cells, including B-cell lymphomas and many types of solid tumors. Expression of CD40 can be increased in antigen presenting cells in response to IL-1βp, IFN-γ, GM-CSF, and LPS induced signaling events.

Humoral and cellular immune responses can be regulated, in part, by CD40. For example, in the absence of CD40 activation by its cognate binding partner, CD40 Ligand (CD40L), antigen presentation can result in tolerance. However, CD40 activation can ameliorate tolerance. In addition, CD40 activation can positively impact immune responses by enhancing antigen presentation by antigen presenting cells (APC), increasing cytokine and chemokine secretion, stimulating expression of and signaling by co-stimulatory molecules, and activating the cytolytic activity of different types of immune cells. Accordingly, the interaction between CD40 and CD40L can be essential to maintain proper humoral and cellular immune responses.

The intracellular effects of CD40 and CD40L interaction can include association of the CD40 cytoplasmic domain with TRAFs (TNF-R associated factors), which can lead to the activation of NFκB and Jun/AP1 pathways. While the response to activation of NFκB and Jun/AP1 pathways can be cell type-specific, often such activation can lead to increased production and secretion of cytokines, including IL-6, IL-8, IL-12, IL-15; increased production and secretion of chemokines, including MIP1α and β and RANTES; and increased expression of cellular adhesion molecules, including ICAM. While the effects of cytokines, chemokines and cellular adhesion molecules can be widespread, such effects can include enhanced survival and activation of T cells.

In addition to the enhanced immune responses induced by CD40 activation, CD40 activation can also be involved in chemokine- and cytokine-mediated cellular migration and differentiation; activation of immune cells, including monocytes; activation of and increased cytolytic activity of immune cells, including cytolytic T lymphocytes and natural killer cells; induction of CD40-positive tumor cell apoptosis and enhanced immunogenicity of CD40-positive tumors. In addition, CD40 can initiate and enhance immune responses by many different mechanisms, including, inducing antigen-presenting cell maturation and increased expression of costimulatory molecules, increasing production of and secretion of cytokines, and enhancing effector functions.

CD40 activation can be effective for inducing immune-mediated antitumor responses. For example, CD40 activation reverses host immune tolerance to tumor-specific antigens which leads to enhanced antitumor responses by T cells. Such antitumor activity can also occur in the absence of immune cells. Similarly, antitumor effects can occur in response to anti-CD40 antibody-mediated activation of CD40 and can be independent of antibody-dependent cellular cytotoxicity. In addition to other CD40-mediated mechanisms of antitumor effects, CD40L-stimulation can cause dendritic cell maturation and stimulation. CD40L-stimulated dendritic cells can contribute to the antitumor response. Furthermore, vaccination strategies including CD40 can result in regression of CD40-positive and CD40-negative tumors.

CD40 activating antibodies (e.g., anti-CD40 activating monoclonal antibodies) can be useful for treatment of tumors. This can occur through one or more mechanisms, including cell activation, antigen presentation, production of cytokines and chemokines, amongst others. For example, CD40 antibodies activate dendritic cells, leading to processing and presentation of tumor antigens as well as enhanced immunogenicity of CD40-positive tumor cells. Not only can enhanced immunogenicity result in activation of CD40-positive tumor specific CD4⁺ and CD8⁺ T cells, but further antitumor activity can include, recruitment and activation monocytes, enhanced cytolytic activity of cytotoxic lymphocytes and natural killer cells as well as induction of apoptosis or by stimulation of a humoral response so as to directly target tumor cells. In addition, tumor cell debris, including tumor-specific antigens, can be presented to other cells of the immune system by CD40-activated antigen presenting cells.

Since CD40 can be important in an immune response, there is a need for enhanced CD40 meditated signaling events to provide reliable and rapid treatment options to patients suffering from diseases which may be ameliorated by treatment with CD40-targeted therapeutic strategies.

The HER2/neu (human epidermal growth factor receptor 2/receptor tyrosine-protein kinase erbB-2) is part of the human epidermal growth factor family. Overexpression of this protein has been shown to play an important role in the progression of cancer, for example, breast cancer. The HER2/neu protein functions as a receptor tyrosine kinase and autophosphorylates upon dimerization with binding partners. HER2/neu can activate several signaling pathways including, for example, mitogen-activated protein kinase, phosphoinositide 3-kinase, phospholipase Cγ, protein kinase C, and signal transducer and activator of transcription (STAT). Several compounds have been developed to inhibit HER2/neu including for example, the monoclonal antibody trastuzumab and the monoclonal antibody pertuzumab.

Immune-stimulatory molecular motifs, such as Pathogen-Associated Molecular Pattern molecules, (PAMPs) can be recognized by receptors of the innate immune system, such as Toll-like receptors (TLRs), Nod-like receptors, C-type lectins, and RIG-I-like receptors. These receptors can be transmembrane and intra-endosomal proteins which can prime activation of the immune system in response to infectious agents such as pathogens. Similar to other protein families, TLRs can have many isoforms, including TLR4, TLR7 and TLR8. Several agonists targeting activation of different TLRs can be used in various immunotherapies, including vaccine adjuvants and in cancer immunotherapies. TLR agonists can range from simple molecules to complex macromolecules. Likewise, the sizes of TLR agonists can range from small to large. TLR agonists can be synthetic or biosynthetic agonists. TLR agonists can also be PAMPs. Additional immune-stimulatory compounds, such as cytosolic DNA and unique bacterial nucleic acids called cyclic dinucleotides, can be recognized by Interferon Regulatory Factor (IRF) or stimulator of interferon genes (STING), which can act a cytosolic DNA sensor. Compounds recognized by Interferon Regulatory Factor (IRF) can play a role in immunoregulation by TLRs and other pattern recognition receptors.

Imiquimod, a synthetic TLR7 agonist, is currently approved for human therapeutic applications. Contained in a cream and marketed under the brand name Aldara, imiquimod serves as a topical treatment for a variety of indications with immune components, such as, actinic keratosis, genital warts, and basal cell carcinomas. In addition, imiquimod is indicated as a candidate adjuvant for enhancing adaptive immune responses when applied topically at an immunization site.

Another type of immune stimulatory molecular motif, damage-associated molecular pattern molecules (DAMPs), can initiate and maintain an immune response occurring as part of the non-infectious inflammatory response. DAMPs can be specially localized proteins that, when detected by the immune system in a location other than where DAMPs should be located, activate the immune system. Often, DAMPs can be nuclear or cytosolic proteins and upon release from the nucleus or cytosol, DAMP proteins can become denatured through oxidation. Examples of DAMP proteins can include chromatin-associated protein high-mobility group box 1 (HMGB1), S100 molecules of the calcium modulated family of proteins and glycans, such as hyaluronan fragments, and glycan conjugates. DAMPs can also be nucleic acids, such as DNA, when released from tumor cells following apoptosis or necrosis. Examples of additional DAMP nucleic acids can include RNA and purine metabolites, such as ATP, adenosine and uric acid, present outside of the nucleus or mitochondria.

Therapeutic application of DAMPs can focus on indications with an immune component, such as arthritis, cancer, ischemia-reperfusion injury, myocardial infarction and stroke. In these indications, the mechanism of action for DAMP therapeutic effects can include the prevention of DAMP release using therapeutic strategies, such as proapoptotic interventions, platinum and ethyl pyruvate, extracellular neutralization or blockade of DAMP release or signaling using therapeutic strategies such as anti-HMGB1, rasburiaspect and sRAGE, as well as direct or indirect blockade of DAMP receptors, and downstream signaling events, using therapeutic strategies such as RAGE small molecule antagonists; TLR4 antagonists and antibodies to DAMP-R.

Additionally, the immune response elicited by TLR agonists can further be enhanced when co-administered with a CD40-agonist antibody. For example, co-administration of a TLR agonist such as poly IC:LC with a CD40-agonist antibody can synergize to stimulate a greater CD8⁺ T cell response than either agonist alone.

However, therapeutic use of PAMPs and DAMPs or other mechanisms of intervention can be limited because systemic activation of PAMP and DAMP signaling pathways can have life-threatening consequences due to cytokine syndrome-induced or cytokine storm-induced toxic shock syndrome. Accordingly, there is a critical need for therapeutic, clinically relevant targeted delivery of PAMP and DAMP agonists for safe and effective strategies to enhance immune responses. The presently described conjugate can be utilized as a safe and effective strategy to enhance immune responses. A conjugate can comprise an antibody construct and an immune-stimulatory compound.

### Antibody Construct

An antibody construct can comprise an antigen binding domain. An antigen binding domain can be a domain that can specifically bind to an antigen. An antigen binding domain can be an antigen-binding portion of an antibody or an antibody fragment. An antigen binding domain can be one or more fragments of an antibody that can retain the ability to specifically bind to an antigen. An antigen binding domain can be any antigen binding fragment. An antigen binding domain can recognize a single antigen. An antigen binding domain can recognize, for example, two, three, four, five, six, seven, eight, nine, ten, or more antigens. An antibody construct can contain, for example, two, three, four, five, six, seven, eight, nine, ten, or more antigen binding domains. An antibody construct can contain two antigen binding domains in which each antigen binding domain can recognize the same antigen. An antibody construct can contain two antigen binding domains in which each antigen binding domain can recognize different antigens. An antigen binding domain can be in a scaffold, in which a scaffold is a supporting framework for the antigen binding domain. An antigen binding domain can be in a non-antibody scaffold. An antigen binding domain can be in an antibody scaffold. An antibody construct can comprise an antigen binding domain in a scaffold. The antibody construct can comprise a Fc fusion protein product. In some embodiments, the antibody construct is a Fc fusion protein product.

The antigen binding domain of an antibody construct can be selected from any domain that binds the antigen including, but not limited to, from a monoclonal antibody, a polyclonal antibody, a recombinant antibody, or a functional fragment thereof, for example, a heavy chain variable domain (V_{H}) and a light chain variable domain (V_{L}), a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor. The antigen binding domain of an antibody construct can be at least 80% homologous to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

An antigen binding domain of an antibody construct, for example an antigen binding domain from a monoclonal antibody, can comprise a light chain and a heavy chain. In one aspect, the monoclonal antibody binds to CD40 and comprises the light chain of an anti-CD40 antibody and the heavy chain of an anti-CD40 antibody, which bind a CD40 antigen. In another aspect, the monoclonal antibody binds to a tumor antigen and comprises the light chain of a tumor antigen antibody and the heavy chain of a tumor antigen antibody, which bind the tumor antigen.

An antibody construct can be an antibody. An antibody can consist of two identical light protein chains and two identical heavy protein chains, all held together covalently by precisely located disulfide linkages. The N-terminal regions of the light and heavy chains together can form the antigen recognition site of an antibody. Structurally, various functions of an antibody can be confined to discrete protein domains (i.e., regions). The sites that can recognize and can bind antigen can consist of three complementarity determining regions (CDRs) that can lie within the variable heavy chain region and variable light chain region at the N-terminal end of the heavy chain and the light chain. The constant domains can provide the general framework of the antibody and may not be involved directly in binding the antibody to an antigen, but can be involved in various effector functions, such as participation of the antibody in antibody-dependent cellular cytotoxicity, and can bind Fc receptors.

The domains of natural light and heavy chains can have the same general structures, and each domain can comprise four framework regions, whose sequences can be somewhat conserved, connected by three hyper-variable regions or CDRs. The four framework regions can largely adopt a β-sheet conformation and the CDRs can form loops connecting, and in some aspects forming part of, the R-sheet structure. The CDRs in each chain can be held in close proximity by the framework regions and, with the CDRs from the other chain, can contribute to the formation of the antigen binding site.

An antibody of an antibody construct can include an antibody of any type, which can be assigned to different classes of immunoglobins, e.g., IgA, IgD, IgE, IgG, and IgM. Several different classes can be further divided into isotypes, e.g., IgG1, IgG2, IgG3, IgG4, IgA1, and IgA2. An antibody can further comprise a light chain and a heavy chain, often more than one chain. The heavy-chain constant regions (Fc) that corresponds to the different classes of immunoglobulins can be α, δ, ε, γ, and μ, respectively. The light chains can be one of either kappa or κ and lambda or λ, based on the amino acid sequences of the constant domains. The Fc region can contain an Fc domain. An Fc receptor can bind an Fc domain. Antibody constructs can also include any fragment or recombinant forms thereof, including but not limited to, single chain variable fragments (scFvs), ‘T-bodies’, anti-calins, centyrins, affibodies, domain antibodies, or peptibodies.

An antibody can comprise an antigen binding domain, which can refer to a portion of an antibody comprising the antigen recognition portion, i.e., an antigenic determining variable region of an antibody sufficient to confer recognition of the antigen and binding of the antigen recognition portion to a target, such as an antigen, i.e., the epitope. Examples of antibody binding domains can include, but are not limited to, Fab, scFv, variable Fv fragment, and other antibody fragments, combinations of fragments or types of fragments known or knowable to one of ordinary skill in the art.

An antibody construct can comprise an antigen binding domain of an antibody. An antigen binding domain of an antibody can comprise one or more light chain (LC) CDRs and one or more heavy chain (HC) CDRs. For example, an antibody binding domain of an antibody can comprise one or more of the following: a light chain complementary determining region 1 (LC CDR1), a light chain complementary determining region 2 (LC CDR2), or a light chain complementary determining region 3 (LC CDR3). For another example, an antibody binding domain can comprise one or more of the following: a heavy chain complementary determining region 1 (HC CDR1), a heavy chain complementary determining region 2 (HC CDR2), or a heavy chain complementary determining region 3 (HC CDR3). As an additional example, an antibody binding domain of an antibody can comprise one or more of the following: LC CDR1, LC CDR2, LC CDR3, HC CDR1, HC CDR2, and HC CDR3.

An antibody construct can comprise an antibody fragment. An antibody fragment can include (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, can be coded for by separate genes, they can be linked by a synthetic linker to be made as a single protein chain in which the V_{L} and V_{H} regions pair to form monovalent molecules.

F(ab′)₂ and Fab′ moieties can be produced by treating immunoglobulin (e.g., monoclonal antibody) with a protease such as pepsin and papain, and can include an antibody fragment generated by digesting immunoglobulin near the disulfide bonds existing between the hinge regions in each of the two H chains. The Fab fragment can also contain the constant domain of the light chain and the first constant domain (C_{H1}) of the heavy chain. Fab′ fragments can differ from Fab fragments by the addition of a few residues at the carboxyl terminus of the heavy chain C_{H1} domain including one or more cysteine(s) from the antibody hinge region.

An Fv can be the minimum antibody fragment which contains a complete antigen-recognition and antigen-binding site. This region can consist of a dimer of one heavy chain and one light chain variable domain in tight, non-covalent association. In this configuration the three hypervariable regions of each variable domain can interact to define an antigen-binding site on the surface of the V_{H}-V_{L} dimer. A single variable domain (or half of an Fv comprising only three hypervariable regions specific for an antigen) can recognize and bind antigen, although the binding can be at a lower affinity than the affinity of the entire binding site.

An antibody used herein can be “humanized.” Humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which can contain minimal sequences derived from non-human immunoglobulin. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence.

An antibody described herein can be a human antibody. As used herein, “human antibodies” can include antibodies having, for example, the amino acid sequence of a human immunoglobulin and can include antibodies isolated from human immunoglobulin libraries or from animals transgenic for one or more human immunoglobulins that do not express endogenous immunoglobulins. Human antibodies can be produced using transgenic mice which are incapable of expressing functional endogenous immunoglobulins, but which can express human immunoglobulin genes. Completely human antibodies that recognize a selected epitope can be generated using guided selection. In this approach, a selected non-human monoclonal antibody, e.g., a mouse antibody, can be used to guide the selection of a completely human antibody recognizing the same epitope.

An antibody described herein can be a bispecific antibody or a dual variable domain antibody (DVD). Bispecific and DVD antibodies can be monoclonal, often human or humanized, antibodies that can have binding specificities for at least two different antigens.

An antibody described herein can be a derivatized antibody. For example, derivatized antibodies can be modified by glycosylation, acetylation, pegylation, phosphorylation, amidation, derivatization by known protecting/blocking groups, proteolytic cleavage, linkage to a cellular ligand or other protein.

An antibody described herein can have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding can be reduced by, for example, mutating the immunoglobulin constant region segment of the antibody at particular regions necessary for FcR interactions.

An antibody described herein can be modified to acquire or improve at least one constant region-mediated biological effector function relative to an unmodified antibody, e.g., to enhance FcγR interactions. For example, an antibody with a constant region that binds FcγRIIA, FcγRIIB and/or FcγRIIIA with greater affinity than the corresponding wild type constant region can be produced according to the methods described herein.

An antibody described herein can bind to tumor cells, such as an antibody against a cell surface receptor or a tumor antigen. An antibody described herein can bind to CD40, such as an antibody that can be a CD40 agonist and bind to CD40.

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

An antibody construct can comprise an anti-CD40 antibody. An antibody construct can comprise an antibody light chain. A light chain can be a light chain of an anti-CD40 antibody which can bind a CD40 antigen. A light chain of an anti-CD40 antibody can be expressed from a DNA sequence comprising ATGAGGCTCCCTGCTCAGCTCCTGGGGCTCCTGCTGCTCTGGTTCCCAGGTTCCAGATGC GACATCCAGATGACCCAGTCTCCATCTTCCGTGTCTGCATCTGTAGGAGACAGAGTCAC CATCACTTGTCGGGCGAGTCAGGGTATTTACAGCTGGTTAGCCTGGTATCAGCAGAAAC CAGGGAAAGCCCCTAACCTCCTGATCTATACTGCATCCACTTTACAAAGTGGGGTCCCA TCAAGGTTCAGCGGCAGTGGATCTGGGACAGATTTCACTCTCACCATCAGCAGCCTGCA ACCTGAAGATTTTGCAACTTACTATTGTCAACAGGCTAACATTTTCCCGCTCACTTTCGG CGGAGGGACCAAGGTGGAGATCAAACGAACTGTGGCTGCACCATCTGTCTTCATCTTCC CGCCATCTGATGAGCAGTTGAAATCTGGAACTGCCTCTGTTGTGTGCCTGCTGAATAAC TTCTATCCCAGAGAGGCCAAAGTACAGTGGAAGGTGGATAACGCCCTCCAATCGGGTA ACTCCCAGGAGAGTGTCACAGAGCAGGACAGCAAGGACAGCACCTACAGCCTCAGCAG CACCCTGACGCTGAGCAAAGCAGACTACGAGAAACACAAAGTCTACGCCTGCGAAGTC ACCCATCAGGGCCTGAGCTCGCCCGTCACAAAGAGCTTCAACAGGGGAGAGTGTTAG (SEQ ID NO: 1). A light chain of an anti-CD40 antibody can be expressed from DNA sequence comprising greater than 70%, greater than 75%, greater than 80%, greater than 85%, greater than 90%, greater than 95%, or greater than 99% homology to SEQ ID NO: 1. A variable region of a light chain of an anti-CD40 antibody can be expressed from a DNA sequence comprising GACATCCAGATGACCCAGTCTCCATCTTCCGTGTCTGCATCTGTAGGAGACAGAGTCAC CATCACTTGTCGGGCGAGTCAGGGTATTTACAGCTGGTTAGCCTGGTATCAGCAGAAAC CAGGGAAAGCCCCTAACCTCCTGATCTATACTGCATCCACTTTACAAAGTGGGGTCCCA TCAAGGTTCAGCGGCAGTGGATCTGGGACAGATTTCACTCTCACCATCAGCAGCCTGCA ACCTGAAGATTTTGCAACTTACTATTGTCAACAGGCTAACATTTTCCCGCTCACTTTCGG CGGAGGGACCAAGGTGGAGATCAA (SEQ ID NO: 3). A variable region of a light chain of an anti-CD40 antibody can be expressed from a DNA sequence comprising greater than 70%, greater than 75%, greater than 80%, greater than 85%, greater than 90%, greater than 95% or greater than 99% homology to SEQ ID NO: 3. Additionally, anti-CD40 antibodies expressed from SEQ ID NO: 1, or expressed from a DNA sequence comprising greater than 70% homology to SEQ ID NO: 1 can have a dissociation constant (K_{d}) for CD40 that is less than 10 nM. Anti-CD40 antibodies expressed from SEQ ID NO: 1, or expressed from a DNA sequence comprising greater than 70% homology to SEQ ID NO: 1 can have a dissociation constant (K_{d}) for CD40 that is less than 1 nM, less than 100 pM, less than 10 pM, less than 1 pM, or less than 0.1 pM. The anti-CD40 light chain can be expressed with any anti-CD40 heavy chain or fragment thereof. The anti-CD40 light chain can also expressed with any anti-CD40 heavy chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct.

A light chain of an anti-CD40 antibody can comprise an amino acid sequence MRLPAQLLGLLLLWFPGSRCDIQMTQSPSSVSASVGDRVTITCRASQGIYSWLAWYQQKPG KAPNLLIYTASTLQSGVPSRFSGSGSGTDFTLTISSLQPEDFATYYCQQANIFPLTFGGGTKVE IKRTVAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO: 4). A light chain of an anti-CD40 antibody can comprise an amino sequence with greater than 70%, greater than 75%, greater than 80%, greater than 85%, greater than 90%, greater than 95% or greater than 99% homology to SEQ ID NO: 4. A variable region of a light chain of an anti-CD40 antibody can comprise an amino acid sequence DIQMTQSPSSVSASVGDRVTITCRASQGIYSWLAWYQQKPGKAPNLLIYTASTLQSGVPSRF SGSGSGTDFTLTISSLQPEDFATYYCQQANIFPLTFGGGTKVEIK (SEQ ID NO: 6). A variable region of a light chain of an anti-CD40 antibody can comprise an amino acid sequence with greater than 70%, greater than 75%, greater than 80%, greater than 85%, greater than 90%, greater than 95% or greater than 99% homology to SEQ ID NO: 6. Additionally, anti-CD40 antibodies comprising SEQ ID NO: 4, or comprising an amino acid sequence with greater than 70% homology to SEQ ID NO: 4 can have a dissociation constant (K_{d}) for CD40 that is less than 10 nM. Anti-CD40 antibodies comprising SEQ ID NO: 4, or comprising an amino acid sequence with greater than 70% homology to SEQ ID NO: 4 can have a dissociation constant (K_{d}) for CD40 that is less than 1 nM, less than 100 pM, less than 10 pM, less than 1 pM, or less than 0.1 pM. The anti-CD40 light chain can be purified, and can be combined with a pharmaceutically acceptable carrier. The anti-CD40 light chain can be combined with any anti-CD40 heavy chain or fragment thereof. The anti-CD40 light chain can also be combined with any anti-CD40 heavy chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to anti-CD40 antibodies created for use in the veterinary sciences and/or in laboratory animals.

An antibody construct can comprise an antibody light chain. A light chain can be a light chain of an anti-CD40 antibody which can bind a CD40 antigen. A light chain of an anti-CD40 antibody can be SBT-040 VL-Ck. SBT-040 VL-Ck can comprise an amino acid sequence DIQMTQSPSSVSASVGDRVTITCRASQGIYSWLAWYQQKPGKAPNLLIYTASTLQSGVPSRF SGSGSGTDFTLTISSLQPEDFATYYCQQANIFPLTFGGGTKVEIKRTVAAPSVFIFPPSDEQLK SGTASVVCLLNNFYPREAKVQWKVDNALQSGNSQESVTEQDSKDSTYSLSSTLTLSKADYE KHKVYACEVTHQGLSSPVTKSFNRGEC (SEQ ID NO: 26). SBT-040 VL-Ck can comprise an amino acid sequence with greater than 70%, greater than 75%, greater than 80%, greater than 85%, greater than 90%, greater than 95% or greater than 99% homology to SEQ ID NO: 26.

A light chain of an anti-CD40 antibody can comprise a CDR. A light chain of an anti-CD40 antibody can comprise a CDR with an amino acid sequence QGIYSW (SEQ ID NO: 27). A light chain of an anti-CD40 antibody can comprise a CDR with an amino acid sequence TAS (SEQ ID NO: 28). A light chain of an anti-CD40 antibody can comprise a CDR with an amino acid sequence QQANIFPLT (SEQ ID NO: 29). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 27. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 28. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 29.

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be Dacetuzumab. Dacetuzumab can comprise an amino acid sequence EVQLVESGGGLVQPGGSLRLSCAASGYSFTGYYIHWVRQAPGKGLEWVARVIPNAGGTSY NQKFKGRFTLSVDNSKNTAYLQMNSLRAEDTAVYYCAREGIYWWGQGTLVTVSSASTKG PSVFPLAPSSKSTSGGTAALGCLVKDYFPEPVTVSWNSGALTSGVHTFPAVLQSSGLYSLSS VVTVPSSSLGTQTYICNVNHKPSNTKVDKKVEPKSCDKTHTCPPCPAPELLGGPSVFLFPPKP KDTLMISRTPEVTCVVVDVSHEDPEVKFNWYVDGVEVHNAKTKPREEQYNSTYRVVSVLT VLHQDWLNGKEYKCKVSNKALPAPIEKTISKAKGQPREPQVYTLPPSREEMTKNQVSLTCL VKGFYPSDIAVEWESNGQPENNYKTTPPVLDSDGSFFLYSKLTVDKSRWQQGNVFSCSVM HEALHNHYTQKSLSLSPGK (SEQ ID NO: 38). Dacetuzumab can comprise an amino acid sequence with greater than 70%, greater than 75%, greater than 80%, greater than 85%, greater than 90%, greater than 95% or greater than 99% homology to SEQ ID NO: 38. A heavy chain of an anti-CD40 antibody can comprise a CDR. A heavy chain of an anti-CD40 antibody can comprise a CDR with an amino acid sequence GYSFTGYY (SEQ ID NO: 39). A heavy chain of an anti-CD40 antibody can comprise a CDR with an amino acid sequence VIPNAGGT (SEQ ID NO: 40). A heavy chain of an anti-CD40 antibody can comprise a CDR with an amino acid sequence AREGIYW (SEQ ID NO: 41). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 39. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 40. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 41. The two-dimensional structure of the dacetuzumab heavy chain is shown in FIG. 16.

An antibody construct can comprise an antibody light chain. A light chain can be a light chain of an anti-CD40 antibody which can bind a CD40 antigen. A light chain of an anti-CD40 antibody can be dacetuzumab. Dacetuzumab can comprise an amino acid sequence DIQMTQSPSSLSASVGDRVTITCRSSQSLVHSNGNTFLHWYQQKPGKAPKLLIYTVSNRFSG VPSRFSGSGSGTDFTLTISSLQPEDFATYFCSQTTHVPWTFGQGTKVEIKRTVAAPSVFIFPPS DEQLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO: 42). Dacetuzumab can comprise an amino acid sequence with greater than 70%, greater than 75%, greater than 80%, greater than 85%, greater than 90%, greater than 95% or greater than 99% homology to SEQ ID NO: 42. A light chain of an anti-CD40 antibody can comprise a CDR. A light chain of an anti-CD40 antibody can comprise a CDR with an amino acid sequence QSLVHSNGNTF (SEQ ID NO: 43). A light chain of an anti-CD40 antibody can comprise a CDR with an amino acid sequence TVS (SEQ ID NO: 44). A light chain of an anti-CD40 antibody can comprise a CDR with an amino acid sequence SQTTHVPWT (SEQ ID NO: 45). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 43. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 44. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 45. The two-dimensional structure of the Dacetuzumab light chain is shown in FIG. 17.

An antibody construct can comprise an antibody heavy chain. A heavy chain can be a heavy chain of an anti-CD40 antibody which can bind a CD40 antigen. A heavy chain of an anti-CD40 antibody can be an IgG4 isotype. A heavy chain of an anti-CD40 antibody can be Bleselumab. Bleselumab can comprise an amino acid sequence QLQLQESGPGLLKPSETLSLTCTVSGGSISSPGYYGGWIRQPPGKGLEWIGSIYKSGSTYHNP SLKSRVTISVDTSKNQFSLKLSSVTAADTAVYYCTRPVVRYFGWFDPWGQGTLVTVSSAST KGPSVFPLAPCSRSTSESTAALGCLVKDYFPEPVTVSWNSGALTSGVHTFPAVLQSSGLYSL SSVVTVPSSSLGTKTYTCNVDHKPSNTKVDKRVESKYGPPCPPCPAPEFEGGPSVFLFPPKPK DTLMISRTPEVTCVVVDVSQEDPEVQFNWYVDGVEVHNAKTKPREEQFNSTYRVVSVLTV LHQDWLNGKEYKCKVSNKGLPSSIEKTISKAKGQPREPQVYTLPPSQEEMTKNQVSLTCLV KGFYPSDIAVEWESNGQPENNYKTTPPVLDSDGSFFLYSRLTVDKSRWQEGNVFSCSVMHE ALHNHYTQKSLSLSLGK (SEQ ID NO: 46). Bleselumab can comprise an amino acid sequence with greater than 70%, greater than 75%, greater than 80%, greater than 85%, greater than 90%, greater than 95% or greater than 99% homology to SEQ ID NO: 46. A heavy chain of an anti-CD40 antibody can comprise a CDR. A heavy chain of an anti-CD40 antibody can comprise a CDR with an amino acid sequence GGSISSPGYY (SEQ ID NO: 47). A heavy chain of an anti-CD40 antibody can comprise a CDR with an amino acid sequence IYKSGST (SEQ ID NO: 48). A heavy chain of an anti-CD40 antibody can comprise a CDR with an amino acid sequence TRPVVRYFGWFDP (SEQ ID NO: 49). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 47. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 48. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 49. The two-dimensional structure of the bleselumab heavy chain is shown in FIG. 18.

An antibody construct can comprise an antibody light chain. A light chain can be a light chain of an anti-CD40 antibody which can bind a CD40 antigen. A light chain of an anti-CD40 antibody can be Bleselumab. Bleselumab can comprise an amino acid sequence AIQLTQSPSSLSASVGDRVTITCRASQGISSALAWYQQKPGKAPKLLIYDASNLESGVPSRFS GSGSGTDFTLTISSLQPEDFATYYCQQFNSYPTFGQGTKVEIKRTVAAPSVFIFPPSDEQLKSG TASVVCLLNNFYPREAKVQWKVDNALQSGNSQESVTEQDSKDSTYSLSSTLTLSKADYEK HKVYACEVTHQGLSSPVTKSFNRGEC (SEQ ID NO: 50). Bleselumab can comprise an amino acid sequence with greater than 70%, greater than 75%, greater than 80%, greater than 85%, greater than 90%, greater than 95% or greater than 99% homology to SEQ ID NO: 50. A light chain of an anti-CD40 antibody can comprise a CDR. A light chain of an anti-CD40 antibody can comprise a CDR with an amino acid sequence QGISSA (SEQ ID NO: 51). A light chain of an anti-CD40 antibody can comprise a CDR with an amino acid sequence DAS (SEQ ID NO: 52). A light chain of an anti-CD40 antibody can comprise a CDR with an amino acid sequence QQFNSYPT (SEQ ID NO: 53). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 51. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 52. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 53. The two-dimensional structure of the bleselumab light chain is shown in FIG. 19.

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. Lucatumumab can comprise an amino acid sequence QVQLVESGGGVVQPGRSLRLSCAASGFTFSSYGMHWVRQAPGKGLEWVAVISYEESNRYH ADSVKGRFTISRDNSKITLYLQMNSLRTEDTAVYYCARDGGIAAPGPDYWGQGTLVTVSSA STKGPSVFPLAPASKSTSGGTAALGCLVKDYFPEPVTVSWNSGALTSGVHTFPAVLQSSGLY SLSSVVTVPSSSLGTQTYICNVNHKPSNTKVDKRVEPKSCDKTHTCPPCPAPELLGGPSVFLF PPKPKDTLMISRTPEVTCVVVDVSHEDPEVKFNWYVDGVEVHNAKTKPREEQYNSTYRVV SVLTVLHQDWLNGKEYKCKVSNKALPAPIEKTISKAKGQPREPQVYTLPPSREEMTKNQVS LTCLVKGFYPSDIAVEWESNGQPENNYKTTPPVLDSDGSFFLYSKLTVDKSRWQQGNVFSC SVMHEALHNHYTQKSLSLSPGK (SEQ ID NO: 54). Lucatumumab can comprise an amino acid sequence with greater than 70%, greater than 75%, greater than 80%, greater than 85%, greater than 90%, greater than 95% or greater than 99% homology to SEQ ID NO: 54. A heavy chain of an anti-CD40 antibody can comprise a CDR. A heavy chain of an anti-CD40 antibody can comprise a CDR with an amino acid sequence GFTFSSYG (SEQ ID NO: 55). A heavy chain of an anti-CD40 antibody can comprise a CDR with an amino acid sequence ISYEESNR (SEQ ID NO: 56). A heavy chain of an anti-CD40 antibody can comprise a CDR with an amino acid sequence ARDGGIAAPGPDY (SEQ ID NO: 57). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 55. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 56. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 57. The two-dimensional structure of the lucatumumab heavy chain is shown in FIG. 20.

An antibody construct can comprise an antibody light chain. A light chain can be a light chain of an anti-CD40 antibody which can bind a CD40 antigen. A light chain of an anti-CD40 antibody can be Lucatumumab. Lucatumumab can comprise an amino acid sequence DIVMTQSPLSLTVTPGEPASISCRSSQSLLYSNGYNYLDWYLQKPGQSPQVLISLGSNRASGV PDRFSGSGSGTDFTLKISRVEAEDVGVYYCMQARQTPFTFGPGTKVDIRRTVAAPSVFIFPPS DEQLKSGTASVVCLLNNFYPREAKVQWKVDNALQSGNSQESVTEQDSKDSTYSLSSTLTLS KADYEKHKVYACEVTHQGLSSPVTKSFNRGEC (SEQ ID NO: 58). Lucatumumab can comprise an amino acid sequence with greater than 70%, greater than 75%, greater than 80%, greater than 85%, greater than 90%, greater than 95% or greater than 99% homology to SEQ ID NO: 59. A light chain of an anti-CD40 antibody can comprise a CDR with an amino acid sequence LGS (SEQ ID NO: 60). A light chain of an anti-CD40 antibody can comprise a CDR with an amino acid sequence MQARQTPFT (SEQ ID NO: 61). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 59. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 60. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 61.

The two-dimensional structure of the lucatumumab light chain is shown in FIG. 21.

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be ADC-1013. ADC-1013 can comprise an amino acid sequence EVQLLESGGGLVQPGGSLRLSCAASGFTFSTYGMHWVRQAPGKGLEWLSYISGGSSYIFYA DSVRGRFTISRDNSENALYLQMNSLRAEDTAVYYCARILRGGSGMDLWGQGTLVTVSSAS TKGPSVFPLAPSSKSTSGGTAALGCLVKDYFPEPVTVSWNSGALTSGVHTFPAVLQSSGLYS LSSWTVPSSSLGTQTYICNVNHKPSNTKVDKKVEPKSCDKTHTCPPCPAPELLGGPSVFLFPP KPKDTLMISRTPEVTCNAVDVSHEDPEVKFNWYVDGVEVHNAKTKPREEQYNSTYRWSVL TVLHQDWLNGKEYKCKVSNKALPAPIEKTISKAKGQPREPQVYTLPPSRDELTKNQVSLTC LVKGFYPSDIAVEWESNGQPENNYKTTPPVLDSDGSFFLYSKLTVDKSRWQQGNVFSCSVM HEALHNHYTQKSLSLSPGK (SEQ ID NO: 62). ADC-1013 can comprise an amino acid sequence with greater than 70%, greater than 75%, greater than 80%, greater than 85%, greater than 90%, greater than 95% or greater than 99% homology to SEQ ID NO: 62. A heavy chain of an anti-CD40 antibody can comprise a CDR. A heavy chain of an anti-CD40 antibody can comprise a CDR with an amino acid sequence GFTFSTYG (SEQ ID NO: 63). A heavy chain of an anti-CD40 antibody can comprise a CDR with an amino acid sequence ISGGSSYI (SEQ ID NO: 64). A heavy chain of an anti-CD40 antibody can comprise a CDR with an amino acid sequence ARILRGGSGMDL (SEQ ID NO: 65). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 63. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 64. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 65. The two-dimensional structure of the ADC-1013 heavy chain is shown in FIG. 22.

An antibody construct can comprise an antibody light chain. A light chain can be a light chain of an anti-CD40 antibody which can bind a CD40 antigen. A light chain of an anti-CD40 antibody can be ADC-1013. ADC-1013 can comprise an amino acid sequence QSVLTQPPSASGTPGQRVTISCTGSSSNIGAGYNVYWYQQLPGTAPKLLIYGNINRPSGVPDR FSGSKSGTSASLAISGLRSEDEADYYCAAWDKSISGLVFGGGTKLTVLGQPKAAPSVTLFPP SSEELQANKATLVCLISDFYPGAVTVAWKADSSPVKAGVETTTPSKQSNNKYAASSYLSLT PEQWKSHRSYSCQVTHEGSTVEKTVAPTECS (SEQ ID NO: 66). ADC-1013 can comprise an amino acid sequence with greater than 70%, greater than 75%, greater than 80%, greater than 85%, greater than 90%, greater than 95% or greater than 99% homology to SEQ ID NO: 66. A light chain of an anti-CD40 antibody can comprise a CDR. A light chain of an anti-CD40 antibody can comprise a CDR with an amino acid sequence SSNIGAGYN (SEQ ID NO: 67). A light chain of an anti-CD40 antibody can comprise a CDR with an amino acid sequence GNI (SEQ ID NO: 68). A light chain of an anti-CD40 antibody can comprise a CDR with an amino acid sequence AAWDKSISGLV (SEQ ID NO: 69). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 67. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 68. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 69. The two-dimensional structure of the ADC-1013 light chain is shown in FIG. 23.

An antibody construct can comprise an antibody heavy chain. A heavy chain can be a heavy chain of an anti-CD40 antibody which can bind a CD40 antigen. A heavy chain of an anti-CD40 antibody can be the humanized rabbit antibody APX005. APX005 can comprise an amino acid sequence QVQLVESGGGVVQPGRSLRLSCAASGFSFSSTYVCWVRQAPGKGLEWIACIYTGDGTNYSA SWAKGRFTISKDSSKNTVYLQMNSLRAEDTAVYFCARPDITYGFAINFWGPGTLVTVSSAST KGPSVFPLAPSSKSTSGGTAALGCLVKDYFPEPVTVSWNSGALTSGVHTFPAVLQSSGLYSL SSVVTVPSSSLGTQTYICNVNHKPSNTKVDKKVEPKSCDKTHTCPPCPAPELLGGPSVFLFPP KPKDTLMISRTPEVTCVVVDVSHEDPEVKFNWYVDGVEVHNAKTKPREEQYNSTYRVVSV LTVLHQDWLNGKEYKCKVSNKALPAPIEKTISKAKGQPREPQVYTLPPSREEMTKNQVSLT CLVKGFYPSDIAVEWESNGQPENNYKTTPPVLDSDGSFFLYSKLTVDKSRWQQGNVFSCSV MHEALHNHYTQKSLSLSPGK (SEQ ID NO: 70). APX005 can comprise an amino acid sequence with greater than 70%, greater than 75%, greater than 80%, greater than 85%, greater than 90%, greater than 95% or greater than 99% homology to SEQ ID NO: 70. A heavy chain of an anti-CD40 antibody can comprise a CDR. A heavy chain of an anti-CD40 antibody can comprise a CDR with an amino acid sequence GFSFSSTY (SEQ ID NO: 71). A heavy chain of an anti-CD40 antibody can comprise a CDR with an amino acid sequence IYTGDGTN (SEQ ID NO: 72). A heavy chain of an anti-CD40 antibody can comprise a CDR with an amino acid sequence ARPDITYGFAINFW (SEQ ID NO: 73). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 71. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 72. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 73. The two-dimensional structure of the APX005 heavy chain is shown in FIG. 24.

An antibody construct can comprise an antibody light chain. A light chain can be a light chain of an anti-CD40 antibody which can bind a CD40 antigen. A light chain of an anti-CD40 antibody can be the humanized rabbit antibody APX005. APX005 can comprise an amino acid sequence DIQMTQSPSSLSASVGDRVTIKCQASQSISSRLAWYQQKPGKPPKLLIYRASTLASGVPSRFS GSGSGTDFTLTISSLQPEDVATYYCQCTGYGISWPIGGGTKVEIKRTVAAPSVFIFPPSDEQLK SGTASVVCLLNNFYPREAKVQWKVDNALQSGNSQESVTEQDSKDSTYSLSSTLTLSKADYE KHKVYACEVTHQGLSSPVTKSFNRGEC (SEQ ID NO: 74). APX005 can comprise an amino acid sequence with greater than 70%, greater than 75%, greater than 80%, greater than 85%, greater than 90%, greater than 95% or greater than 99% homology to SEQ ID NO: 74. A light chain of an anti-CD40 antibody can comprise a CDR. A light chain of an anti-CD40 antibody can comprise a CDR with an amino acid sequence QSISSR (SEQ ID NO: 75). A light chain of an anti-CD40 antibody can comprise a CDR with an amino acid sequence RAS (SEQ ID NO: 76). A light chain of an anti-CD40 antibody can comprise a CDR with an amino acid sequence QCTGYGISWP (SEQ ID NO: 77). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 75. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 76. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 77. The two-dimensional structure of the APX005 light chain is shown in FIG. 25.

An antibody construct can comprise an antibody heavy chain. A heavy chain can be a heavy chain of an anti-CD40 antibody which can bind a CD40 antigen. A heavy chain of an anti-CD40 antibody can be Chi Lob 7/4. Chi Lob 7/4 can comprise an amino acid sequence EVQLQQSGPDLVKPGASVKISCKTSGYTFTEYIMHWVKQSHGKSLEWIGGIIPNNGGTSYNQ KFKDKATMTVDKSSSTGYMELRSLTSEDSAVYYCTRREVYGRNYYALDYWGQGTLVTVS SASTKGPSVFPLAPSSKSTSGGTAALGCLVKDYFPEPVTVSWNSGALTSGVHTFPAVLQSSG LYSLSSVVTVPSSSLGTQTYICNVNHKPSNTKVDKKVEPKSCDKTHTCPPCPAPELLGGPSVF LFPPKPKDTLMISRTPEVTCVVVDVSHEDPEVKFNWYVDGVEVHNAKTKPREEQYNSTYR VVSVLTVLHQDWLNGKEYKCKVSNKALPAPIEKTISKAKGQPREPQVYTLPPSREEMTKNQ VSLTCLVKGFYPSDIAVEWESNGQPENNYKTTPPVLDSDGSFFLYSKLTVDKSRWQQGNVF SCSVMHEALHNHYTQKSLSLSPGK (SEQ ID NO: 78). Chi Lob 7/4 can comprise an amino acid sequence with greater than 70%, greater than 75%, greater than 80%, greater than 85%, greater than 90%, greater than 95% or greater than 99% homology to SEQ ID NO: 78. A heavy chain of an anti-CD40 antibody can comprise a CDR. A heavy chain of an anti-CD40 antibody can comprise a CDR with an amino acid sequence GYTFTEYI (SEQ ID NO: 79). A heavy chain of an anti-CD40 antibody can comprise a CDR with an amino acid sequence IIPNNGGT (SEQ ID NO: 80). A heavy chain of an anti-CD40 antibody can comprise a CDR with an amino acid sequence TRREVYGRNYYALDY (SEQ ID NO: 81). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 79. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 80. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 81. The two-dimensional structure of the Chi Lob 7/4 heavy chain is shown in FIG. 26.

An antibody construct can comprise an antibody light chain. A light chain can be a light chain of an anti-CD40 antibody which can bind a CD40 antigen. A light chain of an anti-CD40 antibody can be Chi Lob 7/4. Chi Lob 7/4 can comprise an amino acid sequence DIQMTQTTSSLSASLGDRVTITCSASQGINNYLNWYQQKPDGTVKLLIYYTSSLHSGVPSRFS GSGSGTDYSLTISNLEPEDIATYYCQQYSNLPYTFGGGTKLEIKRTVAAPSVFIFPPSDEQLKS GTASVVCLLNNFYPREAKVQWKVDNALQSGNSQESVTEQDSKDSTYSLSSTLTLSKADYE KHKVYACEVTHQGLSSPVTKSFNRGEC (SEQ ID NO: 82). Chi Lob 7/4 can comprise an amino acid sequence with greater than 70%, greater than 75%, greater than 80%, greater than 85%, greater than 90%, greater than 95% or greater than 99% homology to SEQ ID NO: 82. A light chain of an anti-CD40 antibody can comprise a CDR. A light chain of an anti-CD40 antibody can comprise a CDR with an amino acid sequence QGINNY (SEQ ID NO: 83). A light chain of an anti-CD40 antibody can comprise a CDR with an amino acid sequence YTS (SEQ ID NO: 84). A light chain of an anti-CD40 antibody can comprise a CDR with an amino acid sequence QQYSNLPYT (SEQ ID NO: 85). A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 83. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 84. A light chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 85. The two-dimensional structure of the Chi Lob 7/4 light chain is shown in FIG. 27.

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be SBT-040-G1WT. SBT-040-G1WT can be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCCCAAATCTTGTGACAAAACTCACACATGC CCACCGTGCCCAGCACCTGAACTCCTGGGGGGACCGTCAGTCTTCCTCTTCCCCCCAAA ACCCAAGGACACCCTCATGATCTCCCGGACCCCTGAGGTCACATGCGTGGTGGTGGACG TGAGCCACGAAGACCCTGAGGTCAAGTTCAACTGGTACGTGGACGGCGTGGAGGTGCA TAATGCCAAGACAAAGCCGCGGGAGGAGCAGTACAACAGCACGTACCGTGTGGTCAGC GTCCTCACCGTCCTGCACCAGGACTGGCTGAATGGCAAGGAGTACAAGTGCAAGGTCTC CAACAAAGCCCTCCCAGCCCCCATCGAGAAAACCATCTCCAAAGCCAAAGGGCAGCCC CGAGAACCACAGGTGTACACCCTGCCCCCATCCCGGGAGGAGATGACCAAGAACCAGG TCAGCCTGACCTGCCTGGTCAAAGGCTTCTATCCCAGCGACATCGCCGTGGAGTGGGAG AGCAATGGGCAGCCGGAGAACAACTACAAGACCACGCCTCCCGTGCTGGACTCCGACG GCTCCTTCTTCCTCTATAGCAAGCTCACCGTGGACAAGAGCAGGTGGCAGCAGGGGAAC GTCTTCTCATGCTCCGTGATGCATGAGGCTCTGCACAACCACTACACGCAGAAGAGCCT CTCCCTGTCCCCGGGTAAATGA (SEQ ID NO: 8). SBT-040-G1WT can be expressed from a DNA sequence comprising greater than 70%, greater than 75%, greater than 80%, greater than 85%, greater than 90%, greater than 95% or greater than 99% homology to SEQ ID NO: 8. A variable region of SBT-040-G1WT can be expressed from a DNA sequence comprising CAGGTGCAGCTGGTGCAGTCTGGGGCTGAGGTGAAGAAGCCTGGGGCCTCAGTGAAGG TCTCCTGCAAGGCTTCTGGATACACCTTCACCGGCTACTATATGCACTGGGTGCGACAG GCCCCTGGACAAGGGCTTGAGTGGATGGGATGGATCAACCCTGACAGTGGTGGCACAA ACTATGCACAGAAGTTTCAGGGCAGGGTCACCATGACCAGGGACACGTCCATCAGCAC AGCCTACATGGAGCTGAACAGGCTGAGATCTGACGACACGGCCGTGTATTACTGTGCGA GAGATCAGCCCCTAGGATATTGTACTAATGGTGTATGCTCCTACTTTGACTACTGGGGC CAGGGAACCCTGGTCACCGTCTCCTCAG (SEQ ID NO: 13). A variable region of SBT-040-G1WT can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1WT expressed from SEQ ID NO: 8, or expressed from a DNA sequence comprising greater than 70% homology to SEQ ID NO: 8 can have a dissociation constant (K_{d}) for CD40 that is less than 10 nM. Anti-CD40 antibodies comprising SBT-040-G1WT expressed from DNA sequence comprising SEQ ID NO: 8, or comprising greater than 70% homology to SEQ ID NO: 8 can have a dissociation constant (K_{d}) for CD40 that is less than 1 nM, less than 100 pM, less than 10 pM, less than 1 pM, or less than 0.1 pM. SBT-040-G1WT can be expressed with any anti-CD40 light chain or fragment thereof. SBT-040-G1WT can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G1WT can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVEPKSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKALPAPI EKTISKAKGQPREPQVYTLPPSREEMTKNQVSLTCLVKGFYPSDIAVEWESNGQPENNYKTT PPVLDSDGSFFLYSKLTVDKSRWQQGNVFSCSVMHEALHNHYTQKSLSLSPGK (SEQ ID NO: 15). SBT-040-G1WT can comprise an amino acid sequence with greater than 70%, greater than 75%, greater than 80%, greater than 85%, greater than 90%, greater than 95% or greater than 99% homology to SEQ ID NO: 15. SBT-040-G1WT can comprise an amino acid sequence QVQLVQSGAEVKKPGASVKVSCKASGYTFTGYYMHWVRQAPGQGLEWMGWINPDSGGT NYAQKFQGRVTMTRDTSISTAYMELNRLRSDDTAVYYCARDQPLGYCTNGVCSYFDYWG QGTLVTVSS (SEQ ID NO: 20). A variable region of SBT-040-G1WT can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1WT with SEQ ID NO: 15 or with an amino acid sequence with greater than 70% homology to SEQ ID NO: 15 can have a dissociation constant (K_{d}) for CD40 that is less than 10 nM. Anti-CD40 antibodies comprising SBT-040-G1WT with SEQ ID NO: 15 or with an amino acid sequence with greater than 70% homology to SEQ ID NO: 15 can have a dissociation constant (K_{d}) for CD40 that is less than 1 nM, less than 100 pM, less than 10 pM, less than 1 pM, or less than 0.1 pM. SBT-040-G1WT can be purified. SBT-040-G1WT can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

An antibody construct can comprise an antibody heavy chain. A heavy chain can be a heavy chain of an anti-CD40 antibody which can bind a CD40 antigen. A heavy chain of an anti-CD40 antibody can be an IgG1 isotype. A heavy chain of an anti-CD40 antibody can be SBT-040 VH-hIgG1 wt. SBT-040 VH-hIgG1 wt can comprise an amino acid sequence with greater than 70%, greater than 75%, greater than 80%, greater than 85%, greater than 90%, greater than 95% or greater than 99% homology to an amino acid sequence QVQLVQSGAEVKKPGASVKVSCKASGYTFTGYYMHWVRQAPGQGLEWMGWINPDSGGT NYAQKFQGRVTMTRDTSISTAYMELNRLRSDDTAVYYCARDQPLGYCTNGVCSYFDYWG QGTLVTVSSASTKGPSVFPLAPCSRSTSESTAALGCLVKDYFPEPVTVSWNSGALTSGVHTF PAVLQSSGLYSLSSVVTVPSSNFGTQTYTCNVDHKPSNTKVDKTVEPKSCDKTHTCPPCPAP ELLGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLTVD KSRWQQGNVFSCSVMHEALHNHYTQKSLSLSPGK (SEQ ID NO: 22). A heavy chain of an anti-CD40 antibody can comprise a CDR. A heavy chain of an anti-CD40 antibody can comprise a CDR with an amino acid sequence GYTFTYY (SEQ ID NO: 23). A heavy chain of an anti-CD40 antibody can comprise a CDR with an amino acid sequence INPDSGGT (SEQ ID NO: 24). A heavy chain of an anti-CD40 antibody can comprise a CDR with an amino acid sequence ARDQPLGYCTNGVCSYFDY (SEQ ID NO: 25). A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 23. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 24. A heavy chain CDR of an anti-CD40 antibody can comprise an amino acid sequence with greater than 70%, greater than 75%, greater than 80%, greater than 85%, greater than 90%, greater than 95% or greater than 99% homology to SEQ ID NO: 25.

A heavy chain of an anti-CD40 antibody can be an IgG2 isotype. A heavy chain of an anti-CD40 antibody can be SBT-040-G2. SBT-040-G2 be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCGCAAATGTTGTGTCGAGTGCCCACCGTGC CCAGCACCACCTGTGGCAGGACCGTCAGTCTTCCTCTTCCCCCCAAAACCCAAGGACAC CCTCATGATCTCCCGGACCCCTGAGGTCACGTGCGTGGTGGTGGACGTGAGCCACGAAG ACCCCGAGGTCCAGTTCAACTGGTACGTGGACGGCGTGGAGGTGCATAATGCCAAGAC AAAGCCACGGGAGGAGCAGTTCAACAGCACGTTCCGTGTGGTCAGCGTCCTCACCGTTG TGCACCAGGACTGGCTGAACGGCAAGGAGTACAAGTGCAAGGTCTCCAACAAAGGCCT CCCAGCCCCCATCGAGAAAACCATCTCCAAAACCAAAGGGCAGCCCCGAGAACCACAG GTGTACACCCTGCCCCCATCCCGGGAGGAGATGACCAAGAACCAGGTCAGCCTGACCT GCCTGGTCAAAGGCTTCTACCCCAGCGACATCGCCGTGGAGTGGGAGAGCAATGGGCA GCCGGAGAACAACTACAAGACCACACCTCCCATGCTGGACTCCGACGGCTCCTTCTTCC TCTACAGCAAGCTCACCGTGGACAAGAGCAGGTGGCAGCAGGGGAACGTCTTCTCATG CTCCGTGATGCATGAGGCTCTGCACAACCACTACACGCAGAAGAGCCTCTCCCTGTCTC CGGGTAAATGA (SEQ ID NO: 7). SBT-040-G2 can be expressed from a DNA sequence comprising greater than 70%, greater than 75%, greater than 80%, greater than 85%, greater than 90%, greater than 95% or greater than 99% homology to SEQ ID NO: 7. A variable region of SBT-040-G2 can be expressed from a DNA sequence comprising SEQ ID NO: 13. A variable region of SBT-040-G2 can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G2 expressed from SEQ ID NO: 7, or expressed from a DNA sequence comprising greater than 70% homology to SEQ ID NO: 7 can have a dissociation constant (K_{d}) for CD40 that is less than 10 nM. Anti-CD40 antibodies comprising SBT-040-G2 expressed from DNA sequence comprising SEQ ID NO: 7, or comprising greater than 70% homology to SEQ ID NO: 7 can have a dissociation constant (K_{d}) for CD40 that is less than 1 nM, less than 100 pM, less than 10 pM, less than 1 pM, or less than 0.1 pM. SBT-040-G2 can be expressed with any anti-CD40 light chain or fragment thereof. SBT-040-G2 can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G2 can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVERKCCVECPPCPAPPVAGPSVFLFPPKPKDTLMISRTPEVTCVVVDVSHEDPEVQFNW YVDGVEVHNAKTKPREEQFNSTFRVVSVLTVVHQDWLNGKEYKCKVSNKGLPAPIEKTISK TKGQPREPQVYTLPPSREEMTKNQVSLTCLVKGFYPSDIAVEWESNGQPENNYKTTPPMLD SDGSFFLYSKLTVDKSRWQQGNVFSCSVMHEALHNHYTQKSLSLSPGK (SEQ ID NO: 14).

SBT-040-G2 can comprise an amino acid sequence with greater than 70%, greater than 75%, greater than 80%, greater than 85%, greater than 90%, greater than 95% or greater than 99% homology to SEQ ID NO: 14. SBT-040-G1WT can comprise an amino acid sequence SEQ ID NO: 20. A variable region of SBT-040-G2 can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G2 with SEQ ID NO: 14 or with an amino acid sequence with greater than 70% homology to SEQ ID NO: 14 can have a dissociation constant (K_{d}) for CD40 that is less than 10 nM. Anti-CD40 antibodies comprising SBT-040-G2 with SEQ ID NO: 14 or with an amino acid sequence with greater than 70% homology to SEQ ID NO: 14 can have a dissociation constant (K_{d}) for CD40 that is less than 1 nM, less than 100 pM, less than 10 pM, less than 1 pM, or less than 0.1 pM. SBT-040-G2 can be purified. SBT-040-G2 can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

An antibody construct can comprise an antibody with modifications occurring at least at one amino acid residue. Modifications can be substitutions, additions, mutations, deletions, or the like. An antibody modification can be an insertion of an unnatural amino acid.

An antibody construct can comprise a light chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An antibody construct can comprise a heavy chain of an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. A heavy chain can be the heavy chain of an anti-CD40 antibody which can bind to the CD40 antigen.

An antibody construct can be an IgG1 isotype. An antibody construct can be an IgG2 isotype. An antibody construct can be an IgG3 isotype. An antibody construct can be an IgG4 isotype. An antibody construct can be of a hybrid isotype comprising constant regions from two or more isotypes. An antibody construct can be an anti-CD40 antibody, in which the anti-CD40 antibody can be a monoclonal human antibody comprising a wild-type sequence of an IgG1 isoform, in particular, at an Fc region of the antibody.

Additional anti-CD40 antibody sequences that can be used in the antibody construct can comprise any sequence as shown below in TABLE 1 or combination thereof:

TABLE 1

| | SEQ | |
|---|---|---|
| Description of | ID | Sequence |
| Sequence | NO: | |
| Heavy Chain | 86 | ATGGAGTTTGGGCTGAGCTGGGTTTTCCTCGTTGCTCTTTTAAGAGGTG |
| DNA sequence | | TCCATGTCAGGTGCAGCTGGTGGAGTCTGGGGGAGGCGTGGTCCAGCC |
| of antibody 3.1.1 | | TGGGAGGTCCCTGAGACTCTCCTGTGCAGCCTCTGGATTCACCTTCAGT |
| | | AGTTATGGCATGCACTGGGTCCGCCAGGCTCCAGGCAAGGGGCTGGA |
| | | GTGGGTGGCAGTTATATCAAAGGATGGAGGTAATAAATACCATGCAG |
| | | ACTCCGTGAAGGGCCGATTCACCATCTCCAGAGACAATTCCAAGAATG |
| | | CGCTGTATCTGCAAATGAATAGCCTGAGAGTTGAAGACACGGCTGTGT |
| | | ATTACTGTGTGAGAAGAGGGCATCAGCTGGTTCTGGGATACTACTACT |
| | | ACAACGGTCTGGACGTCTGGGGCCAAGGGACCACGGTCACCGTCTCCT |
| | | CAGCCTCCACCAAGGGCCCATCGGTCTTCCCCCTGGCGCCCTGCTCCA |
| | | GGAGCACCTCCGAGAGCACAGCGGCCCTGGGCTGCCTGGTCAAGGAC |
| | | TACTTCCCCGAACCGGTGACGGTGTCGTGGAACTCAGGCGCTCTGACC |
| | | AGCGGCGTGCACACCTTCCCAGCTGTCCTACAGTCCTCAGGACTCTAC |
| | | TCCCTCAGCAGCGTGGTGACCGTGCCCTCCAGCAACTTCGGCACCCAG |
| | | ACCTACACCTGCAACGTAGATCACAAGCCCAGCAACACCAAGGTGGA |
| | | CAAGACAGTTGAGCGCAAATGTTGTGTCGAGTGCCCACCGTGCCCAGC |
| | | ACCACCTGTGGCAGGACCGTCAGTCTTCCTCTTCCCCCCAAAACCCAA |
| | | GGACACCCTCATGATCTCCCGGACCCCTGAGGTCACGTGCGTGGTGGT |
| | | GGACGTGAGCCACGAAGACCCCGAGGTCCAGTTCAACTGGTACGTGG |
| | | ACGGCGTGGAGGTGCATAATGCCAAGACAAAGCCACGGGAGGAGCAG |
| | | TTCAACAGCACGTTCCGTGTGGTCAGCGTCCTCACCGTTGTGCACCAG |
| | | GACTGGCTGAACGGCAAGGAGTACAAGTGCAAGGTCTCCAACAAAGG |
| | | CCTCCCAGCCCCCATCGAGAAAACCATCTCCAAAACCAAAGGGCAGC |
| | | CCCGAGAACCACAGGTGTACACCCTGCCCCCATCCCGGGAGGAGATG |
| | | ACCAAGAACCAGGTCAGCCTGACCTGCCTGGTCAAAGGCTTCTACCCC |
| | | AGCGACATCGCCGTGGAGTGGGAGAGCAATGGGCAGCCGGAGAACAA |
| | | CTACAAGACCACACCTCCCATGCTGGACTCCGACGGCTCCTTCTTCCTC |
| | | TACAGCAAGCTCACCGTGGACAAGAGCAGGTGGCAGCAGGGGAACGT |
| | | CTTCTCATGCTCCGTGATGCATGAGGCTCTGCACAACCACTACACGCA |
| | | GAAGAGCCTCTCCCTGTCTCCGGGTAAATGA |
| Heavy Chain | 87 | MEFGLSWVFLVALLRGVQCQVQLVESGGGVVQPGRSLRLSCAASGFTFS |
| protein sequence | | SYGMHWVRQAPGKGLEWVAVISKDGGNKYHADSVKGRFTISRDNSKNA |
| of Antibody | | LYLQMNSLRVEDTAVYYCVRRGHQLVLGYYYYNGLDVWGQGTTVTVS |
| 3.1.1 | | SASTKGPSVFPLAPCSRSTSESTAALGCLVKDYFPEPVTVSWNSGALTSGV |
| | | HTFPAVLQSSGLYSLSSVVTVPSSNFGTQTYTCNVDHKPSNTKVDKTVER |
| | | KCCVECPPCPAPPVAGPSVFLFPPKPKDTLMISRTPEVTCVVVDVSHEDPE |
| | | VQFNWYVDGVEVHNAKTKPREEQFNSTFRVVSVLTVVHQDWLNGKEYK |
| | | CKVSNKGLPAPIEKTISKTKGQPREPQVYTLPPSREEMTKNQVSLTCLVKG |
| | | FYPSDIAVEWESNGQPENNYKTTPPMLDSDGSFFLYSKLTVDKSRWQQG |
| | | NVFSCSVMHEALHNHYTQKSLSLSPGK |
| Light Chain | 88 | ATGAGGCTCCCTGCTCAGCTCCTGGGGCTGCTAATGCTCTGGGTCTCTG |
| DNA sequence | | GATCCAGTGGGGATATTGTGCTGACTCAGTCTCCACTCTCCCTGCCCGT |
| of Antibody | | CACCCCTGGAGAGCCGGCCTCCATCTCCTGCAGGTCTAGTCAGAGCCT |
| 3.1.1 | | CTTGTATAGTAATGGATACAACTTTTTGGATTGGTACCTGCAGAAGCC |
| | | AGGGCAGTCTCCACAGCTCCTGATCTATTTGGGTTCTAATCGGGCCTCC |
| | | GGGGTCCCTGACAGGTTCAGTGGCAGTGGATCAGGCACAGATTTTACA |
| | | CTGAAAATCAGCAGATTGGAGGCTGAGGATGTTGGGGTTTATTACTGC |
| | | ATGCAAGCTCTACAAACTCCTCGGACGTTCGGCCAAGGGACCAAGGTG |
| | | GAAATCAAACGAACTGTGGCTGCACCATCTGTCTTCATCTTCCCGCCA |
| | | TCTGATGAGCAGTTGAAATCTGGAACTGCCTCTGTTGTGTGCCTGCTGA |
| | | ATAACTTCTATCCCAGAGAGGCCAAAGTACAGTGGAAGGTGGATAAC |
| | | GCCCTCCAATCGGGTAACTCCCAGGAGAGTGTCACAGAGCAGGACAG |
| | | CAAGGACAGCACCTACAGCCTCAGCAGCACCCTGACGCTGAGCAAAG |
| | | CAGACTACGAGAAACACAAAGTCTACGCCTGCGAAGTCACCCATCAG |
| | | GGCCTGAGCTCGCCCGTCACAAAGGCTTCAACAGGGGAGAGTGTTAG |
| Light Chain | 89 | MRLPAQLLGLLMLWVSGSSGDIVLTQSPLSLPVTPGEPASISCRSSQSLLYS |
| protein sequence | | NGYNFLDWYLQKPGQSPQLLIYLGSNRASGVPDRFSGSGSGTDFTLKISRL |
| of Antibody | | EAEDVGVYYCMQALQTPRTFGQGTKVEIKRTVAAPSVFIFPPSDEQLKSG |
| 3.1.1 | | TASVVCLLNNFYPREAKVQWKVDNALQSGNSQESVTEQDSKDSTYSLSS |
| | | TLTLSKADYEKHKVYACEVTHQGLSSPVTKSFNRGEC |
| Mature Variable | 90 | CAGGTGCAGCTGGTGGAGTCTGGGGGAGGCGTGGTCCAGCCTGGGAG |
| Domain of | | GTCCCTGAGACTCTCCTGTGCAGCCTCTGGATTCACCTTCAGTAGTTAT |
| Heavy Chain | | GGCATGCACTGGGTCCGCCAGGCTCCAGGCAAGGGGCTGGAGTGGGT |
| DNA Sequence | | GGCAGTTATATCAAAGGATGGAGGTAATAAATACCATGCAGACTCCGT |
| of Antibody | | GAAGGGCCGATTCACCATCTCCAGAGACAATTCCAAGAATGCGCTGTA |
| 3.1.1 | | TCTGCAAATGAATAGCCTGAGAGTTGAAGACACGGCTGTGTATTACTG |
| | | TGTGAGAAGAGGGCATCAGCTGGTTCTGGGATACTACTACTACAACGG |
| | | TCTGGACGTCTGGGGCCAAGGGACCACGGTCACCGTCTCCTCA |
| Mature Variable | 91 | QVQLVESGGGVVQPGRSLRLSCAASGFTFSSYGMHWVRQAPGKGLEWV |
| Domain of | | AVISKDGGNKYHADSVKGRFTISRDNSKNALYLQMNSLRVEDTAVYYCV |
| Heavy Chain | | RRGHQLVLGYYYYNGLDVWGQGTTVTVSS |
| Protein | | |
| Sequence of | | |
| Antibody 3.1.1 | | |
| Mature Variable | 92 | GATATTGTGCTGACTCAGTCTCCACTCTCCCTGCCCGTCACCCCTGGAG |
| Domain of Light | | AGCCGGCCTCCATCTCCTGCAGGTCTAGTCAGAGCCTCTTGTATAGTA |
| Chain DNA | | ATGGATACAACTTTTTGGATTGGTACCTGCAGAAGCCAGGGCAGTCTC |
| Sequence of | | CACAGCTCCTGATCTATTTGGGTTCTAATCGGGCCTCCGGGGTCCCTGA |
| Antibody 3.1.1 | | CAGGTTCAGTGGCAGTGGATCAGGCACAGATTTTACACTGAAAATCAG |
| | | CAGATTGGAGGCTGAGGATGTTGGGGTTTATTACTGCATGCAAGCTCT |
| | | ACAAACTCCTCGGACGTTCGGCCAAGGGACCAAGGTGGAAATCAAA |
| Mature Variable | 93 | DIVLTQSPLSLPVTPGEPAAISCRSSQSLLYSNGYNFLDWYLQKPGQSPQL |
| Domain of Light | | LIYLGSNRASGVPDPYSGSGSGTDFTLKISRLEAEDVGVYYCMQALQTPR |
| Chain Protein | | TFGQGTKVEIK |
| Sequence of | | |
| Antibody 3.1.1 | | |
| Heavy Chain | 94 | CAGGTGCAGCTGGTGGAGTCTGGGGGAGGCGTGGTCCAGCCTGGGAG |
| DNA (variable | | GTCCCTGAGACTCTCCTGTGCAGCCTCTGGATTCACCTTCAGTAGTTAT |
| domain 3.1.1H- | | GGCATGCACTGGGTCCGCCAGGCTCCAGGCAAGGGGCTGGAGTGGGT |
| A78T) | | GGCAGTTATATCAAAGGATGGAGGTAATAAATACCATGCAGACTCCGT |
| | | GAAGGGCCGATTCACCATCTCCAGAGACAATTCCAAGAATACGCTGTT |
| | | CTGCAAATGAATAGCCTGAGAGTTGAAGACACGGCTGTGTATTACTGT |
| | | GTGAGAAGAGGGCATCAGCTGGTTCTGGGATACTACTACTACAACGGT |
| | | CTGGACGTCTGGGGCCAAGGGACCACGGTCACCGTCTCCTCA |
| Heavy Chain | 95 | QVQLVESGGGVVQPGRSLRLSCAASGFTFSSYGMHWVRQAPGKGLEWV |
| Protein (variable | | AVISKDGGNKYHADSVKGRFTISRDNSKNTLYLQMNSLRVEDTAVYYCV |
| domain 3.1.1H- | | RRGHQLVLGYYYYNGLDVWGQGTTVTVSS |
| A78T) | | |
| Heavy Chain | 96 | CAGGTGCAGCTGGTGGAGTCTGGGGGAGGCGTGGTCCAGCCTGGGAG |
| DNA (variable | | GTCCCTGAGACTCTCCTGTGCAGCCTCTGGATTCACCTTCAGTAGTTAT |
| domain 3.1.1H- | | GGCATGCACTGGGTCCGCCAGGCTCCAGGCAAGGGGCTGGAGTGGGT |
| A78T-V88A- | | GGCAGTTATATCAAAGGATGGAGGTAATAAATACCATGCAGACTCCGT |
| V97A) | | GAAGGGCCGATTCACCATCTCCAGAGACAATTCCAAGAATACGCTGTA |
| | | TCTGCAAATGAATAGCCTGAGAGcTGAAGACACGGCTGTGTATTACTG |
| | | TGCGAGAAGAGGGCATCAGCTGGTTCTGGGATACTACTACTACAACGG |
| | | TCTGGACGTCTGGGGCCAAGGGACCACGGTCACCGTCTCCTCA |
| Heavy Chain | 97 | QVQLVESGGGVVQPGRSLRLSCAASGFTFSSYGMHWVRQAPGKGLEWV |
| Protein (variable | | AVISKDGGNKYHADSVKGRFTISRDNSKNTLYLQMNSLRAEDTAVYYCA |
| domain 3.1.1H- | | RRGHQLVLGYYYYNGLDVWGQGTTVTVSS |
| A78T-V88A- | | |
| V97A) | | |
| Light Chain | 98 | GATATTGTGaTGACTCAGTCTCCACTCTCCCTGCCCGTCACCCCTGGAG |
| DNA (variable | | AGCCGGCCTCCATCTCCTGCAGGTCTAGTCAGAGCCTCTTGTATAGTA |
| domain 3.1.1L- | | ATGGATACAACTTTTTGGATTGGTACCTGCAGAAGCCAGGGCAGTCTC |
| L4M-L83V) | | CACAGCTCCTGATCTATTTGGGTTCTAATCGGGCCTCCGGGGTCCCTGA |
| | | CAGGTTCAGTGGCAGTGGATCAGGCACAGATTTTACACTGAAAATCAG |
| | | CAGAGTGGAGGCTGAGGATGTTGGGGTTTATTACTGCATGCAAGCTCT |
| | | ACAAACTCCTCGGACGTTCGGCCAAGGGACCAAGGTGGAAATCAAA |
| Light Chain | 99 | DIVMTQSPLSLPVTPGEPASISCRSSQSLLYSNGYNFLDWYLQKPGQSPQL |
| Protein (variable | | LIYLGSNRASGVPDRFSGSGSGTDFTLKISRVEAEDVGVYYCMQALQTPR |
| domain 3.1.1L- | | TFGQGTKVEIK |
| L4M-L83V) | | |
| Heavy Chain | 100 | ATGGAGTTTGGGCTGAGCTGGGTTTTCCTCGTTGCTCTTTTAAGAGGTG |
| DNA Sequence | | TCCAGTGTCAGGTGCAGCTGGTGGAGTCTGGGGGAGGCGTGGTCCAGC |
| for Antibody | | CTGGGAGGTCCCTGAGACTCTCCTGTGCAGCCTCTGGATTCACCTTCA |
| 7.1.2 | | GTAGCTATGGCATGCACTGGGTCCGCCAGGCTCCAGGCAAGGGGCTG |
| | | GAGTGGGTGGCAGTTATATCAAATGATGGAGATAATAAATACCATGCA |
| | | GACTCCGTGTGGGGCCGATTCACCATCTCCAGAGACAATTCCAGGAGC |
| | | ACGCTTTATCTGCAAATGAACAGCCTGAGAGCTGAGGACACGGCTGTA |
| | | TATTACTGTGCGAGAAGAGGCATGGGGTCTAGTGGGAGCCGTGGGGA |
| | | TTACTACTACTACTACGGTTTGGACGTCTGGGGCCAAGGGACCACGGT |
| | | CACCGTCTCCTCAGCCTCCACCAAGGGCCCATCGGTCTTCCCCCTGGC |
| | | GCCCTGCTCCAGGAGCACCTCCGAGAGCACAGCGGCCCTGGGCTGCCT |
| | | GGTCAAGGACTACTTCCCCGAACCGGTGACGGTGTCGTGGAACTCAGG |
| | | CGCTCTGACCAGCGGCGTGCACACCTTCCCAGCTGTCCTACAGTCCTC |
| | | AGGACTCTACTCCCTCAGCAGCGTGGTGACCGTGCCCTCCAGCAACTT |
| | | CGGCACCCAGACCTACACCTGCAACGTAGATCACAAGCCCAGCAACA |
| | | CCAAGGTGGACAAGACAGTTGAGCGCAAATGTTGTGTCGAGTGCCCA |
| | | CCGTGCCCAGCACCACCTGTGGCAGGACCGTCAGTCTTCCTCTTCCCCC |
| | | CAAAACCCAAGGACACCCTCATGATCTCCCGGACCCCTGAGGTCACGT |
| | | GCGTGGTGGTGGACGTGAGCCACGAAGACCCCGAGGTCCAGTTCAAC |
| | | TGGTACGTGGACGGCGTGGAGGTGCATAATGCCAAGACAAAGCCACG |
| | | GGAGGAGCAGTTCAACAGCACGTTCCGTGTGGTCAGCGTCCTCACCGT |
| | | TGTGCACCAGGACTGGCTGAACGGCAAGGAGTACAAGTGCAAGGTCT |
| | | CCAACAAAGGCCTCCCAGCCCCCATCGAGAAAACCATCTCCAAAACC |
| | | AAAGGGCAGCCCCGAGAACCACAGGTGTACACCCTGCCCCCATCCCG |
| | | GGAGGAGATGACCAAGAACCAGGTCAGCCTGACCTGCCTGGTCAAAG |
| | | GCTTCTACCCCAGCGACATCGCCGTGGAGTGGGAGAGCAATGGGCAG |
| | | CCGGAGAACAACTACAAGACCACACCTCCCATGCTGGACTCCGACGG |
| | | CTCCTTCTTCCTCTACAGCAAGCTCACCGTGGACAAGAGCAGGTGGCA |
| | | GCAGGGGAACGTCTTCTCATGCTCCGTGATGCATGAGGCTCTGCACAA |
| | | CCACTACACGCAGAAGAGCCTCTCCCTGTCTCCGGGTAAATGA |
| Heavy Chain | 101 | MEFGLSWVFLVALLRGVQCQVQLVESGGGVVQPGRSLRLSCAASGFTFS |
| Protein | | SYGMHWVRQAPGKGLEWVAVISNDGDNKYHADSVWGRFTISRDNSRST |
| Sequence for | | LYLQMNSLRAEDTAVYYCARRGMGSSGSRGDYYYYYGLDVWGQGTTV |
| Antibody 7.1.2 | | TVSSASTKGPSVFPLAPCSRSTSESTAALGCLVKDYFPEPVTVSWNSGALT |
| | | SGVHTFPAVLQSSGLYSLSSVVTVPSSNFGTQTYTCNVDHKPSNTKVDKT |
| | | VERKCCVECPPCPAPPVAGPSVFLFPPKPKDTLMISRTPEVTCVVVDVSHE |
| | | DPEVQFNWYVDGVEVHNAKTKPREEQFNSTFRVVSVLTVVHQDWLNGK |
| | | EYKCKVSNKGLPAPIEKTISKTKGQPREPQVYTLPPSREEMTKNQVSLTCL |
| | | VKGFYPSDIAVEWESNGQPENNYKTTPPMLDSDGSFFLYSKLTVDKSRW |
| | | QQGNVFSCSVMHEALHNHYTQKSLSLSPGK |
| Light Chain | 102 | ATGAGGCTCCCTGCTCAGCTCCTGGGGCTGCTAATGCTCTGGGTCTCTG |
| DNA Sequence | | GATCCAGTGGGGATATTGTGATGACTCAGTCTCCACTCTCCCTGCCCGT |
| for Antibody | | CACCCCTGGAGAGCCGGCCTCCATCTCCTGCAGGTCTAGTCAGAGCCT |
| 7.1.2 | | CTTGTATAGTAATGGATACAACTTTTTGGATTGGTACCTGCAGAAGCC |
| | | AGGGCAGTCTCCACAGCTCCTGATCTATTTGGGTTCTAATCGGGCCTCC |
| | | GGGGTCCCTGACAGGTTCAGTGGCAGTGGATCAGGCACAGATTTTACA |
| | | CTGAAAATCAGCAGAGTGGAGGCTGAGGATGTTGGGGTTTATTACTGC |
| | | ATGCAAGCTCTACAAACTCCTCGGACGTTCGGCCAAGGGACCAAGGTG |
| | | GAAATCAAACGAACTGTGGCTGCACCATCTGTCTTCATCTTCCCGCCA |
| | | TCTGATGAGCAGTTGAAATCTGGAACTGCCTCTGTTGTGTGCCTGCTGA |
| | | ATAACTTCTATCCCAGAGAGGCCAAAGTACAGTGGAAGGTGGATAAC |
| | | GCCCTCCAATCGGGTAACTCCCAGGAGAGTGTCACAGAGCAGGACAG |
| | | CAAGGACAGCACCTACAGCCTCAGCAGCACCCTGACGCTGAGCAAAG |
| | | CAGACTACGAGAAACACAAAGTCTACGCCTGCGAAGTCACCCATCAG |
| | | GGCCTGAGCTCGCCCGTCACAAAGAGCTTCAACAGGGGAGAGTGTTA |
| | | G |
| Light Chain | 103 | MRLPAQLLGLLMLWVSGSSGDIVMTQSPLSLPVTPGEPASISCRSSQSLLY |
| Protein | | SNGYNFLDWYLQKPGQSPQLLIYLGSNRASGVPDRFSGSGSGTDFTKISRV |
| Sequence for | | EAEDVGVYYCMQALQTPRTFGQGTKVEIKRTVAAPSVFIFPPSDEQLKSG |
| Antibody 7.1.2 | | TASVVCLLNNFYPREAKVQWKVDNALQSGNSQESVTEQDSKDSTYSLSS |
| | | TLTLSKADYEKHKVYACEVTHQGLSSPVTKSFNRGEC |
| Mature Variable | 104 | CAGGTGCAGCTGGTGGAGTCTGGGGGAGGCGTGGTCCAGCCTGGGAG |
| Domain of | | GTCCCTGAGACTCTCCTGTGCAGCCTCTGGATTCACCTTCAGTAGCTAT |
| Heavy Chain | | GGCATGCACTGGGTCCGCCAGGCTCCAGGCAAGGGGCTGGAGTGGGT |
| DNA Sequence | | GGCAGTTATATCAAATGATGGAGATAATAAATACCATGCAGACTCCGT |
| of Antibody | | GTGGGGCCGATTCACCATCTCCAGAGACAATTCCAGGAGCACGCTTTA |
| 7.1.2 | | TCTGCAAATGAACAGCCTGAGAGCTGAGGACACGGCTGTATATTACTG |
| | | TGCGAGAAGAGGCATGGGGTCTAGTGGGAGCCGTGGGGATTACTACT |
| | | ACTACTACGGTTTGGACGTCTGGGGCCAAGGGACCACGGTCACCGTCT |
| | | CCTCA |
| Mature Variable | 105 | QVQLVESGGGVVQPGRSLRLSCAASGFTFSSYGMHWVRQAPGKGLEWV |
| Domain of | | AVISNDGDNKYHADSVWGRFTISRDNSRSTLYLQMNSLRAEDTAVYYCA |
| Heavy Chain | | RRGMGSSGSRGDYYYYYGLDVWGQGTTVTVSS |
| Protein | | |
| Sequence of | | |
| Antibody 7.1.2 | | |
| Mature Variable | 106 | GATATTGTGATGACTCAGTCTCCACTCTCCCTGCCCGTCACCCCTGGAG |
| Domain of Light | | AGCCGGCCTCCATCTCCTGCAGGTCTAGTCAGAGCCTCTTGTATAGTA |
| Chain DNA | | ATGGATACAACTTTTTGGATTGGTACCTGCAGAAGCCAGGGCAGTCTC |
| Sequence of | | CACAGCTCCTGATCTATTTGGGTTCTAATCGGGCCTCCGGGGTCCCTGA |
| Antibody 7.1.2 | | CAGGTTCAGTGGCAGTGGATCAGGCACAGATTTTACACTGAAAATCAG |
| | | CAGAGTGGAGGCTGAGGATGTTGGGGTTTATTACTGCATGCAAGCTCT |
| | | ACAAACTCCTCGGACGTTCGGCCAAGGGACCAAGGTGGAAATCAAA |
| Mature Variable | 107 | DIVMTQSPLSLPVTPGEPASISCRSSQSLLYSNGYNFLDWYLQKPGQSPQL |
| Domain of Light | | LIYLGSNRASGVPDRFSGSGSGTDFTLKISRVEAEDVGVYYCMQALQTPR |
| Chain Protein | | TFGQGTKVEIK |
| Sequence of | | |
| Antibody 7.1.2 | | |
| Heavy Chain | 108 | ATGAAACACCTGTGGTTCTTCCTCCTGCTGGTGGCAGCTCCCAGATGG |
| DNA Sequence | | GTCCTGTCCCAGGTGCAGCTGCAGGAGTCGGGCCCAGGACTGGTGAA |
| for Antibody | | GCCTTCGGAGACCCTGTCCCTCACCTGCACTGTCTCTGGTGGCTCCATC |
| 10.8.3 | | AGTAGTTACTACTGGATCTGGATCCGGCAGCCCGCCGGGAAGGGACTG |
| | | GAATGGATTGGGCGTGTCTATACCAGTGGGAGCACCAACTACAACCCC |
| | | TCCCTCAAGAGTCGAGTCACCATGTCAGTAGACACGTCCAAGAACCAG |
| | | TTCTCCCTGAAGCTGAGCTCTGTGACCGCCGCGGACACGGCCGTGTAT |
| | | TACTGTGCGAGAGATGGTCTTTACAGGGGGTACGGTATGGACGTCTGG |
| | | GGCCAAGGGACCACGGTCACCGTCTCCTCAGCCTCCACCAAGGGCCCA |
| | | TCGGTCTTCCCCCTGGCGCCCTGCTCCAGGAGCACCTCCGAGAGCACA |
| | | GCGGCCCTGGGCTGCCTGGTCAAGGACTACTTCCCCGAACCGGTGACG |
| | | GTGTCGTGGAACTCAGGCGCTCTGACCAGCGGCGTGCACACCTTCCCA |
| | | GCTGTCCTACAGTCCTCAGGACTCTACTCCCTCAGCAGCGTGGTGACC |
| | | GTGCCCTCCAGCAACTTCGGCACCCAGACCTACACCTGCAACGTAGAT |
| | | CACAAGCCCAGCAACACCAAGGTGGACAAGACAGTTGAGCGCAAATG |
| | | TTGTGTCGAGTGCCCACCGTGCCCAGCACCACCTGTGGCAGGACCGTC |
| | | AGTCTTCCTCTTCCCCCCAAAACCCAAGGACACCCTCATGATCTCCCG |
| | | GACCCCTGAGGTCACGTGCGTGGTGGTGGACGTGAGCCACGAAGACC |
| | | CCGAGGTCCAGTTCAACTGGTACGTGGACGGCGTGGAGGTGCATAATG |
| | | CCAAGACAAAGCCACGGGAGGAGCAGTTCAACAGCACGTTCCGTGTG |
| | | GTCAGCGTCCTCACCGTTGTGCACCAGGACTGGCTGAACGGCAAGGAG |
| | | TACAAGTGCAAGGTCTCCAACAAAGGCCTCCCAGCCCCCATCGAGAA |
| | | AACCATCTCCAAAACCAAAGGGCAGCCCCGAGAACCACAGGTGTACA |
| | | CCCTGCCCCCATCCCGGGAGGAGATGACCAAGAACCAGGTCAGCCTG |
| | | ACCTGCCTGGTCAAAGGCTTCTACCCCAGCGACATCGCCGTGGAGTGG |
| | | GAGAGCAATGGGCAGCCGGAGAACAACTACAAGACCACACCTCCCAT |
| | | GCTGGACTCCGACGGCTCCTTCTTCCTCTACAGCAAGCTCACCGTGGA |
| | | CAAGAGCAGGTGGCAGCAGGGGAACGTCTTCTCATGCTCCGTGATGCA |
| | | TGAGGCTCTGCACAACCACTACACGCAGAAGAGCCTCTCCCTGTCTCC |
| | | GGGTAAATGA |
| Heavy Chain | 109 | MKHLWFFLLLVAAPRWVLSQVQLQESGPGLVKPSETLSLTCTVSGGSISS |
| Protein | | YYWIWIRQPAGKGLEWIGRVYTSGSTNYNPSLKSRVTMSVDTSKNQFSL |
| Sequence for | | KLSSVTAADTAVYYCARDGLYRGYGMDVWGQGTTVTVSSASTKGPSVF |
| Antibody 10.8.3 | | PLAPCSRSTSESTAALGCLVKDYFPEPVTVSWNSGALTSGVHTFPAVLQSS |
| | | GLYSLSSVVTVPSSNFGTQTYTCNVDHKPSNTKVDKTVERKCCVECPPCP |
| | | APPVAGPSVFLFPPKPKDTLMISRTPEVTCVVVDVSHEDPEVQFNWYVDG |
| | | VEVHNAKTKPREEQFNSTFRVVSVLTVVHQDWLNGKEYKCKVSNKGLP |
| | | APIEKTISKTKGQPREPQVYTLPPSREEMTKNQVSLTCLVKGFYPSDIAVE |
| | | WESNGQPENNYKTTPPMLDSDGSFFLYSKLTVDKSRWQQGNVFSCSVMH |
| | | EALHNHYTQKSLSLSPGK |
| Light Chain | 110 | ATGAGGCTCCCTGCTCAGCTCCTGGGGCTCCTGCTGCTCTGGTTCCCAG |
| DNA Sequence | | GTTCCAGATGCGACATCCAGATGACCCAGTCTCCATCTTCCGTGTCTGC |
| for Antibody | | ATCTGTAGGAGACAGAGTCACCATCACTTGTCGGGCGAGTCAGCCTAT |
| 10.8.3 | | TAGCAGCTGGTTAGCCTGGTATCAGCAGAAACCAGGGAAAGCCCCTA |
| | | AACTCCTGATTTATTCTGCCTCCGGTTTGCAAAGTGGGGTCCCATCAAG |
| | | GTTCAGCGGCAGTGGATCTGGGACAGATTTCACTCTCACCATCAGCAG |
| | | CCTGCAGCCTGAAGATTTTGCAACTTACTATTGTCAACAGACTGACAG |
| | | TTTCCCGCTCACTTTCGGCGGCGGGACCAAGGTGGAGATCAAACGAAC |
| | | TGTGGCTGCACCATCTGTCTTCATCTTCCCGCCATCTGATGAGCAGTTG |
| | | AAATCTGGAACTGCCTCTGTTGTGTGCCTGCTGAATAACTTCTATCCCA |
| | | GAGAGGCCAAAGTACAGTGGAAGGTGGATAACGCCCTCCAATCGGGT |
| | | AACTCCCAGGAGAGTGTCACAGAGCAGGACAGCAAGGACAGCACCTA |
| | | CAGCCTCAGCAGCACCCTGACGCTGAGCAAAGCAGACTACGAGAAAC |
| | | ACAAAGTCTACGCCTGCGAAGTCACCCATCAGGGCCTGAGCTCGCCCG |
| | | TCACAAAGAGCTTCAACAGGGGAGAGTGTTAG |
| Light Chain | 111 | MRLPAQLLGLLLLWFPGSRCDIQMTQSPSSVSASVGDRVTITCRASQPISS |
| Protein | | WLAWYQQKPGKAPKLLIYSASGLQSGVPSRFSGSGSGTDFTLTISSLQPED |
| Sequence for | | FATYYCQQTDSFPLTFGGGTKVEIKRTVAAPSVFIFPPSDEQLKSGTASVV |
| Antibody 10.8.3 | | CLLNNFYPREAKVQWKVDNALQSGNSQESVTEQDSKDSTYSLSSTLTLSK |
| | | ADYEKHKVYACEVTHQGLSSPVTKSFNRGEC |
| Mature Variable | 112 | CAGGTGCAGCTGCAGGAGTCGGGCCCAGGACTGGTGAAGCCTTCGGA |
| Domain of | | GACCCTGTCCCTCACCTGCACTGTCTCTGGTGGCTCCATCAGTAGTTAC |
| Heavy Chain | | TACTGGATCTGGATCCGGCAGCCCGCCGGGAAGGGACTGGAATGGAT |
| DNA Sequence | | TGGGCGTGTCTATACCAGTGGGAGCACCAACTACAACCCCTCCCTCAA |
| for Antibody | | GAGTCGAGTCACCATGTCAGTAGACACGTCCAAGAACCAGTTCTCCCT |
| 10.8.3 | | GAAGCTGAGCTCTGTGACCGCCGCGGACACGGCCGTGTATTACTGTGC |
| | | GAGAGATGGTCTTTACAGGGGGTACGGTATGGACGTCTGGGGCCAAG |
| | | GGACCACGGTCACCGTCTCCTCA |
| Mature Variable | 113 | QVQLQESGPGLVKPSETLSLTCTVSGGSISSYYWIWIRQPAGKGLEWIGRV |
| Domain of | | YTSGSTNYNPSLKSRVTMSVDTSKNQFSLKLSSVTAADTAVYYCARDGL |
| Heavy Chain | | YRGYGMDVWGQGTTVTVSS |
| Protein | | |
| Sequence for | | |
| Antibody 10.8.3 | | |
| Mature Variable | 114 | GACATCCAGATGACCCAGTCTCCATCTTCCGTGTCTGCATCTGTAGGA |
| Domain of Light | | GACAGAGTCACCATCACTTGTCGGGCGAGTCAGCCTATTAGCAGCTGG |
| Chain DNA | | TTAGCCTGGTATCAGCAGAAACCAGGGAAAGCCCCTAAACTCCTGATT |
| Sequence for | | TATTCTGCCTCCGGTTTGCAAAGTGGGGTCCCATCAAGGTTCAGCGGC |
| Antibody 10.8.3 | | AGTGGATCTGGGACAGATTTCACTCTCACCATCAGCAGCCTGCAGCCT |
| | | GAAGATTTTGCAACTTACTATTGTCAACAGACTGACAGTTTCCCGCTCA |
| | | CTTTCGGCGGCGGGACCAAGGTGGAGATCAAA |
| Mature Variable | 115 | DIQMTQSPSSVSASVGDRVTITCRASQPISSWLAWYQQKPGKAPKLLIYSA |
| Domain of Light | | SGLQSGVPSRFSGSGSGTDFTLTISSLQPEDFATYYCQQTDSFPLTFGGGTK |
| Chain Protein | | VEIK |
| Sequence for | | |
| Antibody 10.8.3 | | |
| Heavy Chain | 116 | ATGAAACATCTGTGGTTCTTCCTTCTCCTGGTGGCAGCTCCCAGATGGG |
| DNA Sequence | | TCCTGTCCCAGGTGCAGCTGCAGGAGTCGGGCCCAGGACTGGTGAAGC |
| for Antibody | | CTTCGGAGACCCTGTCCCTCACCTGCACTGTCTCTGGTGGCTCCATCAG |
| 15.1.1 | | AAGTTACTACTGGACCTGGATCCGGCAGCCCCCAGGGAAGGGACTGG |
| | | AGTGGATTGGATATATCTATTACAGTGGGAGCACCAACTACAATCCCT |
| | | CCCTCAAGAGTCGAGTCACCATATCAGTAGACATGTCCAAGAACCAGT |
| | | TCTCCCTGAAGCTGAGTTCTGTGACCGCTGCGGACACGGCCGTTTATT |
| | | ACTGTGCGAGAAAGGGTGACTACGGTGGTAATTTTAACTACTTTCACC |
| | | AGTGGGGCCAGGGAACCCTGGTCACCGTCTCCTCAGCCTCCACCAAGG |
| | | GCCCATCGGTCTTCCCCCTGGCGCCCTGCTCCAGGAGCACCTCCGAGA |
| | | GCACAGCGGCCCTGGGCTGCCTGGTCAAGGACTACTTCCCCGAACCGG |
| | | TGACGGTGTCGTGGAACTCAGGCGCTCTGACCAGCGGCGTGCACACCT |
| | | TCCCAGCTGTCCTACAGTCCTCAGGACTCTACTCCCTCAGCAGCGTGGT |
| | | GACCGTGCCCTCCAGCAACTTCGGCACCCAGACCTACACCTGCAACGT |
| | | AGATCACAAGCCCAGCAACACCAAGGTGGACAAGACAGTTGAGCGCA |
| | | AATGTTGTGTCGAGTGCCCACCGTGCCCAGCACCACCTGTGGCAGGAC |
| | | CGTCAGTCTTCCTCTTCCCCCCAAAACCCAAGGACACCCTCATGATCTC |
| | | CCGGACCCCTGAGGTCACGTGCGTGGTGGTGGACGTGAGTCACGAAG |
| | | ACCCCGAGGTCCAGTTCAACTGGTACGTGGACGGCGTGGAGGTGCATA |
| | | ATGCCAAGACAAAGCCACGGGAGGAGCAGTTCAACAGCACGTTCCGT |
| | | GTGGTCAGCGTCCTCACCGTTGTGCACCAGGACTGGCTGAACGGCAAG |
| | | GAGTACAAGTGCAAGGTCTCCAACAAAGGCCTCCCAGCCCCCATCGA |
| | | GAAAACCATCTCCAAAACCAAAGGGCAGCCCCGAGAACCACAGGTGT |
| | | ACACCCTGCCCCCATCCCGGGAGGAGATGACCAAGAACCAGGTCAGC |
| | | CTGACCTGCCTGGTCAAAGGCTTCTACCCCAGCGACATCGCCGTGGAG |
| | | TGGGAGAGCAATGGGCAGCCGGAGAACAACTACAAGACCACACCTCC |
| | | CATGCTGGACTCCGACGGCTCCTTCTTCCTCTACAGCAAGCTCACCGTG |
| | | GACAAGAGCAGGTGGCAGCAGGGGAACGTCTTCTCATGCTCCGTGAT |
| | | GCATGAGGCTCTGCACAACCACTACACGCAGAAGAGCCTCTCCCTGTC |
| | | TCCGGGTAAATGA |
| Heavy Chain | 117 | MKHLWFFLLLVAAPRWVLSQVQLQESGPGLVKPSETLSLTCTVSGGSIRS |
| Protein | | YYWTWIRQPPGKGLEWIGYIYYSGSTNYNPSLKSRVTISVDMSKNQFSLK |
| Sequence for | | LSSVTAADTAVYYCARKGDYGGNFNYFHQWGQGTLVTVSSASTKGPSV |
| Antibody 15.1.1 | | FPLAPCSRSTSESTAALGCLVKDYFPEPVTVSWNSGALTSGVHTFPAVLQS |
| | | SGLYSLSSVVTVPSSNFGTQTYTCNVDHKPSNTKVDKTVERKCCVECPPC |
| | | PAPPVAGPSVFLFPPKPKDTLMISRTPEVTCVVVDVSHEDPEVQFNWYVD |
| | | GVEVHNAKTKPREEQFNSTFRVVSVLTVVHQDWLNGKEYKCKVSNKGL |
| | | PAPIEKTISKTKGQPREPQVYTLPPSREEMTKNQVSLTCLVKGFYPSDIAVE |
| | | WESNGQPENNYKTTPPMLDSDGSFFLYSKLTVDKSRWQQGNVFSCSVMH |
| | | EALHNHYTQKSLSLSPGK |
| Light Chain | 118 | ATGAGGCTCCCTGCTCAGCTCCTGGGGCTGCTAATGCTCTGGGTCTCTG |
| DNA Sequence | | GATCCAGTGGGGATATTGTGATGACTCAGTCTCCACTCTCCCTGCCCGT |
| for Antibody | | CACCCCTGGAGAGCCGGCCTCCATCTCCTGCAGGTCTAGTCAGAGCCT |
| 15.1.1 | | CCTACATACTAATGGATACAACTATTTCGATTGGTACCTGCAGAAGCC |
| | | AGGGCAGTCTCCACAACTCCTGATCTATTTGGGTTCTAATCGGGCCTCC |
| | | GGGGTCCCTGACAGGTTCAGTGGCAGTGGATCAGGCACAGATTTTACA |
| | | CTGAAAATCAGCAGAGTGGAGGCTGAGGATGTTGGGGTTTATTACTGC |
| | | ATGCAAGCTCTACAAACTCCGTACAGTTTTGGCCAGGGGACCAAGCTG |
| | | GAGATCAAACGAACTGTGGCTGCACCATCTGTCTTCATCTTCCCGCCA |
| | | TCTGATGAGCAGTTGAAATCTGGAACTGCCTCTGTTGTGTGCCTGCTGA |
| | | ATAACTTCTATCCCAGAGAGGCCAAAGTACAGTGGAAGGTGGATAAC |
| | | GCCCTCCAATCGGGTAACTCCCAGGAGAGTGTCACAGAGCAGGACAG |
| | | CAAGGACAGCACCTACAGCCTCAGCAGCACCCTGACGCTGAGCAAAG |
| | | CAGACTACGAGAAACACAAAGTCTACGCCTGCGAAGTCACCCATCAG |
| | | GGCCTGAGCTCGCCCGTCACAAAGAGCTTCAACAGGGGAGAGTGTTA |
| | | G |
| Light Chain | 119 | MRLPAQLLGLLMLWVSGSSGDIVMTQSPLSLPVTPGEPASISCRSSQSLLH |
| Protein | | TNGYNYFDWYLQKPGQSPQLLIYLGSNRASGVPDRFSGSGSGTDFTLKIS |
| Sequence for | | RVEAEDVGVYYCMQALQTPYSFGQGTKLEIKRTVAAPSVFIFPPSDEQLK |
| Antibody 15.1.1 | | SGTASVVCLLNNFYPREAKVQWKVDNALQSGNSQESVTEQDSKDSTYSL |
| | | SSTLTLSKADYEKHKVYACEVTHQGLSSPVTKSFNRGEC |
| Mature Variable | 120 | CAGGTGCAGCTGCAGGAGTCGGGCCCAGGACTGGTGAAGCCTTCGGA |
| Domain of | | GACCCTGTCCCTCACCTGCACTGTCTCTGGTGGCTCCATCAGAAGTTAC |
| Heavy Chain | | TACTGGACCTGGATCCGGCAGCCCCCAGGGAAGGGACTGGAGTGGAT |
| DNA Sequence | | TGGATATATCTATTACAGTGGGAGCACCAACTACAATCCCTCCCTCAA |
| for Antibody | | GAGTCGAGTCACCATATCAGTAGACATGTCCAAGAACCAGTTCTCCCT |
| 15.1.1 | | GAAGCTGAGTTCTGTGACCGCTGCGGACACGGCCGTTTATTACTGTGC |
| | | GAGAAAGGGTGACTACGGTGGTAATTTTAACTACTTTCACCAGTGGGG |
| | | CCAGGGAACCCTGGTCACCGTCTCCTCA |
| Mature Variable | 121 | QVQLQESGPGLVKPSETLSLTCTVSGGSIRSYYWTWIRQPPGKGLEWIGYI |
| Domain of | | YYSGSTNYNPSLKSRVTISVDMSKNQFSLKLSSVTAADTAVYYCARKGD |
| Heavy Chain | | YGGNFNYFHQWGQGTLVTVSS |
| Protein | | |
| Sequence for | | |
| Antibody 15.1.1 | | |
| Mature Variable | 122 | GATATTGTGATGACTCAGTCTCCACTCTCCCTGCCCGTCACCCCTGGAG |
| Domain of Light | | AGCCGGCCTCCATCTCCTGCAGGTCTAGTCAGAGCCTCCTACATACTA |
| Chain DNA | | ATGGATACAACTATTTCGATTGGTACCTGCAGAAGCCAGGGCAGTCTC |
| Sequence for | | CACAACTCCTGATCTATTTGGGTTCTAATCGGGCCTCCGGGGTCCCTGA |
| Antibody 15.1.1 | | CAGGTTCAGTGGCAGTGGATCAGGCACAGATTTTACACTGAAAATCAG |
| | | CAGAGTGGAGGCTGAGGATGTTGGGGTTTATTACTGCATGCAAGCTCT |
| | | ACAAACTCCGTACAGTTTTGGCCAGGGGACCAAGCTGGAGATCAAA |
| Mature Variable | 123 | DIVMTQSPLSLPVTPGEPASISCRSSQSLLHTNGYNYFDWYLQKPGQSPQL |
| Domain of Light | | LIYLGSNRASGVPDRFSGSGSGTDFTLKISRVEAEDVGVYYCMQALQTPY |
| Chain Protein | | SFGQGTKLEIK |
| Sequence for | | |
| Antibody 15.1.1 | | |
| Heavy Chain | 124 | ATGGACTGGACCTGGAGGATCCTCTTCTTGGTGGCAGCAGCCACAGGA |
| DNA Sequence | | GCCCACTCCCAGGTGCAGCTGGTGCAGTCTGGGGCTGAGGTGAAGAA |
| for Antibody | | GCCTGGGGCCTCAGTGAAGGTCTCCTGCAAGGCTTCTGGATACACCTT |
| 21.4.1 | | CACCGGCTACTATATGCACTGGGTGCGACAGGCCCCTGGACAAGGGCT |
| | | TGAGTGGATGGGATGGATCAACCCTGACAGTGGTGGCACAAACTATGC |
| | | ACAGAAGTTTCAGGGCAGGGTCACCATGACCAGGGACACGTCCATCA |
| | | GCACAGCCTACATGGAGCTGAACAGGCTGAGATCTGACGACACGGCC |
| | | GTGTATTACTGTGCGAGAGATCAGCCCCTAGGATATTGTACTAATGGT |
| | | GTATGCTCCTACTTTGACTACTGGGGCCAGGGAACCCTGGTCACCGTC |
| | | TCCTCAGCCTCCACCAAGGGCCCATCGGTCTTCCCCCTGGCGCCCTGCT |
| | | CCAGGAGCACCTCCGAGAGCACAGCGGCCCTGGGCTGCCTGGTCAAG |
| | | GACTACTTCCCCGAACCGGTGACGGTGTCGTGGAACTCAGGCGCTCTG |
| | | ACCAGCGGCGTGCACACCTTCCCAGCTGTCCTACAGTCCTCAGGACTC |
| | | TACTCCCTCAGCAGCGTGGTGACCGTGCCCTCCAGCAACTTCGGCACC |
| | | CAGACCTACACCTGCAACGTAGATCACAAGCCCAGCAACACCAAGGT |
| | | GGACAAGACAGTTGAGCGCAAATGTTGTGTCGAGTGCCCACCGTGCCC |
| | | AGCACCACCTGTGGCAGGACCGTCAGTCTTCCTCTTCCCCCCAAAACC |
| | | CAAGGACACCCTCATGATCTCCCGGACCCCTGAGGTCACGTGCGTGGT |
| | | GGTGGACGTGAGCCACGAAGACCCCGAGGTCCAGTTCAACTGGTACG |
| | | TGGACGGCGTGGAGGTGCATAATGCCAAGACAAAGCCACGGGAGGAG |
| | | CAGTTCAACAGCACGTTCCGTGTGGTCAGCGTCCTCACCGTTGTGCAC |
| | | CAGGACTGGCTGAACGGCAAGGAGTACAAGTGCAAGGTCTCCAACAA |
| | | AGGCCTCCCAGCCCCCATCGAGAAAACCATCTCCAAAACCAAAGGGC |
| | | AGCCCCGAGAACCACAGGTGTACACCCTGCCCCCATCCCGGGAGGAG |
| | | ATGACCAAGAACCAGGTCAGCCTGACCTGCCTGGTCAAAGGCTTCTAC |
| | | CCCAGCGACATCGCCGTGGAGTGGGAGAGCAATGGGCAGCCGGAGAA |
| | | CAACTACAAGACCACACCTCCCATGCTGGACTCCGACGGCTCCTTCTT |
| | | CCTCTACAGCAAGCTCACCGTGGACAAGAGCAGGTGGCAGCAGGGGA |
| | | ACGTCTTCTCATGCTCCGTGATGCATGAGGCTCTGCACAACCACTACA |
| | | CGCAGAAGAGCCTCTCCCTGTCTCCGGGTAAATGA |
| Heavy Chain | 125 | MDWTWRILFLVAAATGAHSQVQLVQSGAEVKKPGASVKVSCKASGYTF |
| Protein | | TGYYMHWVRQAPGQGLEWMGWINPDSGGTNYAQKFQGRVTMTRDTSI |
| Sequence for | | STAYMELNRLRSDDTAVYYCARDQPLGYCTNGVCSYFDYWGQGTLVTV |
| Antibody 21.4.1 | | SSASTKGPSVFPLAPCSRSTSESTAALGCLVKDYFPEPVTVSWNSGALTSG |
| | | VHTFPAVLQSSGLYSLSSVVTVPSSNFGTQTYTCNVDHKPSNTKVDKTVE |
| | | RKCCVECPPCPAPPVAGPSVFLFPPKPKDTLMISRTPEVTCVVVDVSHEDP |
| | | EVQFNWYVDGVEVHNAKTKPREEQFNSTFRVVSVLTVVHQDWLNGKEY |
| | | KCKVSNKGLPAPIEKTISKTKGQPREPQVYTLPPSREEMTKNQVSLTCLVK |
| | | GFYPSDIAVEWESNGQPENNYKTTPPMLDSDGSFFLYSKLTVDKSRWQQ |
| | | GNVFSCSVMHEALHNHYTQKSLSLSPGK |
| Light Chain | 126 | ATGAGGCTCCCTGCTCAGCTCCTGGGGCTCCTGCTGCTCTGGTTCCCAG |
| DNA Sequence | | GTTCCAGATGCGACATCCAGATGACCCAGTCTCCATCTTCCGTGTCTGC |
| for Antibody | | ATCTGTAGGAGACAGAGTCACCATCACTTGTCGGGCGAGTCAGGGTAT |
| 21.4.1 | | TTACAGCTGGTTAGCCTGGTATCAGCAGAAACCAGGGAAAGCCCCTAA |
| | | CCTCCTGATCTATACTGCATCCACTTTACAAAGTGGGGTCCCATCAAG |
| | | GTTCAGCGGCAGTGGATCTGGGACAGATTTCACTCTCACCATCAGCAG |
| | | CCTGCAACCTGAAGATTTTGCAACTTACTATTGTCAACAGGCTAACATT |
| | | TTCCCGCTCACTTTCGGCGGAGGGACCAAGGTGGAGATCAAACGAACT |
| | | GTGGCTGCACCATCTGTCTTCATCTTCCCGCCATCTGATGAGCAGTTGA |
| | | AATCTGGAACTGCCTCTGTTGTGTGCCTGCTGAATAACTTCTATCCCAG |
| | | AGAGGCCAAAGTACAGTGGAAGGTGGATAACGCCCTCCAATCGGGTA |
| | | ACTCCCAGGAGAGTGTCACAGAGCAGGACAGCAAGGACAGCACCTAC |
| | | AGCCTCAGCAGCACCCTGACGCTGAGCAAAGCAGACTACGAGAAACA |
| | | CAAAGTCTACGCCTGCGAAGTCACCCATCAGGGCCTGAGCTCGCCCGT |
| | | CACAAAGAGCTTCAACAGGGGAGAGTGTTAG |
| Light Chain | 127 | MRLPAQLLGLLLLWFPGSRCDIQMTQSPSSVSASVGDRVTITCRASQGIYS |
| Protein | | WLAWYQQKPGKAPNLLIYTASTLQSGVPSRFSGSGSGTDFTLTISSLQPED |
| Sequence for | | FATYYCQQANIFPLTFGGGTKVEIKRTVAAPSVFIFPPSDEQLKSGTASVV |
| Antibody 21.4.1 | | CLLNNFYPREAKVQWKVDNALQSGNSQESVTEQDSKDSTYSLSSTLTLSK |
| | | ADYEKHKVYACEVTHQGLSSPVTKSFNRGEC |
| Mature Variable | 128 | AGGTGCAGCTGGTGCAGTCTGGGGCTGAGGTGAAGAAGCCTGGGGCC |
| Domain of | | TCAGTGAAGGTCTCCTGCAAGGCTTCTGGATACACCTTCACCGGCTAC |
| Heavy Chain | | TATATGCACTGGGTGCGACAGGCCCCTGGACAAGGGCTTGAGTGGATG |
| DNA Sequence | | GGATGGATCAACCCTGACAGTGGTGGCACAAACTATGCACAGAAGTTT |
| of Antibody | | CAGGGCAGGGTCACCATGACCAGGGACACGTCCATCAGCACAGCCTA |
| 21.4.1 | | CATGGAGCTGAACAGGCTGAGATCTGACGACACGGCCGTGTATTACTG |
| | | TGCGAGAGATCAGCCCCTAGGATATTGTACTAATGGTGTATGCTCCTA |
| | | CTTTGACTACTGGGGCCAGGGAACCCTGGTCACCGTCTCCTCA |
| Mature Variable | 129 | QVQLVQSGAEVKKPGASVKVSCKASGYTFTGYYMHWVRQAPGQGLEW |
| Domain of | | MGWINPDSGGTNYAQKFQGRVTMTRDTSISTAYMELNRLRSDDTAVYY |
| Heavy Chain | | CARDQPLGYCTNGVCSYFDYWGQGTLVTVSS |
| Protein | | |
| Sequence of | | |
| Antibody 21.4.1 | | |
| Mature Variable | 130 | GACATCCAGATGACCCAGTCTCCATCTTCCGTGTCTGCATCTGTAGGA |
| Domain of Light | | GACAGAGTCACCATCACTTGTCGGGCGAGTCAGGGTATTTACAGCTGG |
| Chain DNA | | TTAGCCTGGTATCAGCAGAAACCAGGGAAAGCCCCTAACCTCCTGATC |
| Sequence of | | TATACTGCATCCACTTTACAAAGTGGGGTCCCATCAAGGTTCAGCGGC |
| Antibody 21.4.1 | | AGTGGATCTGGGACAGATTTCACTCTCACCATCAGCAGCCTGCAACCT |
| | | GAAGATTTTGCAACTTACTATTGTCAACAGGCTAACATTTTCCCGCTCA |
| | | CTTTCGGCGGAGGGACCAAGGTGGAGATCAAA |
| Mature Variable | 131 | DIQMTQSPSSVSASVGDRVTITCRASQGIYSWLAWYQQKPGKAPNLLIYT |
| Domain of Light | | ASTLQSGVPSRFSGSGSGTDFTLTISSLQPEDFATYYCQQANIFPLTFGGGT |
| Chain Protein | | KVEIK |
| Sequence of | | |
| Antibody 21.4.1 | | |
| Heavy Chain | 132 | CAGGTGCAGCTGGTGGAGTCTGGGGGAGGCGTGGTCCAGCCTGGGAG |
| DNA Sequence | | GTCCCTGAGACTCTCCTGTGCAGCCTCTGGATTCACCTTCAGTAGCTAT |
| for Antibody | | GTCATGCACTGGGTCCGCCAGGCTCCAGGCAAGGGGCTGGAGTGGGT |
| 21.2.1 | | GGCAGTTATGTCATATGATGGAAGTAGTAAATACTATGCAAACTCCGT |
| | | GAAGGGCCGATTCACCATCTCCAGAGACAATTCCAAGAACACGCTGTA |
| | | TCTGCAAATAAACAGCCTGAGAGCTGAGGACACGGCTGTGTATTACTG |
| | | TGCGAGAGATGGGGGTAAAGCAGTGCCTGGTCCTGACTACTGGGGCC |
| | | AGGGAATCCTGGTCACCGTCTCCTCAG |
| Heavy Chain | 133 | QVQLVESGGGVVQPGRSLRLSCAASGFTFSSYVMHWVRQAPGKGLEWV |
| Protein | | AVMSYDGSSKYYANSVKGRFTISRDNSKNTLYLQINSLRAEDTAVYYCA |
| Sequence for | | RDGGKAVPGPDYWGQGILVTVSS |
| Antibody 21.2.1 | | |
| Light Chain | 134 | GATATTGTGATGACTCAGTCTCCACTCTCCCTGCCCGTCACCCCTGGAG |
| DNA Sequence | | AGCCGGCCTCCATCTCCTGCAGGTCTAGTCAGAGTGTTCTGTATAGTA |
| for Antibody | | ATGGATACAACTATTTGGATTGGTACCTGCAGAAGCCAGGGCAGTCTC |
| 21.2.1 | | CACAGCTCCTGATCTATTTGGGTTCTAATCGGGCCTCCGGGGTCCCTGA |
| | | CAGGTTCAGTGGCAGTGGATCAGGCACAGATTTTACACTGAAAATCAG |
| | | CAGAGTGGAGGCTGAGGATGTTGGGGTTTATTACTGCATGCAAGTTTT |
| | | ACAAACTCCATTCACTTTCGGCCCTGGGACCAAAGTGGATATCAAAC |
| Light Chain | 135 | DIVMTQSPLSLPVTPGEPASISCRSSQSVLYSNGYNYLDWYLQKPGQSPQL |
| Protein | | LIYLGSNRASGVPDRFSGSGSGTDFTLKISRVEAEDVGVYYCMQVLQTPF |
| Sequence for | | TFGPGTKVDIK |
| Antibody 21.2.1 | | |
| Heavy Chain | 136 | CAGGTGCAGCTGGTGGAGTCTGGGGGAGGCGTGGTCCAGCCTGGGAG |
| DNA Sequence | | GTCCCTGAGACTCTCCTGTGCAGCCTCTGGATTCACCTTCAGTCGCTAT |
| for Antibody | | GGCATGCACTGGGTCCGCCAGGCTCCAGGCAAGGGGCTGGAGTGGGT |
| 22.1.1 | | GGCAGTTATATCATCTGATGGAGGTAATAAATACTATGCAGACTCCGT |
| | | GAAGGGCCGATTCACCATCTCCAGAGACAATTCCAAGAACACGCTGTA |
| | | TCTGCAAATGAACAGCCTGAGAGCTGAGGACACGGCTGTGTATTACTG |
| | | TACGAGAAGAGGGACTGGAAAGACTTACTACCACTACTGTGGTATGG |
| | | ACGTCTGGGGCCAAGGGACCACGGTCACCGTCTCCTCAG |
| Heavy Chain | 137 | QVQLVESGGGVVQPGRSLRLSCAASGFTFSRYGMHWVRQAPGKGLEWV |
| Protein | | AVISSDGGNKYYADSVKGRFTISRDNSKNTLYLQMNSLRAEDTAVYYCT |
| Sequence for | | RRGTGKTYYHYCGMDVWGQGTTVTVSS |
| Antibody 22.1.1 | | |
| Light Chain | 138 | GATATTGTGATGACTCAGTCTCCACTCTCCCTGCCCGTCACCCCTGGAG |
| DNA Sequence | | AGCCGGCCTCCATCTCCTGCAGGTCTAGTCAGAGCCTCCTGTATAGTA |
| for Antibody | | ATGGATATAACTATTTGGATTGGTACCTGCAGAAGCCAGGGCAGTCTC |
| 22.1.1 | | CACACCTCCTGATCTATTTGGGTTCTAATCGGGCCTCCGGGGTCCCTGA |
| | | CAGGTTCAGTGGCAGTGGTTCAGGCACTGATTTTACACTGAAAATCAG |
| | | CAGAGTGGAGGCTGAGGATGTTGGGGTTTATTACTGCATGCAAGCTCT |
| | | ACAAACTCCTCGGACGTTCGGCCAAGGGACCAAGGTGGAAATCAAAC |
| Light Chain | 139 | DIVMTQSPLSLPVTPGEPASISCRSSQSLLYSNGYNYLDWYLQKPGQSPHL |
| Protein | | LIYLGSNRASGVPDRFSGSGSGTDFTLKISRVEAEDVGVYYCMQALQTPR |
| Sequence for | | TFGQGTKVEIK |
| Antibody 22.1.1 | | |
| Heavy Chain | 140 | CAGGTGCAGCTGGTGGAGTCTGGGGGAGGCGTCCAGCCTGGGAGGTC |
| DNA Sequence | | CCTGAGACTCTCCTGTGTAGCCTCTGGATTCACCTTCAGTAACTATGGC |
| for Antibody | | ATGCACTGGGTCCGCCAGGCTCCAGGCAAGGGGCTGGAGTGGGTGGC |
| 23.5.1 | | AATTATATCATATGATGGAAGTAATAAATACTATGCAGACTCCGTGAA |
| | | GGGCCGATTCACCATCTCCAGAGACAATTCCAAGAACACGCTGTATGT |
| | | GCAAATGAACAGCCTGAGAGCTGAGGACACGGCTGTGTATTACTGTGC |
| | | GAGACGCGGTCACTACGGGAGGGATTACTACTCCTACTACGGTTTGGA |
| | | CGTCTGGGGCCAAGGGACCACGGTCACCGTCTCCTCAG |
| Heavy Chain | 141 | QVQLVESGGGVVQPGRSLRLSCVASGFTFSNYGMHWVRQAPGKGLEWV |
| Protein | | AIISYDGSNKYYADSVKGRFTISRDNSKNTLYVQMNSLRAEDTAVYYCAR |
| Sequence for | | RGHYGRDYYSYYGLDVWGQGTTVTVSS |
| Antibody 23.5.1 | | |
| Light Chain | 142 | GATATTGTGATGACTCAGTCTCCACTCTCCCTGCCCGTCACCCCTGGAG |
| DNA Sequence | | AGCCGGCCTCCATCTCCTGCAGGTCTAGTCAGAGCCTCCTGCCTGGTA |
| for Antibody | | ATGGATACAACTATTTGGATTGGTACCTGCAGAAGCCAGGGCAGTCTC |
| 23.5.1 | | CACAGCTCCTGATCTATTTGGGTTCTAATCGGGCCTCCGGGGTCCCTGA |
| | | CAGGTTCAGTGGCAGTGGATCAGGCACAGATTTTACACTGAAAATCAG |
| | | CAGAGTGGAGGCTGAGGATGTTGGGGTTTATTACTGCATGCAAGCTCT |
| | | ACAAACTCCTCGGACGTTCGGCCAAGGGACCAAGGTGGAAATCAAAC |
| Light Chain | 143 | DIVMTQSPLSLPVTPGEPASISCRSSQSLLPGNGYNYLDWYLQKPGQSPQL |
| Protein | | LIYLGSNRASGVPDRFSGSGSGTDFTLKISRVEAEDVGVYYCMQALQTPR |
| Sequence for | | TFGQGTKVEIK |
| Antibody 23.5.1 | | |
| Heavy Chain | 144 | CAGGTGCAGCTGCAGGAGTCGGGCCCAGGACTGGTGAAGCCTTCGGA |
| DNA Sequence | | CACCCTGTCCCTCACCTGCACTGTCTCTGGTGGCTCCATCAGAGGTTAC |
| for Antibody | | TACTGGAGCTGGATCCGGCAGCCCCCTGGGAAGGGACTGGAGTGGAT |
| 23.28.1 | | TGGGTATATCTATTACAGTGGGAGCACCAACTACAACCCCTCCCTCAA |
| | | GAGTCGAGTCACCATATCAGTAGACACGTCCAAGAACCAGTTCTCCCT |
| | | GAAGCTGAACTCTGTGACCGCTGCGGACACGGCCGTGTATTATTGTGC |
| | | GAGAAAGGGGGGCCTCTACGGTGACTACGGCTGGTTCGCCCCCTGGG |
| | | GCCAGGGAACCCTGGTCACCGTCTCCTCAG |
| Heavy Chain | 145 | QVQLQESGPGLVKPSDTLSLTCTVSGGSIRGYYWSWIIRQPPGKGLEWIGY |
| Protein | | IYYSGSTNYNPSLKSRVTISVDTSKNQFSLKLNSVTAADTAVYYCARKGG |
| Sequence for | | LYGDYGWFAPWGQGTLVTVSS |
| Antibody | | |
| 23.28.1 | | |
| Light Chain | 146 | GAAATTGTGTTGACGCAGTCTCCAGGCACCCTGTCTTTGTCTCCAGGG |
| DNA Sequence | | GAAAGAGCCACCCTCTCCTGCAGGGCCAGTCAGAGTGTTAGCAGCAG |
| for Antibody | | CGACTTAGCCTGGCACCAGCAGAAACCTGGCCAGGCTCCCAGACTCCT |
| 23.28.1 | | CATCTATGGTGCATCCAGCAGGGCCACTGGCATCCCAGACAGGTTCAG |
| | | TGGCAGTGGGTCTGGGACAGACTTCACTCTCACCATCAGCAGACTGGA |
| | | GCCTGAAGATTTTGCAGTGTATTACTGTCAGCACTGTCGTAGCTTATTC |
| | | ACTTTCGGCCCTGGGACCAAAGTGGATATCAAAC |
| Light Chain | 147 | EIVLTQSPGTLSLSPGERATLSCRASQSVSSSDLAWHQQKPGQAPRLLIYG |
| Protein | | ASSRATGIPDRFSGSGSGTDFTLTISRLEPEDFAVYYCQHCRSLFTFGPGTK |
| Sequence for | | VDIK |
| Antibody | | |
| 23.28.1 | | |
| Heavy Chain | 148 | CAGGTGCAGCTGCAGGAGTCGGGCCCAGGACTGGTGAAGCCTTCGGA |
| DNA Sequence | | GACCCTGTCCCTCACCTGCACTGTCTCTGGTGGCTCCATCAGAGGTTAC |
| (variable domain | | TACTGGAGCTGGATCCGGCAGCCCCCTGGGAAGGGACTGGAGTGGAT |
| 23.28.1H-D16E) | | TGGGTATATCTATTACAGTGGGAGCACCAACTACAACCCCTCCCTCAA |
| | | GAGTCGAGTCACCATATCAGTAGACACGTCCAAGAACCAGTTCTCCCT |
| | | GAAGCTGAACTCTGTGACCGCTGCGGACACGGCCGTGTATTATTGTGC |
| | | GAGAAAGGGGGGCCTCTACGGTGACTACGGCTGGTTCGCCCCCTGGG |
| | | GCCAGGGAACCCTGGTCACCGTCTCCTCAG |
| Heavy Chain | 149 | QVQLQESGPGLVKPSLTLSLTCTVSGGSIRGYYWSWIRQPPGKGLEWIGYI |
| Protein | | YYSGSTNYNPSLKSRVTISVDTSKNQFSLKLNSVTAADTAVYYCARKGGL |
| Sequence | | YGDYGWFAPWGQGTLVTVSS |
| (variable domain | | |
| 23.28.1H-D16E) | | |
| Heavy Chain | 150 | CAGGTGCAGCTGGTGGAGTCTGGGGGAGGCGTGGTCCAGCCTGGGAG |
| DNA Sequence | | GTCCCTGAGACTCTCCTGTGCAGCCTCTGGATTCACCTTCAGTAGCTAT |
| of Antibody | | GCCATGCACTGGGTCCGCCAGGCTCCAGGCAAGGGGCTGGAGTGGGT |
| 23.29.1 | | GGCAGTTATATCATATGATGGAAGTAATAAATACTATGCAGACTCCGT |
| | | GAAGGGCCGATTCACCATCTACAGAGACAATTCCAAGAACACGCTGT |
| | | ATCTGCAAATGAACAGCCTGAGAGCTGAGGACACGGCTGTGTATTACT |
| | | GTGCGAGACGCGGTCACTACGGGAATAATTACTACTCCTATTACGGTT |
| | | TGGACGTCTGGGGCCAAGGGACCACGGTCACCGTCTCCTCAG |
| Heavy Chain | 151 | QVQLVESGGGVVQPGRSLRLSCAASGFTFSSYAMHWVRQAPGKGLEWV |
| Protein | | AVISYDGSNKYYADSVKGRFTIYRDNSKNTLYLQMNSLRAEDTAVYYCA |
| Sequence for | | RRGHYGNNYYSYYGLDVWGQGTTVTVSS |
| Antibody | | |
| 23.29.1 | | |
| Light Chain | 152 | GATATTGTGATGACTCAGTCTCCACTCTCCCTGCCCGTCACCCCTGGAG |
| DNA Sequence | | AGCCGGCCTCCATCTCCTGCAGGTCTAGTCAGAGCCTCCTGCCTGGTA |
| for Antibody | | ATGGATACAACTATTTGGATTGGTACCTGCAGAAGCCAGGGCAGTCTC |
| 23.29.1 | | CACAGCTCCTGATCTATTTGGGTTCTAATCGGGCCTCCGGGGTCCCTGC |
| | | AGGTTCAGTGGCAGTGGCTCAGGCACAGATTTTACACTGAAAATCAGC |
| | | AGAGTGGAGGCTGAGGATGTTGGGATTTATTACTGCATGCAAGCTCTA |
| | | CAAACTCCTCGGACGTTCGGCCAAGGGACCAAGGTGGAAATCAAAC |
| Light Chain | 153 | DIVMTQSPLSLPVTPGEPASISCRSSQSLLPGNGYNYLDWYLQKPGQSPQL |
| Protein | | LIYLGSNRASGVPDRFSGSGSGTDFTLKISRVEAEDVGIYYCMQALQTPRT |
| Sequence for | | FGQGTKVEIK |
| Antibody | | |
| 23.29.1 | | |
| Heavy Chain | 154 | CAGGTGCAGCTGCAGGAGTCGGGCCCAGGACTGGTGAAGCCTTCGGA |
| DNA Sequence | | GACCCTGTCCCTCACCTGCACTGTCTCTGGTGGCTCCATCAGAGGTTAC |
| for Antibody | | TACTGGAGCTGGATCCGGCAGCCCCCAGGGAAGGGACTGGAGTGGAT |
| 24.2.1 | | TGGGTATATCTATTACAGTGGGAGCACCAACTACAACCCCTCCCTCAA |
| | | GAGTCGAGTCACCATATCAGTAGACACGTCCAAGAACCAGTTCTCCCT |
| | | GAAGCTGAGTTCTGTGACCGCTGCGGACACGGCCGTGTATTACTGTGC |
| | | GAGAAGGGGGGGCCTCTACGGTGACTACGGCTGGTTCGCCCCCTGGG |
| | | GCCAGGGAACCCTGGTCACCGTCTCCTCAG |
| Heavy Chain | 155 | QVQLQESGPGLVKPSETLSLTCTVSGGSIRGYYWSWIRQPPGKGLEWIGYI |
| Protein | | YYSGSTNYNPSLKSRVTISVDTSKNQFSLKLSSVTAADTAVYYCARRGGL |
| Sequence for | | YGDYGWFAPWGQGTLVTVSS |
| Antibody 24.2.1 | | |
| Light Chain | 156 | GAAATTGTGTTGACGCAGTCTCCAGGCACCCTGTCTTTGTCTCCAGGG |
| DNA Sequence | | GAAAGAGCCACCCTCTCCTGCAGGGCCAGTCAGAGTGTTAGCAGCACC |
| for Antibody | | TACTTAGCCTGGTACCAGCAGAAACCTGGCCAGGCTCCCAGGCTCCTC |
| 24.2.1 | | ATCTATGGTGCATCCAGCAGGGCCACTGGCATCCCAGACAGGTTCAGT |
| | | GGCAGTGGGTCTGGGACAGACTTCACTCTCACCATCAGCAGACTGGAG |
| | | CCTGAAGATTTTGCAGTGTATTACTGTCAGCAGTATAGTAGCTTATTCA |
| | | CTTTCGGCCCTGGGACCAAAGTGGATATCAAAC |
| Light Chain | 157 | ETVLTQSPGTLSLSPGERATLSCRASQSVSSTYLAWYQQKPGQAPRLLIYG |
| Protein | | ASSRATGIIPDRFSGSGSGTDFTLTISRLEPEDFAVYYCQQYSSLFTFGPGTK |
| Sequence for | | VDIK |
| Antibody 24.2.1 | | |
| Heavy Chain | 158 | ATGGAGTTTGGGCTGAGCTGGGTTTTCCTCGTTGCTCTTTTAAGAGGTG |
| DNA Sequence | | TCCAGTGTCAGGTGCAGCTGGTGGAGTCTGGGGGAGGCGTGGTCCAGC |
| for Antibody | | CTGGGAGGTCCCTGAGACTCTCCTGTGCAGCCTCTGGATTCACCTTCA |
| 21.2.1 | | GTAGCTATGTCATGCACTGGGTCCGCCAGGCTCCAGGCAAGGGGCTGG |
| | | AGTGGGTGGCAGTTATGTCATATGATGGAAGTAGTAAATACTATGCAA |
| | | ACTCCGTGAAGGGCCGATTCACCATCTCCAGAGACAATTCCAAGAACA |
| | | CGCTGTATCTGCAAATAAACAGCCTGAGAGCTGAGGACACGGCTGTGT |
| | | ATTACTGTGCGAGAGATGGGGGTAAAGCAGTGCCTGGTCCTGACTACT |
| | | GGGGCCAGGGAATCCTGGTCACCGTCTCCTCAGCCTCCACCAAGGGCC |
| | | CATCGGTCTTCCCCCTGGCGCCCTGCTCCAGGAGCACCTCCGAGAGCA |
| | | CAGCGGCCCTGGGCTGCCTGGTCAAGGACTACTTCCCCGAACCGGTGA |
| | | CGGTGTCGTGGAACTCAGGCGCTCTGACCAGCGGCGTGCACACCTTCC |
| | | CAGCTGTCCTACAGTCCTCAGGACTCTACTCCCTCAGCAGCGTGGTGA |
| | | CCGTGCCCTCCAGCAACTTCGGCACCCAGACCTACACCTGCAACGTAG |
| | | ATCACAAGCCCAGCAACACCAAGGTGGACAAGACAGTTGAGCGCAAA |
| | | TGTTGTGTCGAGTGCCCACCGTGCCCAGCACCACCTGTGGCAGGACCG |
| | | TCAGTCTTCCTCTTCCCCCCAAAACCCAAGGACACCCTCATGATCTCCC |
| | | GGACCCCTGAGGTCACGTGCGTGGTGGTGGACGTGAGCCACGAAGAC |
| | | CCCGAGGTCCAGTTCAACTGGTACGTGGACGGCGTGGAGGTGCATAAT |
| | | GCCAAGACAAAGCCACGGGAGGAGCAGTTCAACAGCACGTTCCGTGT |
| | | GGTCAGCGTCCTCACCGTTGTGCACCAGGACTGGCTGAACGGCAAGGA |
| | | GTACAAGTGCAAGGTCTCCAACAAAGGCCTCCCAGCCCCCATCGAGA |
| | | AAACCATCTCCAAAACCAAAGGGCAGCCCCGAGAACCACAGGTGTAC |
| | | ACCCTGCCCCCATCCCGGGAGGAGATGACCAAGAACCAGGTCAGCCT |
| | | GACCTGCCTGGTCAAAGGCTTCTACCCCAGCGACATCGCCGTGGAGTG |
| | | GGAGAGCAATGGGCAGCCGGAGAACAACTACAAGACCACACCTCCCA |
| | | TGCTGGACTCCGACGGCTCCTTCTTCCTCTACAGCAAGCTCACCGTGG |
| | | ACAAGAGCAGGTGGCAGCAGGGGAACGTCTTCTCATGCTCCGTGATGC |
| | | ATGAGGCTCTGCACAACCACTACACGCAGAAGAGCCTCTCCCTGTCTC |
| | | CGGGTAAATGA |
| Heavy Chain | 159 | MEFGLSWVFLVALLRGVQCQVQLVESGGGVVQPGRSLRLSCAASGFTFS |
| Protein | | SYVMHWVRQAPGKGLEWVAVMSYDGSSKYYANSVKGRIFTISRDNSKN |
| Sequence for | | TLYLQINSLRAEDTAVYYCARDGGKAVPGPDYWGQGILVTVSSASTKGP |
| Antibody 21.2.1 | | SVFPLAPCSRSTSESTAALGCLVKDYFPEPVTVSWNSGALTSGVHTFPAVL |
| | | QSSGLYSLSSVVTVPSSNTGTQTYTCNVDHKPSNTKVDKTVERKCCVECP |
| | | PCPAPPVAGPSVFLFPPKPKDTLMISRTPEVTCVVVDVSHEDPEVQFNWY |
| | | VDGVEVHNAKTKPREEQFNSTFRVVSVLTVVHQDWLNGKEYKCKVSNK |
| | | GLPAPIEKTISKTKGQPREPQVYTLPPSREEMTKNQVSLTCLVKGFYPSDIA |
| | | VEWESNGQPENNYKTTPPMLDSDGSFFLYSKLTVDKSRWQQGNVFSCSV |
| | | MHEALHNHYTQKSLSLSPGK |
| Light Chain | 160 | ATGAGGCTCCCTGCTCAGCTCCTGGGGCTGCTAATGCTCTGGGTCTCTG |
| DNA Sequence | | GATCCAGTGGGGATATTGTGATGACTCAGTCTCCACTCTCCCTGCCCGT |
| for Antibody | | CACCCCTGGAGAGCCGGCCTCCATCTCCTGCAGGTCTAGTCAGAGTGT |
| 21.2.1 | | TCTGTATAGTAATGGATACAACTATTTGGATTGGTACCTGCAGAAGCC |
| | | AGGGCAGTCTCCACAGCTCCTGATCTATTTGGGTTCTAATCGGGCCTCC |
| | | GGGGTCCCTGACAGGTTCAGTGGCAGTGGATCAGGCACAGATTTTACA |
| | | CTGAAAATCAGCAGAGTGGAGGCTGAGGATGTTGGGGTTTATTACTGC |
| | | ATGCAAGTTTTACAAACTCCATTCACTTTCGGCCCTGGGACCAAAGTG |
| | | GATATCAAACGAACTGTGGCTGCACCATCTGTCTTCATCTTCCCGCCAT |
| | | CTGATGAGCAGTTGAAATCTGGAACTGCCTCTGTTGTGTGCCTGCTGA |
| | | ATAACTTCTATCCCAGAGAGGCCAAAGTACAGTGGAAGGTGGATAAC |
| | | GCCCTCCAATCGGGTAACTCCCAGGAGAGTGTCACAGAGCAGGACAG |
| | | CAAGGACAGCACCTACAGCCTCAGCAGCACCCTGACGCTGAGCAAAG |
| | | CAGACTACGAGAAACACAAAGTCTACGCCTGCGAAGTCACCCATCAG |
| | | GGCCTGAGCTCGCCCGTCACAAAGAGCTTCAACAGGGGAGAGTGTTA |
| | | G |
| Light Chain | 161 | MRLPAQLLGLLMLWVSGSSGDIVMTQSPLSLPVTPGEPASISCRSSQSVLY |
| Protein | | SNGYNYLDWYLQKPGQSPQLLIYLGSNRASGVPDRFSGSGSGTDFTLKIS |
| Sequence for | | RVEAEDVGVYYCMQVLQTPFTFGPGTKVDWRTVAAPSVFIFPPSDEQLKS |
| Antibody 21.2.1 | | GTASVVCLLNNFYPREAKVQWKVDNALQSGNSQESVTEQDSKDSTYSLS |
| | | STLTLSKADYEKHKVYACEVTHQGLSSPVTKSFNRGEC |

Additional anti-CD40 antibody sequences that can be used in the antibody construct can comprise a sequence with greater than 70%, greater than 75%, greater than 80%, greater than 85%, greater than 90%, greater than 95% or greater than 99% homology to any sequence in TABLE 1.

Antibody constructs disclosed herein can be non-natural, designed, and/or engineered. Antibody constructs disclosed herein can be non-natural, designed, and/or engineered scaffolds comprising an antigen binding domain. Antibody constructs disclosed herein can be non-natural, designed, and/or engineered antibodies. Antibody constructs can be monoclonal antibodies. Antibody constructs can be human antibodies. Antibody constructs can be humanized antibodies. Antibody constructs can be monoclonal humanized antibodies. Antibody constructs can be recombinant antibodies.

An antigen binding domain of an antibody construct can be selected in order to recognize an antigen. For example, an antigen can be a cell surface marker on a target cell associated with a disease or condition. An antigen can be expressed on an immune cell. An antigen can be a peptide or fragment thereof. An antigen can be expressed on an antigen-presenting cell. An antigen can be expressed on a dendritic cell, a macrophage, or a B cell. An antigen can be a peptide presented in a major histocompatibility complex by cell. As another example, a cell surface marker recognized by the antigen binding domain can include macromolecules associated with viral and bacterial diseases or infections, autoimmune diseases and cancerous diseases. An antigen can be CD40 and an antigen binding domain can recognize a CD40 antigen. An antigen can be a tumor antigen or fragment thereof. A tumor antigen can be any antigen listed on tumor antigen databases, such as TANTIGEN, or peptide databases for T cell-defined tumor antigens, such as the Cancer Immunity Peptide database. A tumor antigen can also be any antigen listed in the review by Chen (Chen, Cancer Immun 2004 [updated 2004 Mar. 10; cited 2004 Apr. 1]). Note that the ‘antibody’ can recognize the ‘tumor antigen’ or a peptide derived thereof, bound to an MHC molecule. An antigen can have at least 80% homology to or can be CD5, CD19, CD20, CD25, CD37, CD30, CD33, CD45, CAMPATH-1, BCMA, CS-1, PD-L1, B7-H3, B7-DC, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MART, Ras mutant, gp100, p53 mutant, PR1, bcr-ab1, tyronsinase, survivin, PSA, hTERT, Sarcoma translocation breakpoints, EphA2, PAP, ML-IAP, AFP, ERG, NA17, PAX3, ALK, androgen receptor, cyclin B1, polysialic acid, MYCN, RhoC, TRP-2, fucosyl GM1, mesothelin, PSCA, MAGE A1, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL, MUC16, MAGE A4, MAGE C2, GAGE, or Fos-related antigen 1. An antigen binding domain can be capable of recognizing a single antigen. An antigen binding domain can be capable of recognizing two or more different antigens.

An antibody construct can comprise an antibody heavy chain. A heavy chain can be a heavy chain of an anti-HER2 monoclonal antibody which can bind a HER2 antigen. A heavy chain of an anti-HER2 antibody can be an IgG1 isotype. A heavy chain of an anti-HER2 antibody can be SBT-050 VH-hIgG1 wt (pertuzumab). SBT-050 VH-hIgG1 wt can comprise an amino acid sequence EVQLVESGGGLVQPGGSLRLSCAASGFTFTDYTMDWVRQAPGKGLEWVADVNPNSGGSIY NQRFKGRFTLSVDRSKNTLYLQMNSLRAEDTAVYYCARNLGPSFYFDYWGQGTLVTVSSA STKGPSVFPLAPSSKSTSGGTAALGCLVKDYFPEPVTVSWNSGALTSGVHTFPAVLQSSGLY SLSSVVTVPSSSLGTQTYICNVNHKPSNTKVDKKVEPKSCDKTHTCPPCPAPELLGGPSVFLF PPKPKDTLMISRTPEVTCVVVDVSHEDPEVKFNWYVDGVEVHNAKTKPREEQYNSTYRVV SVLTVLHQDWLNGKEYKCKVSNKALPAPIEKTISKAKGQPREPQVYTLPPSREEMTKNQVS LTCLVKGFYPSDIAVEWESNGQPENNYKTTPPVLDSDGSFFLYSKLTVDKSRWQQGNVFSC SVMHEALHNHYTQKSLSLSPGK (SEQ ID NO: 30). SBT-050 VH-hIgG1 wt can comprise an amino acid sequence with greater than 70%, greater than 75%, greater than 80%, greater than 85%, greater than 90%, greater than 95% or greater than 99% homology to SEQ ID NO: 30. A heavy chain of an anti-HER2 antibody can comprise a CDR. A heavy chain of an anti-HER2 antibody can comprise a CDR with an amino acid sequence GFTFTDYT (SEQ ID NO: 31). A heavy chain of an anti-HER2 antibody can comprise a CDR with an amino acid sequence VNPNSGGS (SEQ ID NO: 32). A heavy chain of an anti-HER2 antibody can comprise a CDR with an amino acid sequence ARNLGPSFYFDY (SEQ ID NO: 33). A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 31. A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 32. A heavy chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 33.

An antibody construct can comprise an antibody light chain. A light chain can be a light chain of a HER2 monoclonal antibody which can bind a HER2 antigen. A light chain of an anti-HER2 antibody can be SBT-050 VL-Ck (pertuzumab). SBT-050 VL-Ck can comprise an amino acid sequence DIQMTQSPSSLSASVGDRVTITCKASQDVSIGVAWYQQKPGKAPKLLIYSASYRYTGVPSRF SGSGSGTDFTLTISSLQPEDFATYYCQQYYIYPYTFGQGTKVEIKRTVAAPSVFIFPPSDEQLK SGTASVVCLLNNFYPREAKVQWKVDNALQSGNSQESVTEQDSKDSTYSLSSTLTLSKADYE KHKVYACEVTHQGLSSPVTKSFNRGEC (SEQ ID NO: 34). SBT-050 VL-Ck can comprise an amino acid sequence with greater than 70%, greater than 75%, greater than 80%, greater than 85%, greater than 90%, greater than 95% or greater than 99% homology to SEQ ID NO: 34. A light chain of an anti-HER2 antibody can comprise a CDR. A light chain of an anti-HER2 antibody can comprise a CDR with an amino acid sequence QDVSIG (SEQ ID NO: 35). A light chain of an anti-HER2 antibody can comprise a CDR with an amino acid sequence SAS (SEQ ID NO: 36). A light chain of an anti-HER2 antibody can comprise a CDR with an amino acid sequence QQYYIYPYT (SEQ ID NO: 37). A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 35. A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 36. A light chain CDR of an anti-HER2 antibody can comprise an amino acid sequence with greater than 70%, greater than 75%, greater than 80%, greater than 85%, greater than 90%, greater than 95% or greater than 99% homology to SEQ ID NO: 37.

An antibody construct can comprise an Fc region with an Fc domain. An Fc domain is a structure that can bind to Fc receptors (FcRs). An antibody construct can comprise an Fc domain. Fc domains can be bound by FcRs. An Fc domain can be from an antibody. An Fc domain can be at least 80% homologous to an Fc domain from an antibody. An Fc region can be in a scaffold. An Fc region with an Fc domain can be in an antibody scaffold. An Fc region with an Fc domain can be in a non-antibody scaffold. An antibody construct can comprise an Fc region with an Fc domain in an antibody scaffold. An antibody construct can comprise an Fc region with an Fc domain in a non-antibody scaffold. An Fc domain can be in a scaffold. An Fc domain can be in an antibody scaffold. An Fc domain can be in a non-antibody scaffold. An antibody construct can comprise an Fc domain in an antibody scaffold. An antibody construct can comprise an Fc domain in a non-antibody scaffold. Fc domains of antibodies, including those of the present disclosure, can be bound by FcRs. Fc domains can be a portion of the Fc region of an antibody. FcRs can bind to an Fc domain of an antibody. FcRs can bind to an Fc domain of an antibody bound to an antigen. FcRs can be organized into classes (e.g., gamma (γ), alpha (a) and epsilon (F)) based on the class of antibody that the FcR recognizes. The FcαR class can bind to IgA and includes several isoforms, FcαRI (CD89) and FcαμR. The FcγR class can bind to IgG and includes several isoforms, FcγRI (CD64), FcγRIIA (CD32a), FcγRIIB (CD32b), FcγRIIIA (CD16a), and FcγRIIIB (CD16b). An FcγRIIIA (CD16a) can be an FcγRIIIA (CD16a) F158 variant. An FcγRIIIA (CD16a) can be an FcγRIIIA (CD16a) V158 variant. Each FcγR isoform can differ in affinity to the Fc region of the IgG antibody. For example, FcγRI can bind to IgG with greater affinity than FcγRII or FcγRIII. The affinity of a particular FcγR isoform to IgG can be controlled, in part, by a glycan (e.g., oligosaccharide) at position CH₂ 84.4 of the IgG antibody. For example, fucose containing CH₂ 84.4 glycans can reduce IgG affinity for FcγRIIIA. In addition, G0 glucans can have increased affinity for FcγRIIIA due to the lack of galactose and terminal GlcNAc moiety.

Binding of an Fc domain to an FcR can enhance an immune response. FcR-mediated signaling that can result from an Fc region binding to an FcR can lead to the maturation of immune cells. FcR-mediated signaling that can result from an Fc domain binding to an FcR can lead to the maturation of dendritic cells. FcR-mediated signaling that can result from an Fc domain binding to an FcR can lead to more efficient immune cell antigen uptake and processing. FcR-mediated signaling that can result from an Fc region binding to an FcR can lead to more efficient dendritic cell antigen uptake and processing. FcR-mediated signaling that can result from an Fc region binding to an FcR can increase antigen presentation. FcR-mediated signaling that can result from an Fc region binding to an FcR can increase antigen presentation by immune cells. FcR-mediated signaling that can result from an Fc region binding to an FcR can increase antigen presentation by antigen presenting cells. FcR-mediated signaling that can result from an Fc domain binding to an FcR can increase antigen presentation by dendritic cells. FcR-mediated signaling that can result from an Fc domain binding to an FcR can promote the expansion and activation of T cells. FcR-mediated signaling that can result from an Fc domain binding to an FcR can promote the expansion and activation of CD8⁺ T cells. FcR-mediated signaling that can result from an Fc domain binding to an FcR can influence immune cell regulation of T cell responses. FcR-mediated signaling that can result from an Fc domain binding to an FcR can influence immune cell regulation of T cell responses. FcR-mediated signaling that can result from an Fc domain binding to an FcR can influence dendritic cell regulation of T cell responses. FcR-mediated signaling that can result from an Fc domain binding to an FcR can influence functional polarization of T cells (e.g., polarization can be toward a T_{H}1 cell response).

The profile of FcRs on a DC can impact the ability of the DC to respond upon stimulation. For example, most DC can express both CD32a and CD32b, which can have opposing effects on IgG-mediated maturation and function of DCs: binding of IgG to CD32a can mature and activate DCs in contrast with CD32b, which can mediate inhibition due to phosphorylation of immunoreceptor tyrosine-based inhibition motif (ITIM), after CD32b binding of IgG. Therefore, the activity of these two receptors can establish a threshold of DC activation. Furthermore, difference in functional avidity of these receptors for IgG can shift their functional balance. Hence, altering the Fc domain binding to FcRs can also shift their functional balance, allowing for manipulation (either enhanced activity or enhanced inhibition) of the DC immune response.

A modification in the amino acid sequence of the antibody construct can alter the recognition and binding of an FcR for the Fc domain. For example, a modification of the amino acid sequence of the Fc domain in an antibody construct can increase the binding affinity and/or avidity of the Fc domain for FcRs. This increase in binding affinity and/or avidity can specific for a type of FcR. However, such modifications can still allow for FcR-mediated signaling. A modification can be a substitution of an amino acid at a residue (e.g., wildtype) for a different amino acid at that residue. For example, a wildtype Fc domain can comprise ASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFL FPPKPKDTLMISRTPEVTCVVVDVSHEDPEVKFNWYVDGVEVHNAKTKPREEQYNSTYRV VSVLTVLHQDWLNGKEYKCKVSNKALPAPIEKTISKAKGQPREPQVYTLPPSREEMTKNQV SLTCLVKGFYPSDIAVEWESNGQPENNYKTTPPVLDSDGSFFLYSKLTVDKSRWQQGNVFS CSVMHEALHNHYTQKSLSLSPGK (SEQ ID NO: 162), and a modified Fe domain can comprise a substitution of an amino acid in comparison with SEQ ID NO: 162. A modification can permit binding of an FcR to a site on the Fc region of an antibody construct that the FcR may not otherwise bind to. A modification can increase binding affinity of an FcR to the Fc domain of an antibody construct that the FcR may have reduced binding affinity for. A modification can decrease binding affinity of an FcR to a site on the Fc domain of an antibody construct that the FcR may have increased binding affinity for. A modification can increase the subsequent FcR-mediated signaling after Fc binding to an FcR.

An antibody construct can comprise an Fc region with at least one amino acid change as compared to the sequence of the wild-type Fc region. A wild-type Fc region can comprise SEQ ID NO: 162. An antibody construct can comprise an Fc domain with at least one amino acid change as compared to the sequence of the wild-type Fc domain. A wild-type Fc domain can comprise SEQ ID NO: 162. For example, an antibody construct can comprise an Fc domain with at least one amino acid change as compared to the sequence of the wild-type Fc domain comprising SEQ ID NO: 162.

An amino acid change in an Fc region of an antibody construct can allow the antibody construct to bind to at least one Fc receptor with greater affinity compared to a wild-type Fc region. An amino acid change in an Fc domain of an antibody construct can allow the antibody to bind to at least one Fc receptor with greater affinity compared to a wild-type Fc domain. An Fc region can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc domain can comprise an amino acid sequence having at least one, two, three, four, five, six, seven, eight, nine or ten modifications but not more than 40, 35, 30, 25, 20, 15 or 10 modifications of the amino acid sequence relative to the natural or original amino acid sequence. An Fc region can be an Fc region of an anti-CD40 antibody. An Fc domain can be an Fc domain of an anti-CD40 antibody. An Fc region can contain an Fc domain. An Fc region can be an Fc domain.

An antibody construct can be an antibody comprising a sequence of the IgG1 isoform that has been modified from the wild type IgG1 sequence. A wild type IgG sequence can comprise SEQ ID NO: 162. A modification can comprise a substitution at more than one amino acid residue such as at 5 different amino acid residues including L235V/F243L/R292P/Y300L/P396L (G1VLPLL). The numbering of amino acids residues described herein can be according to the EU index as in Kabat.

The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (G1AAA). The 3 amino acid residues can be located in a portion of an antibody sequence which can encode an Fc region of the antibody and in particular, can be located in portions of the Fc region that can bind Fc receptors (i.e., the Fc domain).

An antibody construct can be a monoclonal anti-CD40 human antibody comprising a sequence of the IgG1 isoform that has been modified from the wildtype IgG1 sequence. A wildtype IgG1 sequence can comprise SEQ ID NO: 15. A modification can comprise a substitution at more than one amino acid residue such as at 5 different amino acid residues including L235V/F243L/R292P/Y300L/P396L (SBT-040-G1VLPLL). The numbering of amino acids residues described herein can be according to the EU index as in Kabat. The 5 amino acid residues can be located in a portion of an antibody sequence which can encode an Fc region of the antibody and in particular, can be located in portions of the Fc region that can bind to Fc receptors (i.e., the Fc domain). A modification can comprise a substitution at more than one amino acid residue such as at 2 different amino acid residues including S239D/I332E (SBT-040-G1DE). The 2 amino acid residues can be located in a portion of an antibody sequence which encodes an Fc region of the antibody and in particular, are located in portions of the Fc region that can bind to Fc receptors (i.e., the Fc domain). A modification can comprise a substitution at more than one amino acid residue such as at 3 different amino acid residues including S298A/E333A/K334A (SBT-040-G1AAA). The 3 amino acid residues can be located in a portion of an antibody sequence which can encode an Fc region of the antibody and in particular, can be located in portions of the Fc region that can bind Fc receptors (i.e., the Fc domain).

Binding of Fc receptors to an Fc region can be affected by amino acid substitutions. For example, FIG. 4C illustrates SBT-040-G1VLPLL, which is an antibody with an amino acid sequence (SEQ ID NO: 16) of a heavy chain of human anti-CD40 monoclonal antibody with modifications to a wild-type IgG1 Fc domain (L235V/F243L/R292P/Y300L/P396L). Binding of some Fc receptors to the Fc region of SBT-040-G1VLPLL can be enhanced compared to wild-type by as result of the L235V/F243L/R292P/Y300L/P396L amino acid modifications. However, binding of other Fc receptors to the Fc region of SBT-040-G1VLPLL can be reduced compared to wild-type by the L235V/F243L/R292P/Y300L/P396L amino acid modifications. For example, the binding affinities of SBT-040-G1VLPLL to FcγRIIIA and to FcγRIIA can be enhanced compared to wild-type whereas the binding affinity of SBT-040-GVLPLL to FcγRIIB can be reduced compared to wild-type. FIG. 4D illustrates an SBT-040-G1DE antibody, which is an antibody with an amino acid sequence (SEQ ID NO: 17) of a heavy chain of human anti-CD40 monoclonal antibody with modifications to a wild-type IgG1 Fc domain (S239D/I332E). Binding of Fc receptors to the Fc region of SBT-040-DE can be enhanced compared to wild-type as a result of the S239D/I332E amino acid modification. However, binding of some Fc receptors to the Fc region of SBT-040-G1DE can be reduced compared to wild-type by S239D/I332E amino acid modification. For example, the binding affinities of SBT-040-G1DE to FcγRIIIA and to FcγRIIB can be enhanced compared to wild-type. Binding of Fc receptors to an Fc region of are affected by amino acid substitutions. FIG. 4E illustrates an SBT-040-G1AAA antibody, which is an antibody with an amino acid sequence (SEQ ID NO: 18) of a heavy chain of a human anti-CD40 monoclonal antibody with modifications to a wild-type IgG1 Fc domain (S298A/E333A/K334A). Binding of Fc receptors to an Fc region of SBT-040-G1AAA can be enhanced compared to wild-type as a result of the S298A/E333A/K334A amino acid modification. However, binding of some Fc receptors to the Fc region of SBT-040-G1AAA can be reduced compared to wild-type by S298A/E333A/K334A amino acid modification. Binding affinities of SBT-040-G1AAA to FcγRIIIA can be enhanced compared to wild-type whereas the binding affinity of SBT-040-G1AAA to FcγRIIB can be reduced compared to wildtype.

In some embodiments, the heavy chain of a human IgG2 antibody can be mutated at cysteines as positions 127, 232, or 233. In some embodiments, the light chain of a human IgG2 antibody can be mutated at a cysteine at position 214. The mutations in the heavy and light chains of the human IgG2 antibody can be from a cysteine residue to a serine residue.

An antibody construct can be a heavy chain of an anti-CD40 antibody. A heavy chain of an anti-CD40 antibody can be SBT-040-G1VLPLL. SBT-040-G1VLPLL be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCCCAAATCTTGTGACAAAACTCACACATGC CCACCGTGCCCAGCACCTGAACTCGTGGGGGGACCGTCAGTCTTCCTCCTGCCCCCAAA ACCCAAGGACACCCTCATGATCTCCCGGACCCCTGAGGTCACATGCGTGGTGGTGGACG TGAGCCACGAAGACCCTGAGGTCAAGTTCAACTGGTACGTGGACGGCGTGGAGGTGCA TAATGCCAAGACAAAGCCGCCTGAGGAGCAGTACAACAGCACGCTGCGTGTGGTCAGC GTCCTCACCGTCCTGCACCAGGACTGGCTGAATGGCAAGGAGTACAAGTGCAAGGTCTC CAACAAAGCCCTCCCAGCCCCCATCGAGAAAACCATCTCCAAAGCCAAAGGGCAGCCC CGAGAACCACAGGTGTACACCCTGCCCCCATCCCGGGAGGAGATGACCAAGAACCAGG TCAGCCTGACCTGCCTGGTCAAAGGCTTCTATCCCAGCGACATCGCCGTGGAGTGGGAG AGCAATGGGCAGCCGGAGAACAACTACAAGACCACGCCTCTGGTGCTGGACTCCGACG GCTCCTTCTTCCTCTATAGCAAGCTCACCGTGGACAAGAGCAGGTGGCAGCAGGGGAAC GTCTTCTCATGCTCCGTGATGCATGAGGCTCTGCACAACCACTACACGCAGAAGAGCCT CTCCCTGTCCCCGGGTAAATGA (SEQ ID NO: 9) wherein the DNA sequence comprises DNA nucleotide modifications that correspond to L235V, F243L, R292P, Y300L and P396L amino acid residue modifications compared to a wild-type DNA sequence. SBT-040-G1VLPLL can be expressed from a DNA sequence comprising greater than 70%, greater than 75%, greater than 80%, greater than 85%, greater than 90%, greater than 95% or greater than 99% homology to SEQ ID NO: 9. A variable region of SBT-040-G1VLPLL can be expressed from a DNA sequence comprising SEQ ID NO: 13. A variable region of SBT-040-G1VLPLL can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1VLPLL expressed from SEQ ID NO: 9, or expressed from a DNA sequence comprising greater than 70% homology to SEQ ID NO: 9 can have a dissociation constant (K_{d}) for CD40 that is less than 10 nM. Anti-CD40 antibodies comprising SBT-040-G1VLPLL expressed from DNA sequence comprising SEQ ID NO: 9, or comprising greater than 70% homology to SEQ ID NO: 9 can have a dissociation constant (K_{d}) for CD40 that is less than 1 nM, less than 100 pM, less than 10 pM, less than 1 pM, or less than 0.1 pM. SBT-040-G1VLPLL can be expressed with any anti-CD40 light chain or fragment thereof. SBT-040-G1VLPLL can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G1VLPLL can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVEPKSCDKTHTCPPCPAPELVGGPSVFLLPPKPKDTLMISRTPEVTCVVVDVSHEDPEV KFNWYVDGVEVHNAKTKPPEEQYNSTLRVVSVLTVLHQDWLNGKEYKCKVSNKALPAPIE KTISKAKGQPREPQVYTLPPSREEMTKNQVSLTCLVKGFYPSDIAVEWESNGQPENNYKTTP LVLDSDGSFFLYSKLTVDKSRWQQGNVFSCSVMHEALHNHYTQKSLSLSPGK (SEQ ID NO: 16) wherein the amino acid sequence comprises L235V, F243L, R292P, Y300L, and P396L amino acid residue modifications compared to a wild-type amino acid sequence. SBT-040-G1VLPLL can comprise an amino acid sequence with greater than 70%, greater than 75%, greater than 80%, greater than 85%, greater than 90%, greater than 95% or greater than 99% homology to SEQ ID NO: 16. SBT-040-G1VLPLL can comprise an amino acid sequence SEQ ID NO: 20. A variable region of SBT-040-G1VLPLL can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1VLPLL with SEQ ID NO: 16 or with an amino acid sequence with greater than 70% homology to SEQ ID NO: 16 can have a dissociation constant (K_{d}) for CD40 that is less than 10 nM. Anti-CD40 antibodies comprising SBT-040-G1VLPLL with SEQ ID NO: 16 or with an amino acid sequence with greater than 70% homology to SEQ ID NO: 16 can have a dissociation constant (K_{d}) for CD40 that is less than 1 nM, less than 100 pM, less than 10 pM, less than 1 pM, or less than 0.1 pM. SBT-040-G1VLPLL can be purified. SBT-040-G1VLPLL can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

A heavy chain of an anti-CD40 antibody can be SBT-040-G1DE. SBT-040-G1DE be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCCCAAATCTTGTGACAAAACTCACACATGC CCACCGTGCCCAGCACCTGAACTCCTGGGGGGACCGGATGTCTTCCTCTTCCCCCCAAA ACCCAAGGACACCCTCATGATCTCCCGGACCCCTGAGGTCACATGCGTGGTGGTGGACG TGAGCCACGAAGACCCTGAGGTCAAGTTCAACTGGTACGTGGACGGCGTGGAGGTGCA TAATGCCAAGACAAAGCCGCGGGAGGAGCAGTACAACAGCACGTACCGTGTGGTCAGC GTCCTCACCGTCCTGCACCAGGACTGGCTGAATGGCAAGGAGTACAAGTGCAAGGTCTC CAACAAAGCCCTCCCAGCCCCCGAGGAGAAAACCATCTCCAAAGCCAAAGGGCAGCCC CGAGAACCACAGGTGTACACCCTGCCCCCATCCCGGGAGGAGATGACCAAGAACCAGG TCAGCCTGACCTGCCTGGTCAAAGGCTTCTATCCCAGCGACATCGCCGTGGAGTGGGAG AGCAATGGGCAGCCGGAGAACAACTACAAGACCACGCCTCCCGTGCTGGACTCCGACG GCTCCTTCTTCCTCTATAGCAAGCTCACCGTGGACAAGAGCAGGTGGCAGCAGGGGAAC GTCTTCTCATGCTCCGTGATGCATGAGGCTCTGCACAACCACTACACGCAGAAGAGCCT CTCCCTGTCCCCGGGTAAATGA (SEQ ID NO: 10) wherein the DNA sequence comprises DNA nucleotide modifications that correspond to S239D and I332E amino acid residue modifications compared to a wild-type DNA sequence. SBT-040-G1DE can be expressed from a DNA sequence comprising greater than 70%, greater than 75%, greater than 80%, greater than 85%, greater than 90%, greater than 95% or greater than 99% homology to SEQ ID NO: 10. A variable region of SBT-040-G1DE can be expressed from a DNA sequence comprising SEQ ID NO: 13. A variable region of SBT-040-G1DE can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1DE expressed from SEQ ID NO: 10, or expressed from a DNA sequence comprising greater than 70% homology to SEQ ID NO: 10 can have a dissociation constant (K_{d}) for CD40 that is less than 10 nM. Anti-CD40 antibodies comprising SBT-040-G1DE expressed from DNA sequence comprising SEQ ID NO: 10, or comprising greater than 70% homology to SEQ ID NO: 10 can have a dissociation constant (K_{d}) for CD40 that is less than 1 nM, less than 100 pM, less than 10 pM, less than 1 pM, or less than 0.1 pM. SBT-040-G1DE can be expressed with any anti-CD40 light chain or fragment thereof. SBT-040-G1DE can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G1DE can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVEPKSCDKTHTCPPCPAPELLGGPDVFLFPPKPKDTLMISRTPEVTCVVVDVSHEDPEV KFNWYVDGVEVHNAKTKPREEQYNSTYRVVSVLTVLHQDWLNGKEYKCKVSNKALPAPE EKTISKAKGQPREPQVYTLPPSREEMTKNQVSLTCLVKGFYPSDIAVEWESNGQPENNYKTT PPVLDSDGSFFLYSKLTVDKSRWQQGNVFSCSVMHEALHNHYTQKSLSLSPGK (SEQ ID NO: 17) wherein the amino acid sequence comprises S239D and I332E amino acid residue modifications compared to a wild-type amino acid sequence. SBT-040-G1DE can comprise an amino acid sequence with greater than 70%, greater than 75%, greater than 80%, greater than 85%, greater than 90%, greater than 95% or greater than 99% homology to SEQ ID NO: 17. SBT-040-GIDE can comprise an amino acid sequence SEQ ID NO: 20. A variable region of SBT-040-G1DE can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1DE with SEQ ID NO: 17 or with an amino acid sequence with greater than 70% homology to SEQ ID NO: 17 can have a dissociation constant (K_{d}) for CD40 that is less than 10 nM. Anti-CD40 antibodies comprising SBT-040-G1DE with SEQ ID NO: 17 or with an amino acid sequence with greater than 70% homology to SEQ ID NO: 17 can have a dissociation constant (K_{d}) for CD40 that is less than 1 nM, less than 100 pM, less than 10 pM, less than 1 pM, or less than 0.1 pM. SBT-040-G1DE can be purified. SBT-040-G1DE can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

A heavy chain of an anti-CD40 antibody can be SBT-040-G1AAA. SBT-040-G1AAA be expressed from a DNA sequence comprising ATGGACTGGACCTGGAGGATCCTCTTCTTGGTGGCAGCAGCCACAGGAGCCCACTCCCA GGTGCAGCTGGTGCAGTCTGGGGCTGAGGTGAAGAAGCCTGGGGCCTCAGTGAAGGTC TCCTGCAAGGCTTCTGGATACACCTTCACCGGCTACTATATGCACTGGGTGCGACAGGC CCCTGGACAAGGGCTTGAGTGGATGGGATGGATCAACCCTGACAGTGGTGGCACAAAC TATGCACAGAAGTTTCAGGGCAGGGTCACCATGACCAGGGACACGTCCATCAGCACAG CCTACATGGAGCTGAACAGGCTGAGATCTGACGACACGGCCGTGTATTACTGTGCGAGA GATCAGCCCCTAGGATATTGTACTAATGGTGTATGCTCCTACTTTGACTACTGGGGCCA GGGAACCCTGGTCACCGTCTCCTCAGCCTCCACCAAGGGCCCATCGGTCTTCCCCCTGG CGCCCTGCTCCAGGAGCACCTCCGAGAGCACAGCGGCCCTGGGCTGCCTGGTCAAGGA CTACTTCCCCGAACCGGTGACGGTGTCGTGGAACTCAGGCGCTCTGACCAGCGGCGTGC ACACCTTCCCAGCTGTCCTACAGTCCTCAGGACTCTACTCCCTCAGCAGCGTGGTGACC GTGCCCTCCAGCAACTTCGGCACCCAGACCTACACCTGCAACGTAGATCACAAGCCCAG CAACACCAAGGTGGACAAGACAGTTGAGCCCAAATCTTGTGACAAAACTCACACATGC CCACCGTGCCCAGCACCTGAACTCCTGGGGGGACCGTCAGTCTTCCTCTTCCCCCCAAA ACCCAAGGACACCCTCATGATCTCCCGGACCCCTGAGGTCACATGCGTGGTGGTGGACG TGAGCCACGAAGACCCTGAGGTCAAGTTCAACTGGTACGTGGACGGCGTGGAGGTGCA TAATGCCAAGACAAAGCCGCGGGAGGAGCAGTACAACGCCACGTACCGTGTGGTCAGC GTCCTCACCGTCCTGCACCAGGACTGGCTGAATGGCAAGGAGTACAAGTGCAAGGTCTC CAACAAAGCCCTCCCAGCCCCCATCGCCGCTACCATCTCCAAAGCCAAAGGGCAGCCCC GAGAACCACAGGTGTACACCCTGCCCCCATCCCGGGAGGAGATGACCAAGAACCAGGT CAGCCTGACCTGCCTGGTCAAAGGCTTCTATCCCAGCGACATCGCCGTGGAGTGGGAGA GCAATGGGCAGCCGGAGAACAACTACAAGACCACGCCTCCCGTGCTGGACTCCGACGG CTCCTTCTTCCTCTATAGCAAGCTCACCGTGGACAAGAGCAGGTGGCAGCAGGGGAACG TCTTCTCATGCTCCGTGATGCATGAGGCTCTGCACAACCACTACACGCAGAAGAGCCTC TCCCTGTCCCCGGGTAAATGA (SEQ ID NO: 11) wherein the DNA sequence comprises DNA nucleotide modifications that correspond to S298A, E333A, and K334A amino acid residue modifications compared to a wild-type DNA sequence. SBT-040-G1AAA can be expressed from a DNA sequence comprising greater than 70%, greater than 75%, greater than 80%, greater than 85%, greater than 90%, greater than 95% or greater than 99% homology to SEQ ID NO: 11. A variable region of SBT-040-G1AAA can be expressed from a DNA sequence comprising SEQ ID NO: 13. A variable region of SBT-040-G1AAA can be expressed from a DNA sequence comprising greater than 70%, greater than 75%, greater than 80%, greater than 85%, greater than 90%, greater than 95% or greater than 99% homology to SEQ ID NO: 13. Additionally, anti-CD40 antibodies comprising SBT-040-G1AAA expressed from SEQ ID NO: 11, or expressed from a DNA sequence comprising greater than 70% homology to SEQ ID NO: 11 can have a dissociation constant (K_{d}) for CD40 that is less than 10 nM. Anti-CD40 antibodies comprising SBT-040-G1AAA expressed from DNA sequence comprising SEQ ID NO: 11, or comprising greater than 70% homology to SEQ ID NO: 11 can have a dissociation constant (K_{d}) for CD40 that is less than 1 nM, less than 100 pM, less than 10 pM, less than 1 pM, or less than 0.1 pM. SBT-040-G1AAA can be expressed with any anti-CD40 light chain or fragment thereof. SBT-040-G1AAA can also be expressed with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to antibody constructs comprising anti-CD40 antibodies created for use in the veterinary sciences and/or in laboratory animals.

SBT-040-G1AAA can comprise an amino acid sequence MDWTWRILFLVAAATGAHSQVQLVQSGAEVKKPGASVKVSCKASGYTFTGYYMHWVRQ APGQGLEWMGWINPDSGGTNYAQKFQGRVTMTRDTSISTAYMELNRLRSDDTAVYYCAR DQPLGYCTNGVCSYFDYWGQGTLVTVSSASTKGPSVFPLAPCSRSTSESTAALGCLVKDYF PEPVTVSWNSGALTSGVHTFPAVLQSSGLYSLSSVVTVPSSNFGTQTYTCNVDHKPSNTKV DKTVEPKSCDKTHTCPPCPAPELLGGPSVFLFPPKPKDTLMISRTPEVTCVVVDVSHEDPEV KFNWYVDGVEVHNAKTKPREEQYNATYRVVSVLTVLHQDWLNGKEYKCKVSNKALPAPI AATISKAKGQPREPQVYTLPPSREEMTKNQVSLTCLVKGFYPSDIAVEWESNGQPENNYKT TPPVLDSDGSFFLYSKLTVDKSRWQQGNVFSCSVMHEALHNHYTQKSLSLSPGK (SEQ ID NO: 18) wherein the amino acid sequence comprises S298A, E333A, and K334A amino acid residue modifications compared to a wild-type amino acid sequence. SBT-040-G1AAA can comprise an amino acid sequence with greater than 70%, greater than 75%, greater than 80%, greater than 85%, greater than 90%, greater than 95% or greater than 99% homology to SEQ ID NO: 18. SBT-040-G1AAA can comprise an amino acid sequence SEQ ID NO: 20. A variable region of SBT-040-G1AAA can comprise an amino acid sequence with greater than 70%, greater than 75%, greater than 80%, greater than 85%, greater than 90%, greater than 95% or greater than 99% homology to SEQ ID NO: 20. Additionally, anti-CD40 antibodies comprising SBT-040-G1AAA with SEQ ID NO: 18 or with an amino acid sequence with greater than 70% homology to SEQ ID NO: 18 can have a dissociation constant (K_{d}) for CD40 that is less than 10 nM. Anti-CD40 antibodies comprising SBT-040-G1AAA with SEQ ID NO: 18 or with an amino acid sequence with greater than 70% homology to SEQ ID NO: 18 can have a dissociation constant (K_{d}) for CD40 that is less than 1 nM, less than 100 pM, less than 10 pM, less than 1 pM, or less than 0.1 pM. SBT-040-G1AAA can be purified. SBT-040-G1AAA can be combined with any anti-CD40 light chain or fragment thereof to form an anti-CD40 antibody or fragment thereof. The anti-CD40 antibody or fragment thereof can be purified, and can be combined with a pharmaceutically acceptable carrier. The anti-CD40 antibody can be an antibody construct. Additionally, one skilled in the art would recognize that these same concepts could apply to anti-CD40 antibodies created for use in the veterinary sciences and/or in laboratory animals.

While an antibody construct of the present disclosure can comprise an anti-CD40 antibody with wild-type or modified amino acid sequences encoding the Fc region or Fc domain, the modifications of the Fc region or the Fc domain from the wild-type sequence may not significantly alter binding and/or affinity of the anti-CD40 antibody for CD40. For example, binding and/or affinity of SBT-040-G1WT, SBT-040-G1VLPLL, SBT-040-G1DE, and SBT-040-G1AAA may not be significantly altered by modification of an Fc region or Fc domain amino acid sequence compared to a wild-type sequence. Modifications of an Fc region or Fc domain from a wild-type sequence may not alter binding and/or affinity of antibodies that bind to CD40 in an antibody construct. Additionally, the binding and/or affinity of the antibodies described herein that bind to CD40 and are antibody constructs, for example SBT-040-G1WT, SBT-040-G1VLPLL, SBT-040-G1DE, and SBT-040-G1AAA, may be comparable to the binding and/or affinity of wild-type antibodies that can bind to CD40.

Sequences that can be used to produce antibodies for antibody constructs can include leader sequences. Leader sequences can be signal sequences. Leader sequences useful with the compositions and methods described herein can include, but are not limited to, a DNA sequence comprising ATGAGGCTCCCTGCTCAGCTCCTGGGGCTCCTGCTGCTCTGGTTCCCAGGTTCCAGATGC (SEQ ID NO: 2) or ATGGACTGGACCTGGAGGATCCTCTTCTTGGTGGCAGCAGCCACAGGAGCCCACTCC (SEQ ID NO: 12), or an amino acid sequence comprising MRLPAQLLGLLLLWFPGSRC (SEQ ID NO: 5) and MDWTWRILFLVAAATGAHS (SEQ ID NO: 19). Leader sequence can comprise a DNA sequence with greater than 70%, greater than 75%, greater than 80%, greater than 85%, greater than 90%, greater than 95% or greater than 99% homology to SEQ ID NO: 2 or SEQ ID NO: 12. Leader sequence can comprise an amino acid sequence with greater than 70%, greater than 75%, greater than 80%, greater than 85%, greater than 90%, greater than 95% or greater than 99% homology to SEQ ID NO: 5 or SEQ ID NO: 19. Any of the sequences described herein can be used with or without a leader sequence. Additionally, one skilled in the art would recognize that these same concepts can apply to antibody constructs comprising anti-CD40 antibodies created for use in the veterinary sciences and/or in laboratory animals.

Targeting Binding Domain

An antibody construct can further comprise a targeting binding domain. A targeting domain can comprise a domain that binds to a target. A target can be an antigen. A targeting domain can comprise an antigen binding domain. A targeting domain can be a domain that can specifically bind to an antigen A targeting domain can be an antigen-binding portion of an antibody or an antibody fragment. A targeting domain can be one or more fragments of an antibody that can retain the ability to specifically bind to an antigen. A targeting domain can be any antigen binding fragment. A targeting domain can be in a scaffold, in which a scaffold is a supporting framework for the antigen binding domain. A targeting domain can comprise an antigen binding domain in a scaffold.

A targeting domain can comprise an antigen binding domain which can refer to a portion of an antibody comprising the antigen recognition portion, i.e., an antigenic determining variable region of an antibody sufficient to confer recognition and binding of the antigen recognition portion to a target, such as an antigen, i.e., the epitope. Examples of a targeting domain can include, but are not limited to, Fab, single chain variable fragment (scFv), variable Fv fragment and other fragments, combinations of fragments or types of fragments known or knowable to one of ordinary skill in the art. A targeting domain can comprise an antigen binding domain which can refer to a portion of an antibody comprising the antigen recognition portion, i.e., an antigenic determining variable region of an antibody sufficient to confer recognition and binding of the antigen recognition portion to a target, such as an antigen, i.e., the epitope. Examples of a targeting domain can include, but are not limited to, Fab, single chain variable fragment (scFv), variable Fv fragment and other fragments, combinations of fragments or types of fragments known or knowable to one of ordinary skill in the art.

A targeting domain can comprise an antigen binding domain of an antibody. An antigen binding domain of an antibody can comprise one or more light chain (LC) CDRs and one or more heavy chain (HC) CDRs. For example, an antibody binding domain of an antibody can comprise one or more of the following: a light chain complementary determining region 1 (LC CDR1), a light chain complementary determining region 2 (LC CDR2), or a light chain complementary determining region 3 (LC CDR3). For another example, an antibody binding domain can comprise one or more of the following: a heavy chain complementary determining region 1 (HC CDR1), a heavy chain complementary determining region 2 (HC CDR2), or a heavy chain complementary determining region 3 (HC CDR3).

An antibody construct can comprise an antibody fragment. An antibody fragment can include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, can be coded for by separate genes, they can be linked by a synthetic linker to be made as a single protein chain in which the V_{L} and V_{H} regions pair to form monovalent molecules.

F(ab′)₂ and Fab′ moieties can be produced by treating immunoglobulin (monoclonal antibody) with a protease such as pepsin and papain, and can include an antibody fragment generated by digesting immunoglobulin near the disulfide bonds existing between the hinge regions in each of the two H chains.

An Fv can be the minimum antibody fragment which contains a complete antigen-recognition and antigen-binding site. This region can consist of a dimer of one heavy chain and one light chain variable domain in tight, non-covalent association. In this configuration the three hypervariable regions of each variable domain can interact to define an antigen-binding site on the surface of the V_{H}-V_{L} dimer. A single variable domain (or half of an Fv comprising only three hypervariable regions specific for an antigen) can recognize and bind antigen, although at a lower affinity than the entire binding site.

A targeting domain can be at least 80% homologous to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

A targeting domain can comprise an antigen binding domain comprising a light chain and a heavy chain from a monoclonal antibody. In one aspect, a targeting domain binds to CD40 and comprises the light chain of an anti-CD40 antibody and the heavy chain of an anti-CD40 antibody, which bind a CD40 antigen. In another aspect, the targeting domain binds to a tumor antigen comprises the light chain of a tumor antigen antibody and the heavy chain of a tumor antigen antibody, which bind the tumor antigen.

A targeting domain can bet attached to an antibody construct. For example, an antibody construct can be fused with a targeting binding domain to create an antibody construct targeting binding domain fusion. The antibody construct-targeting binding domain fusion can be the result of the nucleic acid sequence of the targeting binding domain being expressed in frame with the nucleic acid sequence of the antibody construct. The antibody construct-targeting binding domain fusion can be the result of an in-frame genetic nucleotide sequence or a contiguous peptide sequence encoding the antibody construct with the targeting binding domain. As another example, a targeting binding domain can be linked to an antibody construct. A targeting binding domain can be linked to an antibody construct by a chemical conjugation. The targeting binding domain can direct the antibody construct to, for example, a particular cell or cell type. A targeting binding domain of an antibody construct can be selected in order to recognize an antigen. For example, an antigen can be expressed on an immune cell. An antigen can be a peptide or fragment thereof. An antigen can be expressed on an antigen-presenting cell. An antigen can be expressed on a dendritic cell, a macrophage, or a B cell. An antigen can be CD40 and a targeting binding domain can recognize a CD40 antigen. A targeting binding domain can be a CD40 agonist. A targeting domain can recognize CD40 on, for example, an antigen-presenting cell. As another example, an antigen can be a tumor antigen. The tumor antigen can be any tumor antigen described herein.

Immune-Stimulatory Compounds

Pattern recognition receptors (PRRs) can recognize pathogen-associated molecular patterns (PAMPs) and damage-associated molecular patterns (DAMPs). A PRR can be membrane bound. A PRR can be cytosolic. A PRR can be a toll-like receptor (TLR). A PRR can be RIG-I-like receptor. A PRR can be a receptor kinase. A PRR can be a C-type lectin receptor. A PRR can be a NOD-like receptor. A PRR can be TLR1, TLR2, TLR3, TLR4, TLR5, TLR6, TLR7, TLR8, TLR9, TLR10, TLR11, TLR12 or TLR13.

A PRR agonist can be pathogen-associated molecular pattern (PAMP) molecule. A PAMP molecule can be a toll-like receptor agonist. A PRR agonist can be a toll-like receptor agonist. A toll-like receptor agonist can be any molecule that acts as an agonist to at least one toll-like receptor. A toll-like receptor agonist can be bacterial lipoprotein. A toll-like receptor agonist can be bacterial peptidoglycans. A toll-like receptor agonist can be double stranded RNA. A toll-like receptor agonist can be lipopolysaccharides. A toll-like receptor agonist can be bacterial flagella. A toll-like receptor agonist can be single stranded RNA. A toll-like receptor can be CpG DNA. A toll-like receptor agonist can be imiquimod. A toll-like receptor agonist can be CL307. A toll-like receptor agonist can be S-27609. A toll-like receptor agonist can be resiquimod. A toll-like receptor agonist can be UC-IV150. A toll-like receptor agonist can be gardiquimod. A toll-like receptor agonist can be motolimod. A toll-like receptor agonist can be VTX-1463. A toll-like receptor agonist can be GS-9620. A toll-like receptor agonist can be GSK2245035. A toll-like receptor agonist can be TMX-101. A toll-like receptor agonist can be TMX-201. A toll-like receptor agonist can be TMX-202. A toll-like receptor agonist can be isatoribine. A toll-like receptor agonist can be AZD8848. A toll-like receptor agonist can be MEDI9197. A toll-like receptor agonist can be 3M-051. A toll-like receptor agonist can be 3M-852. A toll-like receptor agonist can be 3M-052. A toll-like receptor agonist can be 3M-854A. A toll-like receptor agonist can be S-34240. A toll-like receptor agonist can be CL663. A RIG-I agonist can be KIN1148. A RIG-I agonist can be SB-9200. A RIG-I agonist can be KIN700, KIN600, KIN500, KIN100, KIN101, KIN400, or KIN2000. A toll-like receptor agonist can be KU34B.

A PRR agonist can be a damage-associated molecular pattern (DAMP) molecule. A DAMP molecule can be an intracellular protein. A DAMP molecule can be a heat-shock protein. A DAMP molecule can be an HMGB1 protein. A DAMP molecule can be a protein derived from the extracellular matrix that is generated after tissue injury. A DAMP molecule can be a hyaluronan fragment. A DAMP molecule can be DNA. A DAMP molecule can be RNA. A DAMP molecule can be an S100 molecule. A DAMP molecule can be nucleotides. A DAMP molecule can be an ATP. A DAMP molecule can be nucleosides. A DAMP molecule can be an adenosine. A DAMP molecule can be uric acid.

Additionally, stimulator of interferon genes (STING) can act as a cytosolic DNA sensor wherein cytosolic DNA and unique bacterial nucleic acids called cyclic dinucleotides are recognized by STING, and therefore STING agonists. Interferon Regulatory Factor (IRF) agonist can be KIN-100. Non-limiting examples of STING agonists include:

[chemical structure]

wherein in some embodiments, X₁=X₂=O; X₃=G; X₄=G; X₅=CO(CH₂)₁₂CH₃; X₆=2 TEAH; in some embodiments, X₁=X₂=S [Rₚ,Rₚ]; X₃=G; X₄=A; X₅=H; X₆=2 TEAH; in some embodiments, X₁=X₂=S [Rₚ,Rₚ]; X₃=A; X₄=A; X₅=H; X₆=2 Na; in some embodiments, X₁=X₂=S [Rₚ,Rₚ]; X₃=A; X₄=A; X₅=H; X₆=2 NH₄; and in some embodiments, X₁=X₂=O; X₃=G; X₄=A; X₅=H; X₆=2 TEAH,

[chemical structure]

[chemical structure]

wherein R₁=R₂=H; R₁=propargyl, R₂=H; R₁=H, R₂=propargyl; R₁=allyl, R₂=H; R₁=H, R₂=allyl; R₁=methyl, R₂=H; R₁=H, R₂=methyl; R₁=ethyl, R₂=H; R₁=H, R₂=ethyl; R₁=propyl, R₂=H; R₁=H, R₂=propyl; R₁=benzyl, R₂=H; R₁=H, R₂=benzyl; R₁=myristoyl, R₂=H; R₁=H, R₂=myristoyl; R₁=R₂=heptanoyl; R₁=R₂=hexanoyl; or R₁=R₂=pentanoyl,

[chemical structure]

wherein R₁=R₂=H; R₁=propargyl, R₂=H; R₁=H, R₂=propargyl; R₁=allyl, R₂=H; R₁=H, R₂=allyl; R₁=methyl, R₂=H; R₁=H, R₂=methyl; R₁=ethyl, R₂=H; R₁=H, R₂=ethyl; R₁=propyl, R₂=H; R₁=H, R₂=propyl; R₁=benzyl, R₂=H; R₁=H, R₂=benzyl; R₁=myristoyl, R₂=H; R₁=H, R₂=myristoyl; R₁=R₂=heptanoyl; R₁=R₂=hexanoyl; or R₁=R₂=pentanoyl,

[chemical structure]

wherein R₁=R₂=H; R₁=propargyl, R₂=H; R₁=H, R₂=propargyl; R₁=allyl, R₂=H; R₁=H, R₂=allyl; R₁=methyl, R₂=H; R₁=H, R₂=methyl; R₁=ethyl, R₂=H; R₁=H, R₂=ethyl; R₁=propyl, R₂=H; R₁=H, R₂=propyl; R₁=benzyl, R₂=H; R₁=H, R₂=benzyl; R₁=myristoyl, R₂=H; R₁=H, R₂=myristoyl; R₁=R₂=heptanoyl; R₁=R₂=hexanoyl; or R₁=R₂=pentanoyl,

[chemical structure]

wherein each X is independently O or S, and R₃ and R₄ are each independently H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C₁₋₆ alkyl straight or branched chain, benzyl, halogen, trihalomethyl, C₁₋₆ alkoxy, —NO₂, —NH₂, —OH, ═O, —COOR′ where R′ is H or lower alkyl, —CH₂OH, and —CONH₂, wherein R3 and R4 are not both H,

[chemical structure]

wherein X₁=X₂=O; X₁=X₂=S; or X₁=O and X₂=S,

[chemical structure]

[chemical structure]

An immune-stimulatory compound can be a PRR agonist. An immune-stimulatory compound can be a PAMP. An immune-stimulatory compound can be a DAMP. An immune-stimulatory compound can be a TLR agonist. An immune-stimulatory compound can be a STING agonist. An immune-stimulatory compound can be a cyclic dinucleotide.

An immune-stimulatory compound can be a drug.

The specificity of the antigen-binding domain to an antigen of an antibody construct in an antibody construct immune-stimulatory compound conjugate as disclosed herein can be influenced by the presence of an immune-stimulatory compound. The antigen-binding domain of the antibody construct in an antibody construct immune-stimulatory compound conjugate can bind to an antigen with at least about 10%, about 20%, about 30%, about 40%, about 50%, about 60%, about 70%, about 80%, about 85%, about 90%, about 95%, or about 100% of a specificity of the antigen-binding domain to the antigen in the absence of the immune-stimulatory compound.

The specificity of the Fc domain to an Fc receptor of an antibody construct in an antibody construct immune-stimulatory compound conjugate as disclosed herein can be influenced by the presence of an immune-stimulatory compound. The Fc domain of the antibody construct in an antibody construct immune-stimulatory compound conjugate can bind to an Fc receptor with at least about 10%, about 20%, about 30%, about 40%, about 50%, about 60%, about 70%, about 80%, about 85%, about 90%, about 95%, or about 100% of a specificity of the Fc domain to the Fc receptor in the absence of the immune-stimulatory compound.

The affinity of the antigen-binding domain to an antigen of an antibody construct in an antibody construct immune-stimulatory compound conjugate as disclosed herein can be influenced by the presence of an immune-stimulatory compound. The antigen-binding domain of the antibody construct in an antibody construct immune-stimulatory compound conjugate can bind to an antigen with at least about 1%, about 5%, about 10%, about 20%, about 30%, about 40%, about 50%, about 60%, about 70%, about 80%, about 85%, about 90%, about 95%, or about 100% of an affinity of the antigen-binding domain to the antigen in the absence of the immune-stimulatory compound.

The affinity of the Fc domain to an Fc receptor of an antibody construct in an antibody construct immune-stimulatory compound conjugate as disclosed herein can be influenced by the presence of an immune-stimulatory compound. The Fc domain of the antibody construct in an antibody construct immune-stimulatory compound conjugate can bind to an Fc receptor with at least about 1%, about 5%, about 10%, about 20%, about 30%, about 40%, about 50%, about 60%, about 70%, about 80%, about 85%, about 90%, about 95%, or about 100% of an affinity of the Fe domain to the Fc receptor in the absence of the immune-stimulatory compound.

The K_{d} for binding of an antigen-binding domain of an antibody construct immune-stimulatory compound conjugate to an antigen in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen of an antibody construct in the absence of the immune-stimulatory compound. The K_{d} for binding of an antigen-binding domain of an antibody construct immune-stimulatory compound conjugate to an antigen in the presence of the immune-stimulatory compound can be less than 10 nM. The K_{d} for binding of an antigen-binding domain of an antibody construct immune-stimulatory compound conjugate to an antigen in the presence of the immune-stimulatory compound can be less than 100 nM, less than 50 nM, less than 20 nM, less than 5 nM, less than 1 nM, or less than 0.1 nM.

The K_{d} for binding of an Fc domain of an antibody construct immune-stimulatory compound conjugate to a Fc receptor in the presence of the immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-stimulatory compound.

The K_{d} for binding of an Fc domain of an antibody construct immune-stimulatory compound conjugate to an Fc receptor in the presence of the immune-stimulatory compound can be less than 10 nM. The K_{d} for binding of an Fc domain of an antibody construct immune-stimulatory compound conjugate to an Fc receptor in the presence of the immune-stimulatory compound can be less than 10 μM, less than 1 μM, less than 100 nM, less than 50 nM, less than 20 nM, less than 5 nM, less than 1 nM, or less than 0.1 nM.

Affinity can be the strength of the sum total of noncovalent interactions between a single binding site of a molecule, for example, an antibody, and the binding partner of the molecule, for example, an antigen. The affinity can also measure the strength of an interaction between an Fc portion of an antibody and the Fc receptor. Unless indicated otherwise, as used herein, “binding affinity” can refer to intrinsic binding affinity which reflects a 1:1 interaction between members of a binding pair (e.g., antibody and antigen or Fc domain and Fc receptor). The affinity of a molecule X for its partner Y can generally be represented by the dissociation constant (K_{d}). Affinity can be measured by common methods known in the art, including those described herein. Specific illustrative and exemplary embodiments for measuring binding affinity are described in the following.

In some embodiments, an antibody provided herein can have a dissociation constant (K_{d}) of about 1 pM, about 100 nM, about 10 nM, about 5 nM, about 2 nM, about 1 nM, about 0.5 nM, about 0.1 nM, about 0.05 nM, about 0.01 nM, or about 0.001 nM or less (e.g., 10⁻⁸ M or less, e.g., from 10⁻⁸ M to 10⁻¹³ M, e.g., from 10⁻⁹M to 10⁻¹³ M). An affinity matured antibody can be an antibody with one or more alterations in one or more complementarity determining regions (CDRs), compared to a parent antibody, which may not possess such alterations, such alterations resulting in an improvement in the affinity of the antibody for antigen. These antibodies can bind to their antigen with a K_{d} of about 5×10⁻⁹M, about 2×10⁻⁹ M, about 1×10⁻⁹ M, about 5×10⁻¹ M, about 2×10⁻⁹ M, about 1×10⁻¹⁰ M, about 5×10⁻¹¹ M, about 1×10⁻¹¹ M, about 5×10⁻¹² M, about 1×10⁻¹² M, or less. In some embodiments, the antibody construct can have an increased affinity of at least 1.5-fold, 2-fold, 2.5-fold, 3-fold, 4-fold, 5-fold, 10-fold, 20-fold, or greater as compared to an antibody construct without alterations in one or more complementarity determining regions.

K_{d} can be measured by any suitable assay. For example, K_{d} can be measured by a radiolabeled antigen binding assay (RIA). For example, K_{d} can be measured using surface plasmon resonance assays (e.g., using a BIACORE®-2000 or a BIACORE®-3000).

Agonism can be described as the binding of a chemical to a receptor to induce a biological response. A chemical can be, for example, a small molecule, a compound, or a protein. An agonist causes a response, an antagonist can block the action of an agonist, and an inverse agonist can cause a response that is opposite to that of the agonist. A receptor can be activated by either endogenous or exogenous agonists.

The molar ratio of an antibody construct immune-stimulatory compound conjugate can refer to the average number of immune-stimulatory compounds conjugated to the antibody construct in a preparation of an antibody construct immune-stimulatory compound conjugate. The molar ratio can be determined, for example, by Liquid Chromatography/Mass Spectrometry (LC/MS), in which the number of immune-stimulatory compounds conjugated to the antibody construct can be directly determined. Additionally, as non-limiting examples, the molar ratio can be determined based on hydrophobic interaction chromatography (HIC) peak area, by liquid chromatography coupled to electrospray ionization mass spectrometry (LC-ESI-MS), by UV/Vis spectroscopy, by reversed-phase-HPLC (RP-HPLC), or by matrix-assisted laser desorption/ionization time of flight mass spectrometry (MALDI-TOF-MS).

In some embodiments, the molar ratio of immune-stimulatory compound to antibody can be less than 8. In other embodiments, the molar ratio of immune-stimulatory compound to antibody can be 8, 7, 6, 5, 4, 3, 2, or 1.

In some aspects, the present disclosure provides a compound represented by the structure of Formula (I):

[chemical structure]

or a pharmaceutically acceptable salt thereof, wherein:

X¹ is selected from —OR² and —SR²;

X² is selected from —OR³ and —SR³;

B¹ and B² are independently selected from optionally substituted nitrogenous bases;

Y is selected from —OR⁴, —NR⁴R⁴, and halogen;

R¹, R², R³ and R⁴ are independently selected at each occurrence from hydrogen, —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹, R², R³ and R⁴ is independently optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, and C₂₋₆ alkynyl; and

R¹⁰⁰ at each occurrence is independently selected from hydrogen; and C₁₋₁₀ alkyl, C₂₋₁₀ alkenyl, C₂₋₁₀ alkynyl, C₃₋₁₀ carbocycle, and 3- to 10-membered heterocycle each of which is independently optionally substituted at each occurrence with one or more substituents selected from halogen, —CN, —NO₂, ═O, ═S, and haloalkyl.

In some embodiments, the compound of Formula (I) is represented by Formula (IA):

[chemical structure]

or pharmaceutically acceptable salts thereof.

In an alternative embodiment, the compound of Formula (I) is represented by Formula (IB):

[chemical structure]

or a pharmaceutically acceptable salt thereof.

In various embodiments, B¹ and B² are independently selected from optionally substituted

[chemical structure]

purines. In certain embodiments, B¹ and B² are independently selected from: H. In certain embodiments, B¹ and B² are independently selected from optionally substituted pyrimidines.

In some embodiments, optionally substituted purines may include optionally substituted adenine, optionally substituted guanine, optionally substituted xanthine, optionally substituted hypoxaanthine, optionally substituted theobromine, optionally substituted caffeine, optionally substituted uric acid, and optionally substituted isoguanine. In certain embodiments, B¹ and B² are independently selected from:

[chemical structure]

optionally substituted by one or more additional substituents.

In certain embodiments, B¹ and B² are independently selected from:

[chemical structure]

wherein the point of connectivity of B¹ to the remainder of the compound is represented by

[chemical structure]

In a preferred embodiment, B¹ and B² are independently selected from optionally substituted adenine and optionally substituted guanine. In certain embodiments, B¹ and B² are independently selected from:

[chemical structure]

optionally further substituted by one or more substituents. In certain embodiments, B¹ and B² are independently selected from:

[chemical structure]

In some embodiments, B¹ and B² are independently optionally substituted with one or more substituents, wherein the optional substituents on B¹ and B² are independently selected at each occurrence from halogen, ═O, ═S, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, —P(O)(OR¹⁰⁰)₂, —OP(O)(OR¹⁰⁰)₂ and —CN; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle is independently optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, and C₂₋₆ alkynyl.

In certain embodiments, B¹ and B² are independently optionally substituted with one or more substituents, wherein the optional substituents on B¹ and B² are independently selected at each occurrence from halogen, ═O, ═S, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, —P(O)(OR¹⁰⁰)₂, —OP(O)(OR¹⁰⁰)₂, —CN and C₁₋₁₀ alkyl.

In some embodiments, B¹ is an optionally substituted guanine. In certain embodiments, B¹ is

[chemical structure]

In certain embodiments, B¹ is

[chemical structure]

wherein the point of connectivity of B¹ to the remainder of the compound is represented by

[chemical structure]

In some embodiments, B1 is an optionally substituted adenine. In certain embodiments, B¹ is

[chemical structure]

In certain embodiments, B¹ is

[chemical structure]

wherein the point of connectivity of B¹ to the remainder of the compound is represented by

[chemical structure]

In some embodiments, B² is an optionally substituted guanine. In certain, embodiments, B² is

[chemical structure]

In certain embodiments, B² is

[chemical structure]

wherein the point of connectivity on B² is represented by

[chemical structure]

In some embodiments, B² is an optionally substituted adenine. In certain embodiments, B² is

[chemical structure]

In certain embodiments, B² is

[chemical structure]

wherein the point of connectivity on B² is represented by

[chemical structure]

In some embodiments, B¹ is an optionally substituted guanine and B² is an optionally substituted guanine. In some embodiments, B1 is an optionally substituted adenine and B² is an optionally substituted guanine.

In various embodiments, X is selected from —OH and —SH. For example, X¹ may be —OH. In various embodiments, X² is selected from —OH and —SH. For example, X² may be —OH. In some embodiments, X¹ is —OH and X² is —OH. In some embodiments, X¹ is —SH and X² is —SH.

In various embodiments, Y is selected from —OH, —O—C₁₋₁₀ alkyl, —NH(C₁₋₁₀ alkyl), and —NH₂. For example, Y may be —OH.

In various embodiments, R¹⁰⁰ is independently selected at each occurrence from hydrogen and C₁₋₁₀ alkyl optionally substituted at each occurrence with one or more substituents selected from halogen, —CN, —NO₂, ═O, and ═S.

In various embodiments, the compound of Formula (I) is represented by Formula (IC):

[chemical structure]

or a pharmaceutically acceptable salt thereof. In some embodiments, the compound of Formula (IC) is represented by Formula (ID):

[chemical structure]

or a pharmaceutically acceptable salt thereof.

In various embodiments, the compound is a pharmaceutically acceptable salt. In some embodiments, the compound or salt is a modulator of a stimulator of interferon genes (STING). The compound or salt may agonize a stimulator of interferon genes (STING). In certain embodiments, the compound or salt may cause STING to coordinate multiple immune responses to infection, including the induction of interferons and STAT6-dependent response and selective autophagy response. In certain embodiments, the compound or salt may cause STING to mediate type I interferon production.

In some aspects, the present disclosure provides an antibody drug conjugate, comprising a compound or salt previously described, an antibody, and a linker group, wherein the compound or salt is linked, e.g., covalently bound, to the antibody through the linker group. The linker group may be selected from a cleavable or non-cleavable linker. In some embodiments, the linker group is cleavable. In alternative embodiments, the linker group is non-cleavable. Linkers are further described in the present application in the subsequent section, any one of which may be used to connect an antibody to a compound described herein.

In some aspects, the present disclosure provides a compound represented by the structure of Formula (II):

[chemical structure]

or a pharmaceutically acceptable salt thereof, wherein:

X¹ is selected from —OR² and —SR²;

X² is selected from —OR³ and —SR³;

B¹ and B² are independently selected from optionally substituted nitrogenous bases, wherein each optional substituent is independently selected from halogen, —OR, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, R⁶, and —X³;

Y is selected from —OR⁴, —SR⁴, —NR⁴R⁴, and halogen;

Z is selected from —OR⁵, —SR⁵, and —NR⁵R⁵;

R¹, R², R³, R⁴, and R⁵ are independently selected from a —X³; hydrogen, —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R¹, R², R³, R⁴, and R⁵ is optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, C₂₋₆ alkynyl;

R⁶ is independently selected from —C(═O)R¹⁰⁰, —C(═O)OR¹⁰⁰ and —C(═O)NR¹⁰⁰; C₁₋₁₀ alkyl, C₂₋₁₀ alkenyl, C₂₋₁₀ alkynyl, each of which is independently optionally substituted at each occurrence with one or more substituents selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₃₋₁₀ carbocycle and 3- to 10-membered heterocycle; and C₃₋₁₀ carbocycle and 3- to 10-membered heterocycle, wherein each C₃₋₁₀ carbocycle and 3- to 10-membered heterocycle in R⁶ is optionally substituted with one or more substituents selected from halogen, —OR¹⁰⁰, —SR¹⁰⁰, —N(R¹⁰⁰)₂, —S(O)R¹⁰⁰, —S(O)₂R¹⁰⁰—C(O)R¹⁰⁰, —C(O)OR¹⁰⁰, —OC(O)R¹⁰⁰, —NO₂, ═O, ═S, ═N(R¹⁰⁰), —P(O)(OR¹⁰⁰)₂, —OP(O)(OR¹⁰⁰)₂, —CN, C₁₋₆ alkyl, C₂₋₆ alkenyl, C₂₋₆ alkynyl;

R¹⁰⁰ at each occurrence is independently selected from hydrogen; and C₁₋₁₀ alkyl, C₂₋₁₀ alkenyl, C₂₋₁₀ alkynyl, C₃₋₁₀ carbocycle, and 3- to 10-membered heterocycle each of which is independently optionally substituted at each occurrence with one or more substituents selected from halogen, —CN, —NO₂, ═O, ═S, and haloalkyl; and

X³ is a linker moiety, wherein at least one of R¹, R², R³, R⁴, R⁵, X¹, X², a B¹ substituent and a B² substituent is —X³.

In various embodiments, the compound of Formula (II) is represented by a structure of Formula (IIA):

[chemical structure]

or pharmaceutically acceptable salts thereof.

In various embodiments, the compound of Formula (II) is represented by a structure of Formula (IIB):

[chemical structure]

or a pharmaceutically acceptable salt thereof.

In various embodiments, B¹ and B² are independently selected from optionally substituted purines. B¹ and B² may be each, independently selected from one another, adenine, guanine, and derivatives thereof. B¹ and B² may be independently selected from optionally substituted adenine, optionally substituted guanine, optionally substituted xanthine, optionally substituted hypoxanthine, optionally substituted theobromine, optionally substituted caffeine, optionally substituted uric acid, and optionally substituted isoguanine. In a preferred embodiment, B¹ and B² are independently selected from optionally substituted adenine and optionally substituted guanine.

In various embodiments, B¹ is substituted by X³ and optionally one or more additional substituents independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, and R⁶. For example, B¹ may be represented by:

[chemical structure]

and wherein B¹ is optionally further substituted by one or more substituents.

In various embodiments, B² is substituted by X³ and optionally one or more additional substituents independently selected from halogen, —OR¹⁰⁰, —SR¹⁰⁰, —N(R¹⁰⁰)₂, —S(O)R¹⁰⁰, —S(O)₂R¹⁰⁰, —C(O)R¹⁰⁰, —C(O)OR¹⁰⁰, —OC(O)R¹⁰⁰, —NO₂, ═O, ═S, ═N(R¹⁰⁰), —CN, and R⁶. For example, B² may be represented by:

[chemical structure]

and wherein B² is optionally further substituted by one or more substituents.

In some embodiments, B¹ is represented by

[chemical structure]

and B² is represented by

[chemical structure]

In some embodiments, B¹ is represented by

[chemical structure]

N and B² is represented by

[chemical structure]

In various embodiments, X¹ is selected from —O—X³ and —S—X³. In some embodiments, X¹ is selected from —OH and —SH. In some embodiments, X¹ is —SH.

In various embodiments, X² is selected from —O—X³ and —S—X³. In some embodiments, X² is selected from —OH and —SH. In some embodiments, X² is —S—X³.

In some embodiments, X¹ is —SH and X² is —S—X³.

In certain embodiments, Y is selected from —NR⁴X³, —S—X³, and —O—X³. In some embodiments, Y is selected from —OH, —SH, —O—C₁₋₁₀ alkyl, —NH(C₁₋₁₀ alkyl), and —NH₂. In a preferred embodiment, Y is selected from —OH.

In various embodiments, Z is selected from —NR⁴X³, —S—X³, and —O—X³. In some embodiments, Z is selected from —OH, —SH, —O—C₁₋₁₀ alkyl, —NH(C₁₋₁₀ alkyl), and —NH₂.

In various embodiments, —X³ is represented by the formula:

[chemical structure]

In some embodiments, —X³ is represented by the formula:

[chemical structure]

wherein RX comprises a reactive moiety, such a maleimide.

In some embodiments, —X³ is represented by the formula:

[chemical structure]

wherein RX* is a reactive moiety that has reacted with a moiety on an antibody-drug conjugate.

[chemical structure]

wherein RX is a reactive moiety, such as a maleimide.

In some embodiments, —X³ is represented by the formula:

[chemical structure]

wherein RX* is a reactive moiety that has reacted with a moiety on an antibody to form an antibody drug conjugate.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof.

The compound is represented by the formula:

[chemical structure]

or a pharmaceutically acceptable salt thereof.

The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically actable salt thereof. The compound may be represented by the formula:

[chemical structure]

or a pharmaceutically acceptable salt thereof.

In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.
In some embodiments, the compound is represented by the formula:

[chemical structure]

or a pharmaceutically acceptable salt thereof. The compound may be represented by the formula:

[chemical structure]

or a pharmaceutically acceptable salt thereof. The compound may be represented by the formula:

[chemical structure]

or a pharmaceutically acceptable salt thereof.

### Linkers

The compositions and methods described herein can comprise a linker, e.g., a peptide linker. Linkers of the compositions and methods described herein may not affect the binding of active portions of a conjugate (e.g., active portions include antigen binding domains, Fc domains, targeting binding domains, antibodies, agonists or the like) to a target, which can be a cognate binding partner such as an antigen. A linker sequence can form a linkage between different parts of a composition. A composition can be a conjugate. A conjugate can comprise multiple linkers. These linkers can be the same linkers or different linkers.

Attachment via a linker can involve incorporation of a linker between parts of a composition or conjugate. A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker can be a valine-citrulline linker or a valine-alanine linker. A valine-citrulline or valine-alanine linker can contain a pentafluorophenyl group. A valine-citrulline or valine-alanine linker can contain a succimide group. A valine-citrulline or valine-alanine linker can contain a para aminobenzoic acid (PABA) group. A valine-citrulline or valine-alanine linker can contain a PABA group and a pentafluorophenyl group. A valine-citrulline or valine-alanine linker can contain a PABA group and a succinimide group. A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonly)-(NH₂) linker. A linker can be a THIOMAB linker. A THIOMAB linker can be a (maleimidocaproyl)-(valine-citrulline)-(para-aminobenzyloxycarbonyl)-(NH₂) linker. A linker can also be an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG recognition motif (SEQ ID NO: 21) to an N-terminal GGG motif to regenerate a native amide bond. The linker created can therefore link a moiety attached to the LXPTG recognition motif (SEQ ID NO: 21) with a moiety attached to the N-terminal GGG motif A linker can be a link created between an unnatural amino acid on one moiety reacting with oxime bond that was formed by modifying a ketone group with an alkoxyamine on another moiety.

A moiety can be an antibody construct. A moiety can be an antibody. A moiety can be an immune-stimulatory compound. A moiety can be a targeting binding domain. A linker can be a portion of a linker can be unsubstituted or substituted, for example, with a substituent. A substituent can include, for example, hydroxyl groups, amino groups, nitro groups, cyano groups, azido groups, carboxyl groups, carboxaldehyde groups, imine groups, alkyl groups, alkenyl groups, alkynyl groups, alkoxy groups, acyl groups, acyloxy groups, amide groups, and ester groups.

In the antibody construct immune-stimulatory compound conjugate described herein, the immune-stimulatory compound is linked to the antibody construct by way of linkers. The linker linking an immune-stimulatory compound to the antibody of an antibody construct immune-stimulatory compound conjugate can be short, long, hydrophobic, hydrophilic, flexible or rigid, or may be composed of segments that each independently have one or more of the above-mentioned properties such that the linker may include segments having different properties. The linkers can be polyvalent such that they covalently link more than one immune-stimulatory compound to a single site on the antibody construct, or monovalent such that covalently they link a single immune-stimulatory compound to a single site on the antibody construct.

As will be appreciated by skilled artisans, the linkers link the immune-stimulatory compound to the antibody by forming a covalent linkage to the immune-stimulatory compound at one location and a covalent linkage to the antibody construct at another. The covalent linkages are formed by reaction between functional groups on the linker and functional groups on the inhibitors and antibody construct. As used herein, the expression “linker” is intended to include (i) unconjugated forms of the linker that include a functional group capable of covalently linking the linker to an immune-stimulatory compound and a functional group capable of covalently linking the linker to an antibody construct; (ii) partially conjugated forms of the linker that include a functional group capable of covalently linking the linker to an antibody construct and that is covalently linked to an immune-stimulatory compound, or vice versa; and (iii) fully conjugated forms of the linker that is covalently linked to both an immune-stimulatory compound and an antibody construct. In some specific embodiments of intermediate synthons and antibody construct immune-stimulatory compound conjugates described herein, moieties comprising the functional groups on the linker and covalent linkages formed between the linker and antibody construct are specifically illustrated as Rx and LK, respectively. One embodiment pertains to an antibody construct immune-stimulatory compound conjugate formed by contacting an antibody construct that binds a cell surface receptor or tumor associated antigen expressed on a tumor cell with a synthon described herein under conditions in which the synthon covalently links to the antibody construct. One embodiment pertains to a method of making an antibody construct immune-stimulatory compound conjugate formed by contacting a synthon described herein under conditions in which the synthon covalently links to the antibody construct. One embodiment pertains to a method of stimulating immune activity in a cell that expresses CD40, comprising contacting the cell with an antibody construct immune-stimulatory compound conjugate described herein that is capable of binding the cell, under conditions in which the antibody construct immune-stimulatory compound conjugate binds the cell.

Exemplary polyvalent linkers that may be used to link many immune-stimulatory compounds to an antibody construct are described. For example, Fleximer® linker technology has the potential to enable high-DAR antibody construct immune-stimulatory compound conjugate with good physicochemical properties. As shown below, the Fleximer® linker technology is based on incorporating drug molecules into a solubilizing poly-acetal backbone via a sequence of ester bonds. The methodology renders highly-loaded antibody construct immune-stimulatory compound conjugates (DAR up to 20) whilst maintaining good physicochemical properties. This methodology could be utilized with immune-stimulatory compound as shown in the Scheme below.

[chemical structure]

To utilize the Fleximer® linker technology depicted in the scheme above, an aliphatic alcohol can be present or introduced into the immune-stimulatory compound. The alcohol moiety is then conjugated to an alanine moiety, which is then synthetically incorporated into the Fleximer® linker. Liposomal processing of the antibody construct immune-stimulatory compound conjugate in vitro releases the parent alcohol-containing drug.

By way of example and not limitation, some cleavable and noncleavable linkers that may be included in the antibody construct immune-stimulatory compound conjugates described herein are described below.

Cleavable linkers can be cleavable in vitro and in vivo. Cleavable linkers can include chemically or enzymatically unstable or degradable linkages. Cleavable linkers can rely on processes inside the cell to liberate an immune-stimulatory compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate immune-stimulatory compound release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the immune-stimulatory compound once the antibody construct immune-stimulatory compound conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Antibody construct immune-stimulatory compound conjugates including exemplary hydrazone-containing linkers can include, for example, the following structures:

[chemical structure]

wherein D and Ab represent the immune-stimulatory compound and antibody construct, respectively, and n represents the number of immune-stimulatory compound—linkers linked to the antibody construct. In certain linkers such as linker (Ig), the linker can comprise two cleavable groups—a disulfide and a hydrazone moiety. For such linkers, effective release of the unmodified free immune-stimulatory compound can require acidic pH or disulfide reduction and acidic pH. Linkers such as (Ih) and (Ii) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the immune-stimulatory compound upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the immune-stimulatory compound in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Antibody construct immune-stimulatory compound conjugates including exemplary disulfide-containing linkers can include the following structures:

[chemical structure]

wherein D and Ab represent the immune-stimulatory compound and antibody construct, respectively, n represents the number of immune-stimulatory compound-linkers linked to the antibody construct and R is independently selected at each occurrence from hydrogen or alkyl, for example. Increasing steric hindrance adjacent to the disulfide bond can increase the stability of the linker. Structures such as (Ij) and (Il) can show increased in vivo stability when one or more R groups is selected from a lower alkyl such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of an immune-stimulatory compound from an antibody construct can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides such as Gly-Phe-Leu-Gly (SEQ ID NO: 175), Ala-Leu-Ala-Leu (SEQ ID NO: 176) or dipeptides such as Val-Cit, Val-Ala, and Phe-Lys. Dipeptides can have lower hydrophobicity compared to longer peptides.

A variety of dipeptide-based cleavable linkers can be used in the antibody constructs immune-stimulatory compound conjugates described herein.

Enzymatically cleavable linkers can include a self-immolative spacer to spatially separate the immune-stimulatory compound from the site of enzymatic cleavage. The direct attachment of an immune-stimulatory compound to a peptide linker can result in proteolytic release of an amino acid adduct of the immune-stimulatory compound, thereby impairing its activity. The use of a self-immolative spacer can allow for the elimination of the fully active, chemically unmodified immune-stimulatory compound upon amide bond hydrolysis.

One self-immolative spacer can be a bifunctional para-aminobenzyl alcohol group, which can link to the peptide through the amino group, forming an amide bond, while amine containing immune-stimulatory compounds can be attached through carbamate functionalities to the benzylic hydroxyl group of the linker (to give a p-amidobenzylcarbamate, PABC). The resulting pro-immune-stimulatory compound can be activated upon protease-mediated cleavage, leading to a 1,6-elimination reaction releasing the unmodified immune-stimulatory compound, carbon dioxide, and remnants of the linker group. The following scheme depicts the fragmentation of p-amidobenzyl carbamate and release of the immune-stimulatory compound:

[chemical structure]

wherein X-D represents the unmodified immune-stimulatory compound. Heterocyclic variants of this self-immolative group have also been described.

The enzymatically cleavable linker can be a β-glucuronic acid-based linker. Facile release of the immune-stimulatory compound can be realized through cleavage of the β-glucuronide glycosidic bond by the lysosomal enzyme 8-glucuronidase. This enzyme can be abundantly present within lysosomes and can be overexpressed in some tumor types, while the enzyme activity outside cells can be low. β-Glucuronic acid-based linkers can be used to circumvent the tendency of an antibody construct immune-stimulatory compound conjugate to undergo aggregation due to the hydrophilic nature of β-glucuronides. In certain embodiments, β-glucuronic acid-based linkers can link an antibody construct to a hydrophobic immune-stimulatory compound. The following scheme depicts the release of an immune-stimulatory compound (D) from an antibody construct (Ab) immune-stimulatory compound conjugate containing a β-glucuronic acid-based linker:

[chemical structure]

A variety of cleavable β-glucuronic acid-based linkers useful for linking drugs such as auristatins, camptothecin and doxorubicin analogues, CBI minor-groove binders, and psymberin to antibodies have been described. All of these β-glucuronic acid-based linkers may be used in the ADCs described herein. In certain embodiments, the enzymatically cleavable linker is a β-galactoside-based linker. β-Galactoside is present abundantly within lysosomes, while the enzyme activity outside cells is low.

Additionally, immune-stimulatory compounds containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on an immune-stimulatory compound, wherein such ester groups can hydrolyze under physiological conditions to release the immune-stimulatory compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide moiety, for example, a linker comprising structural formula (IVa), (IVb), (IVc), or (IVd):

[chemical structure]

or a salt thereof, wherein: peptide represents a peptide (illustrated N→C, wherein peptide includes the amino and carboxy “termini”) a cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R is hydrogen or C₁₋₄ alkyl-(O)ᵣ(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or sugar moiety; G² is SO₃H, CO₂H, or PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to the immune-stimulatory compound; and * represents the point of attachment to the remainder of the linker.

In certain embodiments, the peptide can be selected from a tripeptide or a dipeptide. In particular embodiments, the dipeptide can be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IVa) that can be included in the antibody construct immune-stimulatory compound conjugates described herein can include the linkers illustrated below (as illustrated, the linkers include a group suitable for covalently linking the linker to an antibody construct):

[chemical structure]

[chemical structure]

Exemplary embodiments of linkers according to structural formula (IVb), (IVc), (IVd) that can include in the anti body construct immune-stimulatory compound conjugates described herein can include the linkers illustrated below (as illustrated, the linkers can include a group suitable for covalently linking the linker to an antibody construct):

[chemical structure]

[chemical structure]

[chemical structure]

[chemical structure]

[chemical structure]

[chemical structure]

[chemical structure]

[chemical structure]

The linker can contain an enzymatically cleavable sugar moiety, for example, a linker comprising structural formula (Va), (Vb), (Vc), (Vd), or (Ve):

[chemical structure]

[chemical structure]

or a salt thereof, wherein: q is 0 or 1; r is 0 or 1; X¹ is CH₂, O or NH; [symbol] represents the point of attachment of the linker to the immune-stimulatory compound; and * represents the point of attachment to the remainder of the linker.

Exemplary embodiments of linkers according to structural formula (Va) that can be included in the antibody construct immune-stimulatory compound conjugates described herein can include the linkers illustrated below (as illustrated, the linkers include a group suitable for covalently linking the linker to an antibody construct):

[chemical structure]

[chemical structure]

[chemical structure]

Exemplary embodiments of linkers according to structural formula (Vb) that may be included in the antibody construct immune-stimulatory compound conjugates described herein include the linkers illustrated below (as illustrated, the linkers include a group suitable for covalently linking the linker to an antibody construct):

[chemical structure]

[chemical structure]

[chemical structure]

Exemplary embodiments of linkers according to structural formula (Vc) that may be included in the antibody construct immune-stimulatory compound conjugates described herein include the linkers illustrated below (as illustrated, the linkers include a group suitable for covalently linking the linker to an antibody construct):

[chemical structure]

[chemical structure]

[chemical structure]

Exemplary embodiments of linkers according to structural formula (Vd) that may be included in the antibody construct immune-stimulatory compound conjugates described herein include the linkers illustrated below (as illustrated, the linkers include a group suitable for covalently linking the linker to an antibody construct).

[chemical structure]

[chemical structure]

Exemplary embodiments of linkers according to structural formula (Ve) that may be included in the antibody construct immune-stimulatory compound conjugates described herein include the linkers illustrated below (as illustrated, the linkers include a group suitable for covalently linking the linker to an antibody construct):

[chemical structure]

Although cleavable linkers can provide certain advantages, the linkers comprising the conjugate described herein need not be cleavable. For non-cleavable linkers, the immune-stimulatory compound release may not depend on the differential properties between the plasma and some cytoplasmic compartments. The release of the immune-stimulatory compound can occur after internalization of the antibody construct immune-stimulatory compound conjugate via antigen-mediated endocytosis and delivery to lysosomal compartment, where the antibody construct can be degraded to the level of amino acids through intracellular proteolytic degradation. This process can release a immune-stimulatory compound derivative, which is formed by the immune-stimulatory compound, the linker, and the amino acid residue to which the linker was covalently attached. The immune-stimulatory compound derivative from antibody construct immune-stimulatory compound conjugates with non-cleavable linkers can be more hydrophilic and less membrane permeable, which can lead to less bystander effects and less nonspecific toxicities compared to antibody construct immune-stimulatory compound conjugates with a cleavable linker. Antibody construct immune-stimulatory compound conjugates with non-cleavable linkers can have greater stability in circulation than antibody construct immune-stimulatory compound conjugates with cleavable linkers. Non-cleavable linkers can be alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

[chemical structure]

or salts thereof, wherein: R^{a} is selected from hydrogen, alkyl, sulfonate and methyl sulfonate; R^{x} is a moiety including a functional group capable of covalently linking the linker to an antibody construct; and [symbol] represents the point of attachment of the linker to the immune-stimulatory compound.

Exemplary embodiments of linkers according to structural formula (VIa)-(VId) that may be included in the conjugates described herein include the linkers illustrated below (as illustrated, the linkers include a group suitable for covalently linking the linker to an antibody construct, and [symbol] represents the point of attachment to an immune-stimulatory compound):

[chemical structure]

Attachment groups that are used to attach the linkers to an antibody can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

One example of a “self-stabilizing” maleimide group that hydrolyzes spontaneously under antibody construct conjugation conditions to give an antibody construct immune-stimulatory compound conjugate species with improved stability is depicted in the schematic below. Thus, the maleimide attachment group is reacted with a sulfhydryl of an antibody construct to give an intermediate succinimide ring. The hydrolyzed form of the attachment group is resistant to deconjugation in the presence of plasma proteins.

[chemical structure]

[chemical structure]

A method for bridging a pair of sulfhydryl groups derived from reduction of a native hinge disulfide bond has been disclosed and is depicted in the schematic below. An advantage of this methodology is the ability to synthesize homogenous DAR4 antibody construct immune-stimulatory compound conjugates by full reduction of IgGs (to give 4 pairs of sulfhydryls) followed by reaction with 4 equivalents of the alkylating agent. Antibody construct immune-stimulatory compound conjugates containing “bridged disulfides” are also claimed to have increased stability.

[chemical structure]

Similarly, as depicted below, a maleimide derivative that is capable of bridging a pair of sulfhydryl groups has been developed.

[chemical structure]

The attachment moiety can contain the following structural formulas (VIIa), (VIIb), or (VIIc):

[chemical structure]

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIIa) and (VIIb) that can be included in the antibody construct immune-stimulatory compound conjugates described herein can include the linkers illustrated below (as illustrated, the linkers can include a group suitable for covalently linking the linker to an antibody construct):

[chemical structure]

[chemical structure]

[chemical structure]

[chemical structure]

Exemplary embodiments of linkers according to structural formula (VIIc) that can be included in the antibody construct immune-stimulatory compound conjugates described herein can include the linkers illustrated below (as illustrated, the linkers can include a group suitable for covalently linking the linker to an antibody construct).

[chemical structure]

[chemical structure]

### Conjugates

A composition as described herein can be a conjugate. A conjugate can comprise an antibody construct, an immune-stimulatory compound, and a linker. A conjugate can comprise an antibody construct, a pattern recognition receptor (PRR) agonist, and a linker. A conjugate can comprise an antibody construct, a pattern-associated molecular pattern (PAMP) molecule, and a linker. A conjugate can comprise an antibody construct, a damage-associated molecular pattern (DAMP) molecule, and a linker. A conjugate can comprise an antibody construct, a STING agonist, and a linker. A conjugate can comprise an antibody construct, a toll-like receptor agonist molecule, and a linker. A conjugate can comprise an antibody construct, imiquimod, and a linker. A conjugate can comprise an antibody construct, S-27609, and a linker. A conjugate can comprise an antibody construct, CL307, and a linker. A conjugate can comprise an antibody construct, resiquimod, and a linker. A conjugate can comprise an antibody construct, gardiquimod, and a linker. A conjugate can comprise an antibody construct, UC-IV150, and a linker. A conjugate can comprise an antibody construct, KU34B, and a linker. A conjugate can comprise an antibody construct, motolimod, and a linker. A conjugate can comprise an antibody construct, VTX-1463, and a linker. A conjugate can comprise an antibody construct, GS-9620, and a linker. A conjugate can comprise an antibody construct, GSK2245035, and a linker. A conjugate can comprise an antibody construct, TMX-101, and a linker. A conjugate can comprise an antibody construct, TMX-201, and a linker. A conjugate can comprise an antibody construct, TMX-202, and a linker. A conjugate can comprise an antibody construct, isatoribine, and a linker. A conjugate can comprise an antibody construct, AZD8848, and a linker. A conjugate can comprise an antibody construct, MEDI9197, and a linker. A conjugate can comprise an antibody construct, 3M-051, and a linker. A conjugate can comprise an antibody construct, 3M-852, and a linker. A conjugate can comprise an antibody construct, 3M-052, and a linker. A conjugate can comprise an antibody construct, 3M-854A, and a linker. A conjugate can comprise an antibody construct, S-34240, and a linker. A conjugate can comprise an antibody construct, CL663, and a linker. A conjugate can comprise an antibody construct, KIN1148, and a linker. A conjugate can comprise an antibody construct, SB-9200, and a linker. A conjugate can comprise an antibody construct, KIN-100, and a linker. A conjugate can comprise an antibody construct, ADU-S100, and a linker. A conjugate can comprise an antibody construct, KU34B, and a linker. An antibody construct of any of the conjugates described herein can have a modified Fc domain of the antibody construct. The modified Fc domain can comprise a substitution at more than one amino acid residue such as at 5 different amino acid residues including L235V/F243L/R292P/Y300L/P396L, as at 2 different amino acid residues including S239D/I332E, or as at 3 different amino acid residues including S298A/E333A/K334A. The numbering of amino acids residues described herein can be according to the EU index as in Kabat.

A conjugate can comprise an antibody construct, a targeting binding domain, an immune-stimulatory compound, and a linker. A conjugate can comprise an antibody construct, a targeting binding domain, a pattern recognition receptor (PRR) agonist, and a linker. A conjugate can comprise an antibody construct, a targeting binding domain, a pattern-associated molecular pattern (PAMP) molecule, and a linker. A conjugate can comprise an antibody construct, a targeting binding domain, a damage-associated molecular pattern (DAMP) molecule, and a linker. A conjugate can comprise an antibody construct, a targeting binding domain, a STING agonist, and a linker. A conjugate can comprise an antibody construct, a targeting binding domain, a toll-like receptor agonist molecule, and a linker. A conjugate can comprise an antibody construct, a targeting binding domain, imiquimod, and a linker. A conjugate can comprise an antibody construct, a targeting binding domain, S-27609, and a linker. A conjugate can comprise an antibody construct, a targeting binding domain, CL307, and a linker. A conjugate can comprise an antibody construct, a targeting binding domain, resiquimod, and a linker. A conjugate can comprise an antibody construct, a targeting binding domain, gardiquimod, and a linker. A conjugate can comprise an antibody construct, a targeting binding domain, UC-IV150, and a linker. A conjugate can comprise an antibody construct, a targeting binding domain, motolimod, and a linker. A conjugate can comprise an antibody construct, a targeting binding domain, VTX-1463, and a linker. A conjugate can comprise an antibody construct, a targeting binding domain, GS-9620, and a linker. A conjugate can comprise an antibody construct, a targeting binding domain, GSK2245035, and a linker. A conjugate can comprise an antibody construct, a targeting binding domain, TMX-101, and a linker. A conjugate can comprise an antibody construct, a targeting binding domain, TMX-201, and a linker. A conjugate can comprise an antibody construct, a targeting binding domain, TMX-202, and a linker. A conjugate can comprise an antibody construct, a targeting binding domain, isatoribine, and a linker. A conjugate can comprise an antibody construct, a targeting binding domain, AZD8848, and a linker. A conjugate can comprise an antibody construct, a targeting binding domain, MEDI9197, and a linker. A conjugate can comprise an antibody construct, a targeting binding domain, 3M-051, and a linker. A conjugate can comprise an antibody construct, a targeting binding domain, 3M-852, and a linker. A conjugate can comprise an antibody construct, a targeting binding domain, 3M-052, and a linker. A conjugate can comprise an antibody construct, a targeting binding domain, 3M-854A, and a linker. A conjugate can comprise an antibody construct, a targeting binding domain, S-34240, and a linker. A conjugate can comprise an antibody construct, a targeting binding domain, CL663, and a linker. A conjugate can comprise an antibody construct, a targeting binding domain, KIN1148, and a linker. A conjugate can comprise an antibody construct, a targeting binding domain, SB-9200, and a linker. A conjugate can comprise an antibody construct, a targeting binding domain, KIN-100, and a linker. A conjugate can comprise an antibody construct, a targeting binding domain, ADU-S100, and a linker. A conjugate can comprise an antibody construct, a targeting binding domain, KU34B, and a linker. An antibody construct of any of the conjugates described herein can have a modified Fe domain of the antibody construct. The modified Fe domain can comprise a substitution at more than one amino acid residue such as at 5 different amino acid residues including L235V/F243L/R292P/Y300L/P396L, as at 2 different amino acid residues including S239D/I332E, or as at 3 different amino acid residues including S298A/E333A/K334A.

The linker can be a linker as described herein. A linker can be cleavable, non-cleavable, hydrophilic, or hydrophobic. A cleavable linker can be sensitive to enzymes. A cleavable linker can be cleaved by enzymes such as proteases. A cleavable linker can be a valine-citrulline or a valine-alanine linker. A valine-citrulline or valine-alanine linker can contain a pentafluorophenyl group. A valine-citrulline or valine-alanine linker can contain a succimide group. A valine-citrulline or valine-alanine linker can contain a PABA group. A valine-citrulline or valine-alanine linker can contain a PABA group and a pentafluorophenyl group. A valine-citrulline or valine-alanine linker can contain a PABA group and a succinimide group. A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonly)-(NH₂) linker. A linker can be a THIOMAB linker. A THIOMAB linker can be a (maleimidocaproyl)-(valine-citrulline)-(para-aminobenzyloxycarbonly)-(NH₂) linker. A linker can also be an alkylene, alkenylene, alkynylene, polyether, polyester, polyamide, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link is created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LXPTG (SEQ ID NO: 21) recognition motif to an N-terminal GGG motif to regenerate a native amide bond. The linker created can therefore link a moiety attached to the LXPTG (SEQ ID NO: 21) recognition motif with a moiety attached to the N-terminal GGG motif A linker can be a link created between an unnatural amino acid on one moiety reacting with oxime bond that was formed by modifying a ketone group with an alkoxyamine on another moiety. A moiety can be an antibody construct. A moiety can be a targeting binding domain. A moiety can be an antibody. A moiety can be an immune-stimulatory compound.

The antibody construct can be as described herein. The antibody construct can be an anti-tumor antigen antibody construct. The antibody construct can be an anti-tumor antigen antibody. An antigen recognized by the antibody construct can be CD5, CD19, CD20, CD25, CD37, CD30, CD33, CD45, CAMPATH-1, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MARTI, Ras mutant, gp100, p53 mutant, PR1, bcr-ab1, tyronsinase, survivin, PSA, hTERT, Sarcoma translocation breakpoints, EphA2, PAP, ML-IAP, AFP, ERG, NA17, PAX3, ALK, androgen receptor, cyclin B1, polysialic acid, MYCN, RhoC, TRP-2, fucosyl GM1, mesothelin, PSCA, MAGE A1, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, or Fos-related antigen 1. The antibody construct can recognize an antigen that can be expressed on a cell. The antibody construct can recognize an antigen that can be expressed by a cell. The antibody construct can recognize an antigen that can be expressed in the context of a Major Histocompatibility Complex. The antibody construct can recognize an antigen that can stimulate activity of a cell. The antibody construct can recognize an antigen that can stimulate an immune response. The antibody construct can recognize an antigen that can reduce an immune response. The antibody construct can recognize an antigen can reduce activity of a cell. The antibody construct can recognize an antigen that can be expressed on an immune cell. The antibody construct can recognize an antigen that can be expressed by an immune cell. The antibody construct can recognize an antigen that can be in the context of a Major Histocompatibility Complex. The antibody construct can recognize an antigen on a cell wherein the antigen can be involved in stimulating activity of a cell. The antibody construct can recognize an antigen on an immune cell that can be involved in the costimulation of an immune cell. The antibody construct can recognize an antigen on an immune cell that can be involved in the costimulation of an immune cell during an immune response. The antibody construct can recognize a receptor. The antibody construct can recognize a receptor on a cell. The antibody construct can recognize a receptor ligand. The antibody construct can recognize a receptor on a cell wherein the receptor can be involved in stimulating activity of a cell. The antibody construct can recognize a receptor on an immune cell. The antibody construct can recognize a receptor on an immune cell that can be involved in stimulating activity of an immune cell. The antibody construct can recognize a receptor on an immune cell that can be involved in the costimulation of an immune cell. The antibody construct can recognize a receptor on an immune cell that can be involved in the costimulation of an immune cell during an immune response. The antibody construct can recognize an antigen that can be expressed on an immune cell and that can stimulate activity of an immune cell. The antibody construct can recognize an antigen that can be expressed on an immune that can reduce activity of an immune cell. The antibody construct can be an anti-CD40 antibody. The antibody construct can comprise a light chain of an SBT-040 antibody. The antibody construct can comprise an SBT-040-G1WT heavy chain. The antibody construct can comprise an SBT-040-G1VLPLL heavy chain. The antibody construct can comprise an SBT-040-G1DE heavy chain. The antibody construct can comprise an SBT-040-G1AAA heavy chain. The antibody construct can comprise an SBT-040-CDR sequence. The antibody construct can be capable of recognizing a single antigen. The antibody construct can be capable of recognizing two or more antigens. The K_{d} for binding of an antigen-binding domain of an antibody construct immune-stimulatory compound conjugate to an antigen in the presence of an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the antigen binding domain to the antigen of an antibody construct in the absence of the immune-stimulatory compound. The K_{d} for binding of an antigen-binding domain of an antibody construct immune-stimulatory compound conjugate to an antigen in the presence of the immune-stimulatory compound can be less than 10 nM. The K_{d} for binding of an antigen-binding domain of an antibody construct immune-stimulatory compound conjugate to an antigen in the presence of the immune-stimulatory compound can be less than 100 nM, less than 50 nM, less than 20 nM, less than 5 nM, less than 1 nM, or less than 0.1 nM.

An antibody construct can further comprise a targeting binding domain. A targeting binding domain of an antibody construct can recognize an antigen. For example, an antigen can be expressed on an immune cell. As another example, an antigen can be expressed by a tumor or cancer cell. An antigen can be a peptide or fragment thereof. An antigen can be expressed on an antigen-presenting cell. An antigen can be expressed on a dendritic cell, a macrophage, or a B cell. An antigen can be CD40 and a targeting binding domain can recognize a CD40 antigen. An antigen can be a tumor antigen and a targeting binding domain can recognize a tumor antigen. A targeting binding domain of an antibody construct can be a CD40 agonist. A targeting binding domain of an antibody construct can bind to a tumor antigen.

The antibody construct can have an Fc domain that can bind to an FcR when linked to an immune-stimulatory compound. The antibody construct can have an Fc domain that can bind to an FcR to initiate FcR-mediated signaling when linked to an immune stimulatory compound. The antibody construct can bind to its antigen when linked to an immune-stimulatory compound. The antibody construct can bind to its antigen when linked to an immune-stimulatory compound and the Fc domain of the antibody construct can bind to an FcR when linked to an immune-stimulatory compound. The antibody construct can bind to its antigen when linked to an immune-stimulatory compound and the Fc domain of the antibody can bind to an FcR to initiate FcR-mediated signaling when linked to an immune stimulatory compound. The Fc domain linked to an immune-stimulatory compound can be a modified Fc domain. The modified Fc domain can comprise a substitution at more than one amino acid residue such as at 5 different amino acid residues including L235V/F243L/R292P/Y300L/P396L, as at 2 different amino acid residues including S239D/I332E, or as at 3 different amino acid residues including S298A/E333A/K334A. The K_{d} for binding of an Fc domain to a Fc receptor when the Fc domain is linked to an immune-stimulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-stimulatory compound. The K_{d} for binding of an Fc domain to an Fc receptor when linked to an immune-stimulatory compound can be less than 10 nM. The K_{d} for binding of an Fc domain to an Fc receptor when linked to an immune-stimulatory compound can be less than 100 nM, less than 50 nM, less than 20 nM, less than 5 nM, less than 1 nM, or less than 0.1 nM.

The PRR agonist can be a toll-like receptor agonist. The toll-like receptor agonist can be a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, a TLR10 agonist, a TLR11 agonist, a TLR12 agonist or a TLR13 agonist. The toll-like receptor agonist can activate two or more TLRs. The PAMP molecule can be a RIG-I agonist.

A conjugate can be formed by a linker that can connect an antibody construct to a PRR. A conjugate can be formed by a linker that can connect an antibody construct to a PAMP molecule. A conjugate can be formed by a linker that can connect an antibody construct and a DAMP molecule. A conjugate can be formed by a linker that can connect an antibody construct to a PRR, and a linker that can connect an antibody construct and a targeting binding domain. A conjugate can be formed by a linker that can connect an antibody construct to a PAMP molecule, and a linker that can connect an antibody construct and a targeting binding domain. A conjugate can be formed by a linker that can connect an antibody construct and a DAMP molecule, and a linker that can connect an antibody construct and a targeting binding domain.

A linker can be connected to an antibody construct by a direct linkage between the antibody construct and the linker. A linker can be connected to an anti-CD40 antibody construct by a direct linkage between the anti-CD40 antibody construct and the linker. A linker can be connected to an anti-CD40 antibody by a direct linkage between the anti-CD40 antibody and the linker. A linker can be connected to an anti-tumor antigen antibody construct by a direct linkage between the anti-tumor antigen antibody construct and the linker. A linker can be connected to an anti-tumor antigen antibody by a direct linkage between the anti-tumor antigen antibody and the linker. A direct linkage can be a covalent bond. For example, a linker can be attached to a terminus of an amino acid sequence of an antibody construct, or could be attached to a side chain modification to the antibody construct, such as the side chain of a lysine, serine, threonine, cysteine, tyrosine, aspartic acid, a non-natural amino acid residue, or glutamic acid residue. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the antibody. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. A linker can be connected to an antibody construct at a hinge cysteine. A linker can be connected to an antibody construct at a light chain constant domain lysine. A linker can be connected to an antibody construct at an engineered cysteine in the light chain. A linker can be connected to an antibody construct at an engineered light chain glutamine. A linker can be connected to an antibody construct at an unnatural amino acid engineered into the light chain. A linker can be connected to antibody construct at an unnatural amino acid engineered into the heavy chain. Amino acids can be engineered into an amino acid sequence of a composition as described herein, for example, a linker of a conjugate. Engineered amino acids can be added to a sequence of existing amino acids. Engineered amino acids can be substituted for one or more existing amino acids of a sequence of amino acids. A linker can be conjugated to antibody construct via a sulfhydryl group. A linker can be conjugated to an antibody construct via a primary amine. A linker can be a link created between an unnatural amino acid on an antibody construct reacting with oxime bond that was formed by modifying a ketone group with an alkoxyamine on an immune-stimulatory compound. When a linker is connected to an antibody construct at the sites described herein, an Fc domain of the antibody construct can bind to Fc receptors. When a linker is connected to an antibody construct at the sites described herein, the antigen binding domain of the antibody construct can bind its antigen. When a linker is connected to an antibody construct at the sites described herein, a targeting binding domain of said antibody construct can bind its antigen.

An antibody with engineered reactive cysteine residues (THIOMAB) can be used to link a targeting binding domain to the antibody. A linker can connect an antibody construct to a targeting binding domain via Sortase A linker. A Sortase A linker can be created by a Sortase A enzyme fusing an LXPTG (SEQ ID NO: 21) recognition motif to an N-terminal GGG motif to regenerate a native amide bond. The linker created can therefore link an antibody construct attached to the LXPTG (SEQ ID NO: 21) recognition motif with a targeting binding domain attached to the N-terminal GGG motif. A targeting binding domain can be connected to a linker by a direct linkage. A direct linkage can be a covalent bond. For example, a linker can be attached to a terminus of an amino acid sequence of a targeting binding domain, or could be attached to a side chain modification to the targeting binding domain, such as the side chain of a lysine, serine, threonine, cysteine, tyrosine, aspartic acid, a non-natural amino acid residue, or glutamic acid residue. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the targeting binding domain. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. Amino acids can be engineered into an amino acid sequence of the targeting binding domain. Engineered amino acids can be added to a sequence of existing amino acids. Engineered amino acids can be substituted for one or more existing amino acids of a sequence of amino acids. A linker can be conjugated to a targeting binding domain via a sulfhydryl group. A linker can be conjugated to a targeting binding domain via a primary amine. A targeting binding domain can be conjugated to the C-terminal of an Fc domain of an antibody construct.

An antibody with engineered reactive cysteine residues (THIOMAB) can be used to link an immune-stimulatory compound to the antibody. A linker can connect an antibody construct to an immune-stimulatory compound via THIOMAB linker. A linker can connect an antibody construct to an immune-stimulatory compound via Sortase A linker. A Sortase A linker can be created by a Sortase A enzyme fusing an LXPTG (SEQ ID NO: 21) recognition motif to an N-terminal GGG motif to regenerate a native amide bond. The linker created can therefore link an antibody construct attached the LXPTG (SEQ ID NO: 21) recognition motif with an immune-stimulatory compound attached to the N-terminal GGG motif A linker can be a link created between an unnatural amino acid on an antibody construct reacting with oxime bond that was formed by modifying a ketone group with an alkoxyamine on an immune-stimulatory compound. The immune-stimulatory compound can comprise one or more rings selected from carbocyclic and heterocyclic rings. The immune-stimulatory compound can be covalently bound to a linker by a bond to an exocyclic carbon or nitrogen atom on said immune-stimulatory compound. A linker can be conjugated to an immune-stimulatory compound via an exocyclic nitrogen or carbon atom of an immune-stimulatory compound. A linker can be connected to a STING agonist, for example:

[chemical structure]

A linker agonist complex can dissociate under physiological conditions to yield an active agonist.

A linker can be connected to a PRR agonist by a direct linkage between the PRR agonist and the linker. A linker can be connected to a PAMP molecule by a direct linkage between the PAMP molecule and the linker. A linker can be connected to a toll-like receptor agonist by a direct linkage between the toll-like receptor agonist and the linker.

Examples of toll-like receptor agonists connected to a linker in a manner able to release an active toll-like receptor agonist under physiologic condition can include:

[symbol]

Examples of RIG-I agonists connected to a linker in a manner able to release an active toll-like receptor agonist under physiologic conditions can include:

[chemical structure]

A linker can be connected to a DAMP molecule by a direct linkage between the DAMP molecule and the linker. A direct linkage can be a covalent bond. For example, a linker can be attached to a terminus of an amino acid sequence of an antibody, or could be attached to a side chain modification to the antibody, such as the side chain of a lysine, serine, threonine, cysteine, tyrosine, aspartic acid, a non-natural amino acid residue, or glutamic acid residue. An attachment can be via any of a number of bonds, for example but not limited to, an amide bond, an ester bond, an ether bond, a carbon-nitrogen bond, a carbon-carbon single double or triple bond, a disulfide bond, or a thioether bond. A linker can have at least one functional group, which can be linked to the antibody construct. Non-limiting examples of the functional groups can include those which form an amide bond, an ester bond, an ether bond, a carbonate bond, a carbamate bond, or a thioether bond, such functional groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups.

An ATAC can be formed by conjugating a noncleavable maleimide-PEG4 linker containing a succinimide group with an immune-stimulatory compound. For example, an ATAC can be N-((4-amino-1-(2-hydroxy-2-methylpropyl)-1H-imidazo[4,5-c]quinolin-2-yl)methyl)-1-(3-(2,5-dioxo-2,5-dihydro-1H-pyrrol-1-yl)propanamido)-N-ethyl-3,6,9,12-tetraoxapentadecan-15-amide (ATAC11); N-(5-(2-amino-3-pentylquinolin-5-yl)pentyl)-1-(3-(2,5-dioxo-2,5-dihydro-1H-pyrrol-1-yl)propanamido)-3,6,9,12-tetraoxapentadecan-15-amide (ATAC12); 1-(3-(2,5-dioxo-2,5-dihydro-1H-pyrrol-1-yl)propanamido)-N-(3-pentylquinolin-2-yl)-3,6,9,12-tetraoxapentadecan-15-amide (ATAC13); 1-(3-(2,5-dioxo-2,5-dihydro-1H-pyrrol-1-yl)propanamido)-N-(1-isobutyl-1H-imidazo[4,5-c]quinolin-4-yl)-3,6,9,12-tetraoxapentadecan-15-amide (ATAC14); 1-(3-(2,5-dioxo-2,5-dihydro-1H-pyrrol-1-yl)propanamido)-N-methyl-N-(2-(3-(7-methylbenzo[1,2-d:3,4-d′]bis(thiazole)-2-yl)ureido)ethyl)-3,6,9,12-tetraoxapentadecan-15-amide (ATAC15); (S)-1-(3-(2,5-dioxo-2,5-dihydro-1H-pyrrol-1-yl)propanamido)-N-(1-((7-methylbenzo[1,2-d:3,4-d′]bis(thiazole)-2-yl)amino)-1-oxo-3-phenylpropan-2-yl)-3,6,9,12-tetraoxapentadecan-15-amide (ATAC16); N-(benzo[d]thiazol-2-yl)-1-(3-(2,5-dioxo-2,5-dihydro-1H-pyrrol-1-yl)propanamido)-N-((8-hydroxyquinolin-7-yl)(4-(trifluoromethoxy)phenyl)methyl)-3,6,9,12-tetraoxapentadecan-15-amide (ATAC17); N-((2R,3R,3aS,7aR,9R,10R,10aS,14aR)-2,9-bis(2-amino-6-oxo-1H-purin-9(6H)-yl)-5,10,12-trihydroxy-5,12-dioxidodecahydrodifuro[3,2-d:3′,2′-j][1,3,7,9,2,8]tetra-oxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC18); N-((2R,3R,3aS,7aR,9R,10R,10aS,14aR)-2,9-bis(2-amino-6-oxo-1H-purin-9(6H)-yl)-10-hydroxy-5,12-dimercapto-5,12-dioxidodecahydrodifuro[3,2-d:3′,2′-j][1,3,7,9,2,8]tetraoxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC19); N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetra-oxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC20); or N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetraoxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC21).

An ATAC can be formed by conjugating a cleavable valine-alanine or valine-citrulline linker containing a PABA group and a succinimide group with an immune-stimulatory compound. For example, an ATAC can be 4-((S)-2-((S)-2-(6-(2,5-dioxo-2,5-dihydro-1H-pyrrol-1-yl)hexanamido)-3-methylbutanamido)propanamido)benzyl ((4-amino-1-(2-hydroxy-2-methyl-propyl)-1H-imidazo[4,5-c]quinolin-2-yl)methyl)(ethyl)carbamate (ATAC22); 4-((S)-2-((S)-2-(6-(2,5-dioxo-2,5-dihydro-1H-pyrrol-1-yl)hexanamido)-3-methyl-butanamido)propanamido)benzyl (5-(2-amino-3-pentylquinolin-5-yl)pentyl)-carbamate (ATAC23); 4-((S)-2-((S)-2-(6-(2,5-dioxo-2,5-dihydro-1H-pyrrol-1-yl)hexanamido)-3-methylbutan-amido)-5-ureidopentanamido)benzyl-(5-(2-amino-3-pentylquinolin-5-yl)pentyl)-carbamate (ATAC24); 4-((S)-2-((S)-2-(6-(2,5-dioxo-2,5-dihydro-1H-pyrrol-1-yl)hexanamido)-3-methylbutanamido)-5-ureidopentanamido)benzyl((4-amino-1-(2-hydroxy-2-methylpropyl)-1H-imidazo[4,5-c]quinolin-2-yl)methyl)(ethyl)carbamate TFA salt (ATAC25); 2-(3-{2-[N-Methyl({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino]ethyl}ureido)-7-methyl-1,6-dithia-3,8-diaza-as-indacene (ATAC26); 2-{[(8-Hydroxy-7-quinolyl)(p-trifluoromethoxyphenyl)methyl]({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino}-1,3-benzothiazole (ATAC27); (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5.12λ5-diphosphatricyclo[13.3.0.06,10]octadecane-3,12-dione (ATAC28); (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}propionylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5.12λ5-diphosphatricyclo[13.3.0.06,10]octadecane-3,12-dione (ATAC29); (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-9-hydroxy-3,12-dimercapto-2.4.7.11.13.16-hexaoxa-3λ5.12λ5-diphosphatricyclo[13.3.0.06,10]octadecane-3,12-dione (ATAC30); {p-[(S)-2-{(S)-2-[6-(2,5-Dioxo-1H-pyrrol-1-yl)hexanoylamino]-3-methylbutyrylamino}-5-ureidovalerylamino]phenyl}methyl 9-{(1S,6R,8R,9S,10S,15R,17R,18S)-8-(2-amino-6-oxo-1,9-dihydropurin-9-yl)-3,12-dihydroxy-9,18-dihydroxy-3,12-dioxo-2.4.7.11.13.16-hexaoxa-3λ5.12λ5-diphosphatricyclo[13.2.1.06,10]octadec-17-yl}-9a-adenineecarboxylate (ATAC31; 1-{6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexyl}-1H-pyrrole-2,5-dione (ATAC32); 1-{[4-({6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexylamino}carbonyl)cyclohexyl]methyl}-1H-pyrrole-2,5-dione (ATAC33); or 1-[(4-{[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-carbonyl}cyclohexyl)methyl]-1H-pyrrole-2,5-dione (ATAC34).

An ATAC can be formed by conjugating a noncleavable maleimide-PEG4 linker containing an activated ester such as a pentafluorophenyl group or an N-hydroxysuccinimide group with an immune-stimulatory compound. For example, an ATAC can be pentafluorophenyl 25-(2-amino-3-pentylquinolin-5-yl)-19-oxo-4,7,10,13,16-pentaoxa-20-azapentacosanoate (ATAC1); perfluorophenyl 3-((4-amino-1-(2-hydroxy-2-methylpropyl)-1H-imidazo[4,5-c]quinolin-2-yl)methyl)-4-oxo-7,10,13,16,19-pentaoxa-3-azadocosan-22-oate (ATAC2); pentafluorophenyl 25-(2-amino-3-pentylquinolin-5-yl)-19-oxo-4,7,10,13,16-pentaoxa-20-azapentacosanoate (ATAC3); or 2,5-Dioxopyrrolidin-1-yl 3-((4-amino-1-(2-hydroxy-2-methylpropyl)-1H-imidazo-[4,5-c]quinolin-2-yl)methyl)-4-oxo-7,10,13,16,19-pentaoxa-3-azadocosan-22-oate (ATAC4).

An ATAC can be formed by conjugating a cleavable valine-alanine or valine-citrulline linker containing a PABA group and an activated ester such as a pentafluorophenyl group or an N-hydroxysuccinimde group to an immune-stimulatory compo pound. For example, an ATAC can be 2,5-dioxopyrrolidin-1-yl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC5); 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC6); 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC7); perfluorophenyl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC8); perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC9); or perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC10).

An antibody construct can comprise an anti-CD40 antibody. An anti-CD40 antibody can comprise two SBT-040-G1WT heavy chains and two light chain from a SBT-040 antibody, which can be referred to as SBT-040-WT or as SBT-040-G1. An anti-CD40 antibody can comprise two SBT-040-G1VLPLL heavy chains and two light chains from a SBT-040 antibody, which can be referred to as SBT-040-VLPLL. An anti-CD40 antibody can comprise two SBT-040-GIDE heavy chains and two light chains from a SBT-040 antibody, which can be referred to as SBT-040-DE. An anti-CD40 antibody can comprise two SBT-040-G1AAA heavy chains and two light chains from a SBT-040 antibody, which can be referred to as SBT-040-AAA. An anti-CD40 antibody can comprise two IgG2 heavy chains and two light chain from a SBT-040 antibody, which can be referred to as SBT-040-G2.

A conjugate can comprise SBT-040-WT-ATAC1. A conjugate can comprise SBT-040-WT-ATAC2. A conjugate can comprise SBT-040-WT-ATAC3. A conjugate can comprise SBT-040-WT-ATAC4. A conjugate can comprise SBT-040-WT-ATAC5. A conjugate can comprise SBT-040-WT-ATAC6. A conjugate can comprise SBT-040-WT-ATAC7. A conjugate can comprise SBT-040-WT-ATAC8. A conjugate can comprise SBT-040-WT-ATAC9. A conjugate can comprise SBT-040-WT-ATAC10. A conjugate can comprise SBT-040-WT-ATAC11. A conjugate can comprise SBT-040-WT-ATAC12. A conjugate can comprise SBT-040-WT-ATAC13. A conjugate can comprise SBT-040-WT-ATAC14. A conjugate can comprise SBT-040-WT-ATAC15. A conjugate can comprise SBT-040-WT-ATAC16. A conjugate can comprise SBT-040-WT-ATAC17. A conjugate can comprise SBT-040-WT-ATAC18. A conjugate can comprise SBT-040-WT-ATAC19. A conjugate can comprise SBT-040-WT-ATAC20. A conjugate can comprise SBT-040-WT-ATAC21. A conjugate can comprise SBT-040-WT-ATAC22. A conjugate can comprise SBT-040-WT-ATAC23. A conjugate can comprise SBT-040-WT-ATAC24. A conjugate can comprise SBT-040-WT-ATAC25. A conjugate can comprise SBT-040-WT-ATAC26. A conjugate can comprise SBT-040-WT-ATAC27. A conjugate can comprise SBT-040-WT-ATAC28. A conjugate can comprise SBT-040-WT-ATAC29. A conjugate can comprise SBT-040-WT-ATAC30. A conjugate can comprise SBT-040-WT-ATAC31. A conjugate can comprise SBT-040-WT-ATAC32. A conjugate can comprise SBT-040-WT-ATAC33. A conjugate can comprise SBT-040-WT-ATAC34. A conjugate can comprise SBT-040-WT-ATAC43. A conjugate can comprise SBT-040-VLPLL-ATAC1. A conjugate can comprise SBT-040-VLPLL-ATAC2. A conjugate can comprise SBT-040-VLPLL-ATAC3. A conjugate can comprise SBT-040-VLPLL-ATAC4. A conjugate can comprise SBT-040-VLPLL-ATAC5. A conjugate can comprise SBT-040-VLPLL-ATAC6. A conjugate can comprise SBT-040-VLPLL-ATAC7. A conjugate can comprise SBT-040-VLPLL-ATAC8. A conjugate can comprise SBT-040-VLPLL-ATAC9. A conjugate can comprise SBT-040-VLPLL-ATAC10. A conjugate can comprise SBT-040-VLPLL-ATAC11. A conjugate can comprise SBT-040-VLPLL-ATAC12. A conjugate can comprise SBT-040-VLPLL-ATAC13. A conjugate can comprise SBT-040-VLPLL-ATAC14. A conjugate can comprise SBT-040-VLPLL-ATAC15. A conjugate can comprise SBT-040-VLPLL-ATAC16. A conjugate can comprise SBT-040-VLPLL-ATAC17. A conjugate can comprise SBT-040-VLPLL-ATAC18. A conjugate can comprise SBT-040-VLPLL-ATAC19. A conjugate can comprise SBT-040-VLPLL-ATAC20. A conjugate can comprise SBT-040-VLPLL-ATAC21. A conjugate can comprise SBT-040-VLPLL-ATAC22. A conjugate can comprise SBT-040-VLPLL-ATAC23. A conjugate can comprise SBT-040-VLPLL-ATAC24. A conjugate can comprise SBT-040-VLPLL-ATAC25. A conjugate can comprise SBT-040-VLPLL-ATAC26. A conjugate can comprise SBT-040-VLPLL-ATAC27. A conjugate can comprise SBT-040-VLPLL-ATAC28. A conjugate can comprise SBT-040-VLPLL-ATAC29. A conjugate can comprise SBT-040-VLPLL-ATAC30. A conjugate can comprise SBT-040-VLPLL-ATAC31. A conjugate can comprise SBT-040-VLPLL-ATAC32. A conjugate can comprise SBT-040-VLPLL-ATAC33. A conjugate can comprise SBT-040-VLPLL-ATAC34. A conjugate can comprise SBT-040-VLPLL-ATAC43. A conjugate can comprise SBT-040-DE-ATAC1. A conjugate can comprise SBT-040-DE-ATAC2. A conjugate can comprise SBT-040-DE-ATAC3. A conjugate can comprise SBT-040-DE-ATAC4. A conjugate can comprise SBT-040-DE-ATAC5. A conjugate can comprise SBT-040-DE-ATAC6. A conjugate can comprise SBT-040-DE-ATAC7. A conjugate can comprise SBT-040-DE-ATAC8. A conjugate can comprise SBT-040-DE-ATAC9. A conjugate can comprise SBT-040-DE-ATAC10. A conjugate can comprise SBT-040-DE-ATAC11. A conjugate can comprise SBT-040-DE-ATAC12. A conjugate can comprise SBT-040-DE-ATAC13. A conjugate can comprise SBT-040-DE-ATAC14. A conjugate can comprise SBT-040-DE-ATAC15. A conjugate can comprise SBT-040-DE-ATAC16. A conjugate can comprise SBT-040-DE-ATAC17. A conjugate can comprise SBT-040-DE-ATAC18. A conjugate can comprise SBT-040-DE-ATAC19. A conjugate can comprise SBT-040-DE-ATAC20. A conjugate can comprise SBT-040-DE-ATAC21. A conjugate can comprise SBT-040-DE-ATAC22. A conjugate can comprise SBT-040-DE-ATAC23. A conjugate can comprise SBT-040-DE-ATAC24. A conjugate can comprise SBT-040-DE-ATAC25. A conjugate can comprise SBT-040-DE-ATAC26. A conjugate can comprise SBT-040-DE-ATAC27. A conjugate can comprise SBT-040-DE-ATAC28. A conjugate can comprise SBT-040-DE-ATAC29. A conjugate can comprise SBT-040-DE-ATAC30. A conjugate can comprise SBT-040-DE-ATAC31. A conjugate can comprise SBT-040-DE-ATAC32. A conjugate can comprise SBT-040-DE-ATAC33. A conjugate can comprise SBT-040-DE-ATAC34. A conjugate can comprise SBT-040-DE-ATAC43. A conjugate can comprise SBT-040-AAA-ATAC1. A conjugate can comprise SBT-040-AAA-ATAC2. A conjugate can comprise SBT-040-AAA-ATAC3. A conjugate can comprise SBT-040-AAA-ATAC4. A conjugate can comprise SBT-040-AAA-ATAC5. A conjugate can comprise SBT-040-AAA-ATAC6. A conjugate can comprise SBT-040-AAA-ATAC7. A conjugate can comprise SBT-040-AAA-ATAC8. A conjugate can comprise SBT-040-AAA-ATAC9. A conjugate can comprise SBT-040-AAA-ATAC10. A conjugate can comprise SBT-040-AAA-ATAC11. A conjugate can comprise SBT-040-AAA-ATAC12. A conjugate can comprise SBT-040-AAA-ATAC13. A conjugate can comprise SBT-040-AAA-ATAC14. A conjugate can comprise SBT-040-AAA-ATAC15. A conjugate can comprise SBT-040-AAA-ATAC16. A conjugate can comprise SBT-040-AAA-ATAC17. A conjugate can comprise SBT-040-AAA-ATAC18. A conjugate can comprise SBT-040-AAA-ATAC19. A conjugate can comprise SBT-040-AAA-ATAC20. A conjugate can comprise SBT-040-AAA-ATAC21. A conjugate can comprise SBT-040-AAA-ATAC22. A conjugate can comprise SBT-040-AAA-ATAC23. A conjugate can comprise SBT-040-AAA-ATAC24. A conjugate can comprise SBT-040-AAA-ATAC25. A conjugate can comprise SBT-040-AAA-ATAC26. A conjugate can comprise SBT-040-AAA-ATAC27. A conjugate can comprise SBT-040-AAA-ATAC28. A conjugate can comprise SBT-040-AAA-ATAC29. A conjugate can comprise SBT-040-AAA-ATAC30. A conjugate can comprise SBT-040-AAA-ATAC31. A conjugate can comprise SBT-040-AAA-ATAC32. A conjugate can comprise SBT-040-AAA-ATAC33. A conjugate can comprise SBT-040-AAA-ATAC34. A conjugate can comprise SBT-040-AAA-ATAC33. A conjugate can comprise SBT-040-AAA-ATAC43. The K_{d} for binding of the CD40 binding domain of any of these conjugates to CD40 can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the CD40 binding domain to CD40 in the absence of the immune-stimulatory compound or ATAC. The K_{d} for binding of the CD40 binding domain of any of these conjugates to CD40 can be less than 10 nM. The K_{d} for binding of the CD40 binding domain of any of the conjugates to CD40 can be less than 100 nM, less than 50 nM, less than 20 nM, less than 5 nM, less than 1 nM, or less than 0.1 nM. The K_{d} for binding of the Fc domain of any of the conjugates to an Fc receptor can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-stimulatory compound or ATAC. The K_{d} for binding of the Fc domain of any of the conjugates to an Fc receptor of an can be less than 10 nM. The K_{d} for binding of the Fc domain of any of the conjugates to an Fc receptor can be less than 100 nM, less than 50 nM, less than 20 nM, less than 5 nM, less than 1 nM, or less than 0.1 nM.

In a conjugate, an antibody can be linked to an immune-stimulatory compound in such a way that the antibody can still bind to an antigen and the Fc domain of the antibody can still bind to an FcR. In a conjugate, an antibody construct is linked to an immune-stimulatory compound in such a way that the linking does not interfere with ability of the antigen binding domain of the antibody construct to bind to antigen, the ability of the Fc domain of the antibody construct to bind to an FcR, or FcR-mediated signaling resulting from the Fc domain of the antibody construct from binding to an FcR. In a conjugate, an immune-stimulatory compound can be linked to an antibody construct in such a way the linking does not interfere with the ability of the immune-stimulatory compound to bind to its receptor. A conjugate can produce stronger immune stimulation and a greater therapeutic window than components of the conjugate alone. In an anti-CD40 antibody linked to a TLR agonist conjugate, the combination of CD40 agonism, TLR agonism, and an accessible Fc domain of the anti-CD40 antibody to allow FcR-mediated signaling can produce stronger immune stimulation and a greater therapeutic window than the CD40 agonism, TLR agonism, or the FcR-mediated signaling alone.

### Methods of Synthesis of Antibody Construct Immune-Stimulatory Compound Conjugate Components

### Synthesis of Immune-Stimulatory Compounds

An immune stimulatory compound can be synthesized as shown in Scheme A1.

[chemical structure]

[chemical structure]

Synthesis of the C-2′ amino cyclic dinucleotide (viii) can be accomplished using a multistep synthesis as outlined in scheme A1 above and described below in EXAMPLE 3.

### Synthesis of ATAC Compounds

An ATAC compound can be synthesized by various methods. For example, ATAC compounds, such as ATAC1-ATAC4, can be synthesized as shown in Scheme B1.

[chemical structure]

A PEGylated carboxylic acid (i) that has been activated for amide bond formation can be reacted with an appropriately substituted amine containing immune-stimulatory compound to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

An ATAC compound can be synthesized by various methods. For example, ATAC compounds, such as ATAC5-ATAC10, can be synthesized as shown in Scheme B2.

[chemical structure]

An activated carbonate such as (i) can be reacted with an appropriately substituted amine containing immune-stimulatory compound to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

An ATAC compound can be synthesized by various methods. For example, ATAC compounds, such as ATAC11-ATAC21, can be synthesized as shown in Scheme B3.

[chemical structure]

An activated carboxylic ester such as (i-a) can be reacted with an appropriately substituted amine containing immune-stimulatory compound to afford amides (ii). Alternatively, carboxylic acids of type (i-b) can be coupled to an appropriately substituted amine containing immune-stimulatory compound in the presence of an amide bond forming agent such as dicyclohexylcarbodiimide (DCC) to provide the desired ATAC compounds.

An ATAC compound can be synthesized by various methods. For example, ATAC compounds, such as ATAC22-ATAC31, can be synthesized as shown in Scheme B4.

[chemical structure]

An activated carbonate such as (i) can be reacted with an appropriately substituted amine containing immune-stimulatory compound to afford carbamates (ii) as the target ATAC compounds.

An ATAC compound can be synthesized by various methods. For example, ATAC compounds, such as ATAC32-ATAC34, can be synthesized as shown in Scheme B5.

[chemical structure]

An activated carboxylic acid such as (i-a, i-b, i-c) can be reacted with an appropriately substituted amine containing immune-stimulatory compound to afford amides (ii-a, ii-b, ii-c) as the target ATAC compounds.

These antibody construct immune-stimulatory conjugates can be made by various methods. It is understood that one skilled in the art may be able to make these compounds by similar methods or by combining other methods known to one skilled in the art. It is also understood that one skilled in the art would be able to make, in a similar manner as described herein by using the appropriate starting materials and modifying the synthetic route as needed. Starting materials and reagents can be obtained from commercial vendors or synthesized according to sources known to those skilled in the art or prepared as described herein.

### Pharmaceutical Formulations

The compositions and methods described herein can be considered useful as pharmaceutical compositions for administration to a subject in need thereof. Pharmaceutical compositions can comprise at least the compositions described herein and one or more pharmaceutically acceptable carriers, diluents, excipients, stabilizers, dispersing agents, suspending agents, and/or thickening agents. The composition can comprise the conjugate having an antibody construct and an agonist. The composition can comprise the conjugate having an antibody construct, a targeting binding domain, and an agonist. The composition can comprise any conjugate described herein. Often, the antibody construct is an anti-CD40 antibody. A conjugate can comprise an anti-CD40 antibody and a PAMP molecule. A conjugate can comprise an anti-CD40 antibody and a DAMP molecule. A pharmaceutical composition can further comprise buffers, antibiotics, steroids, carbohydrates, drugs (e.g., chemotherapy drugs), radiation, polypeptides, chelators, adjuvants and/or preservatives.

Pharmaceutical compositions can be formulated using one or more physiologically-acceptable carriers comprising excipients and auxiliaries. Formulation can be modified depending upon the route of administration chosen. Pharmaceutical compositions comprising a composition as described herein can be manufactured, for example, by lyophilizing the conjugate, mixing, dissolving, emulsifying, encapsulating or entrapping the conjugate. The pharmaceutical compositions can also include the compositions described herein in a free-base form or pharmaceutically-acceptable salt form.

Methods for formulation of the conjugates described herein can include formulating any of the conjugates described herein with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compositions described herein can be lyophilized or in powder form for re-constitution with a suitable vehicle, e.g., sterile pyrogen-free water, before use

Pharmaceutical compositions of the conjugates described herein can comprise at least an active ingredient. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein often further can comprise more than one active compound as necessary for the particular indication being treated. The active compounds can have complementary activities that do not adversely affect each other. For example, the composition can comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The compositions and formulations can be sterilized. Sterilization can be accomplished by filtration through sterile filtration.

The compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions described herein can be lyophilized or in powder form for reconstitution with a suitable vehicle, e.g., sterile pyrogen-free water, before use.

For parenteral administration the conjugates can be formulated in a unit dosage injectable form (e.g., use letter solution, suspension, emulsion) in association with a pharmaceutically acceptable parenteral vehicle. Such vehicles can be inherently nontoxic, and non-therapeutic. A vehicles can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can be also be prepared. Examples of sustained-release preparations can include semipermeable matrices of solid hydrophobic polymers that can contain the antibody, and these matrices can be in the form of shaped articles (e.g., films or microcapsules). Examples of sustained-release matrices can include polyesters, hydrogels (e.g., poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

### Therapeutic Applications

The compositions and methods of the present disclosure can be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The compositions and methods described herein can be useful as a therapeutic, for example a treatment that can be administered to a subject in need thereof. A therapeutic effect of the present disclosure can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically-effective amounts of the compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically-effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition, and can contemplate a range of results directed to that end, including but not restricted to prevention of the condition entirely.

Prevent, preventing and the like can refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

A therapeutically effective amount can be the amount of a composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The conjugates described herein that can be used in therapy can be formulated and dosages established in a fashion consistent with good medical practice taking into account the disorder to be treated, the condition of the individual patient, the site of delivery of the composition, the method of administration and other factors known to practitioners. The conjugates described herein can be prepared according to the description of preparation described herein.

Pharmaceutical compositions can be considered useful with the compositions and methods described herein can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods and compositions described herein can be for administration to a subject in need thereof. Often, administration of the compositions described herein can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition can be administered to a subject by additional routes of administration, for example, by inhalation, oral, dermal, intranasal, or intrathecal administration.

Compositions of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week or more than once per month.

### Diseases, Conditions and the Like

The compositions and methods provided herein can be useful for the treatment of a plurality of diseases, conditions, preventing a disease or a condition in a subject or other therapeutic applications for subjects in need thereof. Often the compositions and methods provided herein can be useful for treatment of hyperplastic conditions, including but not limited to, neoplasms, cancers, tumors and the like. A condition, such as a cancer, can be associated with expression of a molecule on the cancer cells. Often, the molecule expressed by the cancer cells can comprise an extracellular portion capable of recognition by the antibody portion of the conjugate. A molecule expressed by the cancer cells can be a tumor antigen. An antibody portion of the conjugate can recognize a tumor antigen. A tumor antigen can include CD5, CD19, CD20, CD25, CD37, CD30, CD33, CD45, CAMPATH-1, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MARTI, Ras mutant, gp100, p53 mutant, PR1, bcr-ab1, tyronsinase, survivin, PSA, hTERT, Sarcoma translocation breakpoints, EphA2, PAP, ML-IAP, AFP, ERG, NA17, PAX3, ALK, androgen receptor, cyclin B1, polysialic acid, MYCN, RhoC, TRP-2, fucosyl GM1, mesothelin, PSCA, MAGE A1, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL1, MUC16, MAGE A4, MAGE C2, GAGE, or Fos-related antigen 1.

As described herein, an antigen binding domain portion of the conjugate, can be configured to recognize a molecule expressed by a cancer cell, such as for example, a disease antigen, tumor antigen or a cancer antigen. Often such antigens are known to those of ordinary skill in the art, or newly found to be associated with such a condition, to be commonly associated with, and/or, specific to, such conditions. For example, a disease antigen, tumor antigen or a cancer antigen is, but is not limited to, CD5, CD19, CD20, CD25, CD37, CD30, CD33, CD45, CAMPATH-1, BCMA, CS-1, PD-L1, B7-H3, B7-DC, HLD-DR, carcinoembryonic antigen, TAG-72, EpCAM, MUC1, folate-binding protein, A33, G250, prostate-specific membrane antigen, ferritin, GD2, GD3, GM2, Le^{y}, CA-125, CA19-9, epidermal growth factor, p185HER2, IL-2 receptor, de2-7 EGFR, fibroblast activation protein, tenascin, metalloproteinases, endosialin, vascular endothelial growth factor, avB3, WT1, LMP2, HPV E6 E7, EGFRvIII, Her-2/neu, idiotype, MAGE A3, p53 nonmutant, NY-ESO-1, PMSA, GD2, CEA, MelanA/MARTI, Ras mutant, gp100, p53 mutant, PR1, bcr-ab1, tyronsinase, survivin, PSA, hTERT, Sarcoma translocation breakpoints, EphA2, PAP, ML-IAP, AFP, ERG, NA17, PAX3, ALK, androgen receptor, cyclin B1, polysialic acid, MYCN, RhoC, TRP-2, fucosyl GM1, mesothelin, PSCA, MAGE A1, sLe (animal), CYP1B1, PLAV1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 3, Page4, VEGFR2, MAD-CT-1, PDGFR-B, MAD-CT-2, ROR2, TRAIL, MUC16, MAGE A4, MAGE C2, GAGE, EGFR, CMET, HER3, MUC1, MUC15, MSLN, CA6, NAPI2B, TROP2, CLDN18.2, RON, LY6E, FRA, DLL3, PTK7, LIV1, ROR1, MAGE-A3, or Fos-related antigen 1. Additionally, such tumor antigens can be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like.

Non-limiting examples of cancers can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukaemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sézary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma; Thyroid cancer; Thyroid cancer, childhood; Uterine cancer; Vaginal cancer; Waldenström macroglobulinemia; Wilms tumor and any combination thereof.

### Example 1

### Fc Receptor Binding to Anti-CD40 Antibodies

An anti-CD40 antibody is comprised of two SBT-040-G1WT heavy chains and two light chain from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody.

SBT-040-WT antibody, SBT-040-VLPLL antibody, SBT-040-DE antibody, and SBT-040-AAA antibody are produced by standard methods for producing antibodies. These antibodies are purified, and each antibody's affinity for soluble glycosylated ectodomains from all human Fcγ receptors (FcγRs) is measured. These affinities are measured by experiments using surface plasmon resonance. In these experiments, biotinylated soluble glycosylated FcγR ectodomains from all human FcγRs are immobilized on a streptavidin-coated surface. The ability of each antibody to bind to soluble glycosylated FcγR ectodomains from all human FcγRs is then measured by surface plasmon resonance using a Biacore instrument. The data from this experiment shows that the Fc domain of a SBT-040-WT antibody, the Fc domain of a SBT-040-VLPLL antibody, the Fc domain of a SBT-040-DE antibody, and the Fc domain of a SBT-040-AAA antibody are each bound to soluble glycosylated FcγR ectodomains from all human FcγRs. Therefore, the surface plasmon resonance experiments show that the Fc domain of the SBT-040-G1WT antibody and variants of the Fc domain of a SBT-040-G1WT antibody (i.e., the Fc domain of a SBT-040-G1VLPLL antibody, the Fc domain of a SBT-040-DE antibody and the Fc domain of a SBT-040-AAA antibody) are each bound to all human FcγRs. The affinity of each antibody for each human FcγRs is also shown by these experiments.

### Example 2

### Synthesis of Linkers with Immune-Stimulatory Compounds

A linker is linked with an immune-stimulatory compound. A linker linked to an immune-stimulatory compound is formed to make a linker-immune stimulatory compound conjugate (ATAC). Subsequently, an ATAC is conjugated to an antibody, in which the ATAC is any one of ATAC1-ATAC34 or ATAC 43 (each of which is described in the below EXAMPLES).

A linker is linked with an antibody, in which the linker is a pegylated linker, a valine-alanine linker, a valine-citrulline linker, or an N-Maleimidomethylcyclohexane-1-carboxylate (MCC) linker. Subsequently, an immune-stimulatory compound is conjugated to the linker linked with the antibody, in which the immune-stimulatory compound is a TLR ligand, a Nod-like receptor ligand, a RIG-Like receptor ligand, a CLR ligand, a CDS ligand, or an inflammasome inducer.

A linker is linked with an antibody, in which the linker is a pegylated linker, a valine-alanine linker, a valine-citrulline linker, or an N-Maleimidomethylcyclohexane-1-carboxylate (MCC) linker. Subsequently, an immune-stimulatory compound is conjugated to the linker linked with the antibody, in which the immune-stimulatory compound is gardiquimod or an analog of a cyclic dinucleotide.

### Example 3

### Synthesis of (1R,6R,8R,9R,10S,15R,17R,18R)-9-Amino-8,17-bis(2-amino-6-oxo-1,9-dihydropurin-9-yl)-3,12-dihydroxy-18-hydroxy-2.4.7.11.13.16-hexaoxa-3λ5.12λ5-diphosphatricyclo [13.3.0.06,10]octadecane-3,12-dione (Compound 21)

This example shows the synthesis of (1R,6R,8R,9R,10S,15R,17R,18R)-9-Amino-8,17-bis(2-amino-6-oxo-1,9-dihydropurin-9-yl)-3,12-dihydroxy-18-hydroxy-2.4.7.11.13.16-hexaoxa-3λ5.12λ5-diphosphatricyclo[13.3.0.06,10]octadecane-3,12-dione (Compound 21).

[chemical structure]

### Step A: Preparation of Int 2.13-1

[chemical structure]

Guanosine (200 g, 706.71 mmol, 1.00 equiv) was suspended in dry pyridine (4000 mL) under a nitrogen atmosphere, and TBSCl (572 g, 5.30 mol, 7.50 equiv) was added dropwise at 0° C. The reaction was stirred at ambient temperature for 3h then cooled to 0° C. before adding isobutyric anhydride (167 g, 1.06 mol, 1.5 equiv) dropwise over 20 min. The solution was allowed to warm to room temperature and stirred for 16h. The reaction solution was cooled to 0° C. and the reaction was quenched by the addition of water (500 mL). After stirring for 20 min at 0° C., 1000 mL of concentrated aqueous NH₄OH was added dropwise at 0° C. After stirring for an additional 1 h at room temperature, the resulting mixture was concentrated and the residue was dissolved in 3000 mL of water and washed with 1500 mL of EtOAc. The aqueous phase was concentrated to ˜1000 mL whereby the product precipitated from water. The product was filtered to afford 174 g of N-[9-[(2R,3R,4S,5R)-3,4-dihydroxy-5-(hydroxymethyl)oxolan-2-yl]-6-oxo-6,9-dihydro-1H-purin-2-yl]-2-methylpropanamide (Int 2.13-1) as a white solid.

### Step B: Preparation of Int 2.13-2

[chemical structure]

To a stirred suspension of Int 2.13-1 (200.0 g, 566.57 mmol, 1.00 equiv) in pyridine (3 L) under a nitrogen atmosphere was added 4,4′-(chloro(phenyl)methylene)bis(methoxybenzene) (211 g, 623.23 mmol, 1.10 equiv). The resulting mixture was left to stir for 16 h at room temperature. The reaction was quenched with methanol (100 mL) and the mixture was concentrated under vacuum. The residue was dissolved in 3000 mL of dichloromethane, washed with 2×1500 mL of saturated sodium bicarbonate solution and 1500 mL of saturated sodium chloride solution respectively. The organic phase was dried over anhydrous sodium sulfate, filtered and concentrated under vacuum. The crude product was applied onto a silica gel column with DCM/methanol (with 0.05% triethylamine) (50/1-20/1). This resulted in 278 g (75%) of N-[9-[(2R,3R,4S,5R)-5-[[bis(4-methoxyphenyl)(phenyl)methoxy]methyl]-3,4-dihydroxyoxolan-2-yl]-6-oxo-6,9-dihydro-1H-purin-2-yl]-2-methylpropanamide (Int 2.13-2) as a light yellow solid.

### Step C: Preparation of Int 2.13-3a and Int 2.13-3b

[chemical structure]

Compound Int 2.13-2 (150 g, 229 mmol, 1.00 equiv) was dissolved in 1500 mL of pyridine under a nitrogen atmosphere. 1H-imidazole (46.71 g, 687.02 mmol, 3.00 equiv) was added, followed by addition of TBS-Cl (51.6 g, 343.51 mmol, 1.50 equiv) in portions at 25° C. The resulting solution was stirred for 16 h at 25° C. then concentrated and dissolved in 2000 mL of dichloromethane. The organic extract was washed with 2×1000 mL of saturated sodium bicarbonate solution and 1000 mL of saturated sodium chloride solution, respectively. The organic phase was dried over anhydrous sodium sulfate, filtered and concentrated under vacuum. The crude product was applied onto a silica gel column with ethyl acetate/dichloromethane (1/50-1/1) and then purified by flash with the following conditions: silica gel column; ethyl acetate in dichloromethane with 0.05% triethylamine: 15% up to 70% within 10 min and 70% maintained 10 min; This resulted in 62 g (35%) of N-[9-[(2R,3R,4R,5R)-5-[[bis(4-methoxyphenyl)(phenyl)methoxy]methyl]-3-[(tert-butyldimethylsilyl)oxy]-4-hydroxyoxolan-2-yl]-6-oxo-6,9-dihydro-1H-purin-2-yl]-2-methylpropanamide (Int 2.13-3a) as a yellow solid and 44 g (25%) of N-[9-[(2R,3R,4S,5R)-5-[[bis(4-methoxyphenyl)(phenyl)methoxy]methyl]-4-[(tert-butyldimethylsilyl)oxy]-3-hydroxyoxolan-2-yl]-6-oxo-6,9-dihydro-1H-purin-2-yl]-2-methylpropanamide (Int 2.13-3b).

### Step D: Preparation of Int 2.13-4

[chemical structure]

Int 2.13-3a (28 g, 36.41 mmol, 1.00 equiv) was dissolved in 280 mL of dichloromethane under a nitrogen atmosphere. 1H-imidazole-4,5-dicarbonitrile (12.9 g, 109.23 mmol, 3.00 equiv) and 3-(bis[bis(propan-2-yl)amino]phosphanyloxy)propanenitrile (43.84 g, 145.64 mmol, 4.00 equiv) were added in order. The resulting solution was stirred for 1 h at 25° C. and the resulting solution was diluted with 500 mL of dichloromethane and washed with 4×400 mL of saturated sodium bicarbonate solution and 1×400 mL of saturated sodium chloride solution, respectively. The organic phase was dried over anhydrous sodium sulfate, filtered and concentrated under vacuum. The crude product was purified by flash chromatography (C18 silica gel; mobile phase, acetonitrile in water gradient: 40% up to 100% within 8 min and 100% maintained 10 min) to afford 25 g of Int 2.13-4 as a white solid.

### Step E: Preparation of Int 2.13-5

[chemical structure]

To a solution of Int 2.13-1 (190 g, 538.24 mmol) in 3000 mL of pyridine was added 1,1,3,3-tetraisopropyl-1,3-dichlorodisiloxane (152.6 g, 484.42 mmol, 0.9 equiv) dropwise at 0° C. The resulting solution was stirred for 16 h at 25° C. The reaction was quenched by the addition of 30 mL of methanol and the resulting solution was concentrated under vacuum. The residue was applied onto a silica gel column with dichloromethane/methanol (50/1-30/1) to afford 189 g of N-[9-[(6aR,8R,9R,9aS)-9-hydroxy-2,2,4,4-tetrakis(propan-2-yl)-hexahydro-2H-furo[3,2-f][1,3,5,2,4]trioxadisilocin-8-yl]-6-oxo-6,9-dihydro-1H-purin-2-yl]-2-methylpropanamide (Int 2.13-5) as a white solid.

### Step F: Preparation of Int 2.13-6

[chemical structure]

Into a 250-mL 3-necked round-bottom flask purged and maintained with an inert atmosphere of nitrogen, was placed a solution of 195 g (327.73 mmol) of N-[9-[(6aR,8R,9R,9aS)-9-hydroxy-2,2,4,4-tetrakis(propan-2-yl)-hexahydro-2H-furo[3,2-f][1,3,5,2,4]trioxadisilocin-8-yl]-6-oxo-6,9-dihydro-1H-purin-2-yl]-2-methylpropanamide (Int 2.13-5) in 4000 mL of dichloromethane, 129.5 g (5.00 equiv) of pyridine and 4-dimethylaminopyridine (20 g, 163.86 mmol, 0.50 equiv). The solution was cooled to 0° C. and treated with 184.8 g (655.5 mmol, 2.0 equiv) triflic anhydride dropwise. The resulting solution was stirred for 2 h at 0° C. then quenched by the addition of 4000 mL of water/ice. The resulting solution was extracted with 3×4000 mL of dichloromethane and the organic layers were combined. The organic extracts were washed with 2×4000 mL of water/ice and 1×4000 mL of saturated sodium chloride solution, respectively. The organic phase was dried over anhydrous sodium sulfate, filtered and concentrated to provide 213 g (crude) of (6aR,8R,9R,9aR)-8-[2-(2-methylpropanamido)-6-oxo-6,9-dihydro-1H-purin-9-yl]-2,2,4,4-tetrakis(propan-2-yl)-hexahydro-2H-furo[3,2-f][1,3,5,2,4]trioxadisilocin-9-yl trifluoromethanesulfonate (Int 2.13-6) as a yellow solid.

### Step G: Preparation of Int 2.13-7

[chemical structure]

Int 2.13-6 (213 g, crude) was dissolved in 2100 mL of DMF and treated with sodium nitrite (158.3 g, 2.29 mol, 7.00 equiv). After stirring for 16 h the solution was filtered and concentrated under vacuum. The resulting solution was diluted with 6000 mL of DCM and washed with 2×3000 mL of saturated sodium chloride solution, dried over anhydrous sodium sulfate, filtered and concentrated under vacuum. The residue was applied to a silica gel column with dichloromethane/methanol (50/1-30/1) and then purified using the following conditions: C18 silica gel, 50% MeOH/water to 100% water over 10 min then 100% water for 10 min to provide 50 g (26% for 2 steps) of Int 2.13-7 as a white solid.

### Step H: Preparation of Int 2.13-8

[chemical structure]

N-[9-[(6aR,8R,9R,9aS)-9-hydroxy-2,2,4,4-tetrakis(propan-2-yl)-hexahydro-2H-furo[3,2-f][1,3,5,2,4]trioxadisilocin-8-yl]-6-oxo-6,9-dihydro-1H-purin-2-yl]-2-methylpropanamide (Int 2.13-7) (50 g, 84.03 mmol) and DMAP (30.8 g, 252.10 mmol, 3.00 equiv) were dissolved in 500 mL of DCM and the mixture was cooled to 0° C. Triflic anhydride (30.8 g, 109.24 mmol, 1.30 equiv) was then added dropwise with stirring at 0° C. The resulting solution was stirred at this temperature for 1 h. The reaction was then quenched by the addition of 500 mL of ice/water then extracted with 3×500 mL of dichloromethane and the organic layers were combined. The organic layer was washed with 2×100 mL of saturated sodium chloride solution. The solution was dried over anhydrous sodium sulfate, filtered and concentrated under vacuum. The crude product (Int 2.13-8) (55 g) was thus isolated and used directly in the next step.

### Step I: Preparation of Int 2.13-9

[chemical structure]

Int 2.13-8 (55 g, crude) was dissolved in N,N-dimethylformamide (500 mL) then treated with sodium azide (27.8 g, 427.73 mmol, 5.1 equiv). The resulting solution was stirred for 16 h at room temperature. The resulting mixture was filtered and concentrated under vacuum. The residue was applied onto a silica gel column with dichloromethane/methanol (20/1) to afford 15.0 g of Int 2.13-9 as a yellow solid.

### Step J. Preparation of Int 2.13-10

[chemical structure]

Int 2.13-9 (23 g, 1.00 equiv) in TH (230 mL) was treated with tetrabutylammonium fluoride (37 mL, 1.0 equiv). The resulting solution was stirred for 10 min at room temperature then concentrated under vacuum. The residue was applied directly to a silica gel column with dichloromethane/methanol (100/1-20/1). This resulted in 12.4 g (88%) of N-[9-[(2R,3R,4S,5R)-3-azido-4-hydroxy-5-(hydroxymethyl)oxolan-2-yl]-6-oxo-6,9-dihydro-1H-purin-2-yl]-2-methylpropanamide (Int 2.13-10) as a white solid.

### Step K: Preparation of Int 2.13-11

[chemical structure]

Into a 50-mL round-bottom flask, was placed a solution of 11 g of Int 2.13-10 in pyridine (60 mL). 4,4′-(chloro(phenyl)methylene)bis(methoxybenzene) (14.75 g, 1.50 equiv) was added and the resulting solution was stirred for 2 h at room temperature. The reaction was quenched by the addition of 20 mL of methanol and the resulting mixture was concentrated under vacuum. The residue was dissolved in 500 mL of dichloromethane, washed with 2×250 mL of saturated sodium bicarbonate solution and 1×250 mL of saturated sodium chloride solution respectively. The organic phase was dried over sodium sulfate, filtered and concentrated under vacuum. The residue was chromatographed with dichloromethane/methanol with 0.05% triethylamine (100/1-60/1) to afford 17.8 g of Int 2.13-11 as a white solid.

### Step L: Preparation of Int 2.13-12

[chemical structure]

Into a 50-mL round-bottom flask was placed a solution of 10 g of Int 2.13-11 in methanol (150 mL) and 10% anhydrous palladium on carbon (2 g, w/w). The resulting mixture was stirred under an atmosphere of hydrogen for 1 h at room temperature. The mixture was filtered through Celite and concentrated to provide 8.7 g of Int 2.13-12 as a yellow solid which was used directly without further purification.

### Step M: Preparation of Int 2.13-13

[chemical structure]

To a solution of 16.8 g of N-[9-[(2R,3R,4S,5R)-3-amino-5-[[bis(4-methoxyphenyl)(phenyl)-methoxy]methyl]-4-hydroxyoxolan-2-yl]-6-oxo-6,9-dihydro-1H-purin-2-yl]-2-methylpropanamide (Int 2.13-12) in 210 mL of THF/water (4/1) was added sodium bicarbonate (6.46 g, 3.00 equiv) followed by the addition of Cbz-Cl (5.67 g, 1.30 equiv). The resulting solution was stirred for 20 min at room temperature. The reaction was diluted with saturated sodium carbonate solution (100 mL) and extracted with 3×200 mL of dichloromethane. The organic phase was dried over sodium sulfate, filtered and concentrated under vacuum. The residue was chromatographed with dichloromethane/methanol with 0.05% triethylamine (100/1) and then crystallized from dichloromethane (40 mL) to afford 14.8 g (86% over two steps) of Int 2.13-13 as a white solid.

### Step N: Preparation of Int 2.13-14

[chemical structure]

Int 2.13-13 (14.8 g, 18.78 mmol) was dissolved in 150 mL dichloromethane under a nitrogen atmosphere, 3-(bis[bis(propan-2-yl)amino]phosphanyloxy)propanenitrile (22.6 g, 75.12 mmol, 4.00 equiv) and 1H-imidazole-4,5-dicarbonitrile (6.64 g, 56.34 mmol 3.00 equiv) were added in order. The resulting solution was stirred for 1 h at 25° C. and diluted with 400 mL of dichloromethane. The solution was washed with 1×500 mL of saturated sodium bicarbonate solution and 1×500 mL of saturated sodium chloride solution respectively. The organic phase was dried over anhydrous sodium sulfate, filtered and concentrated under vacuum. The crude product was purified by Flash with the following conditions: Column, C18 silica gel; mobile phase, acetonitrile in water: 30% up to 80% within 8 min and 100% maintained 10 min to provide 14.8 g (75%) of N-[9-[(2R,3R,4R,5R)-5-[[bis(4-methoxyphenyl)(phenyl)methoxy]methyl]-3-([[bis(propan-2-yl)amino](2-cyanoethoxy)phosphanyl]oxy)-4-[(tert-butyldimethylsilyl)oxy]oxolan-2-yl]-6-oxo-6,9-dihydro-1H-purin-2-yl]-2-methylpropanamide (Int 2.13-14) as a white solid.

### Step O: Preparation of Int 2.13-15

[chemical structure]

To a solution of Int 2.13-14 (7.2 g, 7.20 mmol) in acetonitrile (600 mL) and water (260 mg) was added pyridinium triflate (1.66 g, 8.38 mmol, 1.20 equiv). The resulting solution was stirred for 10 min at room temperature to provide a solution of Int 2.13-15 which was used directly in the next step.

### Step P: Preparation of Int 2.13-16

[chemical structure]

The solution containing Int 2.13-15 was treated with tert-butylamine (36 mL) for 30 min at room temperature. The mixture was then concentrated under vacuum to afford 7.5 g of Int 2.13-16 as a foam which was used directly at next step without further purification.

### Step Q: Preparation of Int 2.13-17

[chemical structure]

Int 2.13-16 was dissolved in 75 mL of dichloromethane and the solution was treated with 91.5 mL of 6% dichloroacetic acid in dichloromethane. Triethylsilane (150 ml) was added after 10 minutes followed by pyridine (10 mL). The mixture was concentrated and the residue was dissolved in 50 mL of dry acetonitrile and concentrated. This process was repeated twice. The residue was finally dissolved in 20 mL of acetonitrile and used directly at next step.

### Step R: Preparation of Int 2.13-18

[chemical structure]

To the above solution containing 3.0 g of Int 2.13-17 and 3.0 g of 4A MS in 20 mL of MeCN was added a solution of Int 2.13-4 (18 g, 18.56 mmol, 2.5 equiv) in 30 ml of dry acetonitrile. The reaction solution was stirred for 5 min at 25° C. and used directly in the next step.

### Step S: Preparation of Int 2.13-19

[chemical structure]

To above reaction solution (Int 2.13-18) was added 8 mL of tert-butyl hydroperoxide (5.5 M in decane). The resulting solution was stirred for 30 min at 25° C. then diluted with 250 mL of ethyl acetate. The resulting solution was washed with 2×300 mL of water and 1×300 mL of saturated sodium chloride solution. The mixture was dried over anhydrous sodium sulfate, filtered and concentrated under vacuum to afford 21 g of Int 2.13-19 which was used directly in the next step.

### Step T—Preparation of Int 2.13-20

[chemical structure]

Int 2.13-19 (21 g, crude) was dissolved in methylene chloride (250 mL) before adding 210 mL dichloroacetic acid (6% in methylene chloride) to the solution. After stirring for 10 min, 420 mL of triethylsilane was added while stirring at room temperature. 50 mL of pyridine was then added and the mixture was concentrated under vacuum. The resulting residue was purified by flash column chromatography (C18 gel column, mobile phase, acetonitrile/water with 0.04% ammonium bicarbonate: 20% up to 80% within 10 min, then 100% for 5 min). This resulted in 2.5 g (30% overall for 6 steps) of Int 2.13-20 as a white solid.

### Step U. Preparation of Int 2.13-21

[chemical structure]

Compound 2-chloro-5,5-dimethyl-1,3,2-dioxaphosphorinane-2-oxide (1.9 g, 10.39 mmol, 4.7 eq) was added to the solution of Int 2.13-20 (2.5 g, 2.21 mmol) in 50 mL of pyridine and the reaction solution was stirred for 15 min at 25° C. The reaction was quenched by water (600 uL) followed by iodine (0.84 g, 3.31 mmol, 1.5 equiv). After stirring for 20 min, 12.5 mL saturated sodium thiosulfate solution was added. The mixture was concentrated to a foam to provide 2.5 g of Int 2.13-21 which was used directly in the next step.

### Step V: Preparation of Int 2.13-22

[chemical structure]

To a solution of 2.5 g crude Int 2.13-21 in acetonitrile (12.5 mL) was added tert-butylamine (22.5 mL). After stirring for 10 min at 25° C., the reaction solution was concentrated to a yellow foam and the crude product was purified by flash chromatography (C18 gel column, mobile phase, acetonitrile/water with 0.04% ammonium bicarbonate, gradient: 10% up to 50% within 15 min, 100% for 5 min) to provide pure 1.2 g (50% over 2 steps) Int 2.13-22.

### Step W— Preparation of Int 2.13-23

[chemical structure]

A 50-mL round-bottom flask was purged with argon then charged with a solution of Int 2.13-22 (500 mg, 0.46 mmol) in 25 mL of methanol. 10% anhydrous palladium carbon (250 mg, w/w) was then added and hydrogen was bubbled through the solution. The reaction mixture was stirred for 1 h at 25° C. then filtered through Celite and concentrated to provide 380 mg (87%) of Int 2.13-23 as a white solid.

### Step X: Preparation of Int 2.13-24

[chemical structure]

Into a 100-mL round-bottom flask was placed Int 2.13-23 (380 mg, 0.40 mmol) and 25 mL of methylamine (33% in anhydrous ethanol). The resulting solution was stirred for 16 h at 25° C. then concentrated to provide Int 2.13-24 as a white foam which used directly in the next step.

### Step Y: Preparation of Compound 21

[chemical structure]

The above crude Int 2.13-24 was azeotroped with pyridine/triethylamine (9 mL/3 mL) three times then dissolved in 0.8 mL pyridine in a 100 mL round-bottom flask. To this solution at 55° C. was added 6 mL triethylamine and 4 mL triethylamine trihydrofluride simultaneously. After stirring 1 h, the bath was removed and 60 mL anhydrous acetone was added immediately. The mixture was stirred for 20 min and the white solid was collected by filtration. The precipitate was washed with 5 mL of anhydrous acetone. The product was purified by preparative flash chromatography (AQ-C18 silica gel; mobile phase=acetonitrile/water with 0.04% ammonium bicarbonate; gradient 1% to 20% over 20 min, UV detector@210 nm). The resulting solution was lyophilized to provide 100 mg (36% for 2 steps) of Compound 21 as a white solid. LC-MS-SVT-001-1-24: (ESI, m/z): 690 [M+H]⁺. ¹H NMR (D₂O): δ 7.86 (s, 1H); 7.83 (s, 1H); 5.80-5.92 (m, 2H); 4.96-5.09 (m, 1H); 4.84-4.88 (m, 1H); 4.69-4.83 (m, 1H); 4.32-4.39 (m, 1H); 4.11-4.39 (m, 4H); 3.91-4.02 (m, 2H); [0.18 equiv TEA: 2.98-3.12 (q, J=6.6, 1H); 1.05-1.24 (m, J=6.6, 1.6H)]. ³¹P NMR (D₂O) δ −1.28, −1.39.

### Example 4

### Synthesis of ATAC1 and ATAC2

This example shows the synthesis of Pentafluorophenyl 25-(2-amino-3-pentylquinolin-5-yl)-19-oxo-4,7,10,13,16-pentaoxa-20-azapentacosanoate (ATAC1) and Perfluorophenyl 3-((4-amino-1-(2-hydroxy-2-methylpropyl)-1H-imidazo[4,5-c]quinolin-2-yl)methyl)-4-oxo-7,10,13,16,19-pentaoxa-3-azadocosan-22-oate (ATAC2).

[chemical structure]

### Step A: Preparation of Int ATAC1-1

[chemical structure]

To a 0° C. solution containing 271 mg (0.90 mmol) of 5-(5-aminopentyl)-3-pentylquinolin-2-amine in 4 mL of DCM was added 435 mg (1.00 mmol) of the NHS ester in 1 mL of DCM dropwise over 15 minutes. The reaction mixture was allowed to warm to ambient temperature over 19h before it was concentrated and purified by reverse phase chromatography. Pure fractions were lyophilized and dissolved in 3 mL of methanol then treated with 1 mL of 4N HCl in dioxane. The solution was stirred for 1 h then concentrated to afford the desired compound as an HCl salt which was used directly in the next step.

### Step B: Preparation of ATAC1

[chemical structure]

To a stirred solution of 25-(2-amino-3-pentylquinolin-5-yl)-19-oxo-4,7,10,13,16-pentaoxa-20 azapentacosanoic acid hydrochloride (130 mg, 0.198 mmol) and pentafluorophenol (146 mg, 0.792 mmol) in DMF (2.5 ml) at room temperature was added N,N′-Diisopropylcarbodiimide (0.186 ml, 1.189 mmol) dropwise. The reaction was stirred at room temperature for 18h then concentrated. The crude product was added to a 100 g C18 gold reverse phase column and was eluted with water/acetonitrile (0.1% TFA) 10⁻¹⁰⁰%. The fractions were combined and concentrated then freeze dried to give perfluorophenyl 25-(2-amino-3-pentylquinolin-5-yl)-19-oxo-4,7,10,13,16-pentaoxa-20-azapentacosanoate-2,2,2-trifluoroacetate (110 mg, 61.7% yield) as a clear gum. ¹H NMR (DMSO-d⁶) δ 13.7 (s, 1H), 8.37-8.35 (m, 3H), 7.78 (t, J=5.5 Hz, 1H), 7.63 (t, J=7.5 Hz, 1H), 7.53 (d, J=8.5 Hz, 1H), 7.31 (d, J=7.0 Hz, 1H), 3.58 (t, J=6.0 Hz, 2H), 3.63-3.43 (m, 20H), 3.04-2.96 (m, 6H), 2.73 (t, J=7.5 Hz, 2H), 2.27 (t, J=7.5 Hz, 2H), 1.60-1.55 (m, 4H), 1.44-1.33 (m, 9H), 0.88 (t, J=7.5 Hz, 3H). LCMS [M+H]=786.3.

The following compound in TABLE 2 can be prepared using a method similar to that described above for ATAC1.

TABLE 2

| Compound | Structure | IUPAC | M + 1 |
|---|---|---|---|
| ATAC2 | [chemical structure] | Perfluorophenyl 3- ((4-amino- 1-(2-hydroxy-2- methylpropyl)-1H- imidazo[4,5-c]quinolin-2- yl)methyl)-4-oxo- 7,10,13,16,19-pentaoxa-3- azadocosan-22-oate | 800 |

### Example 5

### Synthesis of ATAC3 and ATAC4

This example shows the synthesis of pentafluorophenyl 25-(2-amino-3-pentylquinolin-5-yl)-19-oxo-4,7,10,13,16-pentaoxa-20-azapentacosanoate (ATAC3) and 2,5-Dioxopyrrolidin-1-yl 3-((4-amino-1-(2-hydroxy-2-methylpropyl)-1H-imidazo-[4,5-c]quinolin-2-yl)methyl)-4-oxo-7,10,13,16,19-pentaoxa-3-azadocosan-22-oate (ATAC4).

[chemical structure]

### Step A: Preparation of ATAC3

To a stirred solution of Int ATAC1-1 (185 mg, 0.282 mmol) and N-hydroxysuccinimide (130 mg, 1.128 mmol) in DMF (3 ml) was added N,N′-diisopropylcarbodiimide (0.221 ml, 1.411 mmol) dropwise and the reaction mixture was stirred at room temperature for 18 hours. The reaction mixture was filtered and washed with acetonitrile and the filtrate was evaporated. The resulting residue was purified by silica gel Silica gel column chromatography (DCM/MeOH/HOAc) to give 65 mg of the desired product as the acetic acid salt which was subsequently dissolved in 2 mL of DCM and treated with 2M HCl in diethyl ether. The solution was stirred for 1 h then concentrated and lyophilized to afford the desired compound as the HCl salt. ¹H NMR (CDCl₃) δ 15.2 (s, 1H), 8.15 (d, J=7.8 Hz, 1H), 7.68 (d, J=7.9 Hz, 1H), 7.55 (t, J=8.1 Hz, 1H), 6.55 (bs, 1H), 3.98 (t, J=6.0 Hz, 2H), 3.83-3.55 (m, 18H), 3.33-3.22 (m, 2H), 2.95-2.56 (m, 11H), 2.27 (t, J=7.5 Hz, 2H), 1.60-1.55 (m, 4H), 1.44-1.33 (m, 9H), 0.88 (t, J=7.5 Hz, 3H). LCMS [M+H]=717.3.

The following 2,5-Dioxopyrrolidin-1-yl 3-((4-amino-1-(2-hydroxy-2-methylpropyl)-1H-imidazo-[4,5-c]quinolin-2-yl)methyl)-4-oxo-7,10,13,16,19-pentaoxa-3-azadocosan-22-oate (ATAC4) compound can be prepared using a method similar to that described above for ATAC3.

[chemical structure]

¹H NMR (CDC₃) δ 14.9 (s, 1H), 8.88 (bs, 1H), 8.15 (d, 1H), 7.85 (d, 1H), 7.61 (t, 1H), 7.45 (t, 1H), 4.72 (s, 2H), 3.83 (m, 4H), 3.65-3.45 (m, 18H), 2.90-2.71 (m, 9H), 1.43 (t, J=7.0 Hz, 3H), 1.33 (s, 6H). LCMS [M+H]=731.

### Example 6

### Synthesis of ATAC5, ATAC6 and ATAC7

This example shows the synthesis of 2,5-dioxopyrrolidin-1-yl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC5), 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC6), and 2,5-dioxopyrrolidin-1-yl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC7).

[chemical structure]

### Step A: Preparation of Int ATAC5-1

[chemical structure]

A solution of 5-(5-aminopentyl)-3-pentylquinolin-2-amine (300 mg, 1.00 mmol) in 5 mL DCM was stirred at room temperature under nitrogen for 10 min before tert-butyladipate-valine-alanine-para-aminobenzyl-4-nitrophenylcarbonate (tBuAdip-va-PAB-OPNP, 656 mg, 1.00 mmol) and DIPEA (0.26 ml, 1.5 mmol) in 3 mL of DCM were added and the mixture was stirred at room temperature overnight. The mixture was concentrated and purified by column chromatography. Clean fractions were combined and evaporated and the residue was dissolved in 2 mL of DCM and treated with 2M HCl in diethyl ether. The solution was stirred for 1 h then concentrated and lyophilized to afford the desired compound Int ATAC5-1 as the HCl salt. MS m/z 761 (M)⁺.

### Step B: Preparation of ATAC5

[chemical structure]

To a stirred solution of 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoic acid hydrochloride (221 mg, 0.282 mmol) and N-hydroxysuccinimide (130 mg, 1.12 mmol) in DMF (3 ml) was added N,N′-diisopropylcarbodiimide (0.221 ml, 1.41 mmol) dropwise and the reaction mixture was stirred at room temperature for 5h. HPLC indicated some starting material remained so the reaction was stirred at ambient temperature overnight. The reaction mixture was filtered and washed with acetonitrile. The filtrate was evaporated and the residue was dissolved in DMSO and purified by reverse phase chromatography [water/acetonitrile (0.1% TFA)] from 10% followed by a gradient from 20 to 80%. Pure fractions were combined to give 2,5-dioxopyrrolidin-1-yl 6-(((S)-1-(((S)-1-((4-((((5-(2-amino-3-pentylquinolin-5-yl)pentyl)-carbamoyl)oxy)methyl)phenyl)amino)-1-oxopropan-2-yl)amino)-3-methyl-1-oxobutan-2-yl)amino)-6-oxohexanoate 2,2,2-trifluoroacetate (109 mg, 40% yield) as a white solid. ¹H NMR (DMSO-d⁶) δ 13.6 (s, 1H), 9.92 (s, 1H), 8.32 (d, J=7.5 Hz, 1H), 7.84 (d, J=8.5 Hz, 1H), 7.61-7.55 (m, 4H), 7.30-7.17 (m, 4H), 4.92 (s, 2H), 4.37 (t, J=7.0 Hz, 1H), 4.18 (t, J=7.0 Hz, 1H), 2.96 (m, 4H), 2.81-2.62 (m, 8H), 2.33-2.11 (m, 2H), 1.95 (q, J=7.0 Hz, 1H), 1.63-1.55 (m, 8H), 1.50-1.40 (m, 2H), 1.38-1.33 (m, 4H), 1.29 (d, J=7.0 Hz, 3H), 0.83 (d, J=7.0 Hz, 6H). LCMS [M+H]=844.3.

The following ATAC6 compound and ATAC7 compound in TABLE 3 can be prepared using a method similar to that described above for ATAC5.

TABLE 3

| Compound | Structure | Name | M + 1 |
|---|---|---|---|
| ATAC6 | [chemical structure] | 2,5-dioxopyrrolidin-1- yl 7-(((S)-1-(((S)-1- ((4-(((((4-amino-1-(2- hydroxy-2-methyl propyl)-1H-imidazo [4,5-c]quinolin-2-yl) methyl)(ethyl) carbamoyl) oxy)methyl)phenyl) | 872 |
| | | amino)-1-oxopropan- | |
| | | 2-yl)amino)-3- | |
| | | methyl-1- | |
| | | oxobutan-2-yl)amino)- | |
| | | 7-oxoheptanoate | |
| ATAC7 | [chemical structure] | 2,5-dioxopyrrolidin-1- yl 7-(((S)-1-(((S)-1- ((4-(((((4-amino-1- (2-hydroxy-2- methylpropyl)-1H- imidazo[4,5-c]quinolin- 2-yl)methyl)(ethyl) carbamoyl)oxy)methyl) phenyl)amino)-1- oxo-5-ureidopentan-2- yl)amino)-3-methyl-1- oxobutan-2-yl)amino)- 7-oxoheptanoate | 958 |

### Example 7

### Synthesis of ATAC8, ATAC9, and ATAC10

This example shows synthesis of Perfluorophenyl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate (ATAC8), perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxopropan-2-yl)amino)-3-methyl-1-oxobutan-2-yl)amino)-7-oxoheptanoate (ATAC9), and perfluorophenyl 7-(((S)-1-(((S)-1-((4-(((((4-amino-1-(2-hydroxy-2-methylpropyl)-1H-imidazo[4,5-c]quinolin-2-yl)methyl)(ethyl)carbamoyl)oxy)methyl)phenyl)amino)-1-oxo-5-ureidopentan-2-yl)amino)-3-methyl-1-oxobutan-2-yl)amino)-7-oxoheptanoate (ATAC10).

[chemical structure]

### Step A Preparation of ATAC8

[chemical structure]

To a stirred solution of 6-(((S)-1-(((S)-1-((4-((((5-(2-amino-3-pentyquinolin-5-yl)pentyl)carbamoyl)oxy)methyl)phenyl)amino)-1-oxopropan-2-yl)amino)-3-methyl-1-oxobutan-2-yl)amino)-6-oxohexanoic acid hydrochloride (168 mg, 0.215 mmol) and pentafluorophenol (158 mg, 0.86 mmol) in DMF (3 ml) was added N,N′-diisopropylcarbodiimide (0.166 ml, 1.07 mmol) dropwise and the reaction mixture was stirred at room temperature for 6h. The reaction mixture was concentrated and the residue was dissolved in DMSO and purified by reverse phase chromatography [water/acetonitrile (0.1% TFA)] from 10% followed by a gradient from 20 to 80%. Pure fractions were combined to give perfluorophenyl 6-(((S)-1-(((S)-1-((4-((((5-(2-amino-3-pentylquinolin-5-yl)pentyl)carbamoyl)oxy)methyl)phenyl)amino)-1-oxopropan-2-yl)amino)-3-methyl-1-oxobutan-2-yl)amino)-6-oxohexanoate 2,2,2-trifluoroacetate (122 mg) as a white solid. ¹H NMR (DMSO-d⁶) δ 13.5 (s, 1H), 9.92 (s, 1H), 8.35 (bs, 3H), 8.17 (d, J=7.0 Hz, 1H), 7.87 (d, J=7.0 Hz, 1H), 7.64-7.52 (m, 4H), 7.32-7.18 (m, 4H), 4.91 (s, 2H), 4.37 (t, J=7.0 Hz, 1H), 4.19 (t, J=7.0 Hz, 1H), 3.60-3.50 (m, 4H), 2.97 (m, 4H), 2.79 (t, J=7.0 Hz, 2H), 2.74 (t, J=7.0 Hz, 2H), 2.31-2.22 (m, 2H), 1.96 (q, J7.0 Hz, 1H), 1.71-1.51 (m, 8H), 1.45-1.38 (m, 2H), 1.40-1.27 (m, 9H), 0.90-0.80 (m, 9H). LCMS [M+H]=913.4.

The following compound in TABLE 4 can be prepared using a method similar to that described in above for ATAC8.

TABLE 4

| Compound | Structure | Name | M + 1 |
|---|---|---|---|
| ATAC9 | [chemical structure] | perfluorophenyl 7- (((S)-1-(((S)-1-((4- (((((4-amino-1-(2- hydroxy-2-methyl propyl)-1H-imidazo [4,5-c]quinolin-2- yl)methyl)(ethyl) carbamoyl)oxy) methyl)phenyl) | 941 |
| | | amino)-1-oxopropan- | |
| | | 2-yl)amino)-3- | |
| | | methyl-1-oxobutan- | |
| | | 2-yl)amino)-7- | |
| | | oxoheptanoate | |
| ATAC10 | [chemical structure] | perfluorophenyl 7- (((S)-1-(((S)- 1-((4-(((((4-amino- 1-(2-hydroxy-2- methylpropyl)-1H- imidazo[4,5-c] quinolin-2-yl)methyl) (ethyl)carbamoyl)oxy) methyl)phenyl)amino)- 1-oxo-5-ureidopentan- 2-yl)amino)-3- methyl-1-oxobutan- 2-yl)amino)- | 1027 |
| | | 7-oxoheptanoate | |

### Example 8

### Synthesis of ATAC11

This example shows the synthesis of N-((4-amino-1-(2-hydroxy-2-methylpropyl)-1H-imidazo[4,5-c]quinolin-2-yl)methyl)-1-(3-(2,5-di oxo-2,5-dihydro-1H-pyrrol-1-yl)propanamido)-N-ethyl-3,6,9,12-tetraoxapentadecan-15-amide (ATAC11).

[chemical structure]

### Step A: Preparation of ATAC11

[chemical structure]

A solution of MAL-PEG4-acid (265.7 mg, 0.638 mmol) and N,N′-dicyclohexylcarbodiimide (DCC, 144.8 mg, 0.702 mmol) in dry dichloromethane/acetonitrile (1:1, 5 mL) was stirred at room temperature for 1 h, followed by addition of compound 1 (100 mg, 0.319 mmol) in one portion. After 72h of stirring, volatile organics were removed under vacuum. The residue obtained was purified by flash column chromatography on silica gel, eluting with step gradients of methanol in dichloromethane at a ratio of v/v 1:20, 1:15, and 1:9, to afford the target product N-((4-amino-1-(2-hydroxy-2-methylpropyl)-1H-imidazo[4,5-c]quinolin-2-yl)methyl)-1-(3-(2,5-dioxo-2,5-dihydro-1H-pyrrol-1-yl)propanamido)-N-ethyl-3,6,9,12-tetraoxapentadecan-15-amide (80 mg, 35% yield) as white colored foamy solid oil. ¹H NMR (300 MHz, CDCl₃) δ 8.40-7.82 (br m, 1H), 7.74 (d, J=8.1 Hz, 1H), 7.44 (t, J=7.5 Hz, 1H), 7.30 (t, J=7.4 Hz, 1H), 6.76-6.28 (br m, 2H), 4.82-4.32 (br m, 2H), 4.08-3.64 (br m, 6H), 3.54 (br s, 14H), 3.31 (br s, 3H), 2.63 (br s, 2H), 2.38 (t, J=6.9 Hz, 2H), 1.27 (br s, 4H), 1.20-0.68 (br m, 5H). MS (ESI+) m/z 712 (M+1), 734 (M+Na).

### Example 9

### Synthesis of ATAC12, ATAC13, ATAC14, ATAC15, ATAC16, ATAC17, ATAC18, ATAC19, ATAC20, and ATAC21

This example shows the synthesis of N-(5-(2-amino-3-pentylquinolin-5-yl)pentyl)-1-(3-(2,5-dioxo-2,5-dihydro-1H-pyrrol-1-yl)propanamido)-3,6,9,12-tetraoxapentadecan-15-amide (ATAC12), 1-(3-(2,5-dioxo-2,5-dihydro-1H-pyrrol-1-yl)propanamido)-N-(3-pentylquinolin-2-yl)-3,6,9,12-tetraoxapentadecan-15-amide (ATAC13), 1-(3-(2,5-dioxo-2,5-dihydro-1H-pyrrol-1-yl)propanamido)-N-(1-isobutyl-1H-imidazo[4,5-c]quinolin-4-yl)-3,6,9,12-tetraoxapentadecan-15-amide (ATAC14), 1-(3-(2,5-dioxo-2,5-dihydro-1H-pyrrol-1-yl)propanamido)-N-methyl-N-(2-(3-(7-methylbenzo[1,2-d:3,4-d′]bis(thiazole)-2-yl)ureido)ethyl)-3,6,9,12-tetraoxapentadecan-15-amide (ATAC15), (S)-1-(3-(2,5-dioxo-2,5-dihydro-1H-pyrrol-1-yl)propanamido)-N-(1-((7-methylbenzo[1,2-d:3,4-d′]bis(thiazole)-2-yl)amino)-1-oxo-3-phenylpropan-2-yl)-3,6,9,12-tetraoxapentadecan-15-amide (ATAC16), N-(benzo[d]thiazol-2-yl)-1-(3-(2,5-dioxo-2,5-dihydro-1H-pyrrol-1-yl)propanamido)-N-((8-hydroxyquinolin-7-yl)(4-(trifluoromethoxy)phenyl)methyl)-3,6,9,12-tetraoxapentadecan-15-amide (ATAC17), N-((2R,3R,3aS,7aR,9R,10R,10aS,14aR)-2,9-bis(2-amino-6-oxo-1H-purin-9(6H)-yl)-5,10,12-trihydroxy-5,12-dioxidodecahydrodifuro[3,2-d:3′,2′-j][1,3,7,9,2,8]tetra-oxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC18), N-((2R,3R,3aS,7aR,9R,10R,10aS,14aR)-2,9-bis(2-amino-6-oxo-1H-purin-9(6H)-yl)-10-hydroxy-5,12-dimercapto-5,12-dioxidodecahydrodifuro[3,2-d:3′,2′-j][1,3,7,9,2,8]tetraoxadiphosphacyclododecin-3-yl)-1-(3-(2,5-dioxo-2,5-dihydro-1H-pyrrol-1-yl)propanamido)-3,6,9,12-tetraoxapentadecan-15-amide (ATAC19), N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetra-oxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC20), and N-(9-((2R,3R,3aS,7aR,9R,10R,10aS,14aR)-9-(2-amino-6-oxo-1H-purin-9(6H)-yl)-3,5,10,12-tetrahydroxy-5,12-dioxidodecahydrodifuro[3,2-d:3′,2′-j][1,3,7,9,2,8]tetraoxadiphosphacyclododecin-2-yl)-9H-purin-6-yl)-1-(3-(2,5-dioxo-2,5-dihydro-1H-pyrrol-1-yl)propanamido)-3,6,9,12-tetraoxapentadecan-15-amide (ATAC21).

[chemical structure]

### Step A: Preparation of ATAC12

[chemical structure]

To a stirred solution containing 100 mg (0.33 mmol) of 5-(5-aminopentyl)-3-pentylquinolin-2-amine in 13 mL of CH₂Cl₂ under N₂ was added a solution of MAL-PEG4-NHS [CAS No 756525-99-2] (171 mg, 0.33 mmol) in 3 mL of CH₂Cl2 by syringe pump over 90 mins. The reaction mixture was stirred at room temperature for 16h then evaporated to afford a residue which was purified by silica gel chromatography (CombiFlash Gold (12 g): CH₂C₂/CH₃OH/NH₄OH) to afford a light yellow syrup which was dissolved in 5 mL of CH₃CN and lyophilized to provide 164 mg of the desired compound. ¹H NMR (CD₃OD) δ 7.95 (s, 1H), 7.38 (s, 1H), 7.37 (s, 1H), 7.07 (t, J=8.5 Hz, 1H), 6.78 (s, 2H), 3.75 (t, J=6.0 Hz, 2H), 3.65 (t, J=6.0 Hz, 2H), 3.59-3.52 (m, 12H), 3.46 (t, J=5.5 Hz, 2H), 3.28 (t, J=7.5 Hz, 2H), 3.18 (t, J=7.5 Hz, 2H), 2.98 (t, J=8.5 Hz, 2H), 2.67 (t, J=7.5 Hz, 2H), 2.44 (t, J=7.0 Hz, 2H), 2.40 (t, J=7.0 Hz, 2H), 1.76-1.68 (m, 4H), 1.58-1.52 (m, 2H), 1.46-1.40 (m, 6H), 0.94 (t, J=7.0 Hz, 3H). (MS (ESI+) m/z 698 (M+1).

The following compounds in TABLE 5 can be prepared using a method similar to that as described above for ATAC12.

TABLE 5

| Compound | Structure | Name | M + 1 |
|---|---|---|---|
| ATAC13 | [chemical structure] | 1-(3-(2,5-dioxo-2,5-dihydro-1H- pyrrol-1-yl)propanamido)-N-(3- pentylquinolin-2-yl)-3,6,9,12- tetraoxapentadecan-15-amide | 613 |
| ATAC14 | [chemical structure] | 1-(3-(2,5-dioxo-2,5-dihydro-1H- pyrrol-1-yl)propanamido)-N-(1- isobutyl-1H-imidazo[4,5- c]quinolin-4-yl)-3,6,9,12- tetraoxapentadecan-15-amide | 639 |
| ATAC15 | [chemical structure] | 1-(3-(2,5-dioxo-2,5-dihydro-1H- pyrrol-1-yl)propanamido)-N- methyl-N-(2-(3-(7- methylbenzo[1,2-d:3,4- d′]bis(thiazole)-2-yl)ureido)ethyl)- 3,6,9,12-tetraoxapentadecan-15- amide | 720 |
| ATAC16 | [chemical structure] | (S)-1-(3-(2,5-dioxo-2,5-dihydro- 1H-pyrrol-1-yl)propanamido)-N- (1-((7-methylbenzo[1,2-d:3,4- d′]bis(thiazole)-2-yl)amino)-1- oxo-3-phenylpropan-2-yl)- 3,6,9,12-tetraoxapentadecan-15- amide | 635 |
| ATAC17 | [chemical structure] | N-(benzo[d]thiazol-2-yl)-1-(3- (2,5-dioxo-2,5-dihydro-1H-pyrrol- 1-yl)propanamido)-N-((8- hydroxyquinolin-7-yl)(4- (trifluoromethoxy)phenyl)methyl)- 3,6,9,12-tetraoxapentadecan-15- amide | 734 |
| ATAC18 | [chemical structure] | N- ((2R,3R,3aS,7aR,9R,10R,10aS,14 aR)-2,9-bis(2-amino-6-oxo-1H- purin-9(6H)-yl)-5,10,12- trihydroxy-5,12- dioxidodecahydrodifuro[3,2- d:3′,2′-j][1,3,7,9,2,8]tetra- oxadiphosphacyclododecin-3-yl)- 1-(3-(2,5-dioxo-2,5-dihydro-1H- pyrrol-1-yl)propanamido)- 3,6,9,12-tetraoxapentadecan-15- amide | 1088 |
| ATAC19 | [chemical structure] | N- ((2R,3R,3aS,7aR,9R,10R,10aS,14 aR)-2,9-bis(2-amino-6-oxo-1H- purin-9(6H)-yl)-10-hydroxy-5,12- dimercapto-5,12- dioxidodecahydrodifuro[3,2- d:3′,2′- j][1,3,7,9,2,8]tetraoxadiphosphacyclo dodecin-3-yl)-1-(3-(2,5-dioxo- 2,5-dihydro-1H-pyrrol-1- yl)propanamido)-3,6,9,12- tetraoxapentadecan-15-amide | 1120 |
| ATAC20 | [chemical structure] | N-(9- ((2R,3R,3aS,7aR,9R,10R,10aS,14 aR)-9-(2-amino-6-oxo-1H-purin- 9(6H)-yl)-3,5,10,12-tetrahydroxy- 5,12-dioxidodecahydrodifuro[3,2- d:3′,2′-j][1,3,7,9,2,8]tetra- oxadiphosphacyclododecin-2-yl)- 9H-purin-6-yl)-1-(3-(2,5-dioxo- 2,5-dihydro-1H-pyrrol-1- yl)propanamido)-3,6,9,12- tetraoxapentadecan-15-amide | 1073 |
| ATAC21 | [chemical structure] | N-(9- ((2R,3R,3aS,7aR,9R,10R,10aS,14 aR)-9-(2-amino-6-oxo-1H-purin- 9(6H)-yl)-3,5,10,12-tetrahydroxy- 5,12-dioxidodecahydrodifuro[3,2- d:3′,2′- j][1,3,7,9,2,8]tetraoxadiphosphacyclo dodecin-2-yl)-9H-purin-6-yl)- 1-(3-(2,5-dioxo-2,5-dihydro-1H- pyrrol-1-yl)propanamido)- 3,6,9,12-tetraoxapentadecan-15- amide | 1073 |

### Example 10

### Synthesis of ATAC22, ATAC23, ATAC24, ATAC25, ATAC26, ATAC27, ATAC28, ATAC29, ATAC30, and ATAC31

This example shows the synthesis of 4-((S)-2-((S)-2-(6-(2,5-dioxo-2,5-dihydro-1H-pyrrol-1-yl)hexanamido)-3-methylbutanamido)propanamido)benzyl ((4-amino-1-(2-hydroxy-2-methyl-propyl)-1H-imidazo[4,5-c]quinolin-2-yl)methyl)(ethyl)carbamate (ATAC22), 4-((S)-2-((S)-2-(6-(2,5-dioxo-2,5-dihydro-1H-pyrrol-1-yl)hexanamido)-3-methyl-butanamido)propanamido)benzyl (5-(2-amino-3-pentylquinolin-5-yl)pentyl)-carbamate (ATAC23), 4-((S)-2-((S)-2-(6-(2,5-dioxo-2,5-dihydro-1H-pyrrol-1-yl)hexanamido)-3-methylbutan-amido)-5-ureidopentanamido)benzyl-(5-(2-amino-3-pentylquinolin-5-yl)pentyl)-carbamate (ATAC24), 4-((S)-2-((S)-2-(6-(2,5-dioxo-2,5-dihydro-1H-pyrrol-1-yl)hexanamido)-3-methylbutanamido)-5-ureidopentanamido)benzyl((4-amino-1-(2-hydroxy-2-methylpropyl)-1H-imidazo[4,5-c]quinolin-2-yl)methyl)(ethyl)carbamate TFA salt (ATAC25), 2-(3-{2-[N-Methyl({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino]ethyl}ureido)-7-methyl-1,6-dithia-3,8-diaza-as-indacene (ATAC26), 2-{[(8-Hydroxy-7-quinolyl)(p-trifluoromethoxyphenyl)methyl]({p-[(S)-2-{(S)-2-[6-(2,5-dioxo-1H-pyrrol-1-yl)hexanoylamino]-3-methylbutyrylamino}-5-ureidovalerylamino]phenyl}methoxycarbonyl)amino}-1,3-benzothiazole (ATAC27), (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5.12λ5-diphosphatricyclo[13.3.0.06,10]octadecane-3,12-dione (ATAC28), (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}propionylamino]phenyl}methoxycarbonylamino)-8,17-bis(2-amino-6-oxo-1,9-dihydropurin-9-yl)-3,12-dihydroxy-9-hydroxy-2.4.7.11.13.16-hexaoxa-3λ5.12λ5-diphosphatricyclo[13.3.0.06,10]octadecane-3,12-dione (ATAC29), (1R,6R,8R,9S,10S,15R,17R,18S)-18-({p-[(S)-2-{(S)-2-[6-(2,5-Dioxo-1H-pyrrol-1-yl)hexanoylamino]-3-methylbutyrylamino}-5-ureidovalerylamino]phenyl}methoxycarbonylamino)-8,17-bis(2-amino-6-oxo-1,9-dihydropurin-9-yl)-9-hydroxy-3,12-dimercapto-2.4.7.11.13.16-hexaoxa-3λ5.12λ5-diphosphatricyclo[13.3.0.06,10]octadecane-3,12-dione (ATAC30), and {p-[(S)-2-{(S)-2-[6-(2,5-Dioxo-1H-pyrrol-1-yl)hexanoylamino]-3-methylbutyrylamino}-5-ureidovalerylamino]phenyl}methyl 9-{(1S,6R,8R,9S,10S,15R,17R,18S)-8-(2-amino-6-oxo-1,9-dihydropurin-9-yl)-3,12-dihydroxy-9,18-dihydroxy-3,12-dioxo-2.4.7.11.13.16-hexaoxa-3λ5.12λ5-diphosphatricyclo[13.2.1.06,10]octadec-17-yl}-9a-adenineecarboxylate (ATAC31).

[chemical structure]

### Step A: Preparation of ATAC22

[chemical structure]

A solution of compound 1 (150 mg, 0.479 mmol) and N,N′-diisopropylethylamine (145.4 mg, 1.437 mmol) in dry DMF was stirred at room temperature for 5 min., followed by addition of maleimidocaproyl-valine-alanine-p-aminobenzyl alcohol p-nitrophenyl-carbonate (MC-Val-Ala-PAB-PNP, 343.6 mg, 0.527 mmol). After stirring for 24 h, volatile organics were removed under vacuum. The residue obtained was triturated with dry acetonitrile. The precipitated solid was collected by filtration, washed with acetonitrile and dried under vacuum to obtain unreacted MC-Val-Ala-PAB-PNP (130 mg) as beige solid. The filtrate and washings were combined and concentrated under vacuum. The residue obtained was purified by flash column chromatography on silica gel, eluting with step gradients of MeOH in dichloromethane at a ratio of v/v 1:20, 1:15, and 1:10, to afford the target product me-Val-Ala-PAB-GDQ (70 mg, 18% yield) as beige colored foamy solid. ¹H NMR (DMSO-d⁶) δ 10.1-9.75 (br m, 1H), 8.58-8.24 (br m, 1H), 8.15 (d, J=6.6 Hz, 1H), 8.01 (br s, 1H), 7.81 (d, J=8.4 Hz, 1H), 7.71 (d, J=8.4 Hz, 1H), 7.65-7.48 (m, 2H), 7.46-7.34 (m, 2H), 7.29 (br s, 1H), 7.18 (br s, 1H), 6.99 (s, 2H), 5.03 (br s, 2H), 4.96 (br s, 1H), 4.72 (br s, 1H), 4.48-4.26 (m, 1H), 4.26-4.04 (m, 1H), 2.22-2.02 (m, 2H), 2.02-1.80 (m, 1H), 1.58-1.37 (m, 4H), 1.36-0.92 (br m, 15H), 0.92-0.53 (br m, 7H). MS (ESI+) m/z 826 (M+1).

The following ATAC30, ATAC31, ATAC32, ATAC33, ATAC34, ATAC35, ATAC36, ATAC37, ATAC38, ATAC39, ATAC40, ATAC41, and ATAC42 can be prepared using a method similar to that described above for ATAC29.

### ATAC23: 4-((S)-2-((S)-2-(6-(2,5-dioxo-2,5-dihydro-1H-pyrrol-1-yl)hexanamido)-3-methyl-butanamido)propanamido)benzyl (5-(2-amino-3-pentylquinolin-5-yl)pentyl)-carbamate

[chemical structure]

¹H NMR (CD₃OD) δ 8.35 (s, 1H), 7.63 (t, J=8.5 Hz, 1H), 7.55 (d, J=8.0 Hz, 1H), 7.47 (d, J=8.0 Hz, 1H), 7.33 (d, J=8.0 Hz, 1H), 7.27 (d, J=8.0 Hz, 1H), 6.78 (s, 2H), 5.00 (s, 2H), 4.46 (q, J=7.0 Hz, 2H), 4.13 (d, J=7.0 Hz, 1H), 3.47-3.4 (m, 3H), 3.17 (t, J=7.0 Hz, 2H), 3.05 (t, J=7.0 Hz, 2H), 2.75 (t, J=7.5 Hz, 2H), 2.27 (t, J=7.5 Hz, 2H), 2.07 (q, J=7.0 Hz, 1H), 1.72-1.51 (m, 10H), 1.46-1.35 (m, 8H), 1.32-1.26 (m, 3H), 1.00-0.92 (m, 9H). LCMS [M+H]=812.4.

### ATAC24: 4-((S)-2-((S)-2-(6-(2,5-dioxo-2,5-dihydro-1H-pyrrol-1-yl)hexanamido)-3-methylbutan-amido)-5-ureidopentanamido)benzyl-(5-(2-amino-3-pentylquinolin-5-yl)pentyl)-carbamate

[chemical structure]

¹H NMR (DMSO-d⁶) δ 13.5 (bs, 1H), 10.0 (s, 1H), 8.40 (m, 3H), 8.07 (d, J=7.5 Hz, 1H), 7.80 (d, J=8.5 Hz, 1H), 7.6-7.5 (m, 4H), 7.35-7.25 (m, 2H), 6.01 (m, 1H), 5.42 (s, 1H), 4.89 (s, 2H), 4.41 (q, J=7.0 Hz, 1H), 4.18 (t, J=7.0 Hz, 1H), 3.10-2.90 (m, 6H), 2.75 (t, J=7.5 Hz, 2H), 2.27 (t, J=7.5 Hz, 2H), 2.07 (q, J=7.0 Hz, 1H), 1.72-1.51 (m, 10H), 1.46-1.35 (m, 8H), 1.32-1.26 (m, 3H), 1.00-0.92 (m, 9H). LCMS [M+H]=898.

### ATAC25: 4-((S)-2-((S)-2-(6-(2,5-dioxo-2,5-dihydro-1H-pyrrol-1-yl)hexanamido)-3-methylbutanamido)-5-ureidopentanamido)benzyl((4-amino-1-(2-hydroxy-2-methylpropyl)-1H-imidazo[4,5-c]quinolin-2-yl)methyl)(ethyl)carbamate TFA salt

[chemical structure]

¹H NMR (DMSO-d⁶) δ 13.4 (bs, 1H), 9.99-9.89 (br m, 1H), 9.09-8.40 (m, 3H), 8.07 (d, J=7.5 Hz, 1H), 7.80 (d, J=8.5 Hz, 1H), 7.68 (t, J=8.0 Hz, 1H), 7.59 (bs, 1H), 7.51 (t, J=8.5 Hz, 1H), 7.46-7.14 (m, 2H), 7.00 (s, 1H), 5.99 (br s, 1H), 5.05 (br s, 1H), 4.95 (br s, 1H), 4.37 (q, J=7.0 Hz, 1H), 4.18 (t, J=7.0 Hz, 1H), 3.37 (t, J=7.0 Hz, 2H), 3.03-2.93 (m, 2H), 2.22-2.07 (m, 2H), 1.99-1.92 (m, 1H), 1.75-1.05 (br m, 20H), 0.85 (d, J=8.5 Hz, 3H), 0.81 (d, J=8.5 Hz, 3H). MS (ESI+) m/z 912.5 (M+1).

TABLE 6

| Compound | Structure | Name | M + 1 |
|---|---|---|---|
| ATAC26 | [chemical structure] | 2-(3-{2-[N-Methyl({p-[(S)-2-{(S)-2-[6-(2,5- dioxo-1H-pyrrol-1-yl)hexanoylamino]-3- methylbutyrylamino}-5- ureidovalerylamino]phenyl}methoxycarbonyl) amino]ethyl}ureido)-7-methyl-1,6-dithia-3,8- diaza-as-indacene | 921 |
| ATAC27 | [chemical structure] | 2-{[(8-Hydroxy-7-quinolyl)(p- trifluoromethoxyphenyl)methyl]({p-[(S)-2- {(S)-2-[6-(2,5-dioxo-1H-pyrrol-1- yl)hexanoylamino]-3-methylbutyrylamino}-5- ureidovalerylamino]phenyl}methoxycarbonyl) amino}-1,3-benzothiazole | 1067 |
| ATAC28 | [chemical structure] | (1R,6R,8R,9S,10S,15R,17R,18S)-18-({p-[(S)- 2-{(S)-2-[6-(2,5-Dioxo-1H-pyrrol-1- yl)hexanoylamino]-3-methylbutyrylamino}-5- uriedovalerylamino]phenyl}methoxycarbonyl amino)-8,17-bis(2-amino-6-oxo-1,9- dihydropurin-9-yl)-3,12-dihydroxy-9- hydroxy-2.4.7.11.13.16-hexaoxa-3λ5.12λ5- diphosphatricyclo[13.3.0.06,10]octadecane- 3,12-dione | 1289 |
| ATAC29 | [chemical structure] | (1R,6R,8R,9S,10S,15R,17R,18S)-18-({p-[(S)- 2-{(S)-2-[6-(2,5-Dioxo-1H-pyrrol-1- yl)hexanoylamino]-3- methylbutyrylamino}propionylamino]phenyl} methoxycarbonylamino)-8,17-bis(2-amino-6- oxo-1,9-dihydropurin-9-yl)-3,12-dihydroxy-9- hydroxy-2.4.7.11.13.16-hexaoxa-3λ5.12λ5- diphosphatricyclo[13.3.0.06,10]octadecane- 3,12-dione | 1203 |
| ATAC30 | [chemical structure] | (1R,6R,8R,9S,10S,15R,17R,18S)-18-({p-[(S)- 2-{(S)-2-[6-(2,5-Dioxo-1H-pyrrol-1- yl)hexanoylamino]-3-methylbutryrlamino}-5- ureidovalerylamino]phenyl}methoxycarbonyl amino)-8,17-bis(2-amino-6-oxo-1,9- dihydropurin-9-yl)-9-hydroxy-3,12- dimercapto-2.4.7.11.13.16-hexaoxa-3λ5.12λ5- diphosphatricyclo[13.3.0.06,10]octadecane- 3,12-dione | 1321 |
| ATAC31 | [chemical structure] | {p-[(S)-2-{(S)-2-[6-(2,5-Dioxo-1H-pyrrol-1- yl)hexanoylamino]-3-methylbutyrylamino}-5- ureidovalerylamino]phenyl}methyl 9- {(1S,6R,8R,9S,10S,15R,17R,18S)-8-(2- amino-6-oxo-1,9-dihydropurin-9-yl)-3,12- dihydroxy-9,18-dihydroxy-3,12-dioxo- 2.4.7.11.13.16-hexaoxa-3λ5.12λ5- diphosphatricyclo[13.2.1.06,10]octadec-17- yl}-9a-adenineecarboxylate | 1274 |

### Example 11

### Synthesis of ATAC32

This example shows the synthesis of 1-{6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexyl}-1H-pyrrole-2,5-dione (ATAC32).

[chemical structure]

### Step A: Preparation of ATAC32

[chemical structure]

To an ice-cold solution of 6-(2,5-dioxo-2,5-dihydro-1H-pyrrol-1-yl)hexanoic acid (0.034 g, 0.16 mmol) in DCM (0.800 ml) was added 1-chloro-N,N,2-trimethylprop-1-en-1-amine (0.021 mL, 0.160 mmol) dropwise. This was stirred at 0° C. for 1 h then added to an ice-cold mixture of compound 1 (50 mg, 0.160 mmol) and triethylamine (66.7 μL, 0.479 mmol) in DCM (800 μL). Overall molarity 0.1 M. The mixture was stirred to room temperature overnight and then chromatographed (DCM to 20% MeOH/DCM) without work-up. Fractions containing product were pooled and evaporated then dissolved in 1 mL of acetonitrile and treated with 0.1 mL of trifluoroacetic acid. The resulting material was evaporated to an oil then redissolved in CH₃CN and lyophilized the sample to give ATAC32 (65 mg) as a white solid. ¹H NMR (400 MHz, (DMSO-d⁶) δ 13.3 (s, 1H), 8.54-8.50 (m, 3H), 7.81 (d, J=8.5 Hz, 1H), 7.76 (d, J=7.5 Hz, 1H), 7.51 (d, J=7.5 Hz, 1H), 6.99 (s, 1H), 6.95 (s, 1H), 3.51 (q, J=7.0 Hz, 2H), 3.43-3.31 (m, 3H), 2.36-2.30 (m, 2H), 1.54-1.41 (m, 4H), 1.25-1.00 (m, 10H). ¹⁹F NMR (DMSO-d⁶) δ −74.0. LCMS [M+H]⁺=507.1.

### Example 12

### Synthesis of ATAC33

This example shows the synthesis of 1-{[4-({6-[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-6-oxohexylamino}carbonyl)cyclohexyl]methyl}-1H-pyrrole-2,5-dione (ATAC33).

[chemical structure]

### Step A: Preparation of ATAC33

[chemical structure]

To a stirred solution of 1-(4-amino-2-((ethylamino)methyl)-1H-imidazo[4,5-c]quinolin-1-yl)-2-methylpropan-2-ol (100 mg, 0.319 mmol) in DCM (10 mL) under nitrogen was added via a syringe pump a solution of 2,5-dioxopyrrolidin-1-yl 6-(4-((2,5-dioxo-2,5-dihydro-1H-pyrrol-1-yl)methyl)cyclohexane-1-carboxamido)hexanoate (143 mg, 0.319 mmol) in DCM (5 mL) over a period of 3.5 h. The reaction mixture was stirred at room temperature overnight. The reaction mixture was concentrated and the residue was purified by reverse phase column chromatography. Pure fractions identified by HPLC analysis were pooled and concentrated. The residue was lyophilized from CH₃CN to provide a white solid (52.8, mg) as the TFA salt of ATAC33 as a mixture of cis and trans isomers. ¹H NMR (400 MHz, (CD₃OD) δ 8.54 and 8.48 (d, J=8.3 Hz, 1H), 7.81-7.71 (m, 2H), 7.62-7.55 (m, 1H), 6.80 (s, 2H), 3.66 (q, J=7.0 Hz, 2H), 3.13 and 3.08 (t, J=7.0 Hz, 2H), 2.45 and 2.38 (t, J=7.5 Hz, 2H), 2.1-2.0 (m, 1H), 1.8-1.47 (m, 10H), 1.46-1.15 (m, 16H), 1.54-1.41 (m, 4H), 1.25-1.00 (m, 10H). LCMS [M+H]⁺=646.3.

### Example 13

### Synthesis of ATAC34

This example shows the synthesis of 1-[(4-{[({7-Amino-3-(2-hydroxy-2-methylpropyl)-3.5.8-triazatricyclo[7.4.0.02,6]trideca-1(9),2(6),4,7,10,12-hexaen-4-yl}methyl)-N-ethylamino]-carbonyl}cyclohexyl)methyl]-1H-pyrrole-2,5-dione (ATAC34).

[chemical structure]

### Step A: Preparation of ATAC34

[chemical structure]

To an ice-cold solution of (1r,4r)-4-((2,5-dioxo-2,5-dihydro-1H-pyrrol-1-yl)methyl)cyclohexane-1-carboxylic acid (82 mg, 0.346 mmol) in DCM (1728 μl) was added 1-chloro-N,N,2-trimethylprop-1-en-1-amine (50.3 μL, 0.380 mmol) dropwise. This was stirred at 0° C. for 1 h then added to an ice-cold mixture of 1-(4-amino-2-((ethylamino)methyl)-1H-imidazo[4,5-c]quinolin-1-yl)-2-methylpropan-2-ol (100 mg, 0.319 mmol) and triethylamine (133 μl, 0.957 mmol) in 1.6 mL of DCM. The mixture became a yellow solution as it stirred overnight to room temp. The reaction was concentrated to dryness, redissolved in MeOH/CH₂Cl₂, silica gel was added, then the solvents evaporated. Chromatography (12 g Gold silica, DCM to 20% MeOH/DCM, dry load) gave a solid which was dissolved in CH₃CN, frozen and lyophilized to afford 170 mg of N-((4-((1-(dimethylamino)-2-methylprop-1-en-1-yl)amino)-1-(2-((1-(dimethylamino)-2-methyl-prop-1-en-1-yl)oxy)-2-methylpropyl)-1H-imidazo[4,5-c]quinolin-2-yl)methyl)-4-((2,5-dioxo-2,5-dihydro-1H-pyrrol-1-yl)methyl)-N-ethylcyclohexane-1-carboxamide which was subsequently dissolved in 50% aqueous MeCN containing 0.1% TFA and heated in a microwave reactor at 150° C. for 60 min. The reaction mixture was cooled and the solvents were evaporated and chromatographed to give ATAC34 (72 mg) as a white solid. ¹H NMR (400 MHz, (DMSO-d⁶) δ 13.3 (s, 1H), 8.70-8.50 (m, 3H), 7.83-7.79 (m, 1H), 7.71-7.65 (m, 1H), 7.55-7.48 (m, 1H), 7.00 (s, 1H), 6.98 (s, 1H), 5.13 (bs, 1H), 4.83 (bs, 1H), 3.65 (q, J=7.0 Hz, 2H), 3.38 (m, 1H), 3.25 and 3.18 (d, J=6.5 Hz, 2H), 1.69-1.52 (m, 5H), 1.45-0.88 (m, 13H). ¹⁹F NMR (DMSO-d⁶) δ −73.7. LCMS [M+H]⁺=533.1.

### Example 14

### Fc Receptor Binding to Anti-CD40 Antibody Immune-Stimulatory Compound Conjugates

An anti-CD40 antibody is comprised of two SBT-040-G1WT heavy chains and two light chains from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised of two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody.

Each antibody is purified and then each is conjugated to ATAC1, ATAC2, ATAC3, ATAC4, ATAC5, ATAC6, ATAC7, ATAC8. ATAC1, ATAC2, ATAC3, ATAC4, ATAC5, ATAC6, ATAC7, ATAC8, ATAC9, ATAC10, ATAC11, ATAC12, ATAC13, ATAC14, ATAC15, ATAC16, ATAC17, ATAC18, ATAC19, ATAC20. ATAC21, ATAC22, ATAC23, ATAC24, ATAC25, ATAC26, ATAC27, ATAC28, ATAC29, ATAC30, ATAC31, ATAC32, ATAC33, ATAC34 or ATAC43 as described in EXAMPLE 2. Each of these conjugates is characterized for the ability of their Fc domains to bind to and for their affinity for soluble glycosylated FcγR ectodomains from human FcγRs. This is shown by performing surface plasmon resonance experiments. In these experiments, biotinylated soluble glycosylated FcγR ectodomains from all human FcγRs are immobilized on a streptavidin-coated surface. The ability of each conjugate to bind to soluble glycosylated FcγR ectodomains from all human FcγRs is then measured by surface plasmon resonance using a Biacore instrument. The data from these experiments shows that the Fc domain of any one of the SBT-040-WT-ATAC1-SBT-040-WT-ATAC34 or SBT-040-ATAC43 conjugates, the Fc domain of any one of the SBT-040-VLPLL-ATAC1-SBT-040-VLPLL-ATAC34 or SBT-040-VLPLL-ATAC43 conjugates, the Fc domain of any one of the SBT-040-DE-ATAC1-SBT-040-DE-ATAC34 or SBT-040-DE-ATAC 43 conjugates, the Fc domain of any one of the SBT-040-AAA-ATAC1—the SBT-040-AAA-ATAC34 or SBT-040-AAA-ATAC43 conjugates is bound to soluble glycosylated FcγR ectodomains from human FcγRs. Therefore, the surface plasmon resonance experiments show that the ability of the Fc domain of the antibody component of the conjugate to bind to human FcγRs is not interfered with by the conjugation of the components of the conjugate. The affinity of each conjugate for each human FcγRs is also shown by the surface plasmon resonance experiments. These affinity measurements are compared with the affinity measurements for each antibody alone (as can be shown by EXAMPLE 1). The similarity in affinity of each antibody alone for soluble glycosylated FcγR ectodomains from all human FcγRs with the affinity of each corresponding conjugate for soluble glycosylated FcγR ectodomains from all human FcγRs is shown by this comparison.

### Example 15

### Affinity of Anti-CD40 Antibodies to CD40

An anti-CD40 antibody is comprised of two SBT-040-G1WT heavy chains and two light chain from a SBT-040 antibody, which are referred to as a SBT-040-WT antibody. An anti-CD40 antibody is comprised of two SBT-040-G1VLPLL heavy chains and two light chains from a SBT-040 antibody, which are referred to as a SBT-040-VLPLL antibody. An anti-CD40 antibody is comprised of two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody.

SBT-040-WT antibody, SBT-040-VLPLL antibody, SBT-040-DE antibody, and SBT-040-AAA antibody are each produced by standard methods for producing antibodies. Each antibody is purified, and then is characterized for the ability to bind to CD40. This characterization is shown by experiments using flow cytometry. For these experiments, the human Burkitt's Lymphoma tumor cell lines Raji and Daudi, which are previously shown to be CD40-positive, and the human Chronic Myelogenous Leukemia tumor cell line K562, which is previously shown to be CD40-negative are first evaluated by flow cytometry to assess their relative expression levels of CD40. This is assessed by incubating each cell line with a commercially available CD40 antibody conjugated to a fluorochrome, and then running samples of the incubation on a flow cytometer. The relative fluorescent intensity profiles for each cell line is shown by this data, indicating the level of CD40 expression of each cell line. The relative fluorescent intensity profiles of human Burkitt's Lymphoma tumor cell lines Raji and Daudi show that CD40 is expressed in each of these cell lines, whereas the relative fluorescent intensity profile of the human Chronic Myelogenous Leukemia tumor cell line K562 show that CD40 is not expressed in the cell line. Then, each cell line is separately incubated with purified SBT-040-WT antibody, SBT-040-VLPLL antibody, SBT-040G1DE antibody, SBT-040-AAA antibody or no antibody as a control. Each incubation is further incubated with a secondary anti-human IgG1 antibody conjugated with FITC, which is then each assessed by flow cytometry for the FITC fluorescent intensity profile of each sample. The ability of each antibody to detect CD40 expression on the cell lines is indicated by their FITC fluorescent intensity profile. More specifically, the similarity between the SBT-040-WT antibody fluorescent intensity profile and each antibody with an Fc-enhanced IgG1 isotype after incubation with each of the cell lines is shown by this data. Each Fc-enhanced IgG1 isotype is not altered by the ability of the antibody to bind to CD40-positive cells is also shown by this data.

### Example 16

### Affinity of Anti-CD40 Antibodies to CD40

An anti-CD40 antibody is comprised of two SBT-040-G1WT heavy chains and two light chain from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised two SBT-040-G1DE heavy chains and two light chains from a SBT-040 antibody, which is referred to as a SBT-040-DE antibody. An anti-CD40 antibody is comprised two SBT-040-G1AAA heavy chains and two light chains from a SBT-040 antibody, which is referred to as a SBT-040-AAA antibody.

SBT-040-WT antibody, SBT-040-VLPLL antibody, SBT-040-DE antibody, and SBT-040-AAA antibody are each produced by standard methods for producing antibodies. Each antibody is purified, and each antibody's affinity for CD40 is measured. These affinities are measured by experiments using surface plasmon resonance. In these experiments, biotinylated recombinant CD40 is immobilized on a streptavidin-coated surface. The ability of each antibody to bind to recombinant CD40 is then measured by surface plasmon resonance using a Biacore instrument. The data from these experiments shows that SBT-040-WT antibody, SBT-040-VLPLL antibody, SBT-040-DE antibody, and SBT-040-AAA antibody are each bound to recombinant CD40. Therefore, each antibody's ability to bind to CD40 is not interfered with by the enhanced Fc-enhanced IgG1 isotypes is shown by the surface plasmon resonance data.

Furthermore, surface plasmon resonance is used to show that CD40L binding to CD40 is not blocked by these antibodies. In these experiments, biotinylated recombinant CD40 is immobilized on a streptavidin-coated surface. Surface plasmon resonance using a Biacore instrument is then used to measure the binding affinity of CD40L in the presence of each antibody or without any antibody as a control. The binding affinity of CD40L with recombinant CD40 in presence of each antibody is shown to be the same as the binding affinity of the CD40L with recombinant CD40 in the absence of any antibody. Therefore, CD40 and CD40L binding is unaffected by the presence of SBT-040-WT antibody, SBT-040-G1VLPLL antibody, SBT-040-DE antibody, or SBT-040-AAA antibody.

### Example 17

### Fc Receptor Binding to Anti-CD40 Antibody Immune-Stimulatory Compound Conjugates

An anti-CD40 antibody is comprised of two SBT-040-G1WT heavy chains and two light chain from a SBT-040 antibody, which is referred to as a SBT-040-WT antibody. An anti-CD40 antibody is comprised of two SBT-040-G1VLPLL heavy chains and two light chains from a SBT-040 antibody, which is referred to as a SBT-040-VLPLL antibody. An anti-CD40 antibody is comprised of two SBT-040-G1DE heavy chains and two light chains from a SBT-040 antibody, which is referred to as a SBT-040-DE antibody. An anti-CD40 antibody is comprised of two SBT-040-G1AAA heavy chains and two light chains from a SBT-040 antibody, which is referred to as a SBT-040-AAA antibody.

SBT-040-WT antibody, SBT-040-VLPLL antibody, SBT-040-DE antibody, and SBT-040-AAA antibody are each made following standard methods for antibody production. Each antibody is purified and then each is conjugated to ATAC1, ATAC2, ATAC3, ATAC4, ATAC5, ATAC6, ATAC7, or ATAC8. ATAC1, ATAC2, ATAC3, ATAC4, ATAC5, ATAC6, ATAC7, ATAC8, ATAC9, ATAC10, ATAC11, ATAC12, ATAC13, ATAC14, ATAC15, ATAC16, ATAC17, ATAC18, ATAC19, ATAC20. ATAC21, ATAC22, ATAC23, ATAC24, ATAC25, ATAC26, ATAC27, ATAC28, ATAC29, ATAC30, ATAC31, ATAC32, ATAC33, ATAC34 or ATAC 43 are as described in EXAMPLE 2. The affinity of each conjugate for CD40 is then measured by experiments using surface plasmon resonance. In these experiments, biotinylated recombinant CD40 is immobilized on a streptavidin-coated surface. The ability of each conjugate to bind to recombinant CD40 is then measured by surface plasmon resonance using a Biacore instrument. The data from these experiments shows that any one of the SBT-040-WT-ATAC1-SBT-040-WT-ATAC34 or SBT-040-WT-ATAC43 conjugates, any one of the SBT-040-VLPLL-ATAC1-SBT-040-VLPLL-ATAC34 or SBT-040-VLPLL-ATAC43 conjugates, any one of the SBT-040-DE-ATAC1-SBT-040-DE-ATAC34 or SBT-040-DE-ATAC43 conjugates, or any one of the SBT-040-AAA-ATAC1—the SBT-040-AAA-ATAC34 or SBT-040-AAA-ATAC43 conjugates is bound to recombinant CD40. Therefore, the surface plasmon resonance experiments show that each component antibody's ability to bind to CD40 is not interfered with by the enhanced Fc-enhanced IgG1 isotypes nor the antibody conjugation to ATAC1, ATAC2, ATAC3, ATAC4, ATAC5, ATAC6, ATAC7, or ATAC8. ATAC1, ATAC2, ATAC3, ATAC4, ATAC5, ATAC6, ATAC7, ATAC8, ATAC9, ATAC10, ATAC11, ATAC12, ATAC13, ATAC14, ATAC15, ATAC16, ATAC17, ATAC18, ATAC19, ATAC20. ATAC21, ATAC22, ATAC23, ATAC24, ATAC25, ATAC26, ATAC27, ATAC28, ATAC29, ATAC30, ATAC31, ATAC32, ATAC33, ATAC34 or ATAC43.

Furthermore, surface plasmon resonance is used to show that CD40L binding to CD40 is not blocked in the presence of each conjugate. In these experiments, biotinylated recombinant CD40 is immobilized on a streptavidin-coated surface. Surface plasmon resonance using a Biacore instrument is then used to measure the binding affinity of CD40L in the presence of each conjugate or without any conjugate as a control. The binding affinity of CD40L with recombinant CD40 in presence of each conjugate is shown to be the same as the binding affinity of the CD40L with recombinant CD40 in the absence of any conjugate by these experiments. Therefore, CD40 and CD40L binding is unaffected by the presence of any one of the SBT-040-WT-ATAC1-SBT-040-WT-ATAC34 or SBT-040-WT-ATAC43 conjugates, any one of the SBT-040-VLPLL-ATAC1-SBT-040-VLPLL-ATAC34 or SBT-040-VLPLL-ATAC43 conjugates, any one of the SBT-040-DE-ATAC1-SBT-040-DE-ATAC34 or SBT-040-DE-ATAC43 conjugates, or any one of the SBT-040-AAA-ATAC1—the SBT-040-AAA-ATAC34 or SBT-040-AAA-ATAC43 conjugates.

### Example 18

### Cytokine Production is Enhanced by Anti-CD40 Antibody Immune-Stimulatory Compound Conjugates

This example shows that cytokine production by dendritic cells is enhanced after administration of antibody-immune stimulatory compound conjugates in culture. In this experiment, dendritic cells (DCs) are derived from peripheral blood mononuclear cells (PBMCs). DCs are obtained by putting human PBMCs into a culture dish. The resulting adherent cells are washed with RPMI containing 10% fetal calf serum, and then are incubated for 7 days in complete medium containing 10 ng/mL IL-4 and 100 ng/mL GM-CSF. The non-adherent cells are isolated and are washed. These isolated cells are run by a flow cytometer to ensure CD1c expression, in which the DCs identity as DCs is confirmed by CD1c expression. The DCs are then incubated with either the antibodies as described in Example 1 or the conjugates as described in Example 3. More specifically, the DCs are incubated with any one of SBT-040-WT antibody, SBT-040-VLPLL antibody, SBT-040-DE antibody, SBT-040-AAA antibody, the SBT-040-WT-ATAC1 conjugate—SBT-040-WT-ATAC34 or SBT-040-WT-ATAC43 conjugate, the SBT-040-VLPLL-ATAC1 conjugate—SBT-040-VLPLL-ATAC34 or SBT-040-VLPLL-ATAC43 conjugate, the SBT-040-DE-ATAC1 conjugate—SBT-040-DE-ATAC34 or SBT-040-DE-ATAC43 conjugate, the SBT-040-AAA-ATAC1 conjugate—the SBT-040-AAA-ATAC34 or SBT-040-AAA-ATAC43 conjugate, or a non-binding isotype control antibody. Each culture is then incubated for 24 hours and the supernatant of each culture is analyzed using a cytokine bead array assay. Cytokine expression levels of IFNγ, IL-8, IL-12 and IL-2 are measured by the cytokine bead array assay. The supernatant from the culture containing the non-binding isotype control shows the level of cytokine expression is decreased as compared to the supernatant from cultures containing SBT-040-WT, SBT-040-VLPLL, SBT-040-DE, or SBT-040-AAA. Additionally, the level of cytokine expression in the supernatant from cultures containing SBT-040-WT, SBT-040-VLPLL, SBT-040-DE, or SBT-040-AAA is decreased as compared to the supernatant from cultures containing any one of the SBT-040-WT-ATAC1 conjugate—SBT-040-WT-ATAC34 or SBT-040-WT-ATAC43 conjugate, any one of the SBT-040-VLPLL-ATAC1 conjugate—SBT-040-VLPLL-ATAC34 or SBT-040-VLPLL ATAC43 conjugate, any one of the SBT-040-DE-ATAC1 conjugate-SBT-040-DE-ATAC34 or SBT-040-DE-ATAC43 conjugate, or any one of the SBT-040-AAA-ATAC1 conjugate-SBT-040-AAA-ATAC34 or SBT-040-AAA-ATAC43 conjugate.

### Example 19

### Cytokine Production by Dendritic Cells from Multiple Donors was Enhanced by Anti-CD40 Antibody Immune-Stimulatory Compound Conjugates

Antibody-immune stimulatory compound conjugates enhanced immunostimulatory cytokines produced by human dendritic cells in a concentration dependent manner when added to and incubated with the cells. The human dendritic cells (DCs) from two donors were derived from CD14⁺ monocytes isolated from peripheral blood mononuclear cells (PBMCs) by negative selection using a commercially available kit. The monocytes were cultured in RPMI containing 10% fetal calf serum for seven days in complete medium supplemented with 25 ng/mL IL-4 and 10 ng/mL GM-CSF. The media was replaced with fresh media plus cytokines on day three. On day six anti-CD40 antibody immune-stimulatory compound conjugates SBT-040-G1-ATAC23 and SBT-040-G1-ATAC17 and control antibody were added to individual wells containing the dendritic cells. After 24 hours of further incubation, the supernatants were collected and the cytokines IL-6, TNFα, IL-12p70 and IL-10 produced by the dendritic cells were quantitated by electrochemiluminescence signal by multiplex ELISA using commercially available reagents and plate reader from Meso Scale Discovery. Results are shown for the immune stimulatory cytokines IL-12p70 and TNFα for dendritic cells derived from two donors. FIG. 31A shows the concentration of IL-12p70 produced by DCs from donor 358 after incubation with SBT-040-G1-ATAC23 or SBT-040-G1-ATAC17 as compared with SBT-050-WT. FIG. 31B shows the concentration of IL-12p70 produced by DCs from donor 363 after incubation with SBT-040-G1-ATAC23 or SBT-040-G1-ATAC17 as compared with SBT-050-WT. FIG. 31C shows the concentration of TNFα produced by DCs from donor 358 after incubation with SBT-040-G1-ATAC23 or SBT-040-G1-ATAC17 as compared with an anti-HER2 antibody. FIG. 31D shows the concentration of TNFα produced by DCs from donor 363 after incubation with SBT-040-G1-ATAC23 or SBT-040-G1-ATAC17 as compared with an anti-HER2 antibody.

### Example 20

### Immunostimulatory Cytokine Secretion is Enhanced by Anti-CD40 Antibody Immune-Stimulatory Compound Conjugates with Different Linkers and FcγR Binding

Antibody-immune stimulatory compound conjugates enhanced human dendritic cells cytokine production in a concentration dependent manner when added to and incubated with the cells. The human dendritic cells (DCs) were derived from CD14+ monocytes isolated from peripheral blood mononuclear cells (PBMCs) by negative selection using a commercially available kit. The monocytes were cultured in RPMI containing 10% fetal calf serum for seven days in complete medium supplemented with 25 ng/mL IL-4 and 10 ng/mL GM-CSF. On day six anti-CD40 antibody immune-stimulatory compound conjugates and non-DC binding control antibody SBT-50 G1 and commercially available soluble CD40L were added to individual wells containing the dendritic cells. More specifically, the DCs are incubated with any one of SBT-040-WT-ATAC4, SBT-040-WT-ATAC3, SBT-040-G2-ATAC4, SBT-040-G2-ATAC3, SBT-040-AAA-ATAC29, SBT-040-VLPLL-ATAC29, SBT-040-WT-ATAC1, SBT-040-G2-ATAC1, SBT-040-WT-ATAC12, SBT-040-G2-ATAC12, SBT-040-WT-ATAC30, SBT-040-AAA-ATAC11, SBT-040-VLPLL-ATAC11, SBT-040-VLPLL-ATAC12, SBT-040-AAA-ATAC12, SBT-040-VLPLL-ATAC30, and SBT-040-AAA-ATAC30. After 24 hours further incubation supernatants were collected and the cytokines IL-6, TNFα, IL-12p70 and IL-10 produced by the dendritic cells were quantitated by electrochemiluminescence signal by multiplex ELISA using commercially available reagents and plate reader from Meso Scale Discovery. FIG. 32A shows the concentration of IL-12p70 produced by DCs after incubation with SBT-040-WT-ATAC4, SBT-040-WT-ATAC3, SBT-040-G2-ATAC4, SBT-040-G2-ATAC3, SBT-040-AAA-ATAC29, SBT-040-VLPLL-ATAC29, SBT-040-WT-ATAC1, SBT-040-G2-ATAC1, SBT-040-WT-ATAC12, SBT-040-G2-ATAC12, SBT-040-WT-ATAC30, SBT-040-AAA-ATAC11, SBT-040-VLPLL-ATAC11, SBT-040-VLPLL-ATAC12, SBT-040-AAA-ATAC12, SBT-040-VLPLL-ATAC30, and SBT-040-AAA-ATAC30 as compared with SBT-050-G2 antibody or CD40 ligand. FIG. 32B shows the concentration of IL-6 produced by DCs from donor 2 after incubation with SBT-040-WT-ATAC4, SBT-040-WT-ATAC3, SBT-040-G2-ATAC4, SBT-040-G2-ATAC3, SBT-040-AAA-ATAC29, SBT-040-VLPLL-ATAC29, SBT-040-WT-ATAC1, SBT-040-G2-ATAC1, SBT-040-WT-ATAC12, SBT-040-G2-ATAC12, SBT-040-WT-ATAC30, SBT-040-AAA-ATAC11, SBT-040-VLPLL-ATAC11, SBT-040-VLPLL-ATAC12, SBT-040-AAA-ATAC12, SBT-040-VLPLL-ATAC30, and SBT-040-AAA-ATAC30 as compared with SBT-050-G2 or CD40 ligand. Results are shown for the immune stimulatory cytokines IL-12p70 and IL-6. The treatment concentrations for each molecule, depicted on the x-axis from right to left, were 0.08 ug/mL, 0.310 ug/mL, 1.25 ug/mL and 5.00 ug/mL.

### Example 21

### Anti-CD40 Antibody Immune-Stimulatory Compound Conjugates Increased Cell Surface Expression of Immune Activating Proteins

Human dendritic cells showed increased expression of CD83, CD86, and MHC class II cell surface proteins in after treatment with anti-CD40 antibody immune-stimulatory compound conjugates. The increased expression of these surface proteins was dose dependent.

Human dendritic cells were derived from human PBMCs by isolation of CD14 monocytes followed by culture in RPMI containing 10% fetal calf serum for seven days in complete medium supplemented with 10 ng/mL IL-4 and 100 ng/mL GM-CSF. After three days of culture the media was removed and replaced with fresh media including cytokine supplement. On day six SBT-040-WT-ATAC30, SBT-040-WT-ATAC24, SBT-040-VLPLL-ATAC30, SBT-040-AAA-ATAC30 or a control SBT-050-WT were added to separate aliquots of dendritic cells. After an additional 24 hour incubation the cells were collected and washed by centrifugations then stained for 30 minutes on ice using manufacturer's recommended concentrations of commercially available anti-CD83, anti-CD86 and anti-MHC class II monoclonal antibodies conjugated to laser sensitive fluors. A separate aliquot for each treatment was stained with IgG matched isotype control antibody conjugate for the anti-CD86 antibody, anti-CD83 antibody, and anti-MHC Class II antibody. After washing to remove unbound antibody-fluor molecules, the stained cells were subjected to FACS analysis using a Celesta flow cytometer (BD Biosciences) with gating on live cells. The output was analyzed by FlowJo v10.2 software (FlowJo LLC) and curve fit with Prism 7.01 software (GraphPad Software, Inc.). FIG. 33A shows a dose dependent increase in CD86 expression on dendritic cells after treatment with SBT-040-WT-ATAC30, SBT-040-WT-ATAC24, SBT-040-VLPLL-ATAC30, SBT-040-AAA-ATAC30 as compared to treatment a control SBT-050-WT or staining with an isotype control. FIG. 33B shows a dose dependent increase in CD83 expression on dendritic cells after treatment SBT-040-WT-ATAC30, SBT-040-WT-ATAC24, SBT-040-VLPLL-ATAC30, SBT-040-AAA-ATAC30 as compared to treatment a control SBT-050-WT or staining with an isotype control. FIG. 33C shows a dose dependent increase in MHC class II expression on dendritic cells after treatment with SBT-040-WT-ATAC30, SBT-040-WT-ATAC24, SBT-040-VLPLL-ATAC30, SBT-040-AAA-ATAC30 as compared to treatment a control SBT-050-WT or staining with an isotype control. The graph shows plots of treatment protein concentration on the x axis versus mean fluorescence intensity for the cell surface protein on the y axis.

### Example 22

### Treatment of Cancer by Administering a Conjugate

This example describes treatment of cancer with a conjugate. A human patient is diagnosed with a cancer. A conjugate as shown in the schematic of FIG. 8 is administered to the patient with a pharmaceutically acceptable carrier. FIG. 8 is a conjugate comprising an antibody construct and an immune stimulatory compound. The antibody construct is an antibody, which contains two heavy chains as shown in gray and two light chains as shown in light gray. The antibody comprises two antigen binding sites (810 and 815), and a portion of the heavy chains contain Fc domains (805 and 820). The immune-stimulatory compounds (830 and 840) are conjugated to the antibody by linkers (860 and 870).

As another example, a human patient is diagnosed with a cancer. A conjugate as shown in the schematic of FIG. 9 is administered to the patient with a pharmaceutically acceptable carrier. FIG. 9 is a conjugate comprising an antibody construct, two targeting binding domains, and two immune stimulatory compounds. The antibody construct is an antibody, which contains two heavy chains as shown in gray and two light chains as shown in light gray. The antibody comprises two antigen binding sites (910 and 915), and a portion of the heavy chains contain Fc domains (905 and 920). The immune-stimulatory compounds (930 and 940) are conjugated to the antibody by linkers (960 and 970). The targeting binding domains are conjugated to the antibody (980 and 985).

As an additional example, a human patient is diagnosed with a cancer. A conjugate as shown in the schematic of FIG. 10 is administered to the patient with a pharmaceutically acceptable carrier. FIG. 10 is a conjugate comprising an antibody construct and two immune stimulatory compounds. The antibody construct contains the Fc region of an antibody with the heavy chains shown in gray, and two scaffolds as shown in light gray. The antibody construct comprises two antigen binding sites (1010 and 1015) in the scaffolds, and a portion of the heavy chains contain Fc domains (1005 and 1020). The immune-stimulatory compounds (1030 and 1040) are conjugated to the antibody construct by linkers (1060 and 1070).

As another example, a human patient is diagnosed with a cancer. A conjugate as shown in the schematic of FIG. 11 is administered to the patient with a pharmaceutically acceptable carrier. FIG. 11 is a conjugate comprising an antibody construct, two targeting domains, and two immune stimulatory compounds. The antibody construct contains the Fc region of an antibody with the heavy chains shown in gray, and two scaffolds as shown in light gray. The antibody construct comprises two antigen binding sites (1110 and 1115) in the scaffolds, and a portion of the heavy chains contain Fc domains (1105 and 1120). The immune-stimulatory compounds (1130 and 1140) are conjugated to the antibody construct by linkers (1160 and 1170). The targeting binding domains are conjugated to the antibody construct (1180 and 1185).

As another example, a human patient is diagnosed with a cancer. A conjugate as shown in the schematic of FIG. 12 is administered to the patient with a pharmaceutically acceptable carrier. FIG. 12 is a conjugate comprising an antibody construct and two immune stimulatory compounds. The antibody construct contains the F(ab′)₂ region of an antibody with heavy chains shown in gray and light chains shown in light gray, and two scaffolds as shown in dark gray. The antibody construct comprises two antigen binding sites (1210 and 1215), and a portion of two scaffolds contain Fc domains (1220 and 1245). The immune-stimulatory compounds (1230 and 1240) are conjugated to the antibody construct by linkers (1260 and 1270).

As another example, a human patient is diagnosed with a cancer. A conjugate as shown in the schematic of FIG. 13 is administered to the patient with a pharmaceutically acceptable carrier. FIG. 13 is a conjugate comprising an antibody construct, two targeting binding domains, and two immune stimulatory compounds. The antibody construct contains the F(ab′)₂ region of an antibody with heavy chains shown in gray and light chains shown in light gray, and two scaffolds as shown in dark gray. The antibody construct comprises two antigen binding sites (1310 and 1315), and a portion of two scaffolds contain Fc domains (1320 and 1345). The immune-stimulatory compounds (1330 and 1340) are conjugated to the antibody construct by linkers (1360 and 1370). The targeting binding domains are conjugated to the antibody construct (1380 and 1385).

As another example, a human patient is diagnosed with a cancer. A conjugate as shown in the schematic of FIG. 14 is administered to the patient with a pharmaceutically acceptable carrier. FIG. 14 is a conjugate comprising an antibody construct, and two immune stimulatory compounds. The antibody construct contains two scaffolds as shown in light gray and two scaffolds as shown in dark gray. The antibody construct comprises two antigen binding sites (1410 and 1415), and a portion of the two dark gray scaffolds contain Fc domains (1420 and 1445). The immune-stimulatory compounds (1430 and 1440) are conjugated to the antibody construct by linkers (1460 and 1470).

As another example, a human patient is diagnosed with a cancer. A conjugate as shown in the schematic of FIG. 15 is administered to the patient with a pharmaceutically acceptable carrier. FIG. 15 is a conjugate comprising an antibody construct, two targeting binding domains, and two immune stimulatory compounds. The antibody construct contains two scaffolds as shown in light gray and two scaffolds as shown in dark gray. The antibody construct comprises two antigen binding sites (1510 and 1515), and a portion of the two dark gray scaffolds contain Fc domains (1520 and 1545). The immune-stimulatory compounds (1530 and 1540) are conjugated to the antibody construct by linkers (1560 and 1570). The targeting binding domains are conjugated to the antibody construct (1580 and 1585).

### Example 23

### Determination of K_{d} Values

K_{d} is measured by a radiolabeled antigen binding assay (RIA) performed with the Fab version of an antibody of interest and its antigen as described by the following assay.

Solution binding affinity of Fabs for antigen is measured by equilibrating the Fab with a minimal concentration of (¹²⁵I)-labeled antigen in the presence of a titration series of unlabeled antigen, then capturing bound antigen with an anti-Fab antibody-coated plate (See, e.g., Chen et al., J. Mol. Biol. 293:865-881 (1999)). To establish conditions for the assay, multi-well plates are coated overnight with 5 μg/mL of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant is added, and the plates are counted on a TOPCOUNT™ gamma counter (Packard) for ten minutes. Concentrations of each Fab that give less than or equal to 20% of maximal binding are chosen for use in competitive binding assays.

### Example 24

### Determination of K_{d} Values

K_{d} is measured using surface plasmon resonance assays using a BIACORE®-2000 or a BIACORE®-3000 (BIAcore, Inc., Piscataway, N.J.) at 25° C. with immobilized antigen CM5 chips at ^{˜}10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (^{˜}0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 L/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Example 25

### Lysine-based Bioconjugation

The antibody construct is exchanged into an appropriate buffer, for example, phosphate, borate, PBS, Tris-Acetate at a concentration of about 2 mg/mL to about 10 mg/mL. An appropriate number of equivalents of the immune stimulatory compound-linker construct (ATAC) were added as a solution with stirring. Dependent on the physical properties of the immune stimulatory compound-linker construct, a co-solvent was introduced prior to the addition of the immune stimulatory compound-linker construct to facilitate solubility. The reaction was stirred at room temperature for 2 hours to about 12 hours depending on the observed reactivity. The progression of the reaction was monitored by LC-MS. Once the reaction was deemed complete, the remaining immune stimulatory compound-linker constructs were removed by applicable methods and the antibody construct-immune stimulatory compound conjugate was exchanged into the desired formulation buffer. Lysine-linked conjugates were synthesized starting with 10 mg of antibody (mAb) and 10 equivalents of ATAC1, ATAC2, ATAC3, ATAC4, ATAC5, ATAC6, ATAC7, ATAC8, ATAC9, or ATAC10 using the conditions described in Scheme 34 below (ADC=antibody immune-stimulatory compound conjugate). Monomer content and immune-stimulatory compound-antibody construct ratios (molar ratios) were determined by methods described in EXAMPLES 27-31.

[chemical structure]

TABLE 7

| | | Isolated | % | |
|---|---|---|---|---|
| Scaffold | Name | ADC | Monomer | DAR |
| i | SBT-040-WT-ATAC4 | 9.7 mg | >95% | 4.6 |
| i | SBT-040-G2-ATAC4 | 12.3 mg | >95% | 2.9 |
| ii | SBT-040-WT-ATAC3 | 7.8 mg | >95% | 5.4 |
| ii | SBT-040-G2-ATAC3 | 8.9 mg | >95% | 3.3 |
| ii | SBT-040-WT-ATAC1 | 7.2 mg | >99% | 1.9 |
| ii | SBT-040-G2-ATAC1 | 7.1 mg | 99% | 2.1 |

### Example 26

### Cysteine-Based Bioconjugation

The antibody construct was exchanged into an appropriate buffer, for example, phosphate, borate, PBS, Tris-Acetate at a concentration of about 2 mg/mL to about 10 mg/mL with an appropriate number of equivalents of a reducing agent, for example, dithiothreitol or tris(2-carboxyethyl)phosphine. The resultant solution was stirred for an appropriate amount of time and temperature to effect the desired reduction. The immune stimulatory compound-linker construct was added as a solution with stirring. Dependent on the physical properties of the immune stimulatory compound-linker construct, a co-solvent was introduced prior to the addition of the immune stimulatory compound-linker construct to facilitate solubility. The reaction was stirred at room temperature for about 1 hour to about 12 hours depending on the observed reactivity. The progression of the reaction was monitored by liquid chromatography-mass spectrometry (LC-MS). Once the reaction was deemed complete, the remaining free immune stimulatory compound-linker construct was removed by applicable methods and the antibody construct-immune stimulatory compound conjugate was exchanged into the desired formulation buffer. Such cysteine-based conjugates were synthesized starting with 10 mg of antibody (mAb) and 7 equivalents of ATAC11-ATAC45 using the conditions described in Scheme 35 below (ADC=antibody immune-stimulatory compound conjugate). Monomer content and drug-antibody ratios can be determined by methods described in EXAMPLES 27-31.

[chemical structure]

TABLE 8

| Scaffold | Name | Isolated ADC | % Monomer | DAR |
|---|---|---|---|---|
| i | SBT-040-WT-ATAC11 | 9.0 mg | >95% | 4.5 |
| i | SBT-040-G2-ATAC11 | 8.9 mg | 94% | 4.0 |
| ii | SBT-040-WT-ATAC22 | 9.0 mg | 93% | 4.6 |
| ii | SBT-040-G2-ATAC22 | 9.0 mg | >95% | 4.1 |
| ii | SBT-040-AAA-ATAC22 | 8.5 mg | 98% | 4.1 |
| ii | SBT-040-VLPLL-ATAC22 | 8.5 mg | 94% | 3.9 |
| iii | SBT-040-WT-ATAC12 | 11.7 mg | >95% | 4.0 |
| iii | SBT-040-G2-ATAC12 | 11.4 mg | 96% | 4.1 |
| iv | SBT-040-WT-ATAC23 | 11.5 mg | 93% | 4.7 |
| i | SBT-040-AAA-ATAC11 | 10.2 mg | >95% | 2.9 |
| i | SBT-040-VLPLL-ATAC11 | 9.4 mg | 96% | 2.9 |
| iv | SBT-040-AAA-ATAC23 | 7.1 mg | >95% | 1.9 |
| iv | SBT-040-G1(VLPLL)- | 7.7 mg | >95% | 2.6 |
| | ATAC23 | | | |

### Example 27

### Determination of Molar Ratio

This example illustrates one method by which the molar ratio is determined. One microgram of antibody construct immune-stimulatory compound conjugate is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact antibody construct immune-stimulatory compound conjugates is calculated by the software, which can use top peak height at 25% for the calculation. This data is then imported into another program to calculate the molar ratio of the antibody construct immune-stimulatory compound conjugate such as Agilent molar ratio calculator.

### Example 28

### Determination of Molar Ratio for SBT-040-G1WT Conjugated to a Cys-Targeted Compound

FIG. 28 shows HPLC analysis of SBT-040-G1WT conjugated to a Cys-targeted drug linker tool compound. First, 10 μL of a 5 mg/mL solution of the antibody-drug conjugate was injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method was run in which the mobile phase gradient ran from 100% mobile phase A to 100% mobile phase B over the course of 12 minutes, followed by a six minute re-equilibration at 100% mobile phase A. The flow rate was 0.8 mL/min and the detector was set at 280 nM. Mobile phase A was 1.5 M ammonium sulfate, 25 mM sodium phosphate (pH 7). Mobile phase B was 25% isopropanol in 25 mM sodium phosphate (pH 7). Post-run, the chromatogram was integrated and the molar ratio was determined by summing the weighted peak area. The molar ratio was calculated to be about 4.56 with 7% unconjugated antibody.

### Example 29

### Determination of Molar Ratio for SBT-040-G1WT Conjugated to ATAC11

FIG. 29 shows HPLC analysis of SBT-040-G1WT conjugated to ATAC11, which is a cleavable Maleimide-Val-Ala-PABA-Gardiquimod linker. First, 10 μL of a 5 mg/mL solution of the antibody immune-stimulatory compound conjugate was injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method was run in which the mobile phase gradient ran from 100% mobile phase A to 100% mobile phase B over the course of 12 minutes, followed by a six minute re-equilibration at 100% mobile phase A. The flow rate was 0.8 mL/min and the detector was set at 280 nM. Mobile phase A was 1.5 M ammonium sulfate, 25 mM sodium phosphate (pH 7). Mobile phase B was 25% isopropanol in 25 mM sodium phosphate (pH 7). Post-run, the chromatogram was integrated and the molar ratio was determined by summing the weighted peak area. The molar ratio was calculated to be about 4.5.

### Example 30

### Determination of Molar Ratio for SBT-040-G2WT Conjugated to ATAC11

FIG. 30 shows HPLC analysis of SBT-040-G2WT conjugated to ATAC11, which is a cleavable Maleimide-Val-Ala-PABA-Gardiquimod linker. First, 10 μL of a 5 mg/mL solution of the antibody-immune stimulatory compound conjugate was injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method was run in which the mobile phase gradient ran from 100% mobile phase A to 100% mobile phase B over the course of 12 minutes, followed by a six minute re-equilibration at 100% mobile phase A. The flow rate was 0.8 mL/min and the detector was set at 280 nM. Mobile phase A was 1.5 M ammonium sulfate, 25 mM sodium phosphate (pH 7). Mobile phase B was 25% isopropanol in 25 mM sodium phosphate (pH 7). Post-run, the chromatogram was integrated and the molar ratio was determined by summing the weighted peak area. The molar ratio was calculated to be about 4.0.

### Example 31

### Additional Method for Determination of Molar Ratio

Another method for determination of molar ratio is as follows. First, 10 μL of a 5 mg/mL solution of an antibody construct immune-stimulatory compound conjugate is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Example 32

### Affinity Measurement of Unconjugated Anti-CD40 Antibody or Anti-C40 Antibody Immune-Stimulatory Conjugate to Recombinant CD40 Ectodomain Fcγ Receptors Using BioLayer Interferometry

This examples shows affinity measurements of unconjugated anti-C40 antibody or anti-CD40 antibody immune-stimulatory conjugate to Fc receptors (FcRs). Antibody affinity to its antigen (such as CD40) and the Fcγ Receptors (such as FcγRI, FcγRII, FcγRIII) was quantitated using BioLayer Interferometry (BLI). The anti-CD40 antibodies with different Fc (SBT-040-WT, SBT-040-VLPLL, SBT-040-AAA and SBT-040-G2) were first produced as described in EXAMPLE 1 and then conjugated with an immune-stimulatory compound (any one of ATAC1-ATAC34 and ATAC 43) as described in EXAMPLE 2. Following successful conjugation, their molecular interactions with CD40 Extracellular Domain (ECD) and various human FcγRs were quantitated using BLI.

Analysis of CD40 ECD interaction was performed using Octet Red 96 instrument (ForteBio). The Octet systems use propriety BLI to analyze biomolecular interaction. Unconjugated anti-CD40 antibodies (SBT-040-WT, SBT-040-VLPLL, SBT-040-AAA, SBT-040-G2) and anti-CD40 antibody immune-stimulatory compound conjugates were immobilized on anti-human Fc biosensors and incubated with varying concentration of monomeric human or rhesus CD40 ranging from 1.2 nM to 300 nM in PBS. The experiments were comprised of 5 steps: (1) baseline acquisition (60 s); (2) antibodies and antibody immune-stimulatory compound conjugates loading onto anti-human Fc biosensor (120 s); (3) second baseline acquisition (60 s); (4) association of interacting monomeric CD40 ECD protein for kₒₙ measurement (120 s); (5) Dissociation of interacting monomeric CD40 ECD for k_{off} measurement (360 s). The interacting monomeric CD40 ECD were used at 5-6 concentrations of 3-fold concentration series. Data were analyzed using Octet Data Analysis Sofware 9.0 (ForteBio) and fitted to the 1:1 binding model. Equilibrium dissociation constants (K_{D}) were calculated by the ratio of kₒₙ to k_{off}. Selected data are shown in TABLE 9. All the anti-CD40 antibody immune-stimulatory compound conjugates had similar binding as unconjugated anti-CD40 antibody to monomeric human or rhesus CD40 ECD.

TABLE 9

| | Human CD40 ECD monomer | | | Rhesus CD40 ECD monomer | | |
|---|---|---|---|---|---|---|
| Antibody/Conjugate | KD (nM) | Ka (1/Ms) | Kd (1/s) | KD (nM) | Ka (1/Ms) | Kd (1/s) |
| SBT-040-WT | 8.4 | 1.35E+5 | 1.13E−3 | 14.0 | 7.03E+4 | 9.87E−4 |
| SBT-040-WT-ATAC11 | 8.3 | 1.24E+5 | 1.03E−3 | 13.5 | 7.56E+4 | 1.02E−3 |
| SBT-040-WT-ATAC22 | 8.0 | 1.37E+5 | 1.09E−3 | 13.6 | 7.61E+4 | 1.04E−3 |
| SBT-040-WT-ATAC4 | 9.3 | 1.04E+5 | 9.68E−4 | 12.9 | 6.55E+4 | 8.46E−4 |
| SBT-040-WT-ATAC3 | 9.0 | 9.97E+5 | 8.97E−4 | 13.1 | 6.31E+4 | 8.23E−4 |
| SBT-040-G2 | 7.7 | 1.34E+5 | 1.04E−3 | 12.4 | 7.61E+4 | 9.41E−4 |
| SBT-040-G2-ATAC11 | 7.4 | 1.47E+5 | 1.09E−3 | 14.0 | 8.16E+4 | 1.14E−3 |
| SBT-040-G2-ATAC22 | 7.2 | 1.42E+5 | 1.02E−3 | 15.2 | 7.20E+4 | 1.09E−3 |
| SBT-040-G2-ATAC4 | 8.7 | 9.97E+5 | 8.67E−4 | 13.6 | 5.80E+4 | 7.88E−4 |
| SBT-040-G2-ATAC3 | 7.3 | 1.15E+5 | 8.97E−4 | 11.5 | 7.21E+4 | 8.33E−4 |
| SBT040-VLPLL | 5.6 | 9.37E+4 | 5.26E−4 | Not done | | |
| SBT040-VLPLL ATAC11 | 7.6 | 9.75E+4 | 7.37E-4 | Not done | | |
| SBT040-VLPLL ATAC22 | 7.5 | 1.02E+5 | 7.71E-4 | Not done | | |
| SBT040-AAA | 6.0 | 9.89E+4 | 5.97E−4 | Not done | | |
| SBT040-AAA ATAC11 | 4.7 | 1.61E+5 | 7.53E−4 | Not done | | |
| SBT040-AAA ATAC22 | 5.0 | 1.36E+5 | 6.81E−4 | Not done | | |

Human FcγR interaction analysis was also performed using Octet Red 96 instrument. For human FcγRI and FcγRIIA interactions, unconjugated anti-CD40 antibodies or anti-CD40 antibody immune-stimulatory compound conjugates were immobilized on anti-human Fc biosensors and incubated with varying concentration of monomeric FcγR ranging from 1.2 nM to 1 M in PBS. The experiments were comprised of 5 steps: (1) baseline acquisition (60 s); (2) anti-CD40 antibodies or anti-CD40 antibody immune-stimulatory compound conjugates loading onto anti-human Fc biosensor (120 s); (3) second baseline acquisition (60 s); (4) association of interacting protein for kₒₙ measurement (120 s); (5) dissociation of interacting FcγR for k_{off} measurement (300 s). The interacting monomeric FcγR were used at 5-6 concentrations of 3-fold concentration series. Data were analyzed using Octet Data Analysis Software 9.0 (ForteBio) and fitted to the 1:1 binding model. Equilibrium dissociation constants (K_{D}) were calculated by the ratio of kₒₙ to k_{off}. Selected data are shown in TABLE 10. There is very little to no changes in anti-CD40 antibody immune-stimulatory compound conjugate interaction with human FcγRI and FcγRIIA as compared to unconjugated anti-CD40 antibody interactions with the respective FcγR monomeric protein.

For human FcγRIIB/C, FcγRIIIA F158, FcγRIIIA V158 and FcγRIIIB interaction studies, the proteins were immobilized on anti-his tag biosensors and incubated with varying concentration of unconjugated anti-CD40 antibodies or anti-CD40 antibody immune-stimulatory compound conjugates ranging from 0.04 μM to 8 μM. This format was chosen because of weak interactions if antibodies were captured first and FcγR added afterwards. The experiment comprised of 5 steps: (1) baseline acquisition (60 s); (2) anti-CD40 antibodies or anti-CD40 antibody immune-stimulatory compound conjugates loading onto anti-human Fc biosensor (120 s); (3) second baseline acquisition (60 s); (4) association of interacting protein for kₒₙ measurement (120 s); (5) dissociation of interacting FcγR for k_{off} measurement (300 s). The interacting anti-CD40 antibodies or anti-CD40 antibody immune-stimulatory compound conjugates were used at 4 concentrations of 2 fold concentration series. Data were analyzed using Octet Data Analysis Software 9.0 (ForteBio) and fitted to the avidity binding model. Equilibrium dissociation constants (K_{D}) were calculated by the ratio of kₒₙ to k_{off}. Selected data are shown in TABLE 10. In most cases, there were no changes with the antibody immune-stimulatory compound conjugates with human FcγRIIB/C, FcγRIIIA F158, FcγRIIIA V158 and FcγRIIIB when compared to the parental unconjugated antibody. In some cases, there were small changes usually within 2 fold such as SBT040-G2 ATAC11 interaction with FcγRIIIA F158 when compared to the unconjugated SBT040-G2.

| Antibody/ | FcγRI | FcγRIIA | FcγRIIB/C | FcγRIIIA F158 | FcγRIIIA V158 | FcγRIIIB |
|---|---|---|---|---|---|---|
| Conjugate | K_{D} (1-1) | K_{D} (1-1) | K_{D} (avidity) | K_{D} (avidity) | K_{D} (avidity) | K_{D} (avidity) |
| SBT-040-WT | 0.68 nM | 27 nM | 1.60 uM | 0.86 uM | 0.51 uM | 3.60 uM |
| SBT-040-WT- | 0.79 nM | 35 nM | 2.20 uM | 1.21 uM | 0.62 uM | 5.00 uM |
| ATAC11 | | | | | | |
| SBT-040-WT- | 0.80 nM | 30 nM | 1.68 uM | 0.82 uM | 0.41 uM | 2.71 uM |
| ATAC22 | | | | | | |
| SBT-040-WT- | 0.63 nM | 22 nM | 1.83 uM | 0.83 uM | 0.46 uM | 3.24 uM |
| ATAC4 | | | | | | |
| SBT-040-WT- | 0.77 nM | 22 nM | 0.97 uM | 0.57 uM | 0.30 uM | 1.50 uM |
| ATAC3 | | | | | | |
| SBT-040-G2 | No binding | 22 nM | 2.62 uM | 6.50 uM | 3.65 uM | No binding |
| SBT-040-G2- | No binding | 23 nM | 3.78 uM | 10.6 uM | 4.11 uM | No binding |
| ATAC11 | | | | | | |
| SBT-040-G2- | No binding | 22 nM | 3.16 uM | 7.06 uM | 3.57 uM | No binding |
| ATAC22 | | | | | | |
| SBT-040-G2- | No binding | 22 nM | 3.49 uM | 7.00 uM | 3.84 uM | No binding |
| ATAC4 | | | | | | |
| SBT-040-G2- | No binding | 17 nM | 1.85 uM | 8.09 uM | 4.86 uM | 3.93 uM |
| ATAC3 | | | | | | |
| SBT040-VLPLL | 1.5 nM | 16 nM | 2.30 uM | 0.16 uM | 0.08 uM | 1.73 uM |
| SBT040-VLPLL | 1.3 nM | 24 nM | 3.36 uM | 0.16 uM | 0.08 uM | 2.34 uM |
| ATAC11 | | | | | | |
| SBT040-VLPLL | 1.2 nM | 22 nM | 2.14 uM | 0.14 uM | 0.08 uM | 1.65 uM |
| ATAC22 | | | | | | |
| SBT040-AAA | 0.7 nM | 64 nM | 4.84 uM | 0.47 uM | 0.27 uM | 2.44 uM |
| SBT040-AAA | 0.5 nM | 97 nM | 3.77 uM | 0.59 uM | 0.29 uM | 3.83 uM |
| ATAC11 | | | | | | |
| SBT040-AAA | 0.5 nM | 116 nM | 2.93 uM | 0.45 uM | 0.25 uM | 2.56 uM |
| ATAC22 | | | | | | |

### Example 33

### Stability of Anti-C40 Antibody Immune-Stimulatory Conjugates in IgG Depleted Human Serum

Stability of the anti-CD40 antibody immune-stimulatory conjugates in human serum (IgG depleted) were measured over 96 hours at 37° C. using either a direct HIC-UV analysis approach (Method A) or an affinity capture approach (Method B). SBT-040-G1-ATAC12, SBT-040-G2-ATAC12, or SBT-040-G1-ATAC30 were spiked in IgG-depleted human serum (BBI solutions #SF142-2) in sterile tubes (75% final serum concentration) and samples were split into 4 aliquots of equal size then transferred to a 37° C. incubator. One of the aliquots of each sample was taken from the incubator at each time-point (T=0 h, 24 h, 48 h, 96 h) and average drug-antibody ratios (DAR) were recorded.

### Method A: Direct HIC-UV Analysis

At 0, 24, 48 and 96 hours after the beginning of incubation, the anti-CD40 antibody immune-stimulatory conjugates spiked in IgG depleted human serum were analyzed by analytical hydrophobic interaction chromatography (HIC) using a TOSOH TSKgel Butyl-NPR 4.6 mm×35 mm HIC column (TOSOH Bioscience, #14947) connected to a Dionex Ultimate 3000RS HPLC system (ThermoFisher Scientific, Hemel Hemstead, UK). Results are reported below in TABLE 11.

TABLE 11

| | Average DAR | | |
|---|---|---|---|
| | SBT-040-G1- | SBT-040-G2- | SBT-040-G1- |
| Time (h) | ATAC12 | ATAC12 | ATAC30 |
| 0 | 4.3 | 3.5 | 3.7 |
| 24 | 4.1 | 3.5 | 3.6 |
| 48 | 3.9 | 3.5 | 3.4 |
| 96 | 3.8 | 3.4 | 3.0 |

### Method B: Affinity Capture, De-Glycosylation and RP-ESI-MS Analysis

ADCs were immunocaptured from the IgG depleted human serum using an anti-Human IgG (Fc specific) biotin antibody immobilized on streptavidin beads at 0, 24, 48 and 96 hours after the beginning of incubation. After elution from the beads, the samples were de-glycosylated using agarose-immobilized EndoS (Genovis Inc, USA). The de-glycosylated ADCs were analyzed by reverse phase chromatography hyphenated to electrospray ionization mass spectrometry (RP-ESI-MS) using an Acquity nano UPLC in line with a Xevo G2S Q-TOF (Waters, Elstree, UK). The separation was performed using an Acquity UPLC online coupled to an ESI-MS mass spectrometer. Mass spectrometric analysis was performed in positive ion mode, scanning from 1000 to 4000 m/z in high mass operating mode. The ion envelope produced by each sample was deconvoluted using the MaxEnt1 algorithm provided within the MassLynx software (Waters, Elstree, UK). Results are reported in the table below.

| | Average DAR | |
|---|---|---|
| | SBT-040- | SBT-040- |
| Time (h) | G1-ATAC4 | G1-ATAC3 |
| 0 | 4.4 | 3.8 |
| 24 | 4.7 | 3.8 |
| 48 | 4.6 | 3.7 |
| 96 | 4.5 | 3.7 |

### Example 34

### Synthesis of ATAC 18

This example shows the synthesis of (1R,6R,8R,9S,10S,15R,17R,18S)-8,17-Bis(2-amino-6-oxo-1,9-dihydropurin-9-yl)-18-(3-{2-[2-(2-{2-[3-(2,5-dioxo-1H-pyrrol-1-yl)propionylamino]ethoxy}ethoxy)-ethoxy]ethoxy}propionylamino)-9-hydroxy-3,12-dioxy-2.4.7.11.13.16-hexaoxa-3λ5.12λ5-diphosphatricyclo[13.3.0.06,10]octadecane-3,12-dione, triethylammonium salt (ATAC 18).

[chemical structure]

### Step A: Preparation of Int ATAC 18-1

[chemical structure]

To a solution containing 100 mg (0.106 mmol) of Compound 21 in 5 mL of DMSO and 39 mg (0.106 mmol) of 3-[2-(2-{2-[2-(tert-butoxycarbonylamino)ethoxy]ethoxy}ethoxy)ethoxy]propionic acid was added 22 mg (0.16 mmol) of HOBT and 27 mg (0.212 mmol) of DIC. The reaction mixture was stirred at room temperature for 16h then purified by reverse phase chromatography without work-up. The resulting product fractions were lyophilized to afford 70 mg of product which was covered with 7 mL of a 1:2 mixture of TFA and CH₂Cl₂. The mixture was stirred for 2h at ambient temperature before the solvent was removed. The resulting residue Int ATAC18-1 was used directly in the next step without purification.

### Step B: Preparation of Int ATAC 18-2

[chemical structure]

A solution containing Int ATAC18-1 (50 mg, 0.04 mmol) and 3.0 mL of methylamine (33% in anhydrous ethanol) was stirred for 16h at room temperature. The reaction mixture was concentrated to provide Int ATAC18-2 as a white foam which used directly in the next step. LCMS (ESI, m/z): 1051 [M+H].

### Step C: Preparation of Int ATAC 18-3

[chemical structure]

The above crude Int ATAC18-2 was azeotroped with 3:1 pyridine/triethylamine three times then dissolved in 0.8 mL pyridine. To this solution at 55° C. was added 2 mL triethylamine and 1 mL triethylamine trihydrofluride simultaneously. After stirring 1 h, the bath was removed and anhydrous acetone was added immediately. The mixture was stirred for 20 min and the white solid was collected by filtration. The precipitate was washed with anhydrous acetone. The product was purified by preparative flash chromatography. The resulting solution was lyophilized to provide 3 mg of Int ATAC18-3 as a white solid. LCMS: (ESI, m/z): 937.6 [M+H]⁺. ¹H NMR (DMSO-d⁶): δ 7.99 (s, 1H), 7.89 (s, 1H), 5.73 (m, 2H), 5.06 (m, 1H); 4.84-4.74 (m, 2H); 4.53 (t, J=5.1 Hz, 1H), 4.21 (m, 1H), 2.96 (m, 2H), 3.73-3.29 (m, 16H), 2.87 (q, J=7.2 Hz, 8H), 2.35 (m, 2H), 1.10 (t, J=7.2 Hz, 12H). ³¹P-NMR (DMSO-d⁶) δ 0.25, −1.32.

### Step D: Preparation of ATAC18

[chemical structure]

To a solution containing 2.0 mg (0.002 mmol) of Int ATAC18-3 in 0.2 mL of DCM was added 0.5 mg (0.004 mmol) of DIPEA then 1.06 mg (0.004 mmol) of 2,5-dioxo-1-pyrrolidinyl 3-(2,5-dioxo-1H-pyrrol-1-yl)propionate. The resulting solution was stirred overnight then the solvent was evaporated. Reverse phase column chromatography afforded the desired compound ATAC18 as a white solid. LCMS: (ESI, m/z): 1088 [M+H]⁺. ¹H-NMR (D₂O) δ 8.15 (s, 1H), 8.05 (s, 1H), 7.07 (s, 2H), 5.88-5.84 (m, 2H),5.26 (m, 1H), 5.05-4.91 (m, 2H), 4.76 (m, 1H), 4.35 (m, 1H), 4.29-4.20 (m, 5H), 3.77 (t, 2H), 3.7-3.5 (m, 16H), 3.33 (m, 2H), 3.15 (q, J=7.2 Hz, 15H), 2.52 (m, 1H), 1.25 (t, J=7.2 Hz, 21H). ³¹P-NMR (DMSO-d⁶) δ 1.51, 0.62.

### Example 35

### Synthesis of ATAC 43

This example shows the synthesis of 2,3,4,5,6-Pentafluorophenyl 5-{(1S,6R,8R,9S,10R,15R,17R,18S)-8,17-bis(2-amino-6-oxo-1,9-dihydropurin-9-yl)-18-hydroxy-3,12-dioxo-3,12-dioxy-2.4.7.11.13.16-hexaoxa-3λ5.12λ5-diphosphatricyclo[13.3.0.06,10]octadec-9-ylamino}-5-oxovalerate triethylammonium salt (ATAC 43)

[chemical structure]

### Step A: Preparation of Int ATAC 43-1

[chemical structure]

To a solution containing 50 mg (0.072 mmol) of Compound 2 in 2.5 mL of DMSO was added 57 mg (0.72 mmol) of pyridine and 5 mg of DMAP. The resulting solution was stirred for 10 minutes then treated with 82 mg (0.72 mmol) of glutaric anhydride. The reaction mixture was stirred at room temperature for 16h then purified by reverse phase chromatography without work-up. Fractions containing product were lyophilized over 48h to provide 10 mg of Int ATAC 43-1 as a white solid. LCMS (ESI, m/z): 804 [M+H]. ¹H NMR (DMSO-d⁶) δ 10.6 (bs, 2H), 9.20 (bs, 1H), 7.99 (s, 1H), 7.92 (s, 1H), 7.56 (bs, 1H), 6.56 (m, 4H), 5.76 (dd, J=8.1, 17.7 Hz, 2H), 4.94 (m, 1H), 4.76-4.68 (m, 2H), 4.58 (m, 1H), 4.22 (m, 1H), 4.03-3.91 (m, 6H), 3.97 (q, J=7.0 Hz, 12H), 2.21 (t, J=7.2 Hz, 2H), 2.09-1.99 m, 2H), 1.67-1.60 (m, 2H), 1.20 (t, J=7.2 Hz, 18H). ³¹P NMR (DMSO-d⁶) d 0.44, −1.3.

### Step B: Preparation of ATAC43

[chemical structure]

To a solution containing 7.0 mg (0.009 mmol) of Int ATAC 43-1 and 2.6 mg (0.013 mmol, 1.5 eq) of pentafluorophenol in 300 uL of DMSO was added 1.8 mg (0.012 mmol, 1.3 eq) of EDC and the reaction mixture was stirred for 16h at room temperature then purified by reverse phase chromatography without work-up. Fractions containing product were lyophilized to provide ATAC 43 as a white solid. LCMS (ESI, m/z): 970 [M+H].

### Example 36

### STING Agonist Screening Assay

Biology materials and general procedures. The following reporter cell lines, reagents and ligands were obtained from InvivoGen: THP-1 Dual cells (thpd-nfis); THP-1 Dual KO-STING cells (thpd-kostg); Quanti-Blue (rep-qb1); Quanti-Luc (rep-qlc1); Normocin (ant-nr-1); Zeocin (ant-zn-1); Blasticidin (ant-bl-1); PMA (tlr1-pma); 2′,3′-cGAMP (tlr1-nacga); and 2′3′-c-di-AM(PS)2 (Rp,Rp) (tlr1-nacda2r). 3′,3′-cGAMP (SML 1232) and 3′,5′-cyclic-di-GMP (SML 1228) were purchased from Sigma. THP-1 Dual cells were cultured in RPMI 1640 (Lonza) supplemented with 10% fetal bovine serum, 2 mM glutamine, 50 μg/mL penicillin, 50 U/mL streptomycin (all from Gibco). Cells were passaged at 0.7×10⁶ cell/mL every 2-3 days. 100 μg/mL Normocin, 100 μg/mL Zeocin, and 10 μg/mL Blasticidin were added every other passage to maintain reporter expression according to the manufacturer's instructions.

General procedure for in vitro screening of CDNs for cytokine induction activity. THP-1 Dual or THP-1 Dual KO-STING cells were plated at 50,000 cells per well in 200 μL of culture media in 96-well plates and matured with 150 nM PMA for 16-18 hours. Cells were washed in culture media the following day and supernatants were removed. Cells were stimulated for 30 min at 37° C. in a 5% CO₂ incubator with 150 μL of CDNs prepared in permeabilization buffer with 2 g/mL digitonin (CalBiochem) at four different concentrations (1, 0.1, 0.01 and 0.001 μM). After the incubation, the permeabilization buffer and CDNs were removed and replaced with 150 μL culture media. Cells were incubated an additional 23.5 h at 37° C. in a 5% CO₂ incubator. Prior to supernatant harvest, cells were spun at 300×g for 5 min to remove cell debris. ISG54 activity was indirectly quantified from supernatants using QUANTI-Luc, which was prepared and used according to the manufacturer's instructions. NF-κB secreted alkaline phosphatase pathway activity was indirectly quantified from supernatants using QUANTI-Blue, which was prepared and used according to the manufacturer's instructions. CellTiterGlo (Promega) was used to lyse and assess viability according to the manufacturer's instructions. Plates were analyzed on Envision (Perkin Elmer) or Synergy (BioTek) plate readers.

### Example 37

### Tumor Size in a Mouse Tumor Model is Reduced by Anti-C40 Antibody Immune-Stimulatory Compound Conjugates

The example shows tumor size is reduced in a mouse tumor model after administration of an anti-CD40 antibody immune-stimulatory compound conjugate. The immunocompromised mouse strain NSG (NOD.Cg-Prkdc^{scid}IL-2rg^{tm1Wj1}/SzJ) into which human tumor cells are co-injected with human T cells and myeloid dendritic cells (mDC) is used as the mouse tumor model. In vivo immune-mediated activity of the anti-CD40 antibody immune-stimulatory compound conjugate is assessed by examining human mDCs in this model.

Anti-CD40 antibody immune-stimulatory compound conjugates or anti-CD40 antibody (control) are injected intraperitoneally to mice. Immediately after this injection, Raji cells (a B cell lymphoma tumor cell line), human T cells, and mDCs from the same human donor are co-injected into the mice. The Raji cells are injected subcutaneously at a concentration of 1×10\hat{ }7 cells/mouse. The human T cells are injected at a concentration of 1×10\hat{ }6 cells/mouse, and mDCs are injected at a concentration of 5×10\hat{ }5 cells/mouse. Tumor growth is measured using calipers twice per week, beginning seven days post tumor cell transfer and ending at study termination, approximately 3 weeks after tumor cell inoculation. Tumor size is reduced in mice that received the anti-CD40 antibody immune-stimulatory compound conjugates in comparison to mice that received the anti-CD40 antibody.

### Example 38

### Dendritic Cell Priming of T Cells is Enhanced by Anti-C40 Antibody Immune-Stimulatory Compound Conjugates

This example shows that dendritic cell priming of T cells is enhanced by anti-CD40 antibody immune-stimulatory compound conjugates. After anti-CD40 antibody immune-stimulatory compound conjugates is administered, the upregulation of co-stimulatory molecules and cytokine production by dendritic cells is induced. Priming of a T cell response is therefore enhanced by the stimulation of dendritic cells in this manner. An in vitro human mDC and T cell co-culture assay is used to demonstrate this.

Myeloid dendritic cells (mDCs) and allogeneic T cells are isolated from peripheral blood mononuclear cells (PBMCs), are labeled with a dye to monitor cell division, and are co-cultured for 5 days in the presence of the anti-CD40 antibody immune-stimulatory compound conjugates or an isotype control. T cell activation is assessed as percent dividing cells as measured by flow cytometry using the indicator dye. The percent of dividing T cells is increased in T cells co-cultured with the anti-CD40 antibody immune-stimulatory compound conjugates in comparison to the percent of dividing T cells for T cells co-cultured with the isotype control.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1.-30. (canceled)

31. A conjugate comprising:
(a) a pathogen-associated molecular pattern (PAMP) moiety, wherein the PAMP moiety is motolimod;
(b) an antibody construct comprising an antigen binding domain and an IgG Fc domain, wherein the antigen binding domain specifically binds to human HER2 and comprises a light chain CDR1 having the amino acid sequence set forth in SEQ ID NO: 35, a light chain CDR2 having the amino acid sequence set forth in SEQ ID NO: 36, a light chain CDR3 having the amino acid sequence set forth in SEQ ID NO: 37, a heavy chain CDR1 having the amino acid sequence set forth in SEQ ID NO: 31, a heavy chain CDR2 having the amino acid sequence set forth in SEQ ID NO: 32, and a heavy chain CDR3 having the amino acid sequence set forth in SEQ ID NO: 33; and
(c) a linker covalently attached to an amino acid residue of the antibody construct and to the PAMP moiety, wherein the linker is a Sortase A linker, a linker bound to a lysine residue, a linker bound to a cysteine residue, or a linker bound to an engineered glutamine residue;
wherein the IgG Fc domain having a Kd for binding to a dendritic cell Fc gamma receptor, when the linker is attached to the IgG Fc domain and to the PAMP moiety, that is no greater than about 10 times a Kd for binding of the IgG Fc domain to the Fc gamma receptor in the absence of attachment of the linker and the PAMP moiety;
wherein the IgG Fc domain having a Kd for binding to a dendritic cell FcRn receptor, when the linker is attached to the IgG Fc domain and to the PAMP moiety, that is no greater than about 10 times a Kd for binding of the IgG Fe domain to the FcRn receptor in the absence of attachment of the linker and the PAMP moiety; and
wherein the molar ratio of the PAMP moiety to the antibody construct is less than 8.

32. The conjugate of claim 31, wherein the antigen binding domain comprises heavy and light chain variable regions having the amino acid sequences set forth in SEQ ID NO: 30 and 34, respectively.

33. The conjugate of claim 31, wherein the linker is a cleavable linker.

34. The conjugate of claim 33, wherein the linker is a maleimido-caproyl-valine-citrulline para-amino benzyloxy carbonyl linker or a maleimido-caproyl-valine-alanine para-amino benzyloxy carbonyl linker.

35. The conjugate of claim 31, wherein the linker is a non-cleavable linker.

36. The conjugate of claim 35, wherein the linker is a maleimido-caproyl linker, a maleimide-PEG4 linker, or a maleimidomethylcyclohexane-1-carboxylate linker.

37. The conjugate of claim 31, wherein the IgG Fc domain is an IgG Fc domain variant comprising at least one amino acid residue change as compared to the wild type sequence of the IgG Fc domain.

38. The conjugate of claim 31, wherein the molar ratio of the PAMP moiety to the antibody construct is less than 5.

39. The conjugate of claim 31, wherein the conjugate is in a pharmaceutical formulation.

40. The conjugate of claim 31, wherein the amino acid residue to which the linker is covalently attached is a lysine residue or a cysteine residue.

41. The conjugate of claim 31, wherein the Fc domain is an IgG1.

42. The conjugate of claim 31, wherein the amino acid residue to which the linker is covalently attached is a cysteine residue.

43. The conjugate of claim 31, wherein the amino acid residue to which the linker is attached is not any of the residues of the IgG Fc domain selected from the group consisting of residues 221, 224, 227, 230, 231, 232, 234, 235, 236, 237, 239, 240, 243, 244, 245, 247, 249, 258, 262, 263, 264, 265, 266, 267, 268, 269, 270, 271, 272, 273, 275, 278, 280, 281, 283, 285, 286, 291, 292, 293, 294, 295, 296, 297, 298, 299, 300, 305, 313, 323, 324, 325, 327, 328, 329, 330, 331, 332, 333, 335, 336, 396, and 428, wherein the numbering is according to the EU index as in Kabat and wherein the linker is a Sortase A linker, a linker bound to a lysine residue, a linker bound to an engineered cysteine residue, or a linker bound to an engineered glutamine residue.

44. A method for treating a Her2⁺ cancer in a subject in need thereof, the method comprising subcutaneously administering to the subject a therapeutically effective amount of the conjugate of claim 31.

45. The method of claim 44, wherein the cancer is breast cancer.
